(19) United States
(12) Patent Application Publication
(10) Pub. No.: US 2008/0287396 A1
(43) Pub. Date: Nov. 20, 2008

(54) Phosphonated Fluoroquinolones, Antibacterial Analogs Thereof, and Methods for the Prevention and Treatment of Bone and Joint Infections

(75) Inventors: Daniel Delorme, St. Lazare (CA); Tom Houghton, Montreal (CA); Ting Kang, Kirkland (CA); Kelly Tanaka, Montreal (CA); Yanick LaFontaine, Montreal (CA); Evelyne Dietrich, Laval (CA); Adel Rafai Far, Ville Mont-Royal (CA)

Correspondence Address: MERCHANT & GOULD PC, P.O. BOX 2903, MINNEAPOLIS, MN, 55402-0903, US

(73) Assignee: TARGANTA THERAPEUTICA INC., St. Laurent (CA)

(21) Appl. No.: 11/912,038

(22) PCT Filed: Apr. 21, 2006
(86) PCT No.: PCT/IB06/02922

§ 371 Date: Jun. 20, 2008

Related U.S. Application Data
(60) Provisional application No. 60/673,336, filed on Apr. 21, 2005.

(51) Int. Cl. A61K 31/675; C07D 401/02; A61P 31/04

(52) U.S. Cl. 514/81; 544/363; 514/82; 546/113

(57) ABSTRACT

The present invention relates to phosphonated fluoroquinolones, antibacterial analogs thereof, and methods of using such compounds. These compounds are useful as antibiotics for prevention and/or the treatment of bone and joint infections, especially for the prevention and/or treatment of osteomyelitis.

### CROSS-REFERENCE TO RELATED APPLICATIONS

The present application claims benefit of U.S. provisional application No. 60/673,336, filed Apr. 21, 2005, which is incorporated herein in its entirety.

### BACKGROUND OF THE INVENTION

a) Field of the Invention

The invention relates to phosphonated fluoroquinolones, antibacterial analogs thereof, and methods of using such compounds. These compounds are useful as antibiotics for prevention and/or the treatment of bone and joint infections, especially for the prevention and/or treatment of osteomyelitis.

b) Brief Description of the Related Art

Osteomyelitis is an inflammation of bone caused by a variety of microorganisms, mainly Staphylococcus aureus (Carek et al., American Family Physician (2001), Vol 12, 12:2413-2420). This painful and debilitating disease occurs more commonly in children. Within the adult population, diabetics and kidney dialysis patients are also vulnerable. The acute form of the disease is treatable with antibiotics, but requires a lengthy period of daily therapy. It can, however, revert to a recurrent or chronic form requiring repeated hospital stays and heavy treatment regimens.

Fluoroquinolones are wholly synthetic bactericidal antibiotics which have proven to be very successful economically and clinically. They target the bacterial topoisomerase II (DNA gyrase) and topoisomerase IV enzymes and form a ternary complex consisting of drug, DNA and enzyme that interferes with DNA transcription, replication, and repair and promotes its cleavage, leading to rapid bacterial cell death (Mitscher L. A., Chem. Rev. (2005), 105:559-592). Very popular older and newer marketed fluoroquinolones include norfloxacin (Noroxin®; U.S. Pat. No. 4,146,719), ciprofloxacin (Cipro®; U.S. Pat. No. 4,670,444), gatifloxacin (Tequin®; U.S. Pat. No. 4,980,470) and moxifloxacin (Avelox®; U.S. Pat. No. 4,990,517). Most fluoroquinolones present an extremely attractive profile with broad antimicrobial spectrum, significant to outstanding bioavailability, good pharmacokinetic properties, and few side effects. Fluoroquinolones also have a proven record of efficacy in the oral treatment of osteomyelitis (Lazzarini et al., Journal of Bone and Joint Surgery (2004), 86A(10):2305-18).

Bisphosphonates are well-characterized bone-seeking agents. These compounds are recognized for having a high affinity to the bones due to their ability to bind the Ca²⁺ ions found in the hydroxyapatite mineral forming the bone tissues (Hirabayashi and Fujisaki, Clin. Pharmacokinet. (2003) 42(15): 1319-1330). Therefore, many different types of bisphosphonate-conjugated compounds have been made for targeting drugs selectively to the bone, including proteins (Uludag et al., Biotechnol Prog. (2000) 16:1115-1118), vitamins (U.S. Pat. No. 6,214,812, US 2003/0129194 and WO 02/083150), tyrosine kinase inhibitors (WO 01/44258 and WO 01/44259), hormones (U.S. Pat. No. 5,183,815 and US 2004/0116673) and bone scanning agents (U.S. Pat. No. 4,810,486). These and other bisphosphonate derivatives have been used as therapeutic agents for bone diseases such as arthritis (U.S. Pat. No. 4,746,654), osteoporosis (U.S. Pat. No. 5,428,181 and U.S. Pat. No. 6,420,384), hypercalcemia (U.S. Pat. No. 4,973,576), and bone cancers (U.S. Pat. No. 6,548,042).

Several strategies have also been investigated for targeted delivery of antibiotics (U.S. Pat. No. 5,900,410, US 2002/0142994; US 2004/0033969, US 2005/026864). For bone-targeted delivery of antibiotics, some have suggested the use of bisphosphonated-antibiotics. However, only a few of such compounds have actually being synthesized, including tetracyclines, β-lactams and fluoroquinolones (U.S. Pat. No. 5,854,227; U.S. Pat. No. 5,880,111; DE 195 32 235; Pieper and Keppler, Phosphorus, Sulfur and Silicon (2001) 170:5-14; and Herczegh et al. J. Med. Chem. (2002) 45:2338-41). Furthermore, none of these compounds have been administered in vivo or shown to have any bone-targeting activity.

Despite the progress which has been made in the past years, bone-specific delivery is still limited by the unique anatomical features of the bones. Although bisphosphonate modification might be a promising method, there is no certainty of success because several decades of progress have demonstrated that therapeutically optimized bisphosphonate derivatives have to be designed and optimized on a compound-to-compound basis (Hirabasashi and Fujisaki, Clin Pharmakokinet (2003), 42(15):1319-1330).

In view of the above, there is a need for better administrable drugs for the prevention and treatment of bone and joint infections. More particularly, there is a need for highly active phosphonated fluoroquinolones capable of achieving both time-controlled (or sustained) and spatially controlled (or targeted) drug delivery to the bones.

The present invention fulfills these needs and also other needs as will be apparent to those skilled in the art upon reading the following specification.

### SUMMARY OF THE INVENTION

The present invention is directed to antimicrobial compounds which have an affinity for binding bones. More particularly, the invention is directed to phosphonated fluoroquinolones, antibacterial analogs thereof, and methods of using such compounds. These compounds are useful as antibiotics for the prevention, prophylaxis or treatment of bone and joint infections, especially for the prevention, prophylaxis and treatment of osteomyelitis.

In one embodiment, the compounds of the invention are represented by Formula (I):

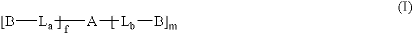

as well as pharmaceutically acceptable salts, metabolites, solvates and prodrugs thereof, where:

f is 0 or 1;

m is 0 or 1;

A is a fluoroquinolone molecule or an antibacterial analog thereof;

B is a phosphonated group; and

Lₐ and L_{b} are cleavable linkers for coupling B to A.

Preferably the linker covalently couples B to A. Preferably the phosphonated group B has a high affinity to osseous tissues.

In preferred embodiments of the compounds of Formula (I), the fluoroquinolone molecule or analog thereof A is represented by Formulae A1a and A1b:

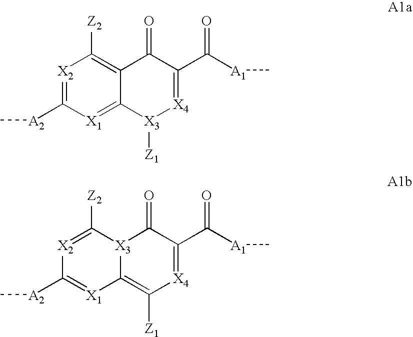

wherein:

linker Lₐ is attached at A₂ when f=1, and linker L_{b} is attached at A₁ when m=1;

A₂ is an amino radical when f=1, and A₂ is hydrogen, halogen, alkyl, aryl, pyridinyl, —O-alkyl or an amino radical when f=0;

A₁ is O or S when m=1, and A₁ is OH when m=0;

Z₁ is alkyl, aryl or —O-alkyl;

Z₂ is hydrogen, halogen or an amino radical;

X₁ is N or —CY₁—, wherein Y₁ is hydrogen, halogen, alkyl, —O-alkyl, —S-alkyl, or X₁ forms a bridge with Z₁;

X₂ is N or —CY₂—, wherein Y₂ is hydrogen, halogen, alkyl, —O-alkyl, —S-alkyl, or X₂ forms a bridge with A₂;

X₃ is N or CH;

X₄ is N or CH.

Preferably, Z₁ is cyclopropyl and X₂ is —CY₂—, wherein Y₂ is fluorine, in the compounds of Formulae A1a and A1b.

In further preferred embodiments of the compounds of Formula (I), the fluoroquinolone molecule or analog thereof A is represented by Formula A2:

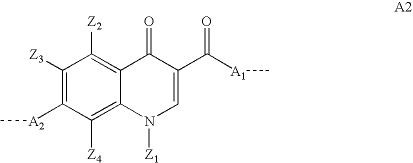

wherein:

linker Lₐ is attached at A₂ when f=1, and linker L_{b} is attached at A₁ when m=1;

A₂ is an amino radical when f=1, and A₂ is hydrogen, halogen, alkyl, aryl, pyridinyl, —O-alkyl or an amino radical when f=0;

A₁ is O or S when m=1, and A₁ is OH when m=0;

Z₁ is alkyl, aryl or —O-alkyl;

Z₂ is hydrogen, halogen or an amino radical;

Z₃ is hydrogen or halogen; and

Z₄ is hydrogen, halogen, alkyl, —O-alkyl or —S-alkyl or forms a bridge with Z₁.

Preferably, Z₁ is cyclopropyl and Z₃ is fluorine in the compound of Formula A2.

In additional preferred embodiments of the compounds of Formula (I), the fluoroquinolone molecule or analog thereof A is represented by Formula A3:

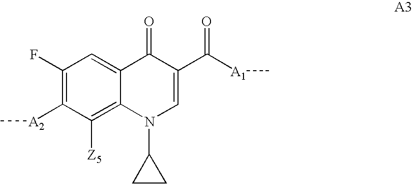

wherein:

linker Lₐ is attached at A₂ when f=1, and linker L_{b} is attached at A₁ when m=1;

A₂ is an amino radical when f=1, and A₂ is hydrogen, halogen, alkyl, aryl, pyridinyl, —O-alkyl or an amino radical when f=0;

A₁ is O or S when m=1, and A₁ is OH when m=0; and

Z₅ is hydrogen, halogen, alkyl or —O-alkyl.

In preferred embodiments of the compounds of Formulae A1a, A1b, A2 and A3, the amino radical is a N-linked substituted nitrogenous heterocyclic radical, more preferably the amino radical is a radical selected from the group consisting of pyrroles, pyrrolidines, piperidines, piperazines, morpholines, thiomorpholines, 1,4-diazepanes, dihydropyrrolidines, dihydropyridines and tetrahydropyridines.

In preferred embodiments of the compounds of Formula (I), each B is a bisphosphonate, more preferably each B is a bisphosphonate independently selected from:

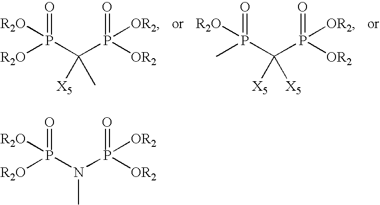

wherein:
each R₂ is independently H, lower alkyl, cycloalkyl, aryl or heteroaryl, with the proviso that at least two R₂ are H;
each X₅ is independently H, OH, NH₂, or a halo group.

In preferred embodiments of the compounds of Formula (I), L_{b} is a cleavable linker selected from the group consisting of:

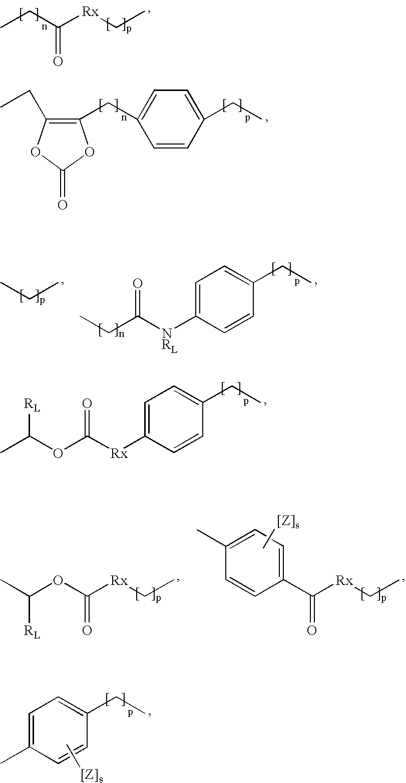

and Lₐ is a cleavable linker selected from the group consisting of:

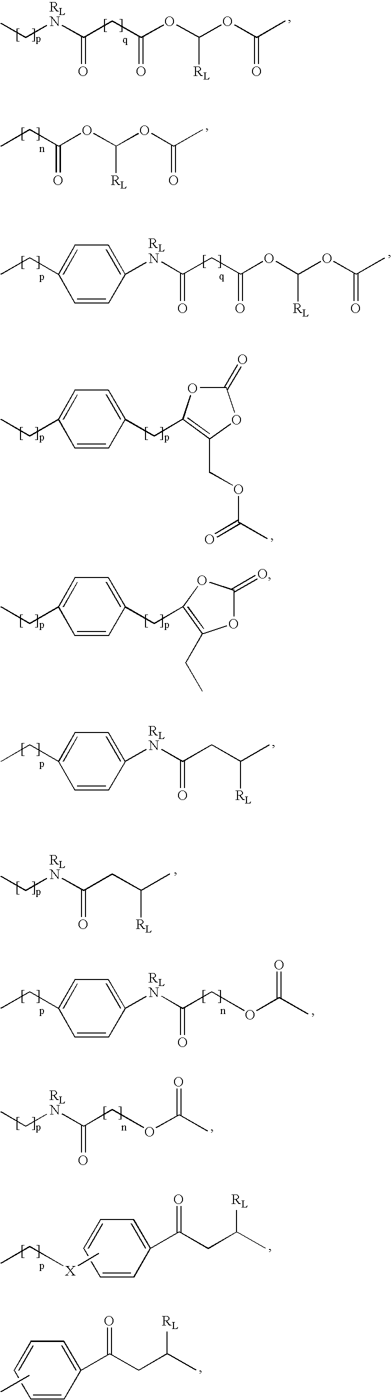

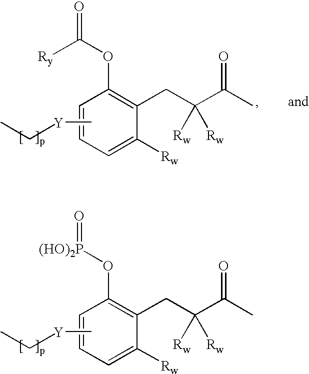

wherein:
n is an integer ≦10;
each p is independently 0 or an integer ≦10;
R_{L} is H, ethyl or methyl;
Rₓ is S, NR_{L} or O;
each Rₓ is independently H or methyl;
R_{y} is CₐH_{b} such that a is an integer from 0 to 20 and b is an integer between 1 and 2a+1;
each Z is independently selected from the group consisting of hydrogen, halogen, alkyl, alkoxy, acyl, acyloxy, carboxy, carbamoyl, sulfuryl, sulfinyl, sulfenyl, sulfonyl, mercapto, amino, hydroxyl, cyano and nitro, and s is 1, 2, 3 or 4;
q is 2 or 3;
X is CH₂, —CONR_{L}—, —CO—O—CH₂—, or —CO—O—; and
Y is O, S, S(O), SO₂, C(O), CO₂, CH₂ or absent.

Preferably, in each linker Lₐ and L_{b}, n is 1, 2, 3 or 4, more preferably n is 1 or 2; each p is independently 0, 1, 2, 3, or 4, more preferably 0 or 1; R_{L} is H; and Rₓ is NR_{L}, more preferably H.

In a preferred embodiment of the compounds of Formula (I), the fluoroquinolone molecule or analog A is ciprofloxacin or an antibacterial analog thereof.

In another preferred embodiment of the compounds of Formula (I), the fluoroquinolone molecule or analog A is gatifloxacin or an antibacterial analog thereof.

In a further preferred embodiment of the compounds of Formula (I), the fluoroquinolone molecule or analog A is moxifloxacin or an antibacterial analog thereof.

In another embodiment of the invention, the compounds of the invention are represented by Formula (II) or pharmaceutically acceptable salts, metabolites, solvates or prodrugs thereof:

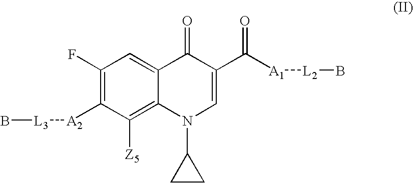

wherein:

the dashed lines represent bonds to optional groups B-L₃ and L₂-B, wherein at least one of B-L₃ and L₂-B is present;

Z₅ is hydrogen, halogen, alkyl or —O-alkyl;

A₁ is a O or S when L₂-B is attached at A₁, and A₁ is OH when L₂-B is not attached at A₁;

A₂ is an amino radical when B-L₃ is attached at A₂, and A₂ is hydrogen, halogen, alkyl, aryl, pyridinyl, —O-alkyl or an amino radical when B-L₃ is not attached at A₂;

each B is independently a phosphonated group of the formula:

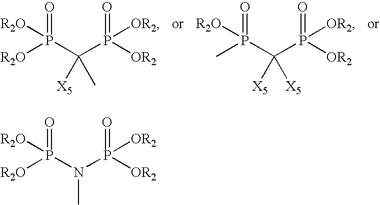

wherein:

each R₂ is independently H, lower alkyl, cycloalkyl, aryl or heteroaryl, with the proviso that at least two R₂ are H;

each X₅ is independently H, OH, NH₂, or a halo group; and L₂ is a linker of the formula:

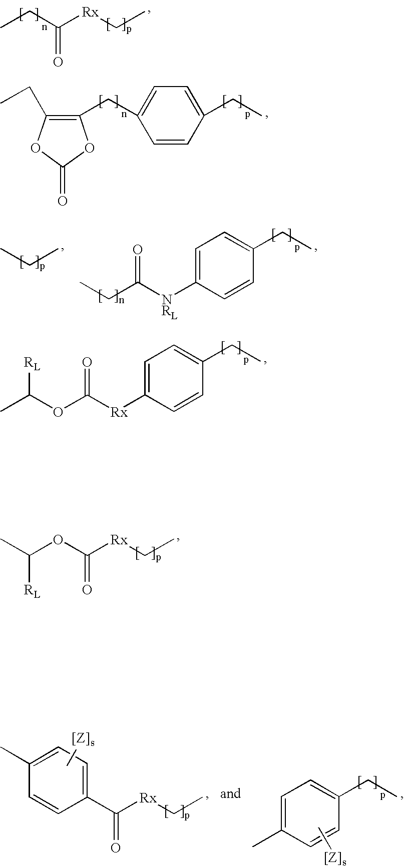

wherein:
n is an integer ≦10;
p is 0 or an integer ≦10;
R_{L} is H, ethyl or methyl;
Rₓ is S, NR_{L} or O; and
each Z is independently selected from the group consisting of hydrogen, halogen, alkyl, alkoxy, acyl, acyloxy, carboxy, carbamoyl, sulfuryl, sulfinyl, sulfenyl, sulfonyl, mercapto, amino, hydroxyl, cyano and nitro, and s is 1, 2, 3 or 4;
L₃ is a linker of the formula:

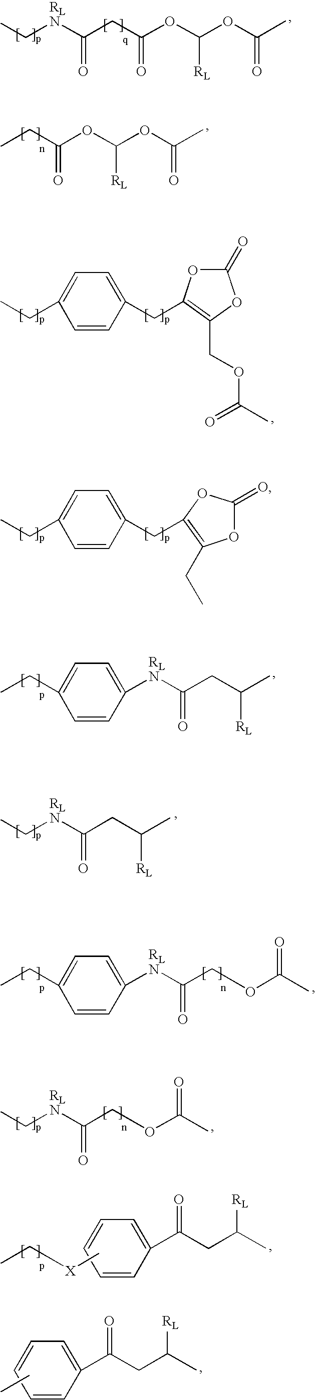

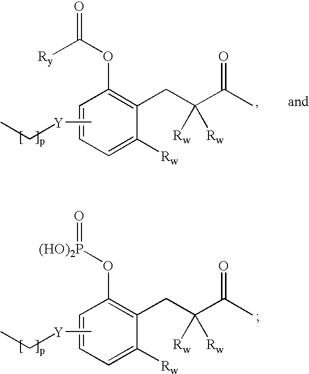

wherein:

n is an integer ≦10;

each p is independently 0 or an integer ≦10;

q is 2 or 3;

R_{L} is H, ethyl or methyl;

each R_{w} is independently H or methyl;

R_{y} is CₐH_{b} such that a is an integer from 0 to 20 and b is an integer between 1 and 2a+1;

X is CH₂, —CONR_{L}—, —CO—O—CH₂—, or —CO—O—; and

Y is O, S, S(O), SO₂, C(O), CO₂, CH₂ or absent

Preferably, in each linker L₂ and L₃, n is 1, 2, 3 or 4, more preferably 1 or 2; each p is independently 0, 1, 2, 3 or 4, more preferably 0 or 1; R_{L} is H; Rₓ is NR_{L}, more preferably NH; and each Z is independently selected from the group consisting of hydrogen, halogen, alkyl, alkoxy, and nitro.

Preferably, in the compounds of Formula (II), the amino radical is a N-linked substituted nitrogenous heterocyclic radical, more preferably the amino radical selected from the group consisting of pyrroles, pyrrolidines, piperidines, piperazines, morpholines, thiomorpholines, 1,4-diazepanes, dihydropyrrolidines, dihydropyridines and tetrahydropyridines.

In a further embodiment, the present invention includes the following compounds:

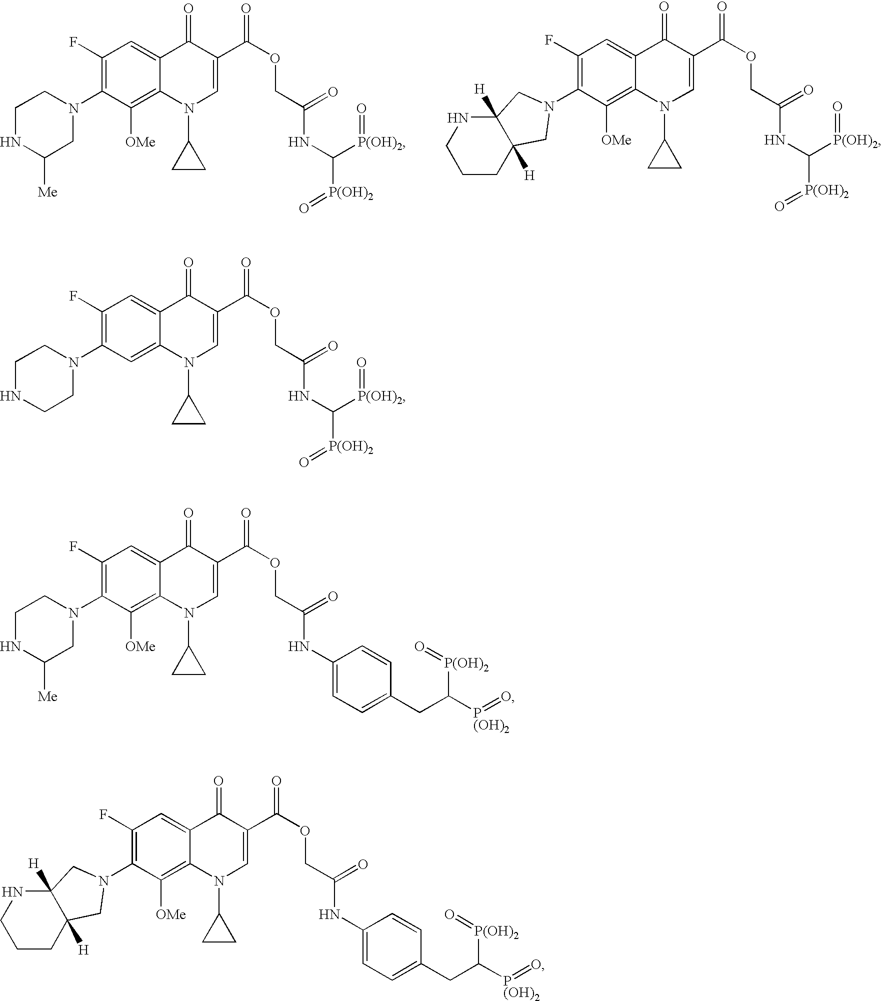

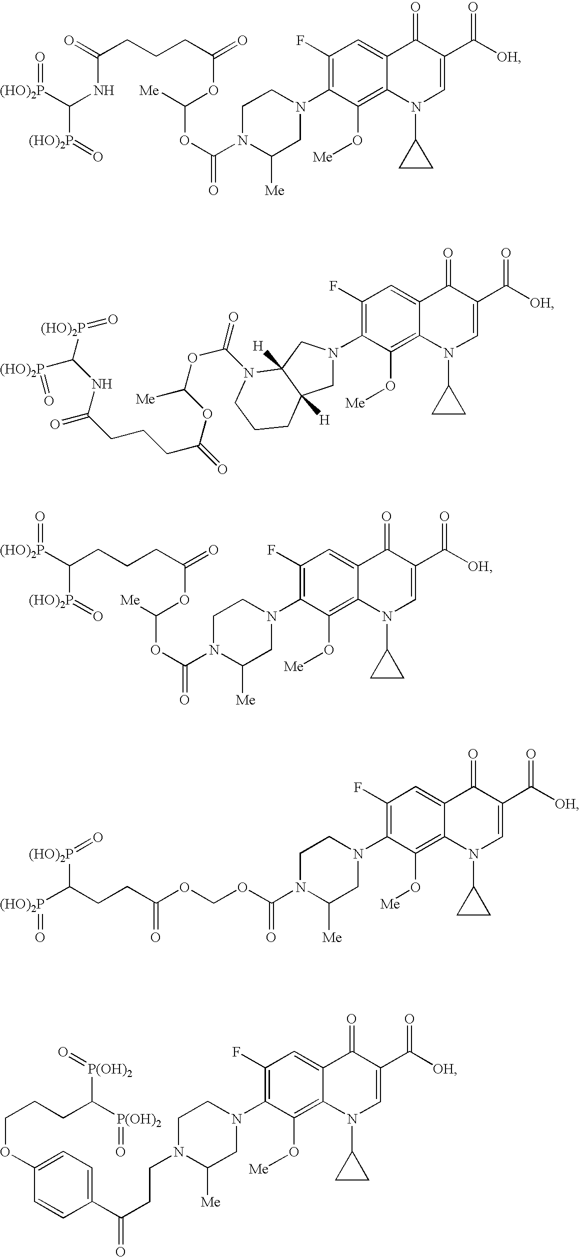

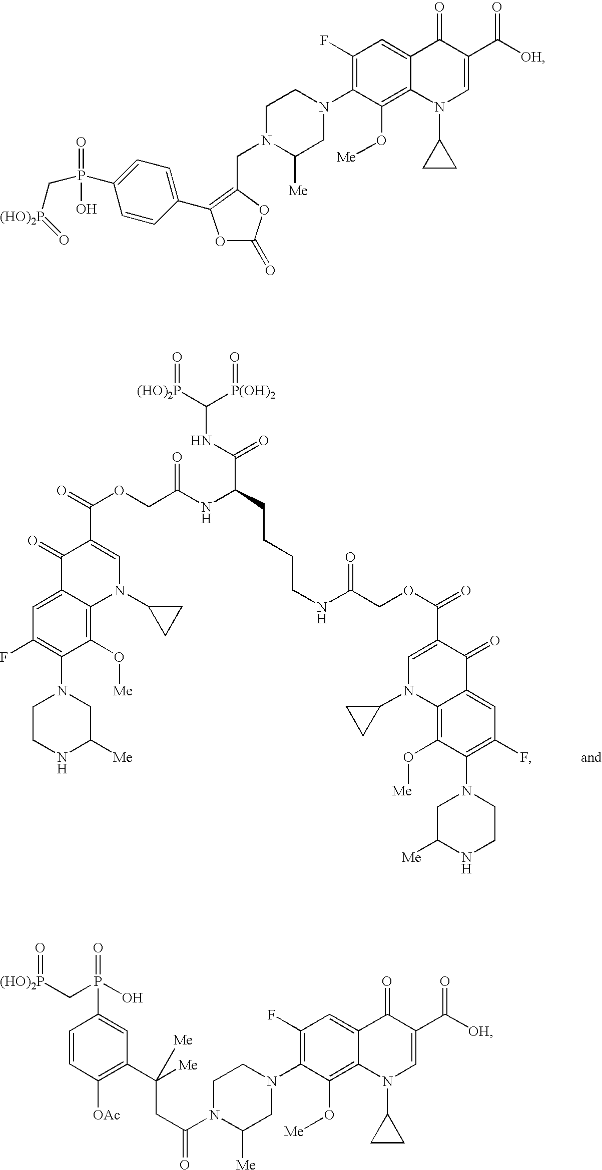

or pharmaceutically acceptable salt, metabolite, solvate or prodrug thereof.

In another aspect of the present invention there are disclosed pharmaceutical compositions comprising a compound of the invention in combination with a pharmaceutically acceptable carrier or excipient. Preferably, the pharmaceutical compositions comprise a therapeutically effective amount of a compound of the invention.

The invention also concerns a method for treating a bacterial infection in a subject, comprising administering to the subject a pharmaceutical composition comprising a pharmaceutically effective amount of a first antibacterial compound as defined herein. Preferably the subject is a mammal, more preferably the subject is a human.

According to a related aspect, the invention also concerns a method for treating a bacterial infection in a subject, comprising administering to the subject a pharmaceutical composition comprising a pharmaceutically effective amount of a first antibacterial compound as defined herein, and a second antibacterial compound. Preferably, the second antibacterial compound is a rifamycin analog, tetracycline, tygecycline, or a tetracycline, glycycycline or minocycline analog.

The invention also concerns a method for preventing a bacterial infection in a subject, comprising administering to the subject a pharmaceutical composition comprising a pharmaceutically effective amount of an antibacterial compound as defined herein. Preferably the subject is a mammal, more preferably the subject is a human.

The invention further provides a method for accumulating a compound of the present invention in a subject. Preferably the subject is a mammal, more preferably the subject is a human. Preferably the compounds of the present invention accumulate in the bones of the subject.

In a further aspect of the present invention there are provided processes for the preparation of the compounds of the present invention, such as those of Formula (I) and/or Formula (II) of the present invention.

An advantage of the invention is that it provides antimicrobial compounds having an increased binding affinity for bone. The invention also provides methods for the unmet medical need of prevention and treatment of bone and joint infections.

Additional objects, advantages and features of the present invention will become more apparent upon reading of the following non-restrictive description of preferred embodiments with reference to the accompanying drawings which are exemplary and should not be interpreted as limiting the scope of the present invention.

### BRIEF DESCRIPTION OF THE DRAWINGS

FIG. 1 is a line graph showing concentration of compound 52 in rat tibia at 7-28 days after an IV bolus injection at 15.8 mg/Kg.

FIG. 2 is a line graph showing concentration of compound 54 in rat tibia at 7-28 days after an IV bolus injection at 17.4 mg/Kg.

FIG. 3 is a line graph showing concentration of compound 52 in rat tibia at 5 min to 24 h after an IV bolus injection of at 15.8 mg/Kg.

FIG. 4 is a line graph showing concentration of compound 49 in rat tibia at 0-120 hours after an IV bolus injection at 18.8 mg/Kg.

FIG. 5 is a line graph demonstrating a rapid clearance from the blood circulation of rats of bisphosphonated moxifloxacin prodrug 52.

FIG. 6 is a bar graph showing a prophylactic effect of 15.8 mg/kg bisphosphonated moxifloxacin prodrug 52 on bacterial titer in bone infection at different time points prior to infection.

FIG. 7 is a bar graph showing a prophylactic effect of 32 mg/kg bisphosphonated moxifloxacin prodrug 52 on bacterial titer in bone infection at different time points prior to infection.

FIG. 8 is a bar graph showing a prophylactic effect on bacterial titer in bone infection of bisphosphonated gatifloxacin prodrug 54 injected intravenously 48 h prior to infection, but at different doses.

FIG. 9 is a bar graph comparing amounts of regenerated moxifloxacin 3 in infected and uninfected rat tibiae one day and six days following an IV Injection of 15.8 or 31.6 mg/kg of prodrug 52 in infected animals.

FIG. 10 is a bar graph showing a significant prophylactic effect of a combination of 20 mg/kg rifampicin and 34 mg/kg bisphosphonated prodrug 49 on bacterial titer in bone infection 43 days post infection, as compared to 20 mg/kg rifampicin alone.

### DETAILED DESCRIPTION OF THE INVENTION

### A) General Overview of the Invention

The present invention discloses phosphonated fluoroquinolones and antibacterial analogs thereof, as shown in Formula (I) and Formula (II) as defined herein, and the specific embodiments shown herein. These compounds are useful antimicrobial agents effective against a number of human and veterinary pathogens.

The essence of the invention lies in the presence of a phosphonated group tethered to a fluoroquinolone antibiotic via a cleavable linker. Since phosphonic acid derivatives are known to have a high affinity to bone due to their ability to bind the Ca²⁺ ions found in the hydroxyapatite mineral forming bone tissues, the present inventors hypothesized and confirmed that the binding affinity, adsorption and retention of fluoroquinolone antibiotics by the bones could be increased by tethering a phosphonated group to such an antibiotic. Achieving high concentrations of fluoroquinolones in bone (in comparison with the concentrations achieved by administration of a non-phosphonated antibiotic), while permitting gradual release of the antimicrobial drug through cleavage of a cleavable linker or release of the compound from the bone, could prove to increase the concentration of the antibiotic in contiguous devascularized bones (sequestrum) to a level sufficient to eradicate microbes present in this locus. Furthermore, the present inventors have hypothesized that the release of the antimicrobial drug from the phosphonated molecule through cleavage is necessary to obtain antimicrobial activity in vivo.

The present inventors have synthesized such phosphonated fluoroquinolones and antibacterial analogs thereof, and have demonstrated that these derivatives have an increased affinity for osseous materials. The present inventors have also shown that in vivo these phosphonated compounds (prodrugs) accumulate in bones in amounts greater than amounts of the non-phosphonated parent drugs used in formulating the compounds of the present invention, and that it is possible to prolong the presence of fluoroquinolone antimicrobials in the bones by administering phosphonated fluoroquinolones and antibacterial analogs thereof according to the invention. In addition, the present inventors have also shown significant in vivo prophylactic protection against bone infection, up to 20 days prior the infection, for animals injected with the phosphonated compounds according to the invention. Accordingly, the compounds of the invention are particularly useful for the prevention, prophylaxis and/or the treatment of bone and joint-related infections and bone-related diseases such as osteomyelitis.

### B) Definitions

In order to provide an even clearer and more consistent understanding of the specification and the claims, including the scope given herein to such terms, the following general definitions are provided:

The term “alkyl” refers to saturated aliphatic groups including straight-chain, branched-chain, cyclic groups, and combinations thereof, having the number of carbon atoms specified, or if no number is specified, having 1 to 12 carbon atoms (preferably 1 to 6). Examples of alkyl groups include, but are not limited to groups such as methyl, ethyl, n-propyl, isopropyl, n-butyl, isobutyl, sec-butyl, t-butyl, n-pentyl, neopentyl, cyclopropyl, cyclobutyl, cyclopentyl, cyclohexyl, cyclobutylmethyl, cyclobutylethyl, cyclopentylmethyl, cyclopentylethyl, and adamantyl. Cyclic alkyl groups (e.g. cycloalkyl or heterocycloalkyl) can consist of one ring, including, but not limited to, groups such as cycloheptyl, or multiple fused rings, including, but not limited to, groups such as adamantyl or norbornyl.

The term “alkylaryl” refers to an alkyl group having the number of carbon atoms designated, appended to one, two, or three aryl groups.

The term “N-alkylaminocarbonyl” refers to the radical —C(O)NHR where R is an alkyl group.

The term “N,N-dialkylaminocarbonyl” refers to the radical —C(O)NRₐR_{b} where Rₐ and R_{b} are each independently an alkyl group.

The term “alkylthio” refers to the radical —SR where R is an alkyl group.

The term “alkoxy” as used herein refers to an alkyl, alkenyl, or alkynyl linked to an oxygen atom and having the number of carbon atoms specified, or if no number is specified, having 1 to 12 carbon atoms (preferably 1 to 6). Examples of alkoxy groups include, but are not limited to, groups such as methoxy, ethoxy, tert-butoxy, and allyloxy. The term “alkoxycarbonyl” refers to the radical —C(O)OR where R is an alkyl. The term “alkylsulfonyl” refers to the radical —SO₂R where R is an alkyl group.

The term “alkylene” means a saturated divalent aliphatic group including straight-chain, branched-chain, cyclic groups, and combinations thereof, having the number of carbon atoms specified, or if no number is specified, having 1 to 12 carbon atoms (preferably 1 to 6), e.g., methylene, ethylene, 2,2-dimethylethylene, propylene, 2-methyl-propylene, butylene, pentylene, cyclopentylmethylene, and the like.

The term “substituted alkyl” means an alkyl group as defined above that is substituted with one or more substituents, preferably one to three substituents selected from the group consisting of halogen, alkyl, aryl, alkoxy, acyloxy, amino, mono or dialkylamino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. The phenyl group may optionally be substituted with one to three substituents selected from the group consisting of halogen, alkyl, aryl, alkoxy, acyloxy, amino, mono or dialkylamino, hydroxyl, mercapto, carboxy, benzyloxy, benzyl, cyano, nitro, thioalkoxy, carboxaldehyde, carboalkoxy and carboxamide. Examples of substituted alkyl groups include, but are not limited to —CF₃, —CF₂—CF₃, hydroxymethyl, 1- or 2-hydroxyethyl, methoxymethyl, 1- or 2-ethoxyethyl, carboxymethyl, 1- or 2-carboxyethyl, methoxycarbonylmethyl, 1- or 2-methoxycarbonyl ethyl, benzyl, pyrdinylmethyl, thiophenylmethyl, imidazolinylmethyl, dimethylaminoethyl and the like.

The term “substituted alkylene” means an alkylene group as defined above that is substituted with one or more substituents, preferably one to three substituents, selected from the group consisting of halogen, alkyl, aryl, alkoxy, acyloxy, amino, mono or dialkylamino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. The phenyl group may optionally be substituted with one to three substituents selected from the group consisting of halogen, alkyl, aryl, alkoxy, acyloxy, amino, mono or dialkylamino, hydroxyl, mercapto, carboxy, benzyloxy, benzyl, cyano, nitro, thioalkoxy, carboxaldehyde, carboalkoxy and carboxamide. Examples of substituted alkyl groups include, but are not limited to —CF₂—, —CF₂—CF₂—, hydroxymethylene, 1- or 2-hydroxyethylene, methoxymethylene, 1- or 2-ethoxyethylene, carboxymethylene, 1- or 2-carboxyethylene, and the like.

The term “alkenyl” refers to unsaturated aliphatic groups including straight-chain, branched-chain, cyclic groups, and combinations thereof, having the number of carbon atoms specified, or if no number is specified, having 1 to 12 carbon atoms (preferably 1 to 6), which contain at least one double bond (—C═C—). Examples of alkenyl groups include, but are not limited to allyl vinyl, —CH₂—CH═CH—CH₃, —CH₂—CH₂-cyclopentenyl and —CH₂—CH₂— cyclohexenyl where the ethyl group can be attached to the cyclopentenyl, cyclohexenyl moiety at any available carbon valence.

The term “alkenylene” refers to unsaturated divalent aliphatic groups including straight-chain, branched-chain, cyclic groups, and combinations thereof, having the number of carbon atoms specified, or if no number is specified, having 1 to 12 carbon atoms (preferably 1 to 6), which contain at least one double bond (—C═C—). Examples of alkenylene groups include, but are not limited to —CH═CH—, —CH₂—CH═CH—CH₂—, —CH₂—CH(cyclopentenyl)- and the like.

The term “alkynyl” refers to unsaturated aliphatic groups including straight-chain, branched-chain, cyclic groups, and combinations thereof, having the number of carbon atoms specified, or if no number is specified, having 1 to 12 carbon atoms (preferably 1 to 6), which contain at least one triple bond (—C≡C—). Examples of alkynyl groups include, but are not limited to acetylene, 2-butynyl, and the like.

The term “alkynylene” refers to unsaturated divalent aliphatic groups including straight-chain, branched-chain, cyclic groups, and combinations thereof, having the number of carbon atoms specified, or if no number is specified, having 1 to 12 carbon atoms (preferably 1 to 6), which contain at least one triple bond (—C≡C—). Examples of alkynylene groups include, but are not limited to —C≡C—, —C≡C—CH₂—, and the like.

The term “substituted alkenyl” or “substituted alkynyl” refers to the alkenyl and alkynyl groups as defined above that are substituted with one or more substituents selected from the group consisting of halogen, alkyl, aryl, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkenyl and alkynyl groups include, but are not limited to —CH═CF₂, methoxyethenyl, methoxypropenyl, bromopropynyl, and the like.

The term “substituted alkenylene” or “substituted alkynylene” refers to the alkenylene and alkynylene groups as defined above that are substituted with one or more substituents selected from the group consisting of halogen, alkyl, aryl, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

The term “aryl” or “Ar” refers to an aromatic carbocyclic group of 6 to 14 carbon atoms having a single ring (including but not limited to groups such as phenyl) or multiple condensed rings (including but not limited to groups such as naphthyl or anthryl), and includes both unsubstituted and substituted aryl groups. Substituted aryl is an aryl group that is substituted with one or more substituents, preferably one to three substituents, selected from the group consisting of alkyl, aryl, alkenyl, alkynyl, halogen, alkoxy, acyloxy, amino, mono or dialkylamino, hydroxyl, mercapto, carboxy, benzyloxy, phenyl, aryloxy, benzyl, cyano, nitro, thioalkoxy, carboxaldehyde, carboalkoxy and carboxamide, or a functionality that can be suitably blocked, if necessary for purposes of the invention, with a protecting group. Representative examples include, but are not limited to naphthyl, phenyl, chlorophenyl, iodophenyl, methoxyphenyl, carboxyphenyl, and the like. The term “aryloxy” refers to an aryl group linked to an oxygen atom at one of the ring carbons. Examples of alkoxy groups include, but are not limited to, groups such as phenoxy, 2-, 3-, or 4-methylphenoxy, and the like. The term “arylthio group” refers to the radical —SR_{c} where R_{c} is an aryl group. The term “heteroarylthio group” refers to the radical —SR_{d} where R_{d} is a heteroaryl.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂.

The term “N-alkylamino” and “N,N-dialkylamino” means a radical —NHR and —NRR′ respectively where R and R′ independently represent an alkyl group as defined herein. Representative examples include, but are not limited to N,N-dimethylamino, N-ethyl-N-methylamino, N,N-di(1-methylethyl)amino, N-cyclohexyl-N-methylamino, N-cyclohexyl-N-ethylamino, N-cyclohexyl-N-propylamino, N-cyclohexylmethyl-N-methylamino, N-cyclohexylmethyl-N-ethylamino, and the like.

The term “thioalkoxy” means a radical —SR where R is an alkyl as defined above e.g., methylthio, ethylthio, propylthio, butylthio, and the like.

The term “acyl group” means a radical —C(O)R, where R is hydrogen, halogen, alkyl, aryl, heteroaryl, alkoxy, aryloxy, N-alkylamino, N,N-dialkylamino, N-arylamino, thioalkoxy, thioaryloxy or substituted alkyl wherein alkyl, aryl, heteroaryl, and substituted alkyl are as defined herein.

The term “thioacyl group” means a radical —C(S)R, where R is hydrogen, halogen, alkyl, aryl, heteroaryl, alkoxy, aryloxy, N-alkylamino, N,N-dialkylamino, N-arylamino, thioalkoxy, thioaryloxy or substituted alkyl wherein alkyl, aryl, heteroaryl, and substituted alkyl are as defined herein.

The term “sulfonyl group” means a radical —SO₂R, where R is hydrogen, halogen, alkyl, aryl, heteroaryl, alkoxy, aryloxy, N-alkylamino, N,N-dialkylamino, N-arylamino, thioalkoxy, thioaryloxy or substituted alkyl wherein alkyl, aryl, heteroaryl, and substituted alkyl are as defined herein.

The term “acyloxy” means a radical —OC(═O)R, where R is hydrogen, alkyl, aryl, heteroaryl or substituted alkyl wherein alkyl, aryl, heteroaryl, and substituted alkyl are as defined herein. Representative examples include, but are not limited to formyloxy, acetyloxy, cylcohexylcarbonyloxy, cyclohexylmethylcarbonyloxy, benzoyloxy, benzylcarbonyloxy, and the like.

The term “heteroalkyl,” “heteroalkenyl,” and “heteroalkynyl” refers to alkyl, alkenyl, and alkynyl groups respectively as defined above, that contain the number of carbon atoms specified (or if no number is specified, having 1 to 12 carbon atoms, preferably 1 to 6) which contain one or more heteroatoms, preferably one to three heteroatoms, as part of the main, branched, or cyclic chains in the group. Heteroatoms are independently selected from the group consisting of —NR—, —NRR, —S—, —S(O)—, —S(O)₂—, —O—, —SR, —S(O)R, —S(O)₂R, —OR —PR—, —PRR, —P(O)R— and —P(O)RR; (where each R is hydrogen, alkyl or aryl) preferably —NR where R is hydrogen or alkyl and/or O. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₃, 1-ethyl-6-propylpiperidino, 2-ethylthiophenyl, piperazino, pyrrolidino, piperidino, morpholino, and the like. Examples of heteroalkenyl groups include, but are not limited to groups such as —CH═CH—CH₂—N(CH₃)₂, and the like.

The term “heteroaryl” or “HetAr” refers to an aromatic monovalent monocyclic, bicyclic, or tricyclic radical containing 4, 5, 6, 7, 8, 9, 10, 11, 12, 13, 14, 15, 16, 17, or 18-member ring atoms, including 1, 2, 3, 4, or 5 heteroatoms, preferably one to three heteroatoms including, but not limited to heteroatoms such as N, O, P, or S, within the ring. Representative examples include, but are not limited to single ring such as imidazolyl, pyrazolyl, pyrazinyl, pyridazinyl, pyrimidinyl, pyrrolyl, pyridyl, thiophene, and the like, or multiple condensed rings such as indolyl, quinoline, quinazoline, benzimidazolyl, indolizinyl, benzothienyl, and the like.

The heteroalkyl, heteroalkenyl, heteroalkynyl and heteroaryl groups can be unsubstituted or substituted with one or more substituents, preferably one to three substituents, selected from the group consisting of alkyl, alkenyl, alkynyl, benzyl, halogen, alkoxy, acyloxy, amino, mono or dialkylamino, hydroxyl, mercapto, carboxy, benzyloxy, phenyl, aryloxy, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, pyrrolidine, morpholine, or piperidine, substituted at a nitrogen or carbon by a phenyl or benzyl group, and attached to the remainder of the molecule by any available valence on a carbon or nitrogen, —NH—S(═O)₂-phenyl, —NH—(C═O)O-alkyl, —NH—C(═O)O-alkyl-aryl, and the like. The heteroatom(s) as well as the carbon atoms of the group can be substituted. The heteroatom(s) can also be in oxidized form.

The term “heteroarylene” refers to the diradical group derived from heteroaryl (including substituted heteroaryl), as defined above, and is exemplified by the groups 2,6-pyridinylene, 2,4-pyridinylene, 1,2-quinolinylene, 1,8-quinolinylene, 1,4-benzofuranylene, 2,5-pyridinylene, 2,5-indolenylene, and the like.

The term “heteroalkylene”, “heteroalkenylene”, and “heteroalkynylene” refers to the diradical group derived from heteroalkyl, heteroalkenyl, and heteroalkynyl (including substituted heteroalkyl, heteroalkenyl, and heteroalkynyl) as defined above.

The term “carboxaldehyde” means —CHO.

The term “carboalkoxy” means —C(═O)OR where R is alkyl as defined above and include groups such as methoxycarbonyl, ethoxycarbonyl, and the like.

The term “carboxamide” means —C(═O)NHR or —C(═O)NRR′ where R and R′ are independently hydrogen, aryl or alkyl as defined above. Representative examples include groups such as aminocarbonyl, N-methylaminocarbonyl, N,N-dimethylaminocarbonyl, and the like.

The term “carboxy” refers to the radical —C(O)OH.

The term “carbamoyl” refers to the radical —C(O)NH₂.

The term “halogen” or “halo” as used herein refer to Cl, Br, F or I substituents, preferably fluoro or chloro.

The term “hydroxy” refers to a —OH radical.

“Isomers”: Compounds that have the same molecular formula (or elemental composition) but differ in the nature or sequence of bonding of their atoms or the arrangement of their atoms in space are termed “isomers”. Isomers in which the connectivity between atoms is the same but which differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example which is bonded to four different groups, a pair of enantiomers is possible. An enantiomer can be characterized by the absolute configuration of its asymmetric center and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either an individual enantiomer or as a mixture thereof. A mixture containing equal proportions of the enantiomers is called a “racemic mixture”.

The compounds of this invention may possess one or more asymmetric centers. Such compounds can therefore be produced as individual (R) — or (S)-stereoisomers or as mixtures thereof. For example, the piperazine functionality in compounds 15, 18, 28 and 49 as described in the Exemplification section bears a carbon on which a hydrogen atom, a methyl group, a methylene group and an amino group are attached, and therefore this carbon is an asymmetric center. The compounds 15, 18, 28 and 49 can exist as (R) — or (S)-stereoisomers. Unless indicated otherwise, the description or naming of a particular compound in the specification and claims is intended to include both individual enantiomers and mixtures, racemic or otherwise, thereof. For compounds 36, 39 and 44, the description is intended to include all possible diastereomers and mixtures thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

“Optically pure”: As generally understood by those skilled in the art, an optically pure compound is one that is enantiomerically pure. As used herein, the term “optically pure” is intended to mean a compound which comprises at least a sufficient amount of a single enantiomer to yield a compound having the desired pharmacological activity. Preferably, “optically pure” is intended to mean a compound that comprises at least 90% of a single isomer (80% enantiomeric excess), preferably at least 95% (90% e.e.), more preferably at least 97.5% (95% e.e.), and most preferably at least 99% (98% e.e.). Preferably, the compounds of the invention are optically pure.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York). Preferred amino protecting groups include, but are not limited to, benzyloxycarbonyl (CBz), t-butyloxycarbonyl (Boc), t-butyldimethylsilyl (TBDMS),9-fluorenylmethyl-oxycarbonyl (Fmoc), or suitable photolabile protecting groups such as 6-nitroveratryloxycarbonyl (Nvoc), nitropiperonyl, pyrenylmethoxycarbonyl, nitrobenzyl, dimethyl dimethoxybenzil, 5-bromo-7-nitroindolinyl, and the like. Preferred hydroxyl protecting groups include acetyl (Ac), benzoyl (Bz), benzyl (Bn), Tetrahydropyranyl (THP), TBDMS, photolabile protecting groups (such as nitroveratryl oxymethyl ether (Nvom)), Mom (methoxy methyl ether), and Mem (methoxy ethoxy methyl ether). Particularly preferred protecting groups include NPEOC (4-nitrophenethyloxycarbonyl) and NPEOM (4-nitrophenethyloxy-methyloxycarbonyl).

“Prodrug”: refers to a pharmaceutical composition that can undergo processing to release an active drug molecule. Compounds of Formula (I) and Formula (II) according to the invention are in the form of a prodrug as the linker L (such as any of Lₐ, L_{b}, L₂ and L₃) may be cleaved to release a fluoroquinolone molecule. In particular, prodrugs of the present invention include compounds which release, in vivo, an active parent drug (i.e., compounds of Formulae A1a, A1b, Formula A2, and Formula A3 as defined herein) when such prodrug is administered to a mammalian subject. Phosphonated fluoroquinolone prodrugs according to the invention are prepared by modifying functional groups present in selected fluoroquinolones in such a way that the modifications may be cleaved in vivo to release the parent fluoroquinolone molecule. Prodrugs include compounds of Formula (I) and Formula (II), and specific embodiments thereof shown herein, wherein a carboxy or amino group in fluoroquinolones of Formulae A1a, A1b, Formula A2 and Formula A3 is bonded to any group that may be cleaved in vivo to regenerate the free carboxyl or amino group, respectively. Examples of prodrugs include, but are not limited to esters (e.g., acetate, formate, and benzoate derivatives), carbamates (e.g., N,N-dimethylaminocarbonyl) of hydroxy functional groups of the selected fluoroquinolone molecule.

“Prodrugs” also include pharmaceutical compositions that undergo two or more events in prodrug processing. According to this embodiment, more complex prodrugs would release, upon processing, a prodrug of Formula (I) or Formula (II) that in turn undergoes cleavage to release a desired fluoroquinolone molecule.

A “pharmaceutically acceptable prodrug” is intended to mean a compound of Formula (I) or Formula (II) that may be converted under physiological conditions or by solvolysis to a bioactive compound as defined herein. Such “pharmaceutically acceptable prodrug” includes more complex forms of the compounds of Formula (I) and (II) that undergo initial processing to produce a compound of Formula (I) or (II), that in turn undergoes cleavage to release a desired parent fluoroquinolone molecule.

A “pharmaceutically acceptable active metabolite” is intended to mean a pharmacologically active product produced through metabolism in the body of a compound of Formula (I) or Formula (II) as defined herein.

A “pharmaceutically acceptable solvate” is intended to mean a solvate that retains the biological effectiveness and properties of the biologically active components of compounds of Formula (I) or Formula (II). Examples of pharmaceutically acceptable solvates include, but are not limited to water, isopropanol, ethanol, methanol, DMSO, ethyl acetate, acetic acid, and ethanolamine.

A “pharmaceutically acceptable carrier or excipient” means a carrier or excipient that is useful in preparing a pharmaceutical composition that is generally safe, non-toxic and neither biologically nor otherwise undesirable, may present pharmacologically favorable profiles and includes carriers and excipient that are acceptable for veterinary use as well as human pharmaceutical use. A “pharmaceutically acceptable carrier or excipient” as used in the specification and claims includes one and more than one such carrier and/or excipient. Such carriers include, but are not limited to saline, buffered saline, dextrose, water, glycerol, ethanol, and combinations thereof.

A “pharmaceutically acceptable salt” of a compound means a salt that retains or improves the biological effectiveness and properties of the free acids and bases of the parent compound as defined herein or that takes advantage of an intrinsically charged functionality on the molecule and that is not biologically or otherwise undesirable. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 3-phenyl propionic acid, trimethylacetic acid, tertiary butylacetic acid, lauryl sulfuric acid, gluconic acid, glutamic acid, hydroxynapthoic acid, salicylic acid, stearic acid, muconic acid, and the like;

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like; or

(3) salts formed when a charged functionality is present on the molecule and a suitable counterion is present, such as a tetraalkyl(aryl)ammonium functionality and an alkali metal ion, a tetraalkyl(aryl)phosphonium functionality and an alkali metal ion, an imidazolium functionality and an alkali metal ion, and the like.

As used herein, the terms “bone”, “bone tissues” or “osseous tissues” refer to the dense, semi rigid, porous, calcified connective tissue forming the major portion of the skeleton of most vertebrates. It also encompasses teeth, osteo-articular tissues and calcifications that are frequently seen in the walls of atherosclerotic vessels.

The terms “fluoroquinolone antimicrobial molecule”, “fluoroquinolone molecule”, “fluoroquinolone” and related terms refer to broad-spectrum antimicrobial agents which are part of the well known class “fluoroquinolones” as described in more detail herein. “Derivatives of fluoroquinolones” and “antibacterial analogs” of fluoroquinolone molecules refers to chemical analogs of fluoroquinolones that have antimicrobial (e.g., antibacterial) activity. The “derivatives” and “analogs” will be understood by the skilled artisan to be similar in structure to fluoroquinolones, but also include those chemical compounds not traditionally defined as “fluoroquinolones.” As used herein, the term “derivatives of fluoroquinolones” and “antibacterial analogs” of fluoroquinolones have the same meaning. All references herein to “fluoroquinolones” or “fluoroquinolone molecules” is intended to include derivatives of fluoroquinolones and antibacterial analogs of fluoroquinolones as well.

The term “antibacterial” includes those compounds that inhibit, halt or reverse growth of bacteria, those compounds that inhibit, halt, or reverse the activity of bacterial enzymes or biochemical pathways, those compounds that kill or injure bacteria, and those compounds that block or slow the development of a bacterial infection.

The term “phosphonated group” is intended to mean any compound non-toxic to humans having at least one phosphorus atom bonded to at least three oxygen atoms and having a measurable high affinity to osseous tissues as described hereinafter.

The terms “treating” and “treatment” are intended to mean at least the mitigation of a disease condition associated with a bacterial infection in a mammal, such as a human, that is alleviated by a reduction of growth, replication, and/or propagation of any bacterium such as Gram-positive organisms, and includes curing, healing, inhibiting, relieving from, improving and/or alleviating, in whole or in part, the disease condition.

The term “prophylaxis” is intended to mean at least a reduction in the likelihood that a disease condition associated with a bacterial infection will develop in a mammal, preferably a human. The terms “prevent” and “prevention” are intended to mean blocking or stopping a disease condition associated with a bacterial infection from developing in a mammal, preferably a human. In particular, the terms are related to the treatment of a mammal to reduce the likelihood or prevent the occurrence of a bacterial infection, such as bacterial infection that may occur during or following a surgery involving bone reparation or replacement. The terms also include reducing the likelihood of or preventing a bacterial infection when the mammal is found to be predisposed to having a disease condition but not yet diagnosed as having it. For example, one can reduce the likelihood of prevent a bacterial infection in a mammal by administering a compound of Formula (I) and/or Formula (II), or a pharmaceutically acceptable prodrug, salt, active metabolite, or solvate thereof, before occurrence of such infection.

### C) Compounds of the invention

As will be described hereinafter in the Exemplification section, the inventors have prepared phosphonated derivatives of fluoroquinolones having a high binding affinity to osseous tissues.

### General Formula

In one embodiment, the compounds of the invention are represented by Formula (I):

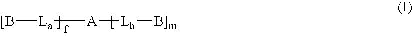

as well as pharmaceutically acceptable salts, metabolites, solvates and prodrugs thereof, where:

f is 0 or 1;

m is 0 or 1;

A is a fluoroquinolone molecule or an antibacterial analog thereof;

B is a phosphonated group, preferably having a high affinity to osseous tissues; and

Lₐ and L_{b} are cleavable linkers for coupling, preferably covalently, B to A.

As mentioned previously, the essence of the invention lies in the presence of a phosphonated group tethered to a fluoroquinolone antibiotic via a cleavable linker for the purpose of increasing the affinity, binding, accumulation and/or retention time of the fluoroquinolone antibiotic to or within the bones, while permitting its gradual release through the cleavage of the cleavable linker or release of the compound from the bone.

### Phosphonates

All non-toxic phosphonated groups having a high affinity to the bones due to their ability to bind the Ca²⁺ ions found in the hydroxyapatite mineral forming the bone tissues are suitable according to the present invention. Suitable examples of phosphonated groups can be found in WO 04/026315 (Ilex Oncology Research), U.S. Pat. No. 6,214,812 (MBC Research), U.S. Pat. No. 5,359,060 (Pfizer), U.S. Pat. No. 5,854,227 and U.S. Pat. No. 6,333,424 (Elizanor Pharm.), U.S. Pat. No. 6,548,042 (Arstad and Skattelbol) and WO 2004/089925 (Semaphore Pharmaceuticals). Specific examples of bisphosphonate and trisphosphonate groups suitable for the present invention include but are not limited to those having the formula:

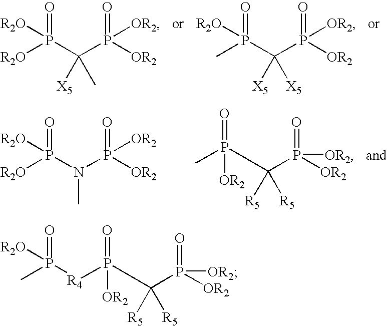

wherein:
each R₂ is independently H, lower alkyl, cycloalkyl, aryl or heteroaryl, with the proviso that at least two R₂, preferably at least three R₂, are H;
R₄ is CH₂, O, S, or NH;
each R₅ is independently H, R₆, OR₆, NR₆, or SR₆, wherein R₆ is H, lower alkyl, cycloalkyl, aryl, heteroaryl or NH₂; and
each X₅ is independently H, OH, NH₂, or a halo group.

Although monophosphonates, bisphosphonates, and tris- or tetraphosphonates could potentially be used, bisphosphonates are preferred. More preferably, the bisphosphonate group is the bisphosphonate —CH(P(O)(OH)₂)₂. As shown in Example 4 hereinafter, fluoroquinolone derivatives possessing such a bisphosphonate group have a strong binding affinity for hydroxyapatite bone powder. Of course, other types of phosphonated group could be selected and synthesized by those skilled in the art. For instance the phosphonated group may be an esterase-activated bisphosphonate radical (Vepsäläinen J., Current Medicinal Chemistry, 9, 1201-1208, 2002) or be any other suitable prodrug thereof. These and other suitable phosphonated groups are encompassed by the present invention.

### Fluoroquinolones

Fluoroquinolones are a well known class of synthetic broad spectrum (Gram-positive and Gram-negative) antimicrobial agents. Ciprofloxacin (Cipro®; U.S. Pat. No. 4,670,444), gatifloxacin (Tequin®; U.S. Pat. No. 4,980,470) and moxifloxacin (Avelox®; U.S. Pat. No. 4,990,517) are among the best known compounds in this class. The three drugs have proven to be very successful both economically and clinically. The present invention is not restricted to a specific fluoroquinolone, but encompasses additional fluoroquinolone molecules having a suitable antimicrobial activity including, but not limited to balofloxacin, benofloxacin, clinafloxacin, danofloxacin, difloxacin, enoxacin, enrofloxacin, fleroxacin, flumequine, garenoxacin, gemifloxacin, grepafloxacin, irloxacin, levofloxacin, lomefloxacin, lomefloxacin, nadifloxacin, norfloxacin, ofloxacin, olamufloxacin, pazufloxacin, pefloxacin, premafloxacin, prulifloxacin, rufloxacin, sarafloxacin, sitafloxacin, sparfloxacin, temafloxacin, tosufloxacin, trovafloxacin (Mitsher L. A., Chem. Rev. (2005), 105:559-592) and other fluoroquinolone derivatives and hybrids such as the oxazolidinone-fluoroquinolone hybrids disclosed by Vicuron Pharmaceuticals (Gordeev et al., Bioorg. Med. Chem. Lett. (2003), 13:4213-16) or by Morphochem Inc. (Hubschewerlen et al., Bioorg. Med. Chem. Lett. (2003) 13:4229-33). Also included in the present invention are antibacterial analogs of fluoroquinolones. Those skilled in the art will can readily prepare the fluoroquinolone antimicrobial molecules and antibacterial analogs thereof according to the invention. If necessary they could refer to the numerous literatures found in the art, including the US patents, PCT patent applications and scientific publications listed hereinbefore, and incorporated herein by reference.

According to one embodiment, the fluoroquinolone antimicrobial molecule A for use according to the invention is selected from compounds represented by Formulae A1a and A1b:

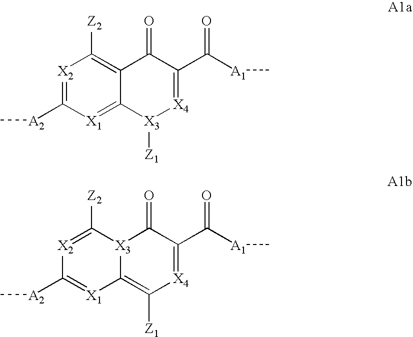

wherein:

linker Lₐ is attached at A₂ when f=1, and linker L_{b} is attached at A₁ when m=1;

A₂ is an amino radical when f=1, and A₂ is hydrogen, halogen, alkyl, aryl, pyridinyl, —O-alkyl or an amino radical when f=0; the amino radical includes, but is not limited to, N-linked substituted nitrogenous heterocyclic radicals, particularly pyrroles, pyrrolidines, piperidines, piperazines, morpholines, thiomorpholines, 1,4-diazepanes, dihydropyrrolidines, dihydropyridines and tetrahydropyridines;

A₁ is O or S when m=1, and A₁ is OH when m=0;

Z₁ is alkyl, aryl or —O-alkyl, preferably cyclopropyl;

Z₂ is hydrogen, halogen or an amino radical;

X₁ is N or —CY₁—, wherein Y₁ is hydrogen, halogen, alkyl, —O-alkyl, —S-alkyl, or X₁ forms a bridge with Z₁;

X₂ is N or —CY₂—, wherein Y₂ is hydrogen, halogen (preferably fluorine), alkyl, —O-alkyl, —S-alkyl, or X₂ forms a bridge with A₂;

X₃ is N or CH;

X₄ is N or CH.

According to a more specific embodiment, the fluoroquinolone antimicrobial molecule A of the invention is a compound of Formula A2:

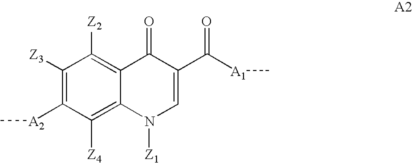

wherein:

linker Lₐ is attached at A₂ when f=1, and linker L_{b} is attached at A₁ when m=1;

A₂ is an amino radical when f=1, and A₂ is hydrogen, halogen, alkyl, aryl, pyridinyl, —O-alkyl or an amino radical when f=0; the amino radical includes, but is not limited to, N-linked substituted nitrogenous heterocyclic radicals, particularly pyrroles, pyrrolidines, piperidines, piperazines, morpholines, thiomorpholines, 1,4-diazepanes, dihydropyrrolidines, dihydropyridines and tetrahydropyridines;

Z₁ is alkyl, aryl or —O-alky, preferably cyclopropyl;

Z₂ is hydrogen, halogen or an amino radical;

Z₃ is hydrogen or halogen, preferably fluorine; and

Z₄ is hydrogen, halogen, alkyl, —O-alkyl or —S-alkyl or forms a bridge with Z₁.

According to an even more specific embodiment, the fluoroquinolone antimicrobial molecule A of the invention is a compound of Formula A3:

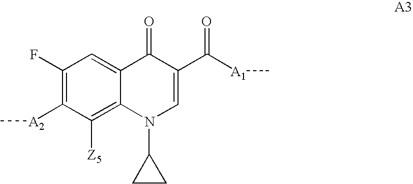

wherein:

linker Lₐ is attached at A₂ when f=1, and linker L_{b} is attached at A₁ when m=1;

A₂ is an amino radical when f=1, and A₂ is hydrogen, halogen, alkyl, aryl, pyridinyl, —O-alkyl or an amino radical when f=0; the amino radical includes, but is not limited to, N-linked substituted nitrogenous heterocyclic radicals, particularly pyrroles, pyrrolidines, piperidines, piperazines, morpholines, thiomorpholines, 1,4-diazepanes, dihydropyrrolidines, dihydropyridines and tetrahydropyridines; and

Z₅ is hydrogen, halogen, alkyl or —O-alkyl.

According to one particular embodiment, the fluoroquinolone antimicrobial molecule is moxifloxacin. According to another particular embodiment, the fluoroquinolone antimicrobial molecule is gatifloxacin. According to a third particular embodiment, the fluoroquinolone antimicrobial molecule is a ciprofloxacin. The chemical structures of these three molecules are illustrated hereinafter. Arrows indicate preferred sites for attachment of the phosphonated group via the linkers described herein.

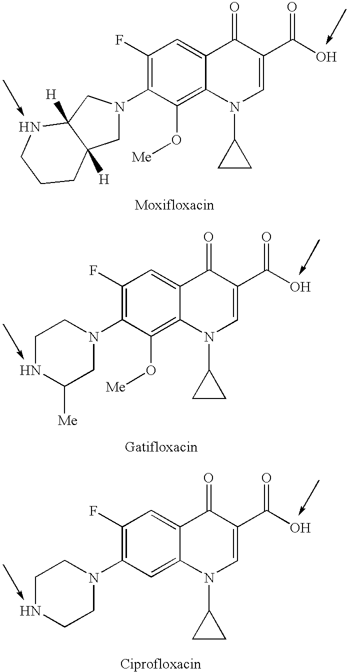

Specific examples of phosphonated derivatives of gatifloxacin, moxifloxacin and ciprofloxacin according to the invention are shown in the Exemplification section. The invention encompasses phosphonated fluoroquinolones and antibacterial analogs thereof having more than just one phosphonated group (one at each end of the moxifloxacin molecule for instance). As mentioned previously, the above identified sites of attachment are only preferred sites for tethering a phosphonated group and all other potential sites (for instance on the benzene group (i.e. at position Z₂ of Formulae A1a and A1 b, A2 of olamufloxacin, at position Z₁ of Formula A2, or at position Z₅ of Formula A3) are covered by the present invention.

### Linkers

A cleavable linker L (such as any of Lₐ, L_{b}, L₂ and L₃) covalently couples the phosphonated group B to the fluoroquinolone antibiotic A. As used herein, the term “cleavable” refers to a group that is chemically or biochemically unstable under physiological conditions. The chemical instability preferably results from spontaneous decomposition due to an intramolecular chemical reaction or hydrolysis (i.e. splitting of the molecule or group into two or more new molecules or groups due to the net insertion of one or more water molecules) when it depends on an intermolecular chemical reaction. The invention expressly excludes chemically or biochemically stable linkers and linkers precluding the in vivo release from the phosphonated group of an active (or in vivo activatable) fluoroquinolone antimicrobial molecule.

Cleavage of the linker may range from being very rapid to being very slow. For instance, the half-life of the cleavable linker may be about 1 minute, about 15 minutes, about 30 minutes, about 1 hour, about 5 hours, about 10 hours, about 15 hours, about 1 day or about 48 hours or longer. The cleavable linker may be an enzyme-sensitive linker that is cleavable only by selected specific enzymes (e.g. amidase, esterase, metalloproteinase, etc) or may be susceptible to cleavage by other chemical means, such as but not limited to acid catalysis or self-cleavage. For instance, it is conceivable according to the invention to have an esterase-sensitive linker that is cleavable only by bone-specific esterases (Goding et al. Biochim Biophys Acta (2003), 1638(1):1-19) or bone-specific metalloproteinase (MMP) (Kawabe et al., Clin Orthop. (1986) 211:244-51; Tuckermann et al., Differentiation (2001), 69(1):49-57; Sellers et al., Biochem J. (1978) 171 (2):493-6), thereby releasing the fluoroquinolone antibiotic at its desired site of action. Similarly, it is conceivable to use a cleavable linker which is not too easily cleavable in the plasma, thereby permitting a sufficient amount of the phosphonated fluoroquinolone compound to reach and accumulate within the osseous tissues before being cleaved to release the fluoroquinolone antibiotic. For instance, the linker may be selected such that only 1%, 5%, 10%, 15%, 20%, 25%, 30%, 35%, 40%, 50%, 60%, or 70% of the bone-bonded fluoroquinolone antibiotic is released through a time period extending to 1 minute, 15 minutes, 30 minutes, 1 hour, 5 hours, 10 hours, 15 hours, 1 day, 2 days, 3 days, 4 days, 5 days, 6 days 7 days, one week, two weeks, three weeks or more following administration of the compound of the invention. Preferably, the linker is selected such that only about 1% to about 25% of the bone-bonded fluoroquinolone antibiotic is released per day. The choice of the linker may vary according to factors such as (i) the site of attachment of the phosphonated group to the fluoroquinolone molecule, (ii) the type of phosphonated group used; (iii) the type of fluoroquinolone used, and (iv) the desired ease of cleavage of the linker and associated release of the fluoroquinolone antibiotic.

When the phosphonated group is tethered to a carboxylic moiety of the fluoroquinolone molecule, useful cleavable linkers include, but are not limited to, those having the structures:

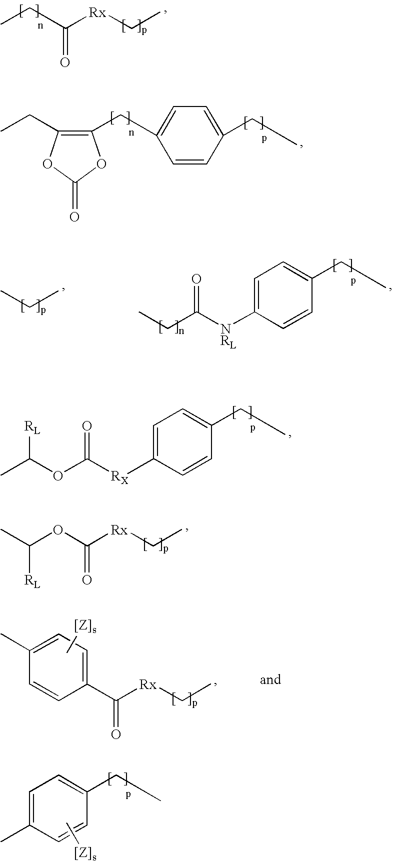

wherein:
n is an integer ≦10, preferably 1, 2, 3 or 4, more preferably 1 or 2;
p is 0 or an integer ≦10, preferably 0, 1, 2, 3 or 4, more preferably 0 or 1;
R_{L} is H, ethyl or methyl, preferably H;
Rₓ is S, NR_{L} or O, preferably NR_{L}, more preferably NH; and
each Z is independently selected from the group consisting of hydrogen, halogen, alkyl, alkoxy, acyl, acyloxy, carboxy, carbamoyl, sulfuryl, sulfinyl, sulfenyl, sulfonyl, mercapto, amino, hydroxyl, cyano and nitro, and s is 1, 2, 3 or 4;
B is a phosphonated group as described herein; and
A₁ is a fluoroquinolone antimicrobial molecule or antibacterial analog thereof as described herein.

When the phosphonated group is tethered to the amine group of the fluoroquinolone molecule, useful cleavable linkers include, but are not limited to, those having the structures:

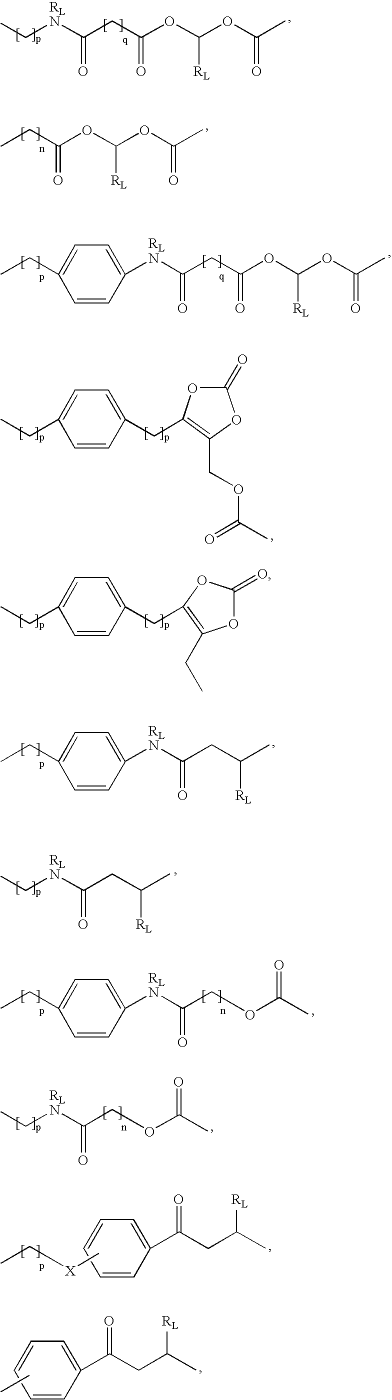

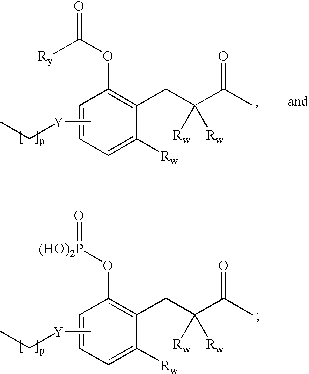

wherein:

n is an integers 10, preferably 1, 2, 3 or 4, more preferably 1 or 2;

each p is independently 0 or an integer ≦10, preferably 0, 1, 2, 3 or 4, more preferably 0 or 1;

q is 2 or 3;

R_{L} is H, ethyl or methyl;

each R_{w} is independently H or methyl;

R_{y} is CₐH_{b} such that a is an integer from 0 to 20 and b is an integer between 1 and 2a+1;

X is CH₂, —CONR_{L}—, —CO—O—CH₂—, or —CO—O—; and

Y is O, S, S(O), SO₂, C(O), CO₂, CH₂ or absent.

According to another particular embodiment, the compounds of the invention are represented by Formula (II) and pharmaceutically acceptable salts, metabolites, solvates and prodrugs thereof:

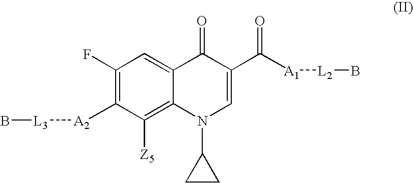

wherein:

the dashed lines represent bonds to optional groups B-L₃ and L₂-B, wherein at least one of B-L₃ and L₂-B is present;

Z₅ is hydrogen, halogen, alkyl or —O-alkyl;

A₁ is a O or S when L₂-B is attached at A₁, and A₁ is OH when L₂-B is not attached at A₁;

A₂ is an amino radical when B-L₃ is attached at A₂, and A₂ is hydrogen, halogen, alkyl, aryl, pyridinyl, —O-alkyl or an amino radical when B-L₃ is not attached at A₂; the amino radical includes, but is not limited to, N-linked substituted nitrogenous heterocyclic radicals, particularly pyrroles, pyrrolidines, piperidines, piperazines, morpholines, thiomorpholines, 1,4-diazepanes, dihydropyrrolidines, dihydropyridines and tetrahydropyridines;

each B is independently a phosphonated group of the formula:

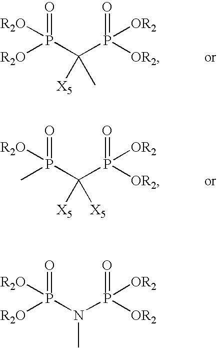

wherein:

each R₂ is independently H, lower alkyl, cycloalkyl, aryl or heteroaryl, with the proviso that at least two R₂ are H;

each X₅ is independently H, OH, NH₂, or a halo group; and

L₂ is a linker of the formula:

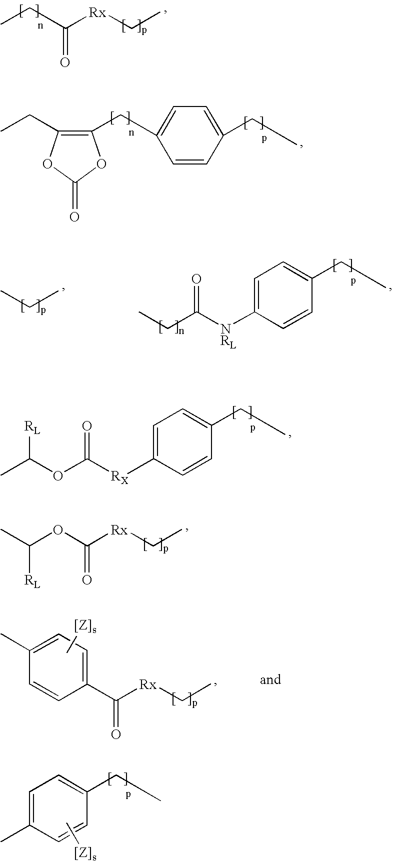

wherein:
n is an integer ≦10, preferably 1, 2, 3, or 4, more preferably 1 or 2;
p is 0 or an integer ≦10, preferably 1, 2, 3, or 4, more preferably 0 or 1;
R_{L} is H, ethyl or methyl, preferably H;
Rₓ is S, NR_{L} or O, preferably NR_{L}, more preferably NH; and
each Z is independently selected from the group consisting of hydrogen, halogen, alkyl, alkoxy, acyl, acyloxy, carboxy, carbamoyl, sulfuryl, sulfinyl, sulfenyl, sulfonyl, mercapto, amino, hydroxyl, cyano and nitro, and s is 1, 2, 3 or 4;
   L₃ is a linker of the formula:

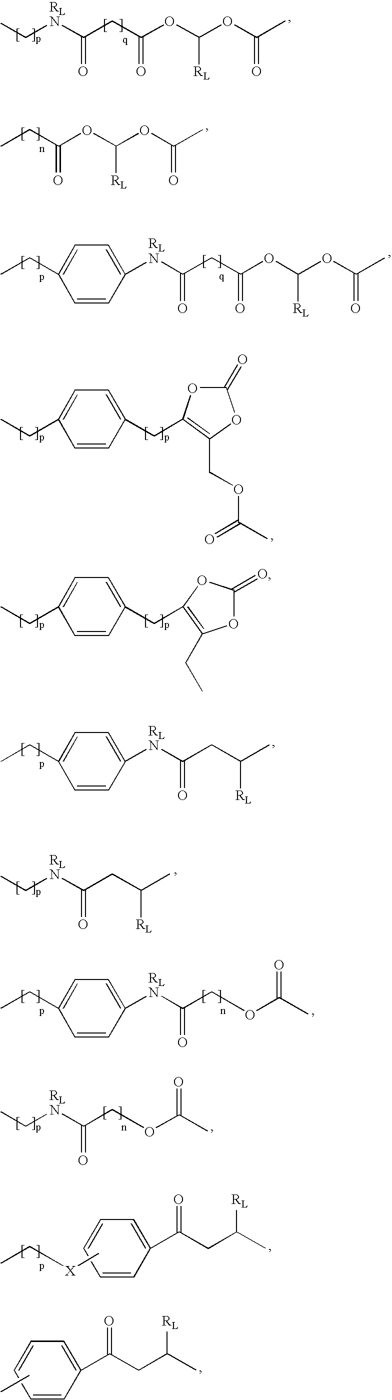

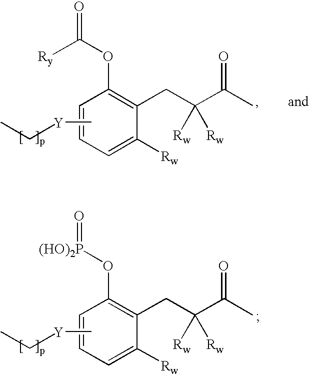

wherein:

n is an integer ≦10, preferably 1, 2, 3 or 4, more preferably 1 or 2;

each p is independently 0 or an integer ≦10, preferably 1, 2, 3, or 4, more preferably 0 or 1;

q is 2 or 3;

R_{L} is H, ethyl or methyl, preferably H;

each R_{w} is independently H or methyl;

R_{y} is CₐH_{b} such that a is an integer from 0 to 20 and b is an integer between 1 and 2a+1;

X is CH₂, —CONR_{L}—, —CO—O—CH₂—, or —CO—O—; and

Y is O, S, S(O), SO₂, C(O), CO₂, CH₂ or absent.

The invention also includes compounds comprising a single phosphonated group tethered to two or more fluoroquinolone molecules. In such circumstances, the fluoroquinolone molecules may be the same (e.g. two molecules of ciprofloxacin) or different (e.g. one molecule of ciprofloxacin and one molecule of gatifloxacin). The phosphonated group may also be tethered to similar groups (e.g. the carboxyl groups) or to different groups (e.g. the carboxyl group of one fluoroquinolone molecule and the amine group of the other fluoroquinolone molecule). Examples of potentially useful cleavable multi-fluoroquinolone linkers according to the invention include, but are not limited to, those having the structures:

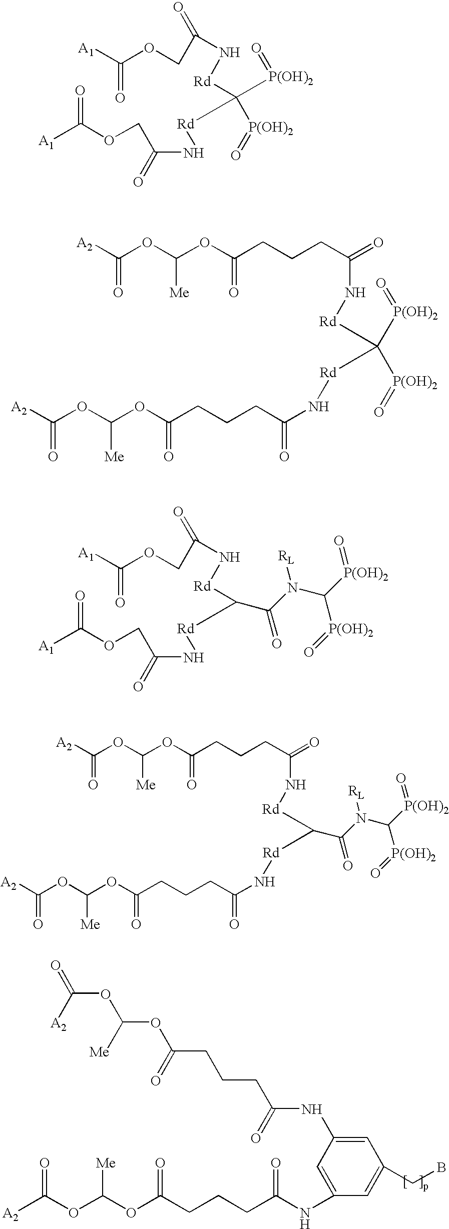

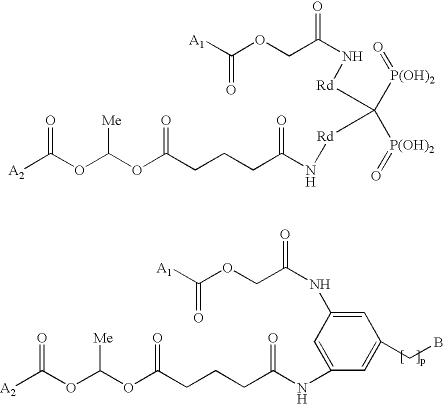

wherein:

each R_{d} is independently an alkyl or an aryl group;

R_{L} is H, ethyl or methyl, preferably H;

p is 0 or an integer ≦10, preferably 0, 1, 2, 3 or 4, more preferably 0 or 1.

A₁ and A₂ are the sites of attachment to fluoroquinolone molecules described herein, and B is the site of attachment to the bisphosphonates defined herein.

Because of its high affinity to osseous tissues, the phosphonated group B will likely remain bound to bone for an extended period of times (up to several years). Therefore, it is very important that the phosphonated group be endowed with low or preferably no measurable toxicity. According to another embodiment, the phosphonated group B and the linker are selected such that the linker is hydrolyzed or cleaved in vivo (preferably mostly in osseous tissues) thereby releasing: (i) the fluoroquinolone antimicrobial molecule A and (ii) a chosen non-toxic phosphonated molecule having a proven bone therapeutic activity. Such compounds would thus have a double utility that is to: 1) provide locally to the bones for an extended period of time and/or at increased concentrations, an antibiotic useful in preventing and/or treating a bacterial bone infection, and 2) provide to the bones a drug stimulating bone regeneration or inhibiting bone resorption, thereby facilitating bone recovery from damages caused by an infection or other injury. Suitable phosphonated molecules with proven bone therapeutic activity useful according to the invention include but are not limited to risedronate and olpadronate, but also to others such as pamidronate, alendronate, incadronate, etidronate, ibandronate, zolendronate or neridronate), these molecules being well known bisphosphonate bone resorption inhibitors commonly used for the treatment of osteoporosis.

The scheme below illustrates the principles of that embodiment if the bisphosphonated moiety possesses a free hydroxyl group:

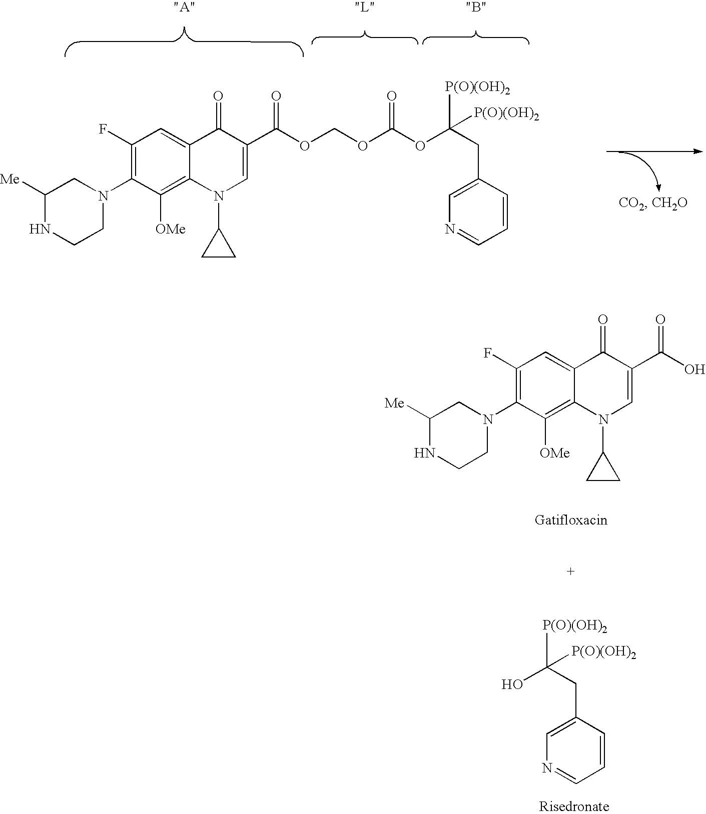

Additional specific examples of bisphosphonate derivatives according to the invention, derived from risendronate and olpadronate, are shown hereinafter:

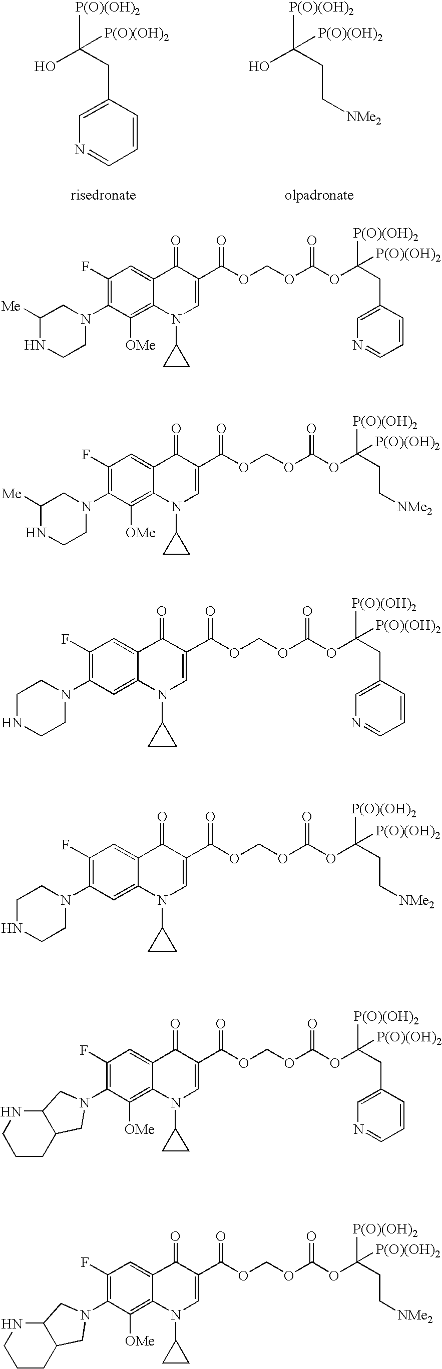

A similar illustration of that embodiment is represented by the scheme below, if the bisphosphonated moiety possesses a primary or secondary amino group:

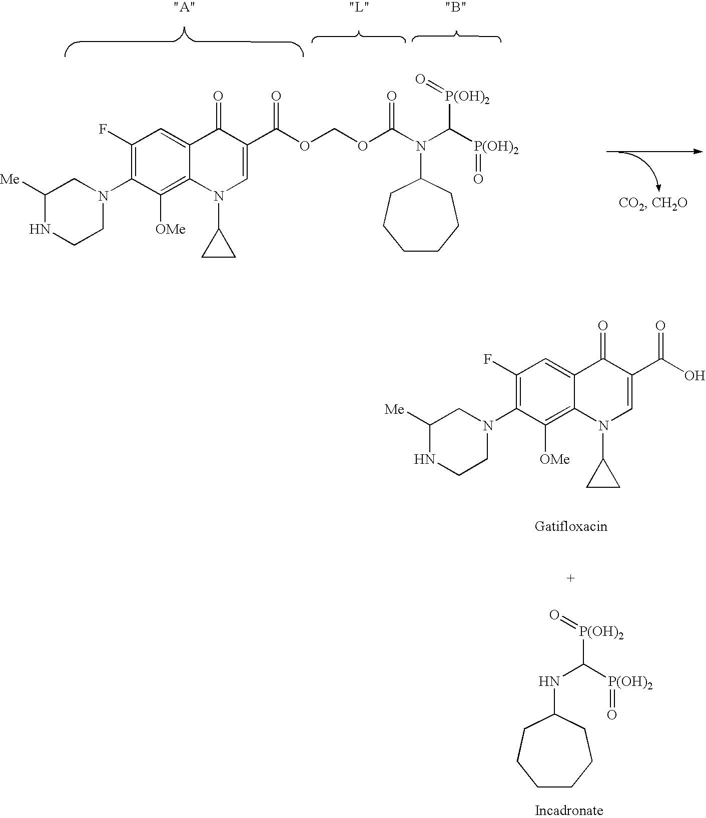

Additional specific examples of bisphosphonate derivatives according to the invention, derived from incadronate and pamidronate, are shown hereinafter:

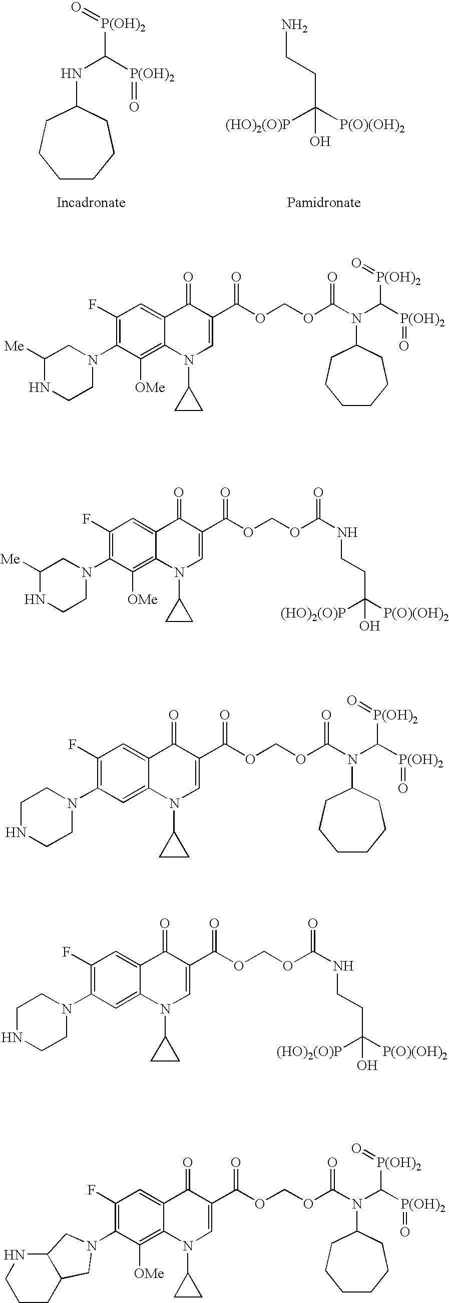

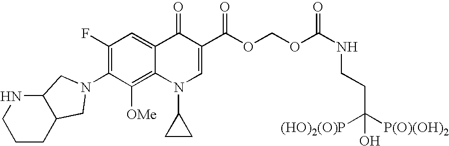

The present invention also includes the use of a pH-sensitive linker that is cleaved only at a predetermined range of pH. In one embodiment, the pH-sensitive linker is a base-sensitive linker that is cleaved at a basic pH ranging from about 7 to about 9. According to another embodiment, the linker is an acid-sensitive linker that is cleaved at an acidic pH ranging from about 7.5 to about 4, preferably from about 6.5 and lower. It is hypothesized that such an acid-sensitive linker would allow a specific release of the fluoroquinolone antibiotic mostly at a site of bacterial infection because it is known that acidification of tissues commonly occurs during infection (O'Reilly et al., Antimicrobial Agents and Chemotherapy (1992), 36(12): 2693-97).

Of course, other types of linkers could be selected and synthesized by those skilled in the art. For instance the linker may also contain an in vivo hydrolyzable phosphonated group having an affinity to bones as disclosed by Ilex Oncology Research in WO 04/026315. The linker may also contain an active group (e.g. a releasable group stimulating bone formation or decreasing bone resorption). These and other suitable linkers are encompassed by the present invention.

In a further embodiment, the present invention includes the following compounds:

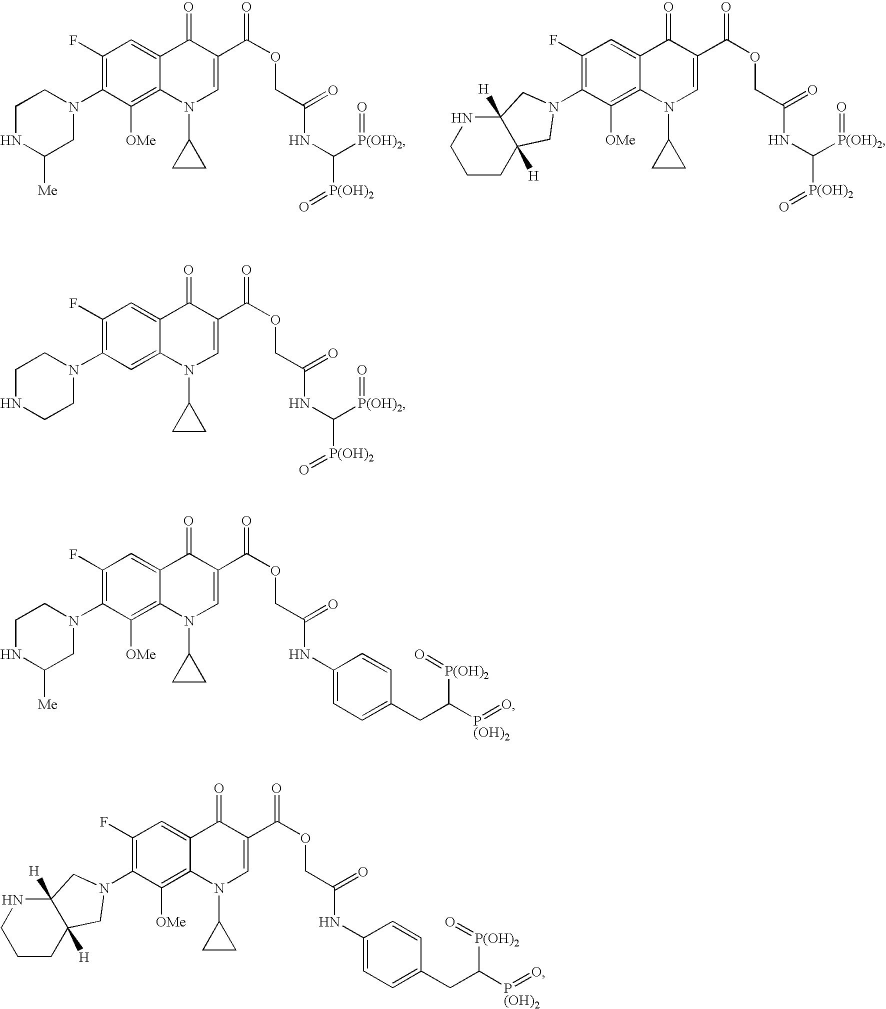

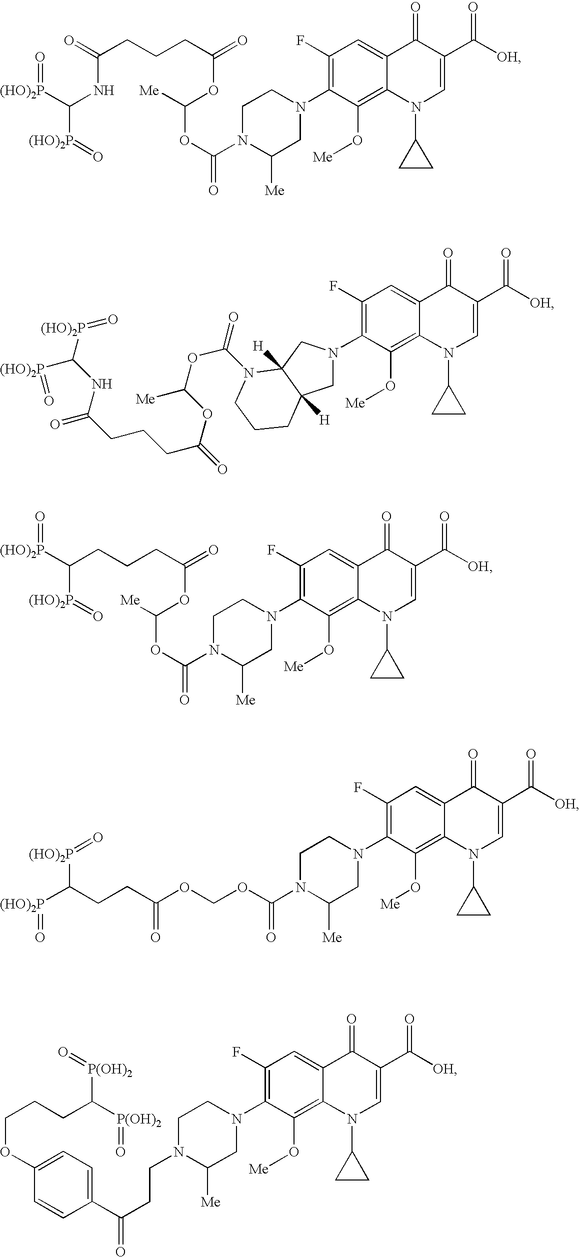

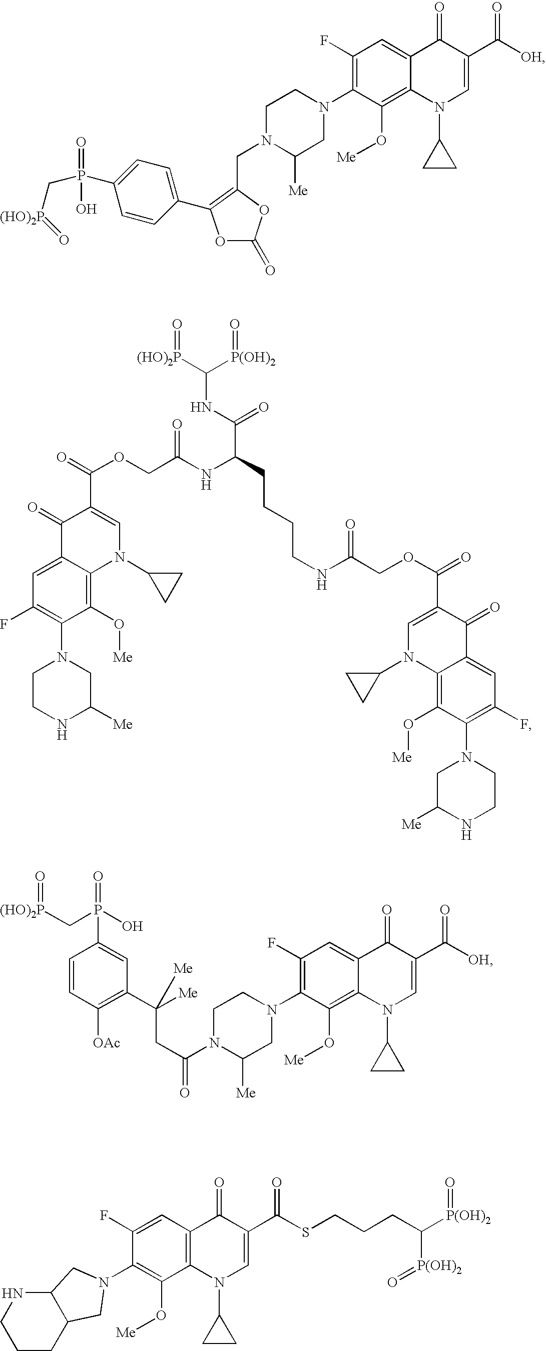

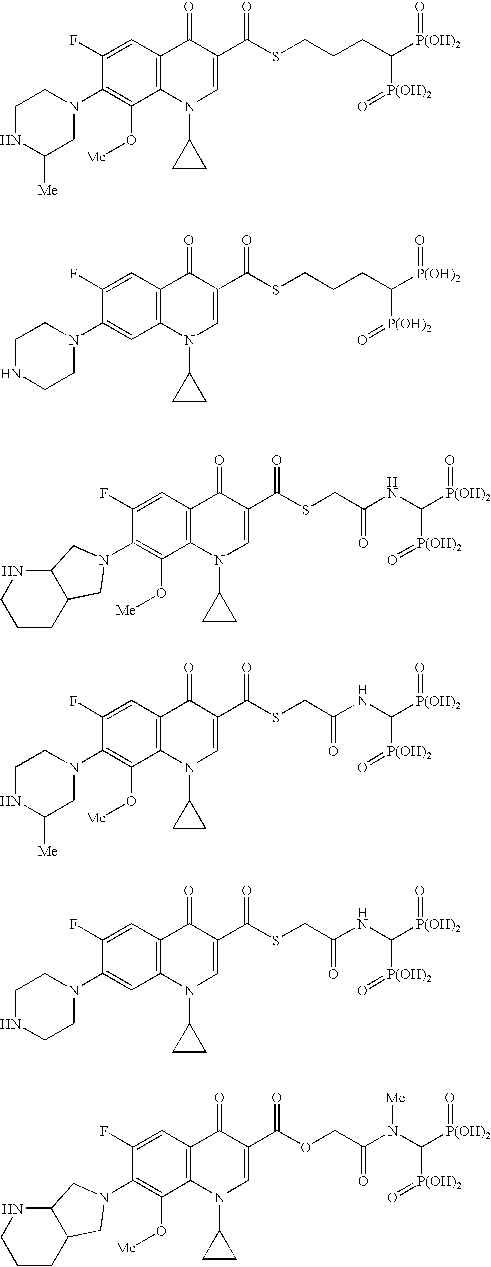

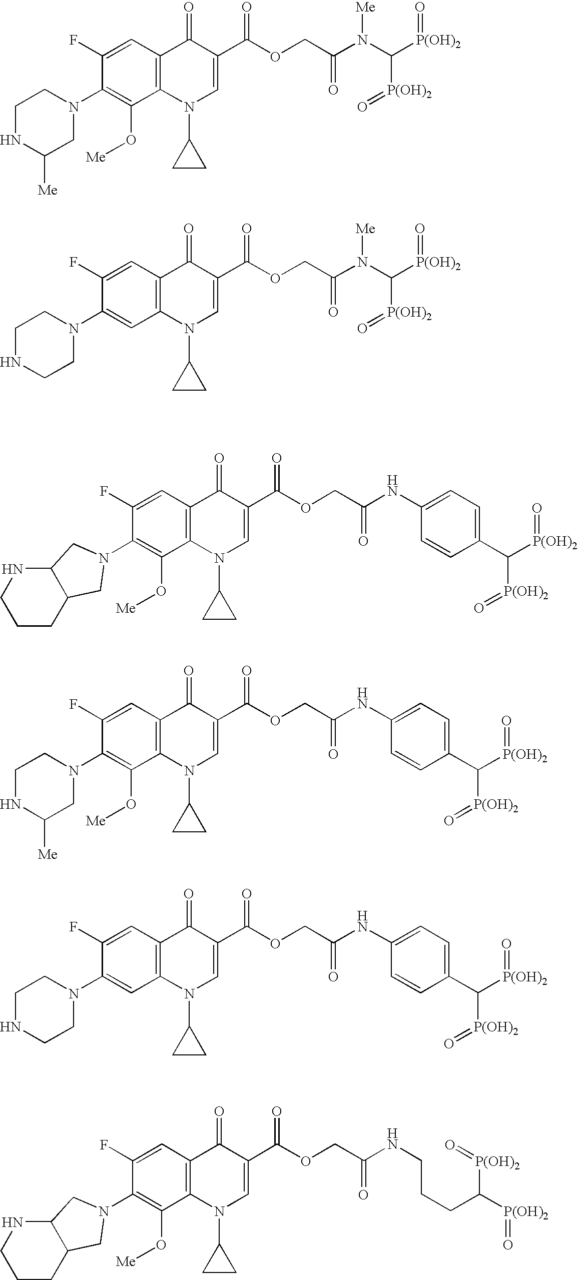

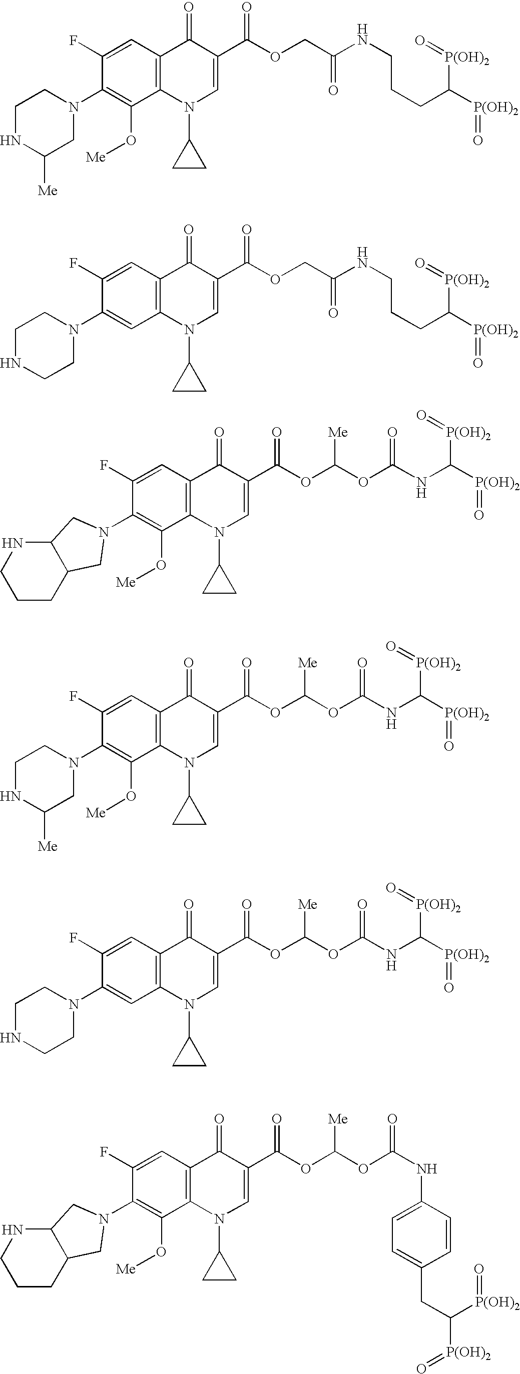

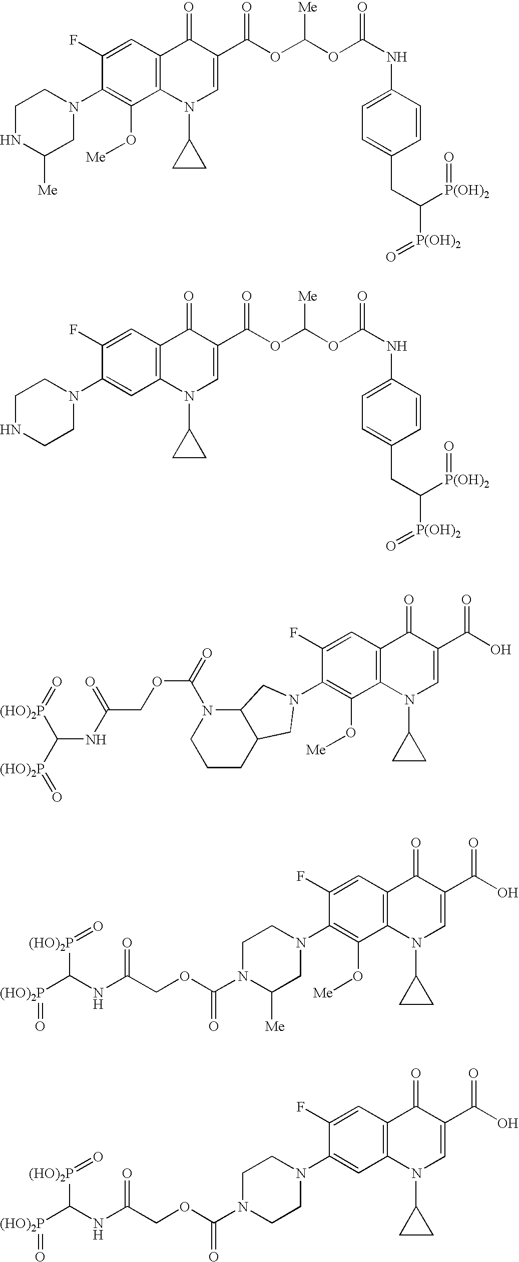

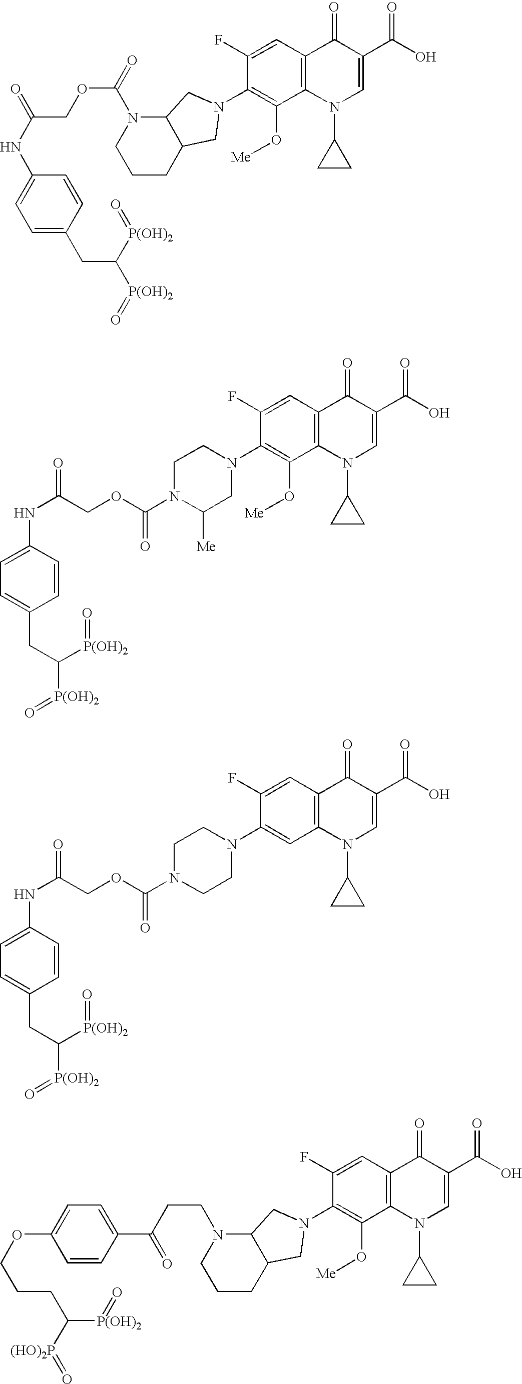

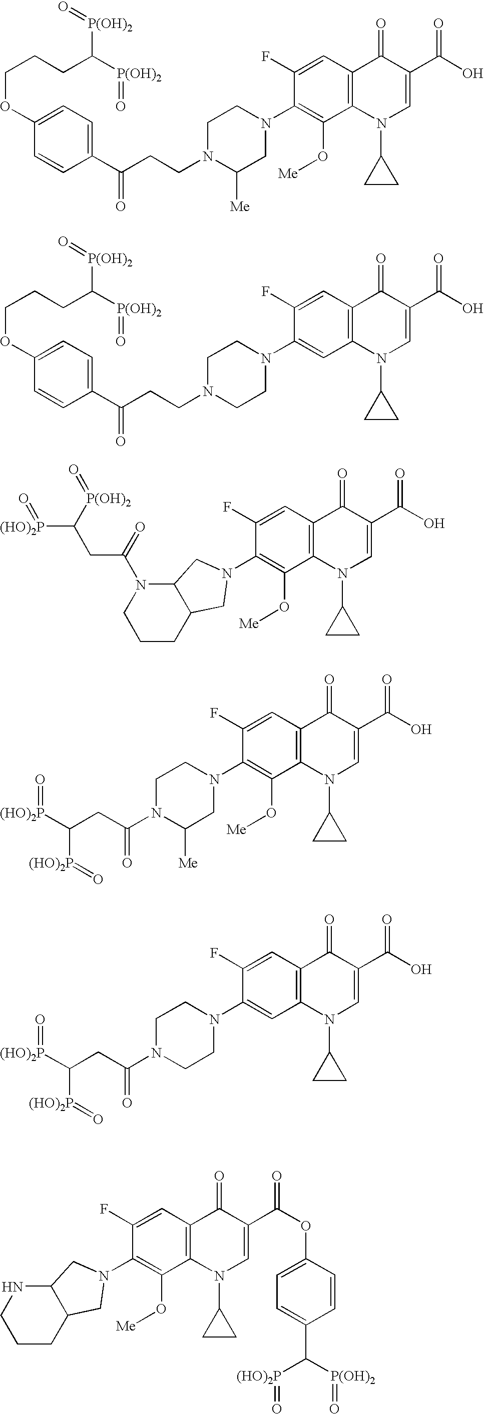

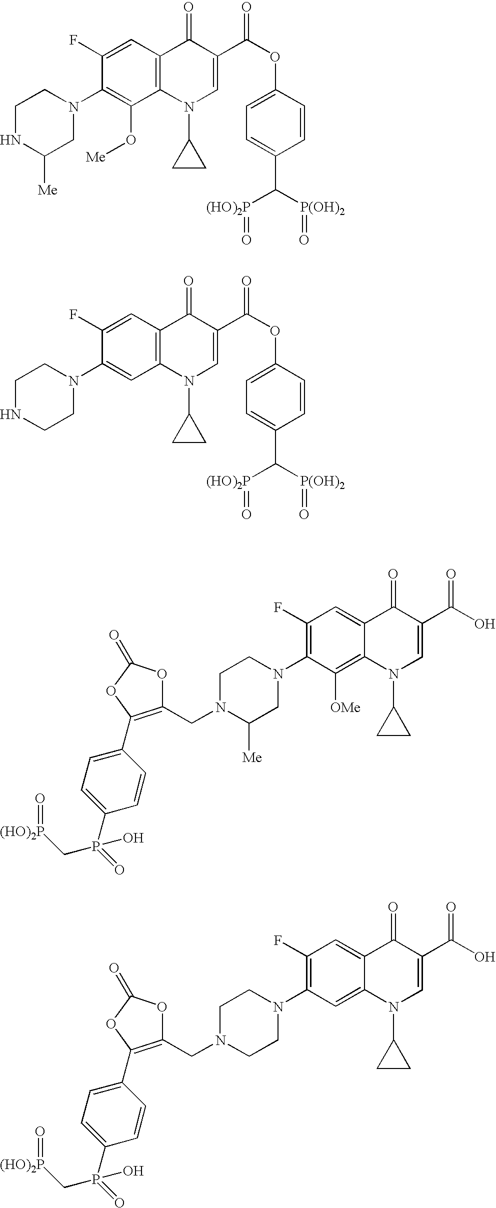

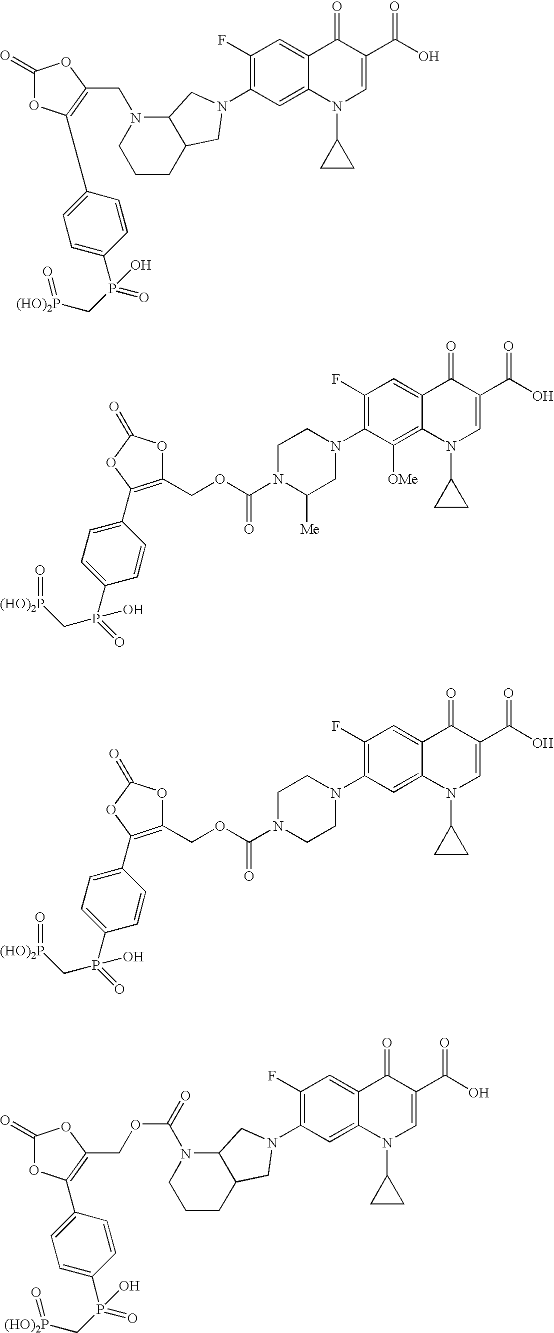

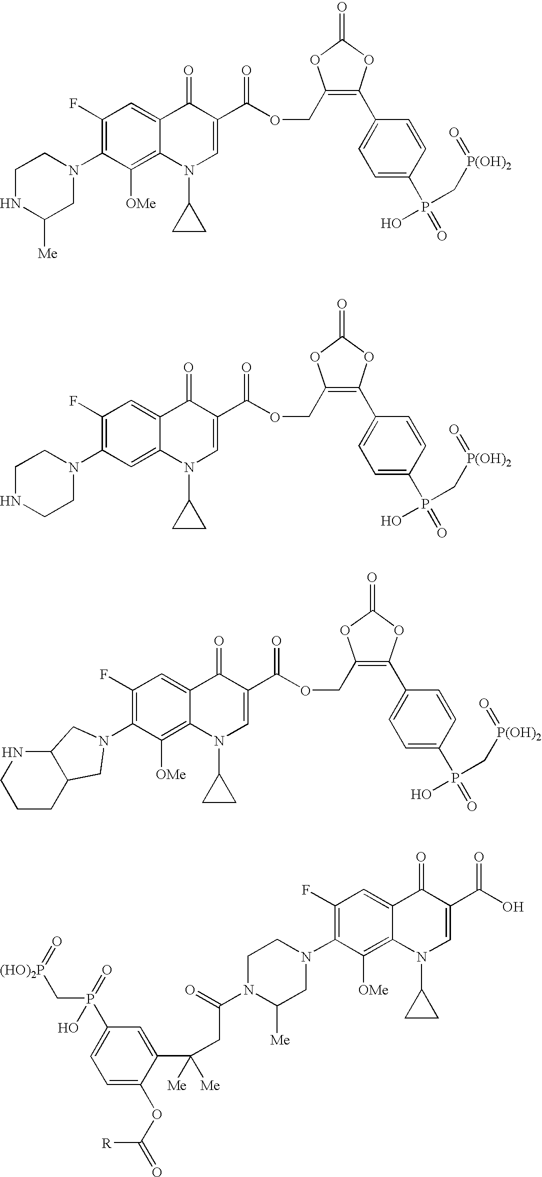

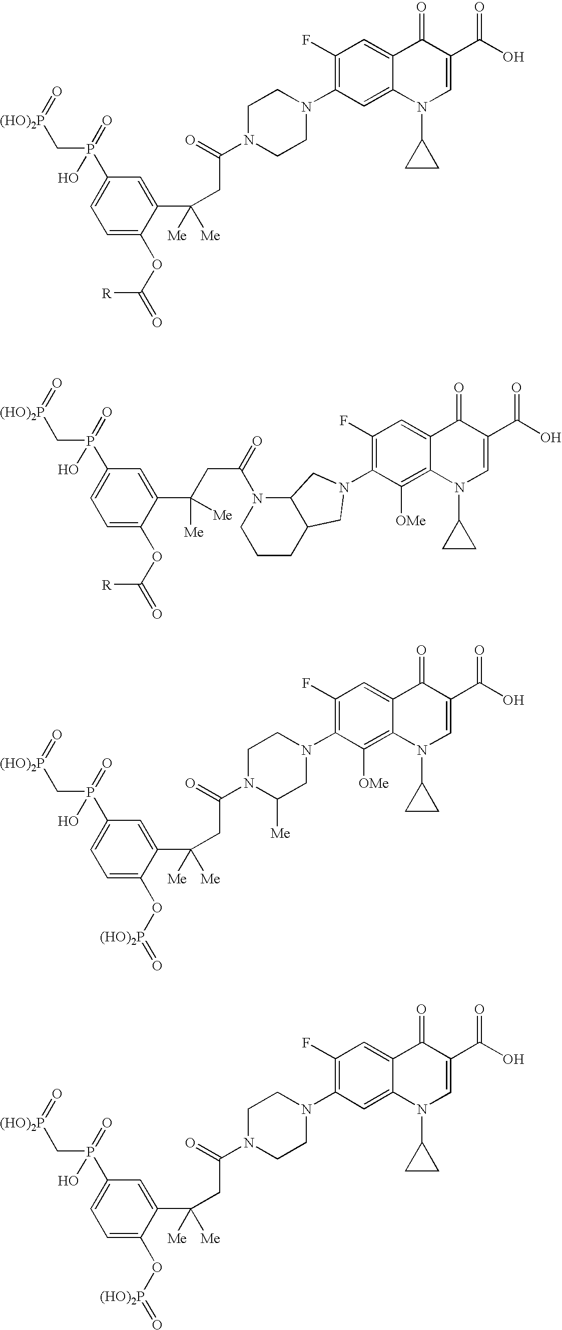

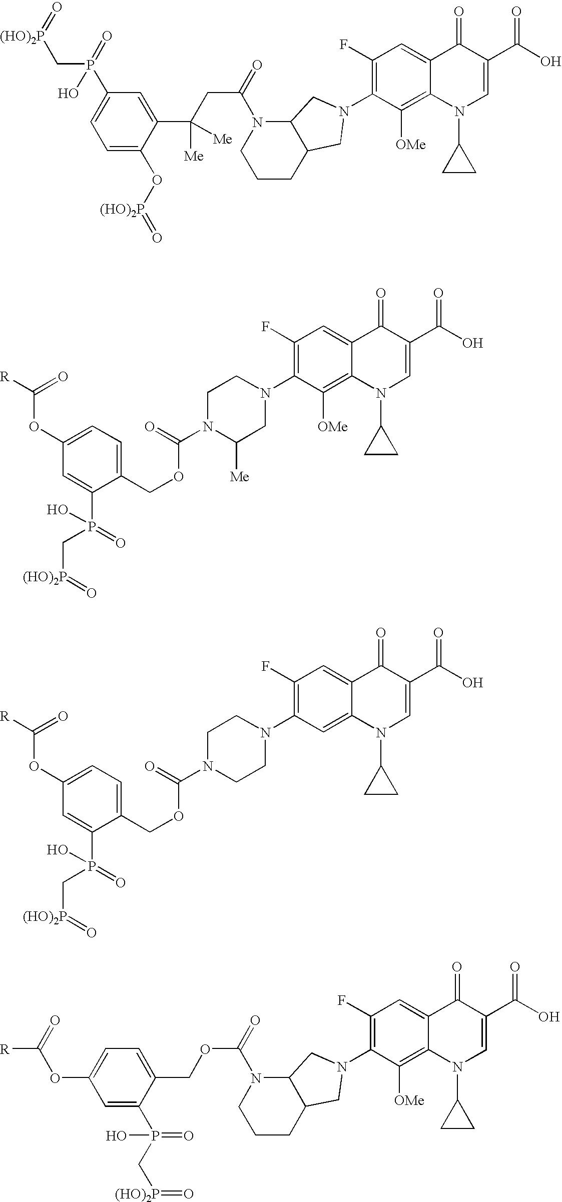

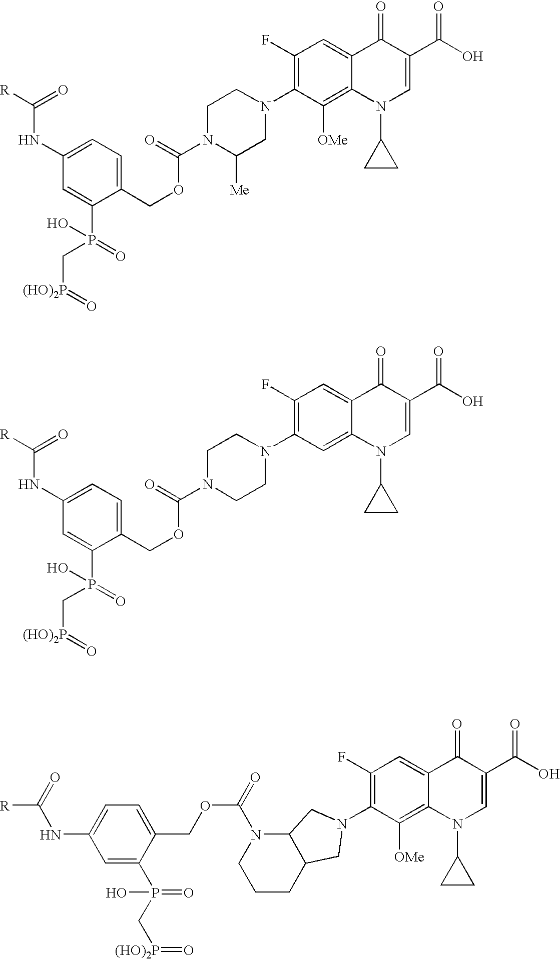

where

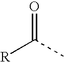

is simple alkanoyl of formula CₙHₘCO where n is an integer between 0 and 20 and m is an integer between 1 and 2n+1 or α-amino-acyl or β-amino acyl.

Further, the present invention covers the compounds of Formula I and of Formula II, as well as pharmaceutically acceptable salts, metabolites, solvates and prodrugs thereof. Examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycolates, tartrates, methanesulfonates, propanesulfonates, naphthalene-1-sulfonates, naphthalene-2-sulfonates, and mandelates.

If the inventive compound is a base, the desired salt may be prepared by any suitable method known to the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such as p-toluenesulfonic acid or ethanesulfonic acid, or the like.

If the inventive compound is an acid, the desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum, and lithium.

In the case of compounds, salts, prodrugs or solvates that are solids, it is understood by those skilled in the art that the inventive compounds, salts, and solvates may exist in different crystal forms, all of which are intended to be within the scope of the present invention.

The inventive compounds may exist as single stereoisomers, racemates and/or mixtures of enantiomers and/or diastereomers. All such single stereoisomers, racemates and mixtures thereof are intended to be within the scope of the present invention. Preferably, the inventive compounds are used in optically pure form.

The compounds of Formula I and/or of Formula II may be administered in the form of a prodrug which is broken down in the human or animal body to give a compound of the Formula I or of Formula II. Examples of prodrugs include in vivo hydrolyzable esters of a compound of the Formula I and/or of Formula II.

An in vivo hydrolyzable ester of a compound of the Formula I and/or of Formula II containing carboxy or hydroxy group is, for example, a pharmaceutically-acceptable ester which is hydrolyzed in the human or animal body to produce the parent acid or alcohol. Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolen-2-onylmethyl esters for example 5-methyl-1,3-dioxolen-2-onylmethyl; and (1-6C)alkoxycarbonyloxyethyl esters for example 1-methoxycarbonyloxyethyl and may be formed at any carboxy group in the compounds of this invention.

An in vivo hydrolyzable ester of a compound of the Formula I and/or of Formula II containing a hydroxy group includes inorganic esters such as phosphate esters and alpha-acyloxyalkyl ethers and related compounds which as a result of in vivo hydrolysis of the ester break down to give the parent hydroxy group. Examples of alpha-acyloxyalkyl ethers include acetoxymethoxy and 2,2-dimethylpropionyloxymethoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

### D) Antimicrobial Compositions and Methods of Treatment

A related aspect of the invention concerns the use of compounds of the invention as an active ingredient in a therapeutic or anti-bacterial composition for treatment or prevention purposes.

### Pharmaceutical Compositions

The compounds of the present invention may be formulated as pharmaceutically acceptable compositions.

The present invention provides for pharmaceutical compositions comprising a therapeutically effective amount of the inventive compound as described herein in combination with a pharmaceutically acceptable carrier or excipient. Such carriers include, but are not limited to saline, buffered saline, dextrose, water, glycerol, ethanol, and combinations thereof.

Acceptable methods of preparing suitable pharmaceutical forms of the pharmaceutical compositions according to the invention are known to those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

The compounds and compositions of the invention are conceived to have a broad spectrum of activity against bacteria, including activity against bacterial strains resistant to antibiotics such as Methicillin, rifampicin, Isoniazid, Streptomycin and Vancomycin (Woodcock, J. M. et al. Antimicrob. Agents Chemother (1997), 41:101-106; Donskey et al, Antimicrob. Agents Chemother. (2004), 48:326-328 and references cited therein), as well as activity against Gram-positive bacteria (e.g. Staphylococcus aureus, Staphylococcus epidermis, Streptococcus pyogenes, Enterococcus faecalis) and Gram-negative bacteria (e.g. E. coli, Chlamydia pneumoniae, Enterobacter sp., H. influenza, K pneumoniae, Legionella pneumoniae, P. aeruginosa) (refer to Mitscher L. A., Chem. Rev. (2005), 105:559-592).

### Pharmaceutical Compositions Comprising Additional Antibiotics

A wide range of second antibiotics can be used in combination with the fluoroquinolone compounds, compositions and methods of the present invention. Such second antibiotics may act by interfering with cell wall synthesis, plasma membrane integrity, nucleic acid synthesis, ribosomal function, folate synthesis, etc. A non-limiting list of useful second antibiotics with which the compounds and compositions might be combined includes: sulfonamides, beta-lactams, tetracyclines, chloramphenicol, aminoglycosides, macrolides, glycopeptides, streptogramins, quinolones, fluoroquinolones, oxazolidinones and lipopeptides.

Preferably, the second antibiotic is a rifamycin analog, such as rifampicin (U.S. Pat. No. 3,342,810), rifapentin (U.S. Pat. No. 4,002,752), rifabutin (U.S. Pat. No. 4,219,478), rifalazil (U.S. Pat. No. 4,983,602), rifandin (U.S. Pat. No. 4,353,826), rifaximin (U.S. Pat. No. 4,341,785), or other rifamycin derivatives and hybrids, such as those described in United States patent application publication 2005/0043298. Or preferably the second antibiotic is tetracycline or tygecycline or other tetracycline, glycycycline and minocycline derivatives.

### Methods for Inhibiting Bacterial Growth

According to a related aspect, the present invention concerns methods of inhibiting bacterial growth, and more particularly growth of Gram-positive bacteria. The method comprises contacting the bacteria for the purpose of such inhibition with an effective amount of a phosphonated fluoroquinolone compound or antibacterial analog thereof according to the invention (or a pharmaceutically acceptable prodrug, salt, active metabolite, or solvate thereof). For example, one can inhibit bacterial topoisomerase II (DNA gyrase) and/or bacterial topoisomerase IV enzyme-dependent DNA transcription, replication, and/or repair in bacteria by contacting a bacterium with a compound of the invention.

The activity of the inventive compounds as inhibitors of DNA transcription, replication, and/or repair may be measured by any of the methods available to those skilled in the art, including in vivo and in vitro assays. Some examples of supercoiling or decatenation assays of bacterial topoisomerase II (DNA gyrase) and bacterial topoisomerase IV enzymes have been described by Domagala and coworkers (J. Med. Chem. (1986), 29:394-404), Mizuuchi and coworkers (J. Biol. Chem. (1984), 258:9199-9201) and Tanaka and coworkers (Antimicrob. Agents Chemother. (1997), 41:2362-2366).

The contacting may be carried out in vitro (in biochemical and/or cellular assays), in vivo in a non-human animal, in vivo in mammals, including humans and/or ex vivo (e.g. for sterilization purposes).

The pharmaceutical compositions may be administered in any effective, convenient manner including, for instance, administration by topical, parenteral, oral, anal, intravaginal, intravenous, intraperitoneal, intramuscular, intraocular, subcutaneous, intranasal, intrabronchial, or intradermal routes among others.

In therapy or as a prophylactic, the compound(s) of the invention and/or pharmaceutically acceptable prodrugs, salts, active metabolites and solvates may be administered to an individual as an injectable composition, for example as a sterile aqueous dispersion, preferably isotonic. Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

Alternative means for systemic administration include transmucosal and transdermal administration using penetrants such as bile salts or fusidic acids or other detergents. In addition, if a compound of the present invention can be formulated in an enteric or an encapsulated formulation, oral administration may also be possible. Administration of these compounds may also be topical and/or localized, in the form of salves, pastes, gels, and the like.

While the treatment can be administered in a systemic manner through the means described above, it may also be administered in a localized manner. For example, the treatment may be administered directly to a bone, such as through an injection into a bone. The treatment may also be administered in other localized manners, such as application to a wound through a topical composition or directly into a subcutaneous or other form of wound.

The active compound(s) and its pharmaceutically acceptable prodrugs, salts, metabolites and solvates may be also administered to an individual as part of a bone substitute or bone-repair compound such as bone cements or fillers (e.g. Skelite™, Millenium Biologics, Kingston, ON, Canada) and calcium or hydroxyapatite beads.

A dose of the pharmaceutical composition contains at least a pharmaceutically- or therapeutically-effective amount of the active compound (i.e., a compound of Formula I, of Formula II and/or a pharmaceutically acceptable prodrug, salt, active metabolite, or solvate thereof), and is preferably made up of one or more pharmaceutical dosage units. The selected dose may be administered to a mammal, for example, a human patient, in need of treatment. A “therapeutically effective amount” is intended to mean that amount of a compound of Formula I and/or of Formula II (and/or a pharmaceutically acceptable prodrug, salt, active metabolite, or solvate thereof) that confers a therapeutic effect on the subject treated. The therapeutic effect may be objective (i.e. measurable by some test or marker (e.g. lower bacterial count)) or subjective (i.e. the subject gives an indication of or feels an effect).

The amount that will correspond to a “therapeutically effective amount” will vary depending upon factors such as the particular compound, the route of administration, excipient usage, the disease condition and the severity thereof, the identity of the mammal in need thereof, and the possibility of co-usage with other agents for treating a disease. Nevertheless the therapeutically effective amount can be readily determined by one of skill in the art. For administration to mammals, and particularly humans, it is expected that the daily dosage level of the active compound will be from 0.1 mg/kg to 200 mg/kg, typically around 1-5 mg/kg. The physician in any event will determine the actual dosage that will be most suitable for an individual and will vary with the age, weight and response of the particular individual. The above dosages are exemplary of the average case. There can, of course, be individual instances where higher or lower dosage ranges are merited, and such are within the scope of this invention.

The invention provides a method of treating a subject in need of treatment wherein a phosphonated fluoroquinolone molecule having high affinity to osseous tissues is administered to the subject. Preferably, the phosphonated group is coupled to the fluoroquinolone molecule through a cleavable linker. Preferably the subject is a mammal, such as a human. The method of treatment may also be applied in a veterinary aspect, to animals such as farm animals including horses, cattle, sheep, and goats, and pets such as dogs, cats and birds.

Although the invention is preferably directed to the prevention and/or treatment of bone-related infections, the invention encompasses therapeutic and prophylactic methods against other diseases caused by or related to bacterial infection, including but not limited to otitis, conjunctivitis, pneumonia, bacteremia, sinusitis, pleural emphysema and endocarditis, low grade infections in the vicinity of calcifications of atherosclerotic vessels, and meningitis. In such methods, an effective therapeutic or prophylactic amount of an antibacterial compound and/or composition as defined hereinbefore, is administered to a mammal (preferably a human) in an amount sufficient to provide a therapeutic effect and thereby prevent or treat the infection of the mammal. Exact amounts can be routinely determined by one skilled in the art and will vary depending on several factors, such as the particular bacterial strain involved and the particular antibacterial compound used.

### Prophylaxis and Prevention

An additional use that is particularly contemplated for the compounds invention is for prophylaxis and prevention purposes. Indeed, many orthopedic surgeons consider that humans with prosthetic joints should be considered for antibiotic prophylaxis before a treatment that could produce a bacteremia. Deep infection is a serious complication sometimes leading to loss of the prosthetic joint and is accompanied by significant morbidity and mortality. The compounds and compositions of the invention may therefore be used as a replacement for prophylactic antibiotics in this situation. For instance, the compounds and/or compositions of the invention may be administered by injection to achieve a systemic and/or local effect against relevant bacteria shortly before an invasive medical treatment, such as surgery or insertion of an in-dwelling device (e.g. joint replacement (hip, knee, shoulder, etc.), bone grafting, fracture repair, dental operation or implant. Treatment may be continued after invasive medical treatment, such as post-operatively or during the in-body time of the device.

In addition, the compound and/or composition may also be administered before the invasive medical treatment to permit the accumulation of the compound into the bone tissues prior to the treatment.

In each instance, the compound(s) of the invention could be administered once, twice, thrice or more, from 1, 2, 3, 4, 5, 6, 7 days or more, to 10, 9, 8, 7, 6, 5, 4, 3, 2, or 1 hour or less before surgery for permitting an advisable systemic or local presence of the compounds, and/or accumulation in the bones, preferably in the areas potentially exposed to bacterial contamination during the surgical procedure. Even more preferably, the phosphonated derivatives of the invention would be administered such that they can reach a local concentration of about 5, 10, 20, 30, 40, 50, 75, 100, 500 or even 1000 fold higher concentration than the concentration that would normally be achieved during the administration of the unmodified parent fluoroquinolones, i.e. a non-phosphonated equivalent. The compound(s) may be administered after the invasive medical treatment for a period of time, such as 1, 2, 3, 4, 5 or 6 days, 1, 2, 3 or more weeks, or for the entire time in which the device is present in the body.

Therefore, the invention provides a method of inducing accumulation of a fluoroquinolone molecule in bones of a mammal wherein a phosphonated fluoroquinolone molecule having high affinity to osseous tissues is administered to a mammal. The phosphonated fluoroquinolone binds osseous tissues and accumulates in bones of the mammal in amounts greater than amounts of a non-phosphonated equivalent of the fluoroquinolone molecule. Preferably, the phosphonated group is coupled to the fluoroquinolone molecule through a cleavable linker.

The invention further provides a method for prolonging the presence of a fluoroquinolone antimicrobial molecule in bones of a mammal wherein a phosphonated fluoroquinolone molecule having a high affinity to osseous tissues is administered to a mammal. The phosphonated group is coupled to the fluoroquinolone molecule through a cleavable linker. The phosphonated fluoroquinolone binds osseous tissues and accumulates in bones of the mammal, and the linker is cleaved gradually within the bones thereby releasing the fluoroquinolone molecule and prolonging the presence of the fluoroquinolone molecule in the bones.

### E) In-Dwelling Devices and Products Coated with the Phosphonated Fluoroquinolone Derivatives of the Invention

The invention further encompasses in-dwelling devices coated with the compounds of the invention. As used herein, the term “in-dwelling device” refers to surgical implants, orthopedic devices, prosthetic devices and catheters, i.e., devices that are introduced to the body of an individual and remain in position for an extended time. Such devices include, but are not limited to, artificial joints and implants, heart valves, pacemakers, vascular grafts, vascular catheters, cerebrospinal fluid shunts, urinary catheters, continuous ambulatory peritoneal dialysis (CAPD) catheters.

According to one embodiment, the in-dwelling device is bathed in or sprayed with a concentration of about 1 mg/ml to about 10 mg/ml of the compound and/or the composition of the invention, before its insertion in the body.

According to another embodiment, the in-dwelling device is made of, or pre-coated with, an osseous-like type of material (e.g. calcium phosphate, Ca-ion and hydroxyapatite (Yoshinari et al., Biomaterials (2001), 22(7): 709-715)). Such material is likely to advantageously improve binding of the compounds of the invention to the in-dwelling device, either during the coating of the device with the compounds of the invention and/or after their local or systemic administration. The in-dwelling devices may also be coated with an osseous material pre-loaded with or containing bound bone-targeting compound(s) according to the invention. For the above-mentioned embodiments, hydroxyapatite would be preferred as the osseous material. More details on coating methods, uses and advantages of hydroxyapatite-coated prostheses are found in the review by Dumbleton and Manly (The Journal of Bone & Joint Surgery (2004) 86A:2526-40) which is incorporated herein by reference.

### F) Methods of Preparation

The inventive compounds, and their salts, solvates, crystal forms, active metabolites, and prodrugs, may be prepared by employing the techniques available in the art using starting materials that are readily available. Certain novel and exemplary methods of preparing the inventive compounds are described in the Exemplification section below. Such methods are within the scope of this invention.

### EXAMPLES

The Examples set forth herein provide exemplary syntheses of representative compounds of the invention. Also provided are exemplary methods for assaying the compounds of the invention for their bone-binding activity, assays for determining the minimum inhibitory concentration (MIC) of the compounds of the invention against microorganisms, and methods for testing in vivo activity and cytotoxicity.

### Example 1

### Synthesis of Moxifloxacin, Gatifloxacin and Ciprofloxacin Bisphosphonate Conjugates

### A) General Experimental Procedures

The synthetic methods for the preparation of quinolone antibiotics are reviewed in Chem. Rev. (2005), 105: 559-592. The syntheses of moxifloxacin, gatifloxacin and cirpofloxacin are described in U.S. Pat. No. 4,990,517, U.S. Pat. No. 4,980,470 and U.S. Pat. No. 4,670,444 respectively.

### A 1) Preparation of Bisphosphonate Building Blocks

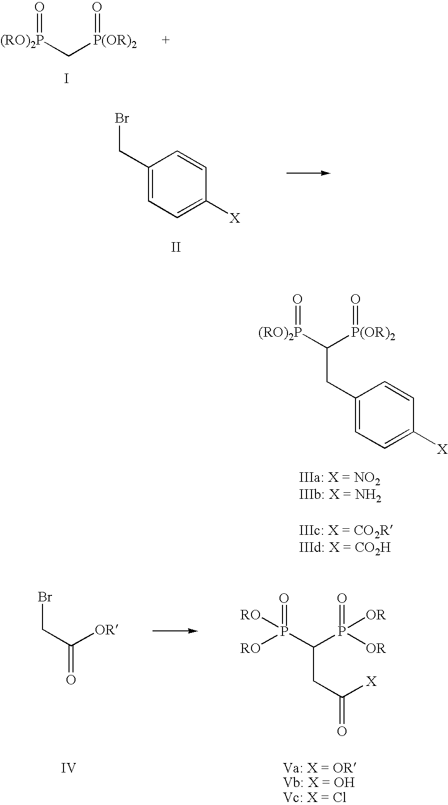

Following protocols described in Bioorg. Med. Chem. (1999), 7: 901-919, benzyl substituted bisphosphonate building blocks of the general structures III and V can be obtained by alkylation of the anion of I with 4-substituted benzyl bromide II or bromoacetate IV. Nitro compound IIIa can be converted to aniline IIIb by reduction of the nitro group under hydrogenation conditions, using a catalyst such as PtO₂. Esters like IIIc and Va can be converted to the corresponding acids IIId or Vb via ester cleavage. For example, ester IIIc where R′=t-Bu can be treated with TFA to afford the corresponding acid IIId. Under similar conditions, ester Va where X=Ot-Bu can be converted to acid Vb.

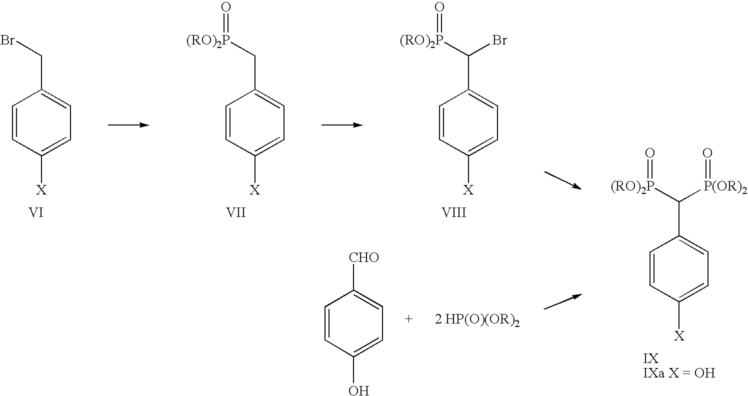

Aryl substituted methylene bisphosphonates of general formula IX can be obtained from the parent benzylic halides VI in a sequence of two Arbuzov reactions separated by a benzylic halogenation. The hydroxyl substituted parent molecule IXa can be obtained by the nucleophilic addition of the alkali metal salt of a dialkyl phosphite to 4-hydroxybenzaldehyde as described in Org. Biomol. Chem. (2004), 21:3162-3166.

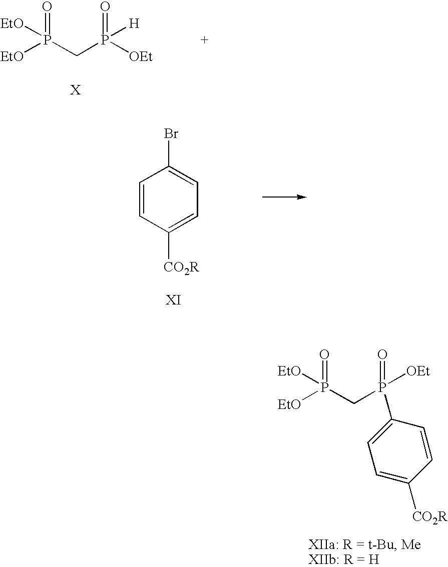

Diethyl (ethoxyphosphinyl)methylphosphonate X can be prepared using the procedure described in Synth. Comm. (2002), 32: 2951-2957 and patent U.S. Pat. No. 5,952,478 (1999). It can be coupled with a 4-substituted bromobenzene (XI) to access acid XIIb, following cleavage of the ester intermediate XIIa.

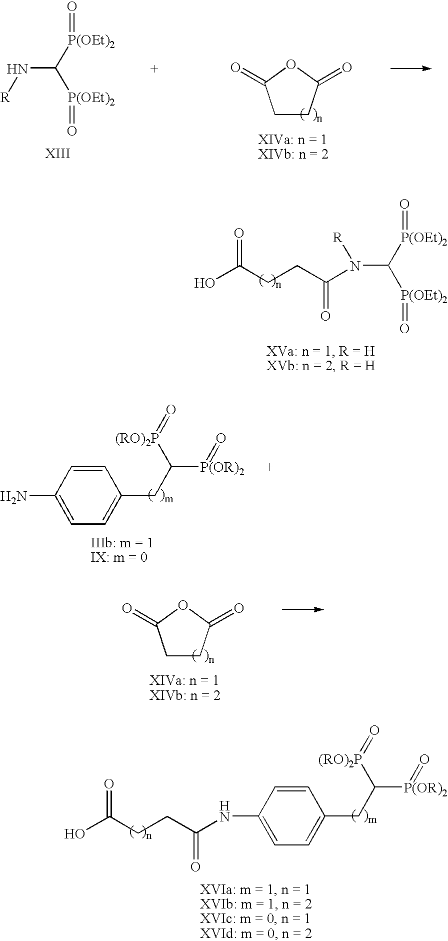

Amines of the general formula XIII can be prepared from dibenzylamine, diallylamine, or other N-benzyl and N-allyl secondary amines, diethyl phosphite and triethyl orthoformate following a protocol described in Synth. Comm. (1996), 26: 2037-2043. Acylation of Xil with succinic an hydride XIVa or glutaric an hydride XIVb can provide acids XVa and XVb respectively (J. Drug Targeting (1997), 5: 129-138). In a similar fashion, treatment of the previously described IIIb or IX with XIV(a-b) results in the succinamic and glutaramic acids XVI(a-d).

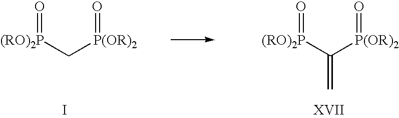

Olefin XVII can be prepared from 1 following a protocol described in J. Org. Chem. (1986), 51: 3488-3490.

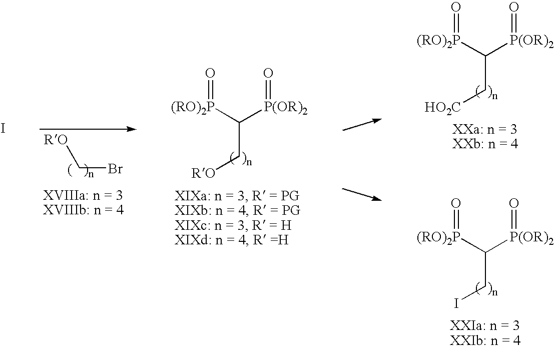

As described in Phosphorus, Sulfur and Silicon (1998), 132: 219-229, alcohols of general structure XIX(c-d) and iodides of general structure XXI can be prepared by alkylation of the anion of I by protected ω-hydroxy bromides of various chain length XVIII. After deprotection, alcohols can be converted to the corresponding iodides via treatment with in situ generated triphenylphosphine:iodine complex. These alcohols XIX(c-d) may additionally be converted to acids of general structure XX by conventional methods of oxidation, such as treatment with pyridinium dichromate.

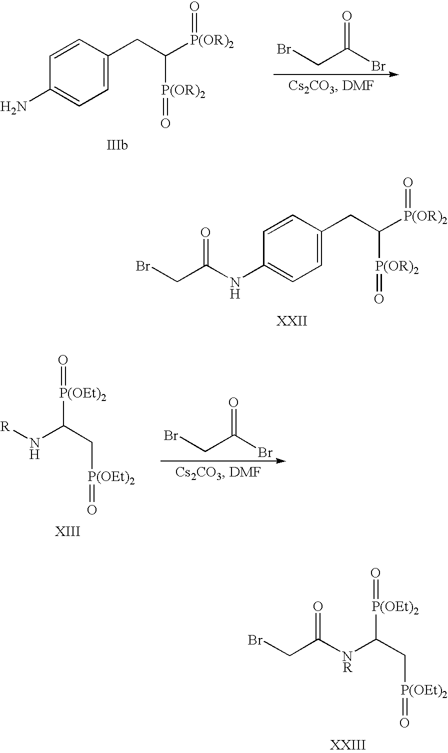

Bromoacetamides XXII and XXIII from the parent amines IIIb and XIII can be prepared according to a modification of the procedure described in J. Drug Targeting (1995), 3: 273-282.

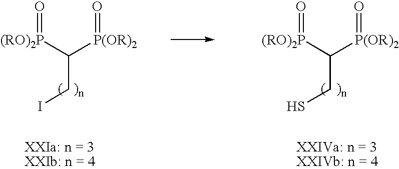

Thiols XXIV(a-b) can be prepared by alkylation of the anion of I with a protected 3-iodopropane-1-thiol following the protocol described in Bioorg. Med. Chem. (1999), 7: 901-919. Or they can be prepared from iodides XXI(a-b) and an appropriately chosen reagent able to supply the sulfhydryl group, including reagents such as thiourea followed by hydrolysis and thioacetic acid followed by hydrolysis or reduction.

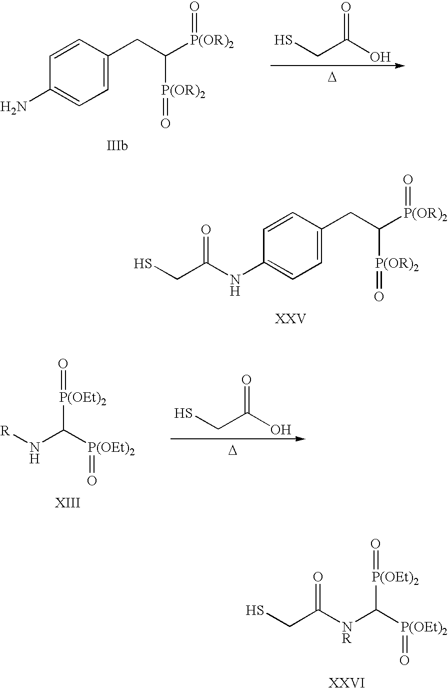

Thioglycolamides XXV and XXVI can be made through the condensation of amine functionalized bisphosphonates such as IIIb and XIII with activated forms of thioglycolic acid, or with thioglycolic acid itself as described for other amines in J. Ind. Chem. Soc. (1997), 74: 679-682.

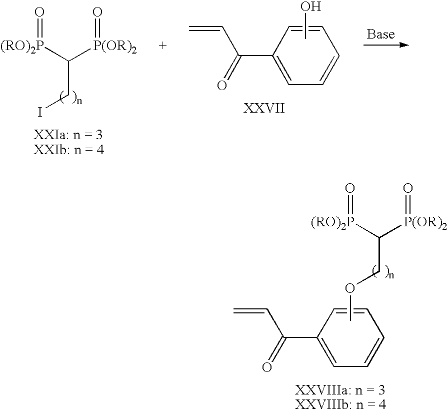

Vinyl ketones such as XXVIII(a-b) can be prepared through the condensation of the parent (hydroxyphenyl) vinyl ketone XXVII with iodides XXI(a-b) in the presence of an appropriately chosen base.

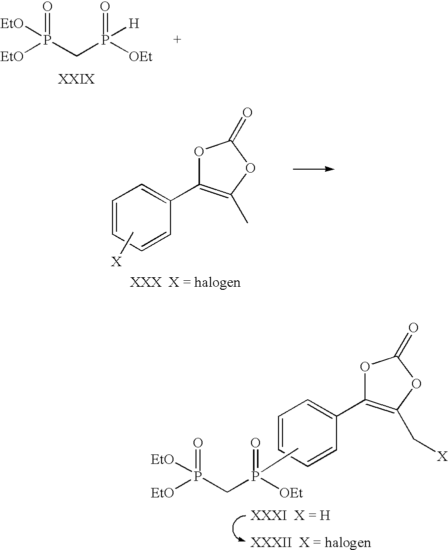

Diethyl (ethoxyphosphinyl)methylphosphonate XXIX can be prepared using the procedure described in Synth. Comm. (2002), 32: 2951-2957 and patent U.S. Pat. No. 5,952,478 (1999). It can be coupled with a halogenated 1,3-dioxolone XXX to furnish bisphosphonate XXXI. This can be followed by a radical halogenation reaction to provide bisphosphonate XXXII.

The bisphosphonate building blocks described in this section are in the form of their phosphonic esters, R being Me, Et, i-Pr, allyl or Bn; or as the free bisphosphonic acids and/or free bisphosphonate salts.

### A 2) Synthesis of Fluoroquinolone-Bisphosphonate Conjugates

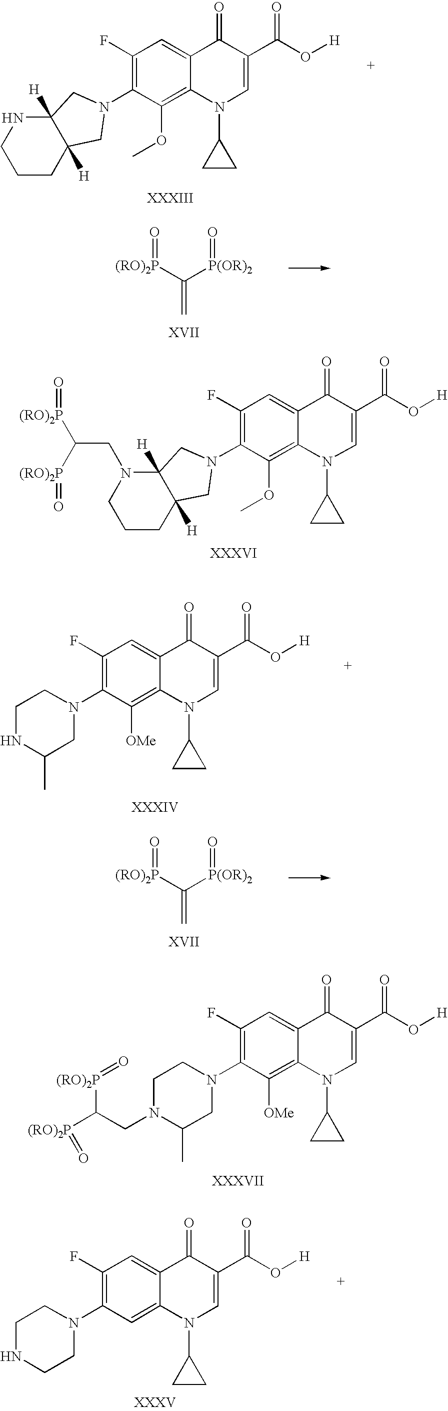

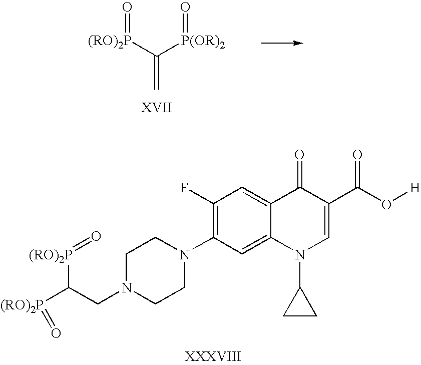

Treatment of a fluoroquinolone possessing a primary or secondary amine functionality on the C-7 substituent with vinylidene bisphosphonate XVII under conditions of nucleophilic catalysis provides bisphosphonated adducts, as described in J. Med. Chem. (2002), 45: 2338-2341. Hence moxifloxacin XXXIII, gatifloxacin XXXIV and ciprofloxacin XXXV can be converted to aminomethylated methylenebisphosphonates XXXVI-XXXVIII.

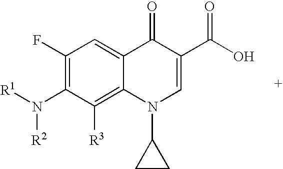

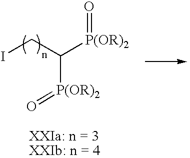

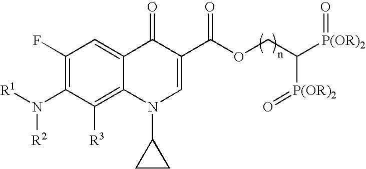

| XXXIX | R¹R²N— | R³— |
|---|---|---|
| a | 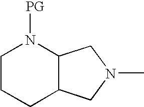 | MeO— |
| b | 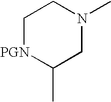 | MeO— |
| c | 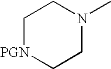 | H— |

| XL | R¹R²N— | R³— | n |
|---|---|---|---|
| ab | 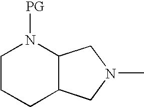 | MeO—MeO— | 34 |
| cd | 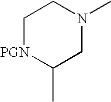 | MeO—MeO— | 34 |
| ef | 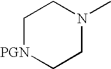 | H—H— | 34 |

Protection of the secondary amino groups of XXXIII-XXXV followed by treatment with co-iodoalkylbisphosphonates XXI(a-b) gives the fluoroquinolone bisphosphonate adduct XL(a-f).

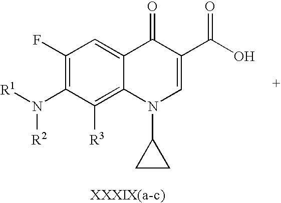

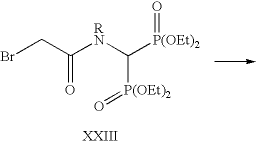

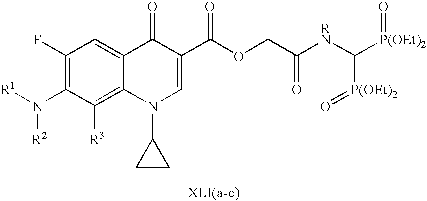

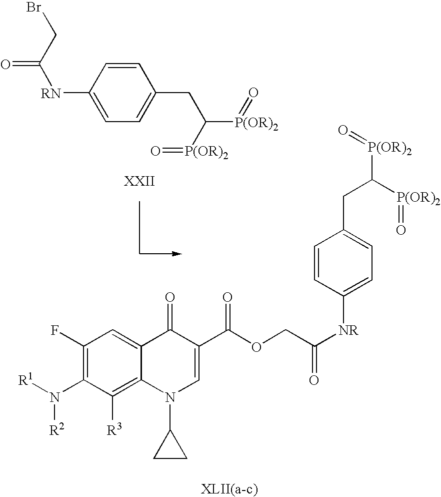

| XLI/XLII | R¹R²N— | R³— |
|---|---|---|
| a | 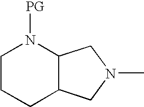 | MeO— |
| b | 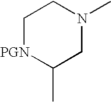 | MeO— |
| c | 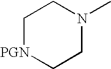 | H— |

A similar reaction of the protected fluoroquinolones with bisphosphonated alkyl halides such as XXII and XXIII provides their parent bisphosphonated glycoamide prodrugs XLI(a-c) and XLII(a-c) in a manner similar as described in J. Drug Targeting (1995), 3: 273-282.

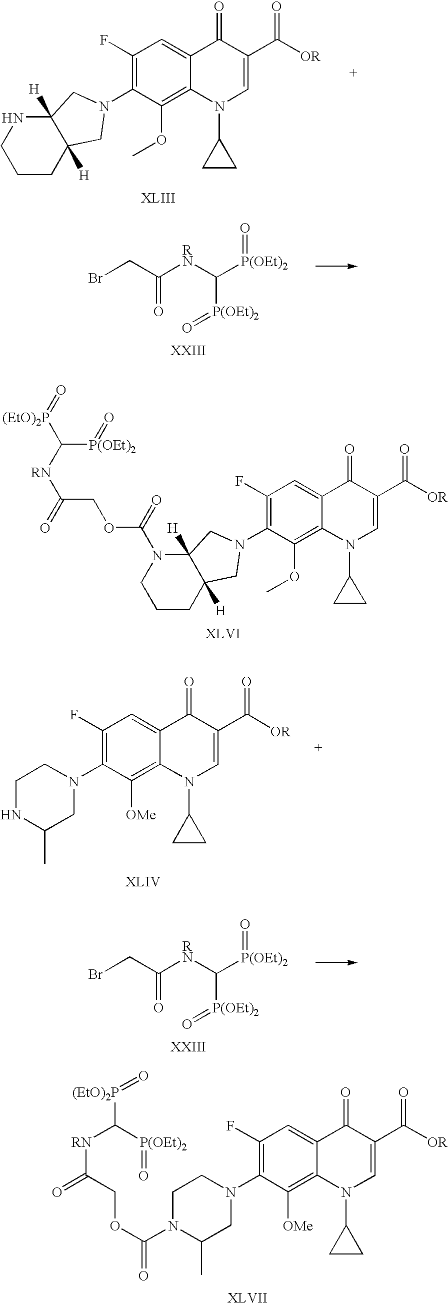

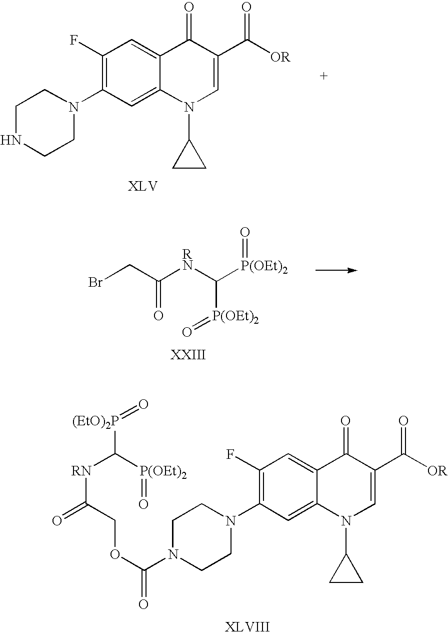

Protection of the carboxy group of fluoroquinolones yields compounds XLIII-XLV. These can be treated with bromoacetamide XXIII and carbon dioxide in the presence of a base as described in Synlett (1994): 894 to result in bisphosphonated carbamates XLVI-XLVIII. A similar treatment but substituting XXIII with XXII results in similar compounds IL-LI:

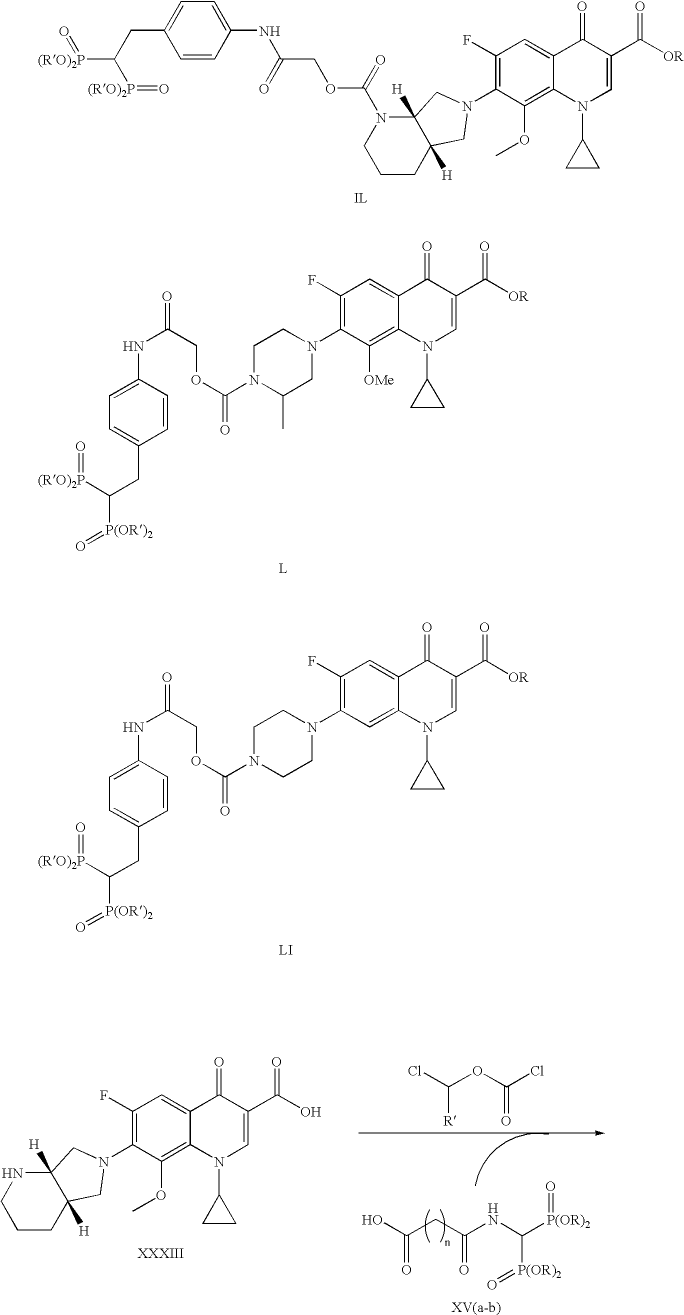

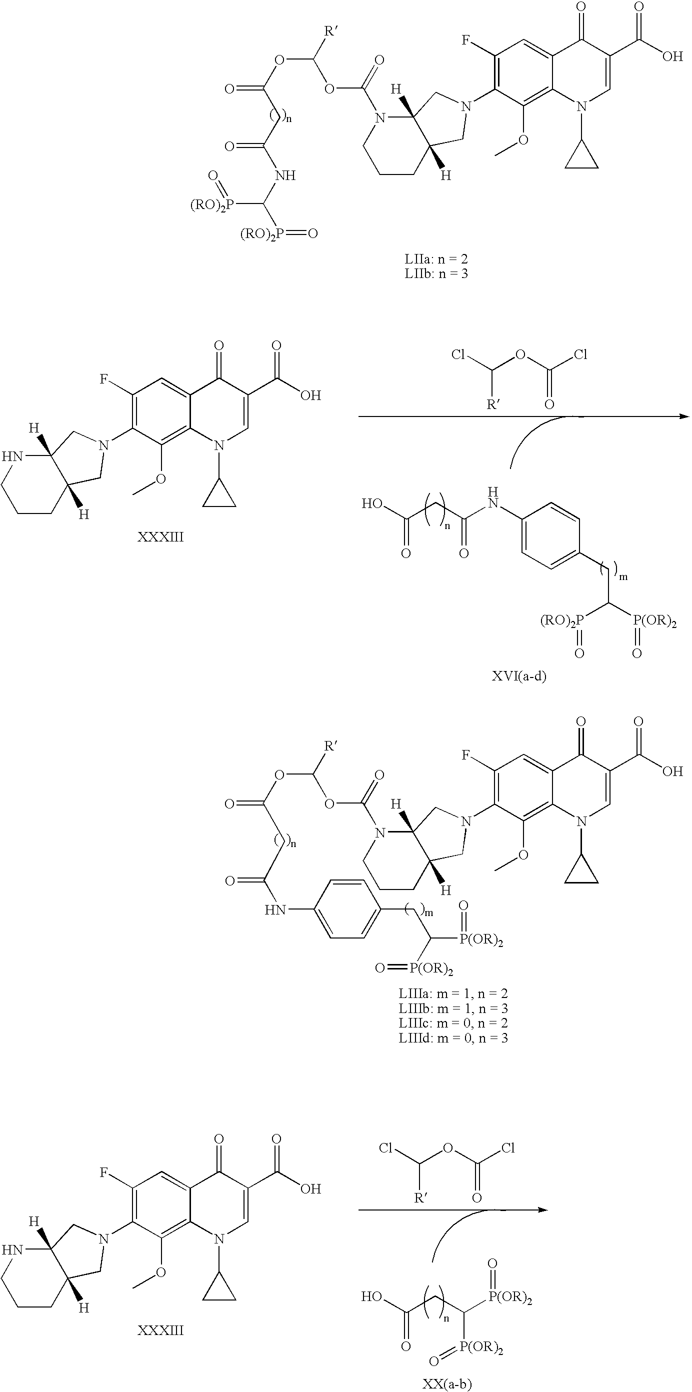

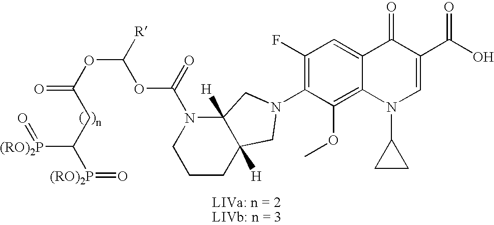

Treatment of moxifloxacin XXXIII with a 1-chloroalkyl chloroformate in a manner described for other compounds in J. Med. Chem. (1991), 34: 78-81 results in the formation of the parent 1-chloroalkyl carbamate which reacts with a salt of XV(a-b), XVI(a-d), or XX(a-b) to generate the bisphosphonated adducts LII(a-b), LIII(a-d) or LIV(a-b) respectively. Similarly, gatifloxacin is converted to LV(a-f) and LVI(a-b) and ciprofloxacin to LVII(a-f) and LVIII(a-b) respectively via the same sequence of reactions.

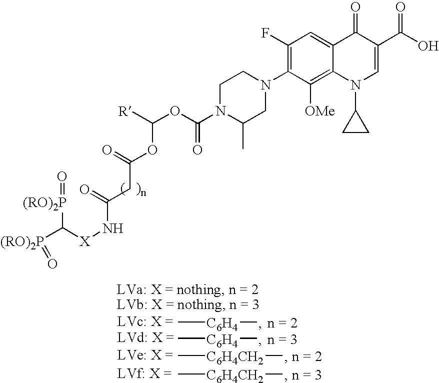

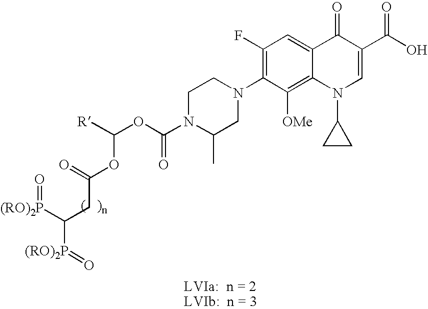

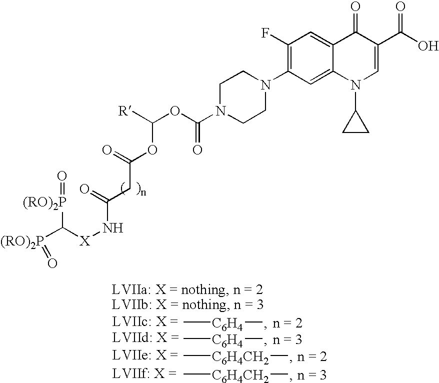

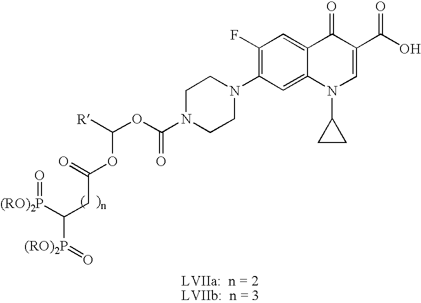

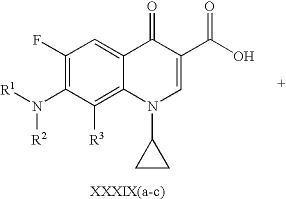

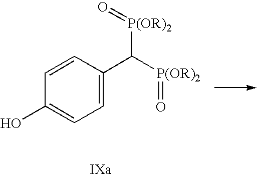

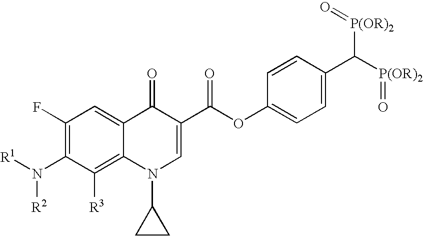

| LIX | R¹R²N— | R³— |
|---|---|---|
| a | 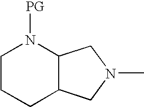 | MeO— |
| b | 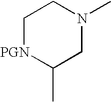 | MeO— |
| c | 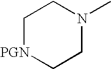 | H— |

Bisphosphonate phenyl esters can be prepared by the condensation of protected fluoroquinolones XXXIX(a-c) with the bisphosphonated phenol IXa in the presence of standard coupling reagents.

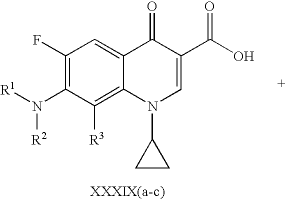

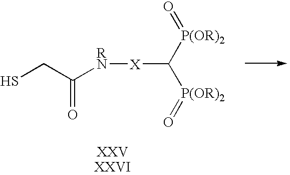

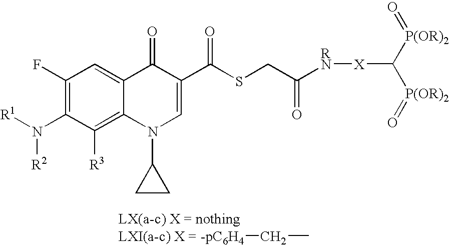

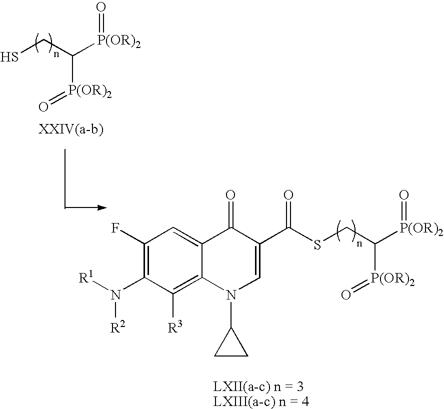

| LX/LXI/LXII/LXIII | R¹R²N— | R³— |
|---|---|---|
| a | 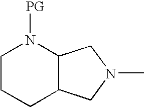 | MeO— |
| b | 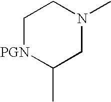 | MeO— |
| c | 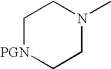 | H— |

Similarly, XXXIX(a-c) can be reacted with thiols XXIV(a-b), XXV or XXVI in the presence of an appropriately selected standard coupling reagent to furnish bisphosphonated thioesters of the general structures LX(a-c), LXI(a-c), LXII(a-c) and LXIII(a-c).

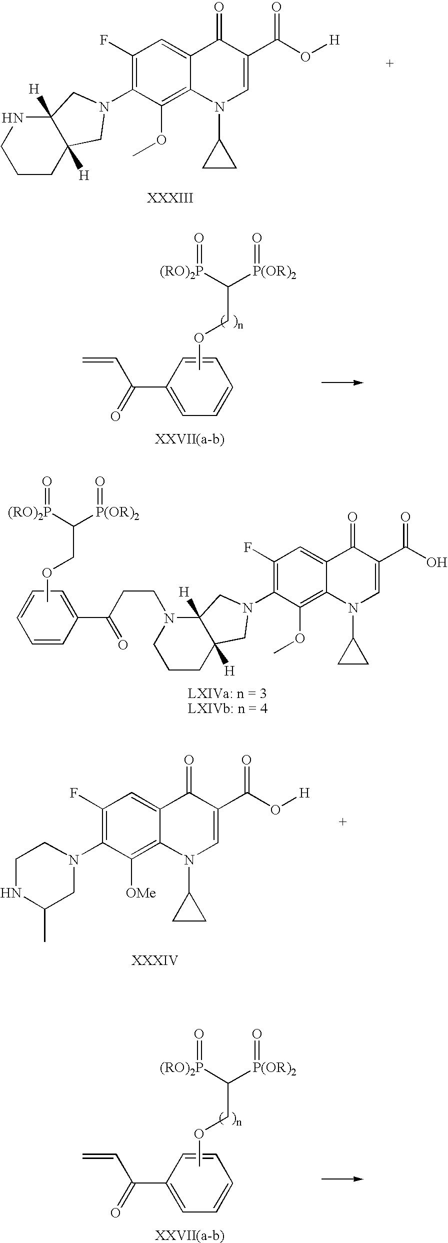

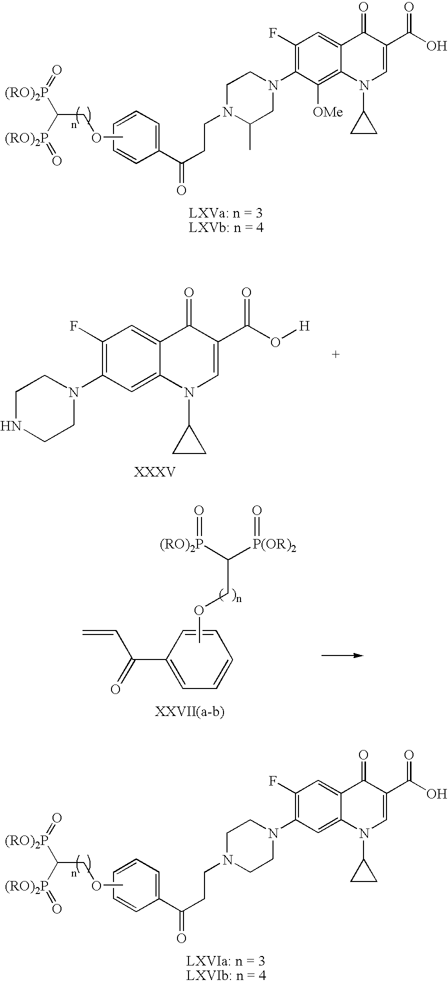

The condensation of fluoroquinolones such as XXXIII, XXXIV and XXXV with bisphosphonated vinyl ketones, such as XXVII(a-b), gives the bisphosphonated fluoroquinolone prodrugs LXIV(a-b), LXV(a-b) and LXVI(a-b).

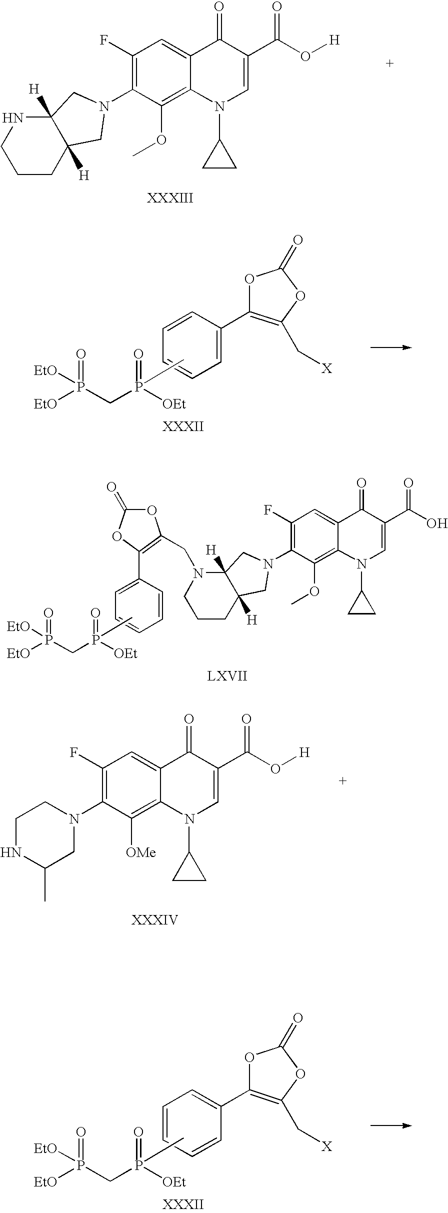

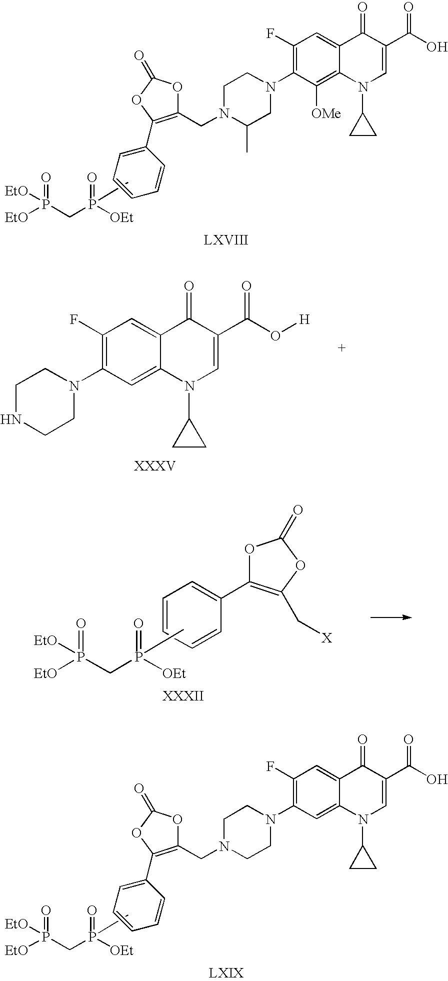

Treatment of fluoroquinolones XXXIII-XXXV with the bisphosphonated halomethyldioxolone XXXII in the presence of a non nucleophilic base furnishes the bisphosphonated dioxolonylmethyl fluoroquinolones LXVII, LXVIII and LXIX.

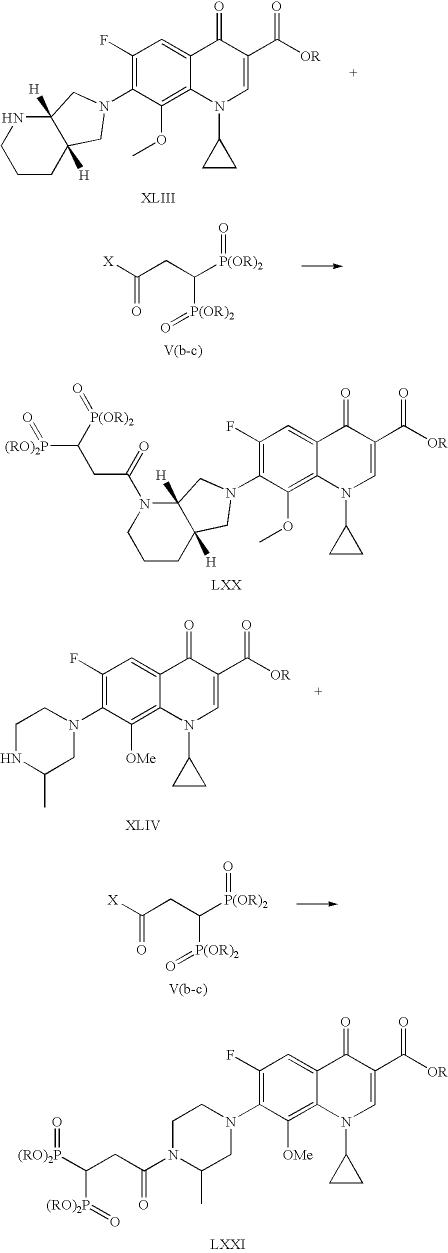

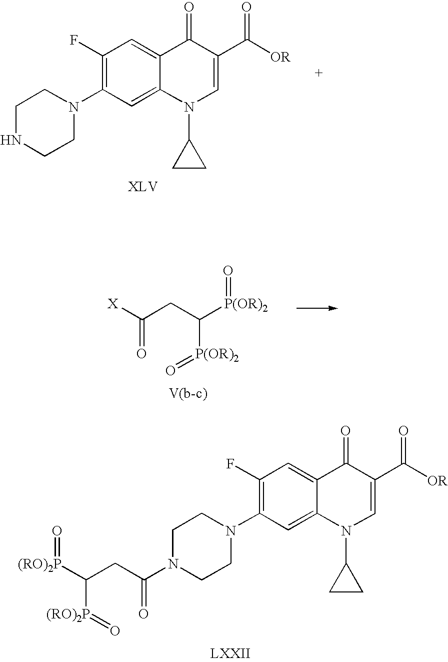

Bisphosphonated amides LXX-LXXII can be prepared from the parent protected fluoroquinolones XLIII-XLV by treatment either with carboxylic acid Vb in the presence of a coupling agent or with acid chloride Vc in the presence of a base.

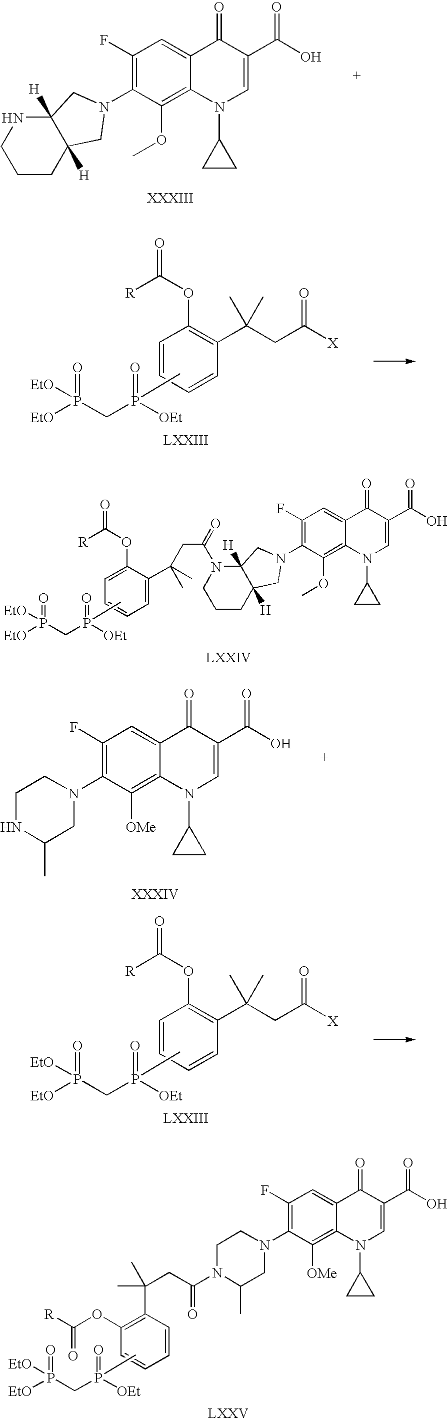

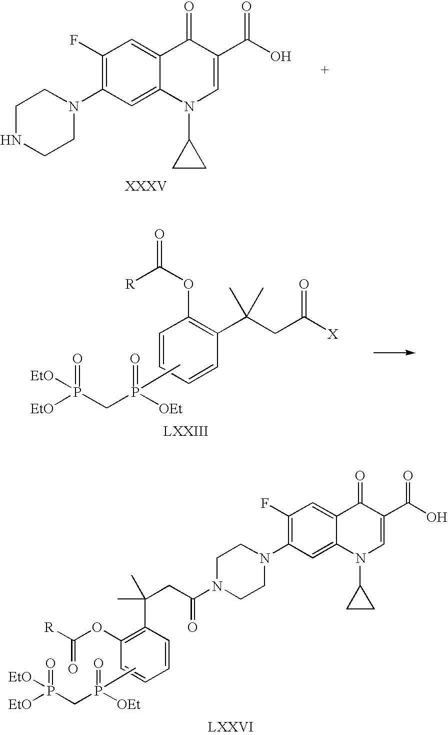

Bisphosphonated amides LXXIV-LXXVI can be prepared from the parent protected fluoroquinolones XLIII-XLV by treatment either with a bisphosphonated 3-(2-acyloxyphenyl)-3-methylbutanoic acid (LXXIII X═OH) under standard dehydrative coupling conditions or with the parent acyl halides (LLXIII X=halogen) in the presence of a suitable base.

The analogous compounds LXXVIII-LXXX can be prepared in a similar fashion using the suitably protected bisphosphonated 3-(2-phosphoryloxyphenyl)-3-methylbutanoic acid (LLXVII X═OH) or its parent acyl halide (LXXVII X=halogen).

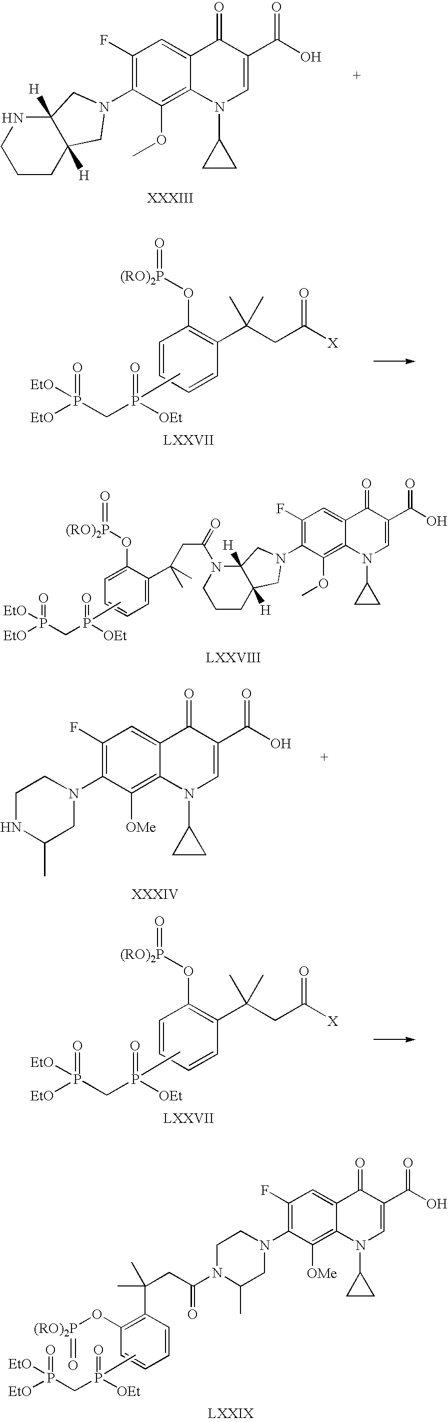

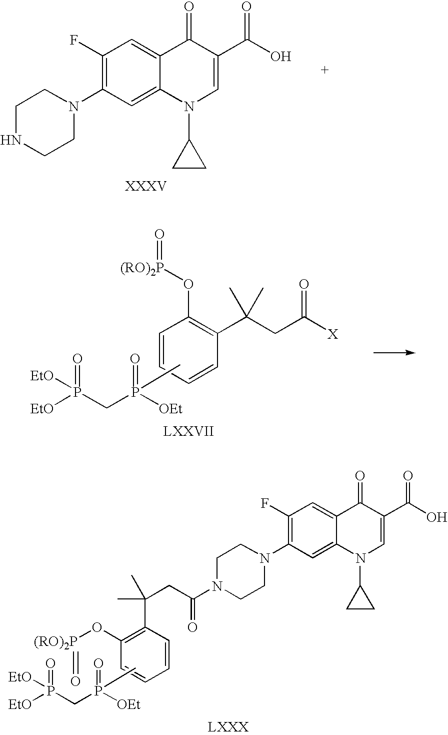

The bisphosphonate building blocks described in this section are in the form of their phosphonic esters, R being Me, Et, i-Pr, allyl or Bn; or as the free bisphosphonic acids and/or free bisphosphonate salts. The bisphosphonic esters may be converted to the free acids and acid salts by conventional methods, such as the treatment with trimethylsilyl bromide or Iodide in the presence or the absence of a base, hydrogenation when the bisphosphonate esters are benzyl bisphosphonates, by treatment with a palladium catalyst and a nucleophile when the bisphosphonate esters are allyl bisphosphonates.

The other protecting groups used can be put on and removed using the conventional methods described in the literature, for instance as reviewed in “Protective Groups in Organic Synthesis”, Greene, T. W. and Wuts, P. M. G., Wiley-Interscience, New York, 1999.

### B) Detailed Experimental Procedures

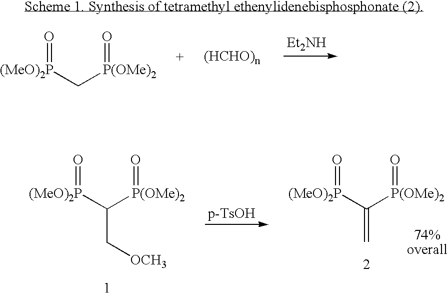

Tetramethyl ethenylidenebisphosphonate (2): Compound 2 was prepared as described in J. Org. Chem. 1986, 51, 3488-3490.2 was obtained as a clear liquid in 74% overall yield. ¹H NMR (400 MHz, CDCl₃) δ 3.78-3.81 (m, 12H), 6.94-7.12 (m, 2H).

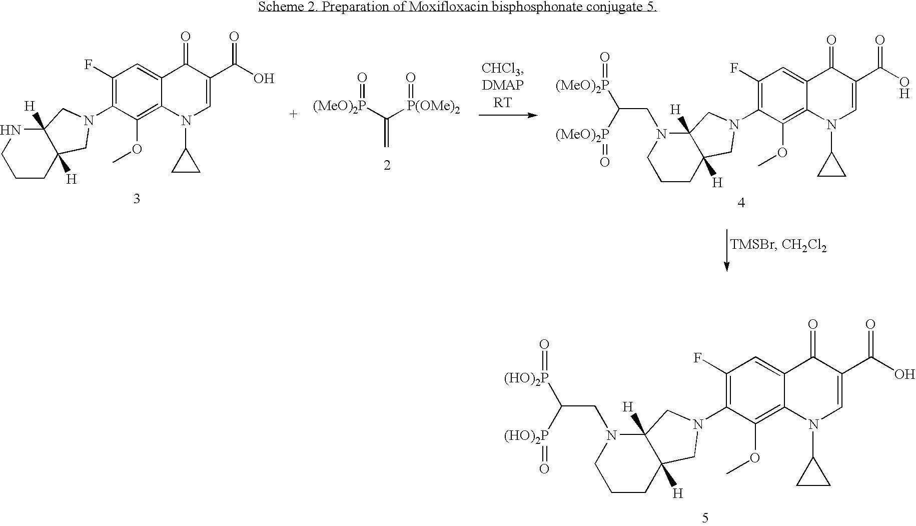

7-((4aS,7aS)-1-(2,2-bis(dimethylphosphono)ethyl)-octahydropyrrolo[3,4-b]pyridin-6-yl)-1-cyclopropyl-6-fluoro-1,4-dihydro-8-methoxy-4-oxoquinoline-3-carboxylic acid (4): Moxifloxacin 3 (0.800 g, 1.99 mmol) was dissolved in dry CHCl₃ (30 mL). To this solution was added tetramethyl ethenylidenebisphosphonate 2 (0.515 g, 2.11 mmol) and a catalytic quantity of DMAP. The reaction mixture was stirred at room temperature for 3.5 h, then evaporated at 40° C. A 1.022 g portion of the crude product was purified by the following procedure. It was treated with a small volume of ethyl acetate. The insoluble material was filtered off, and the product was precipitated with hexanes, washed with hexanes, and dried to give pure 4 (0.448 g, 45%). ¹H NMR (400 MHz, CDCl₃) δ 0.86-0.87 (m, 1H), 0.94-1.05 (m, 2H), 1.10-1.35 (m, 4H), 1.55-1.85 (m, 5H), 2.25-2.40 (m, 2H), 2.64 (tt, J=23.9, 6.0, 1H), 2.75-2.95 (m, 2H), 3.05-3.25 (m, 2H), 3.54 (s, 3H), 3.56-3.72 (m, 6H), 3.74-4.01 (m, 8H), 7.77 (d, J=14.1, 1H), 8.76 (s, 1H).

7-((4aS,7aS)-1-(2,2-bisphosphonoethyl)-octahydropyrrolo[3,4-b]pyridin-6-yl)-1-cyclopropyl-6-fluoro-1,4-dihydro-8-methoxy-4-oxoquinoline-3-carboxylic acid (5): TMSBr (0.76 mL, 5.76 mmol) was added in one portion to a stirring solution of 4 (371 mg, 0.575 mmol) in CH₂Cl₂ (10 mL) and the resulting mixture was stirred at room temperature for 24 h. The solvent was removed under reduced pressure and solid was dried under high vacuum for 1 h. The solid was then suspended in H₂O (15 mL) and the pH was immediately adjusted to pH 7 by the addition of 1M NaOH, with concomitant dissolution of the product. The product was obtained essentially pure following evaporation (quantitative). A 112 mg portion was purified on a C18 Sep-Pak™ (H₂O) to give pure 5 (66 mg, 59% recovery). ¹H NMR (400 MHz, D₂O) δ 0.78-1.36 (m, 4H), 1.56-2.15 (m, 4H), 2.36-2.53 (m, 1H), 2.60-2.85 (m, 1H), 3.32-3.85 (m, 7H), 3.62 (s, 3H), 4.05-4.38 (m, 4H), 7.59 (d, J=13.7, 1H), 8.59 (s, 1H).

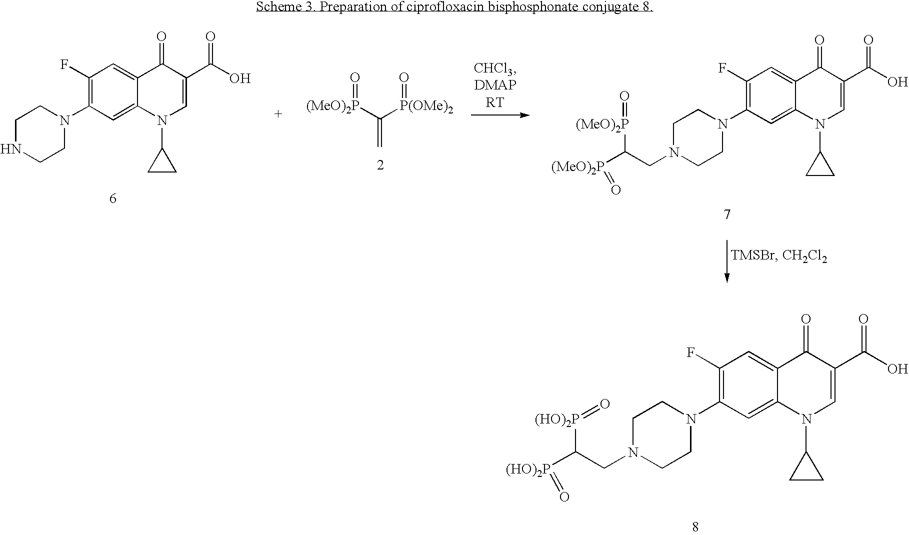

7-(4-(2,2-bis(dimethylphosphono)ethyl)piperazin-1-yl)-1-cyclopropyl-6-fluoro-1,4-dihydro-4-oxoquinoline-3-carboxylic acid (7): Ciprofloxacin 6 (0.40 g, 1.21 mmol) was suspended in dry CHCl₃ (50 mL). To this suspension was added tetramethyl ethenylidenebisphosphonate 2 (0.301 g, 1.23 mmol) and a catalytic quantity of DMAP. The reaction mixture was stirred at room temperature for 2 h, then evaporated at 40° C. The crude product was heated with boiling toluene (50 mL). The insoluble product 8 was obtained as a white powder (0.309 g, 44%). ¹H NMR (400 MHz, CDCl₃) δ 1.17-1.21 (m, 2H), 1.36-1.43 (m, 2H), 1.63 (bs, 1H), 2.67-2.84 (m, 5H), 2.95-3.07 (m, 2H), 3.35 (bs, 4H), 3.48-3.56 (m, 1H), 3.80-3.88 (m, 12H), 7.34 (d, J=7.0, 1H), 8.02 (d, J=12.9, 1H), 8.77 (s, 1H).

7-(4-(2,2-bisphosphonoethyl)piperazin-1-yl)-1-cyclopropyl-6-fluoro-1,4-dihydro-4-oxoquinoline-3-carboxylic acid (8): TMSBr (0.58 mL, 4.39 mmol) was added in one portion to a stirring solution of 7 (252 mg, 0.438 mmol) in CH₂Cl₂ (10 mL) and the resulting mixture was stirred at room temperature for 16 h. The solvent was removed under reduced pressure and solid was dried under high vacuum for 1 h. The solid was suspended in H₂O (30 mL) and the pH was immediately adjusted to pH 7.6 by the addition of 1M NaOH, with concomitant dissolution of the product. The product solution was washed with CHCl₃ (2×25 mL), filtered and evaporated to give 8 in quantitative yield. ¹H NMR (400 MHz, D₂O) δ 1.14 (bs, 2H), 1.32-1.38 (m, 2H), 2.35-2.50 (m, 1H), 3.36-3.90 (m, 11H), 7.66 (d, J=7.0, 1H), 7.93 (d, J=13.1, 1H), 8.51 (s, 1H).

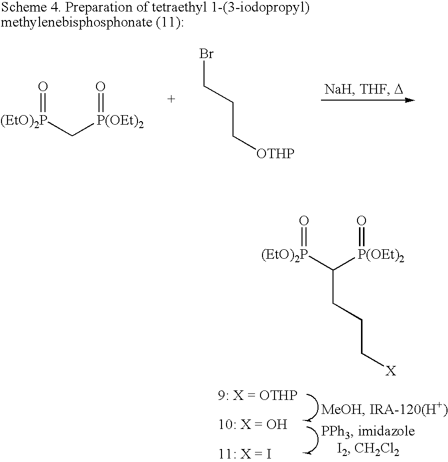

Tetraethyl 4-(2-Tetrahydro-2H-pyranyloxy)butylene-1,1-bisphosphonate (9): To a suspension of NaH (60% suspension in mineral oil, 900 mg, 22.0 mmol) in dry THF (20 mL) was added dropwise tetraethyl methylenebisphosphonate (6.46 g, 22.4 mmol). The resulting clear solution was stirred 15 min at room temperature, after which 2-(3-bromopropoxy)tetrahydro-2H-pyran (5.05 g, 22.6 mmol) was added dropwise. The reaction mixture was heated to reflux for 6 h, diluted with CH₂Cl₂ (75 mL) and washed with brine (2×50 mL), dried (MgSO₄) and evaporated. It was used as such in the following step.

Tetraethyl 4-hydroxybutylene-1,1-bisphosphonate (10): To a stirred solution of the crude product 9 (max. 22.4 mmol) in MeOH (40 mL) was added Amberlite IR-120 (0.6 g). The reaction mixture was heated to 50° C. for 4 h, filtered and evaporated. The crude product was purified by flash chromatography on silica gel with gradient elution from 5-10% methanol/ethyl acetate to give pure 10 (2.67 g, 34% from tetraethyl methylenebisphosphonate). ¹H NMR (400 MHz, CDCl₃) δ 1.34 (t, J=7.1 Hz, 12H), 1.81 (quint, J=6.5 Hz, 2H), 1.99-2.13 (m, 2H), 2.37 (tt, J=24.4, 5.6 Hz, 1H), 2.51 (t, J=5.9 Hz, 2H), 3.66 (q, J=5.9 Hz, 2H), 4.13-4.22 (m, 8H).

Tetraethyl 4-iodobutylene-1,1-bisphosphonate (11): To a solution of 10 (1.52 g, 4.39 mmol) in CH₂Cl₂ (50 mL) were added triphenylphosphine (1.32 g, 5.033 mmol) and imidazole (0.45 g, 6.61 mmol). The reaction mixture was cooled to 0° C., before the addition of iodine (1.22 g, 4.81 mmol). The mixture was then removed from the cooling bath, stirred for 2 h, diluted with hexanes (100 mL) and filtered washing the precipitate with further hexanes (2×30 mL). The filtrate was evaporated and purified by flash chromatography on silica gel with gradient elution from 0-10% methanol/ethyl acetate to give pure 11 (1.6 g, 80%). ¹H NMR (400 MHz, CDCl₃) δ 1.32-1.38 (m, 12H), 1.95-2.15 (m, 4H), 2.28 (tt, J=24.1, 6.1, 1H), 3.18 (t, J=6.6, 2H), 4.12-4.24 (m, 8H).

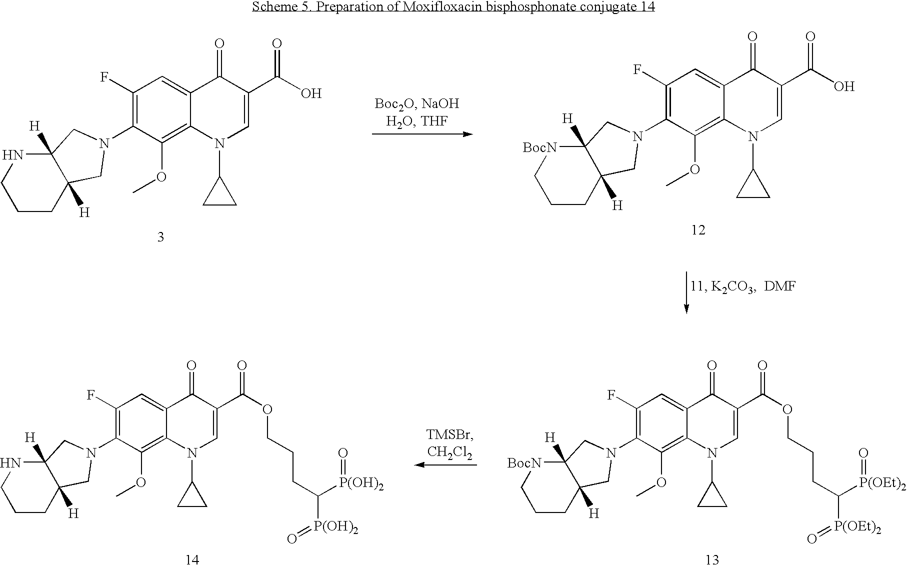

7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylic acid (12): The mixture of moxifloxacin (3, 834 mg, 2.078 mmol), Boc₂O (459.1 mg, 2.082 mmol) and 4.2 mL of 1 M NaOH aqueous solution in 20 mL of THF was stirred at room temperature overnight. After the removal of the organic solvent, the residue was neutralized with saturated ammonium chloride aqueous solution. The mixture was extracted with ethyl acetate (3×) and dried over anhydrous sodium sulfate. Removal of the solvent yielded a yellow foam 12 (947 mg, 91%) that contained trace amount of impurity as indicated by ¹H NMR and was used directly in the next step without purification. ¹H NMR (400 MHz, CDCl₃): [symbol] 0.79-0.86 (m, 1H), 1.03-1.18 (m, 2H), 1.23-1.34 (m, 2H), 1.44-1.54 (m, 1H), 1.49 (s, 9H), 1.76-1.84 (m, 2H), 2.25-2.29 (m, 1H), 2.89 (t, J=11.8, 1H), 3.22-3.30 (m, 1H), 3.38 (bs, 1H), 3.57 (s, 3H), 3.88 (dt, J=2.7, 10.0, 1H), 3.96-4.01 (m, 1H), 4.07-4.12 (m, 2H), 4.79 (bs, 1H), 7.82 (d, J=13.7, 1H), 8.79 (s, 1H) ppm.

4,4-bis(diethylphosphono)butyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (13): A mixture of 12 (576 mg, 1.15 mmol), iodo bisphosphonate 11 (497 mg, 1.09 mmol) and potassium carbonate (151 mg, 1.09 mmol) in 10 mL anhydrous DMF was stirred at room temperature for 21 h. Ethyl acetate (100 mL) was added, and the organics extracted with water (3×20 mL) and brine (20 mL), and dried over MgSO₄. Flash chromatography on silica gel with gradient elution from 5-10% methanol/ethyl acetate afforded the pure product (518 mg, 57%). ¹H NMR (400 MHz, CDCl₃) δ 0.72-0.80 (m, 1H), 0.92-1.10 (m, 1H), 1.16-1.30 (m, 2H), 1.33 (t, J=7.0, 12H), 1.47 (s, 9H), 1.71-1.83 (m, 4H), 2.05-2.16 (m, 4H), 2.18-2.28 (m, 1H), 2.32-2.50 (m, 1H), 2.81-2.94 (m, 1H), 3.13-3.26 (m, 1H), 3.28-3.42 (m, 1H), 3.55 (s, 3H), 3.78-3.90 (m, 2H), 3.98-4.08 (m, 2H), 4.12-4.23 (m, 8H), 4.25-4.36 (m, 2H), 4.76 (bs, 1H), 7.79 (d, J=4.3, 1H), 8.51 (s, 1H).

4,4-bisphosphonobutyl 7-((4aS,7aS)-octahydropyrrolo[3,4-b]pyridin-6-yl)-1-cyclopropyl-6-fluoro-1,4-dihydro-8-methoxy-4-oxoquinoline-3-carboxylate (14): TMSBr (0.82 mL, 6.21 mmol) was added in one portion to a stirring solution of 13 (518 mg, 0.624 mmol) in CH₂Cl₂ (50 mL) and the resulting mixture was stirred at room temperature for 23 h. The solvent was removed under reduced pressure and solid was dried under high vacuum for 1 h. The solid was then re-suspended in H₂O (200 mL) and the pH was immediately adjusted to pH 7 by the addition of 1M NaOH, with concomitant dissolution of the product. The product solution was washed with CHCl₃ (2×50 mL), filtered and evaporated to give the product in quantitative yield. ¹H NMR (400 MHz, D₂O) δ 0.83 (bs, 1H), 0.98 (bs, 1H), 1.08 (bs, 1H), 1.21 (bs, 1H), 1.65-2.15 (m, 9H), 2.61 (bs, 1H), 2.93 (bs, 1H), 3.20-3.35 (m, 1H), 3.43-3.64 (m, 2H), 3.52 (s, 3H), 3.75 (bs, 2H), 3.84-4.17 (m, 2H), 4.31 (bs, 2H), 7.41 (d, J=12.1, 1H), 8.80 (s, 1H).

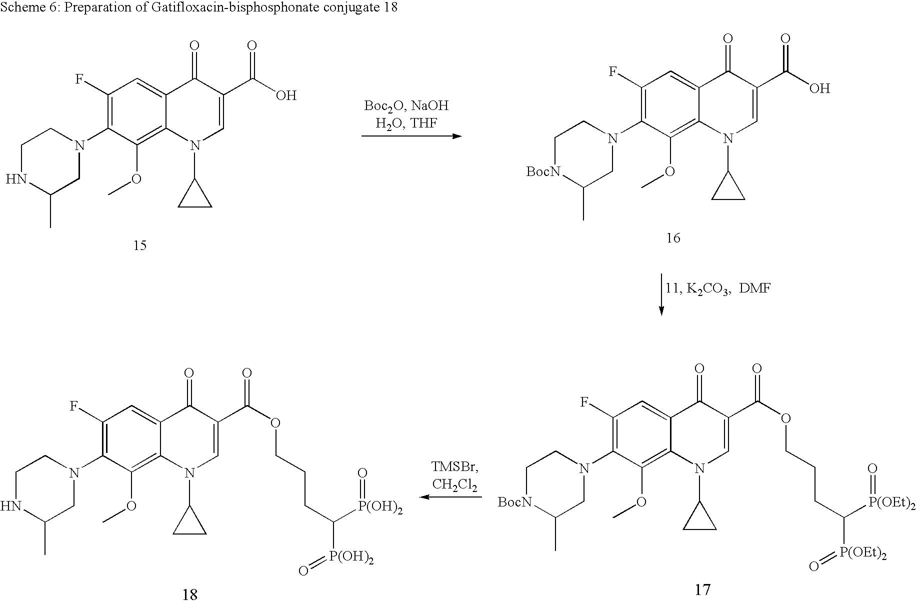

7-(4-(tert-butoxycarbonyl)-3-methylpiperazin-1-yl)-1-cyclopropyl-6-fluoro-1,4-dihydro-8-methoxy-4-oxoquinoline-3-carboxylic acid (16): The mixture of gatifloxacin (15, 335.1 mg, 0.8927 mmol), Boc₂O (202 mg, 0.9163 mmol) and 1.9 mL of 1 M NaOH aqueous solution in 10 mL of THF was stirred at room temperature overnight. After the removal of the organic solvent, the residue was neutralized with saturated ammonium chloride aqueous solution. The mixture was extracted with ethyl acetate (3×) and dried over anhydrous sodium sulfate. Removal of the solvent yielded a white solid 16 (403 mg, 95%). ¹H NMR (400 MHz, CDCl₃): [symbol] 0.94-1.04 (m, 2H), 1.19-1.26 (m, 2H), 1.33 (d, J=6.9, 3H), 1.50 (s, 9H), 3.23-3.37 (m, 3H), 3.44-3.51 (m, 2H), 3.73 (s, 3H), 3.95-4.03 (m, 2H), 4.36 (bs, 1H), 7.89 (d, J=11.4, 1H), 8.83 (s, 1H) ppm.

4,4-bis(diethylphosphono)butyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (17): A mixture of 16 (476 mg, 1.00 mmol), iodo bisphosphonate 11 (465 mg, 1.02 mmol) and potassium carbonate (180 mg, 1.30 mmol) in 10 mL anhydrous DMF was stirred at room temperature for 22 h. The solvent was evaporated at 70° C., and the residue purified by flash chromatography (2×) on silica gel with 5% methanol/CH₂Cl₂ to give pure 17 (460 mg, 57%). ¹H NMR (400 MHz, CDCl₃) δ 0.86-0.96 (m, 2H), 1.08-1.18 (m, 2H), 1.28-1.40 (m, 15H), 1.50 (s, 9H), 2.00-2.16 (m, 4H), 2.32-2.49 (m, 1H), 3.17-3.50 (m, 5H), 3.71 (s, 3H), 3.84-3.98 (m, 2H), 4.12-4.24 (m, 8H), 4.28-4.38 (m, 3H), 7.86 (d, J=12.3, 1H), 8.54 (s, 1H).

4,4-bisphosphonobutyl 7-(3-methylpiperazin-1-yl)-1-cyclopropyl-6-fluoro-1,4-dihydro-8-methoxy-4-oxoquinoline-3-carboxylate (18): TMSBr (0.76 mL, 5.76 mmol) was added in one portion to a stirring solution of 17 (460 mg, 0.573 mmol) in CH₂Cl₂ (50 mL) and the resulting mixture was stirred at room temperature for 15 h. The solvent was removed under reduced pressure and solid was dried under high vacuum for 1 h. The solid was then re-suspended in H₂O (200 mL) and the pH was immediately adjusted to pH 7.35 by the addition of 1M NaOH, with concomitant dissolution of the product. The product solution was washed with CHCl₃ (2×100 mL), filtered and evaporated to give the crude product (300 mg, 77% recovery based on tetrasodium salt of product). The crude material was purified on a C18 Sep-Pak™ (H₂O) to give pure 18 (89 mg, 23%). ¹H NMR (400 MHz, D₂O) δ 0.88-1.03 (m, 2H), 1.10-1.24 (m, 2H), 1.36 (d, J=6.3, 3H), 1.80-2.12 (m, 5H), 3.18-3.61 (m, 7H), 3.72 (s, 3H), 4.02-4.15 (m, 1H), 4.34 (t, J=6.6, 2H), 7.48 (d, J=12.3, 1H), 8.83 (s, 1H).

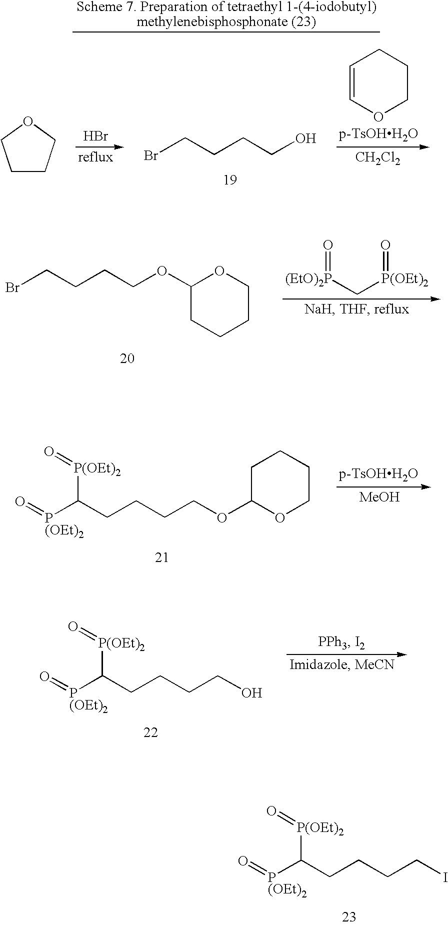

4-Bromo-1-butanol (19): To 67.5 mL (832.2 mmol) of refluxing tetrahydrofuran was added 31 mL (274 mmol) of 48% hydrobromic acid dropwise and the yellow solution was allowed to reflux for another 2 h. After cooled to room temperature, the reaction was carefully neutralized with saturated sodium bicarbonate aqueous solution. The resultant mixture was extracted with diethyl ether (3×) and dried over anhydrous sodium sulfate. Removal of the solvent afforded the product 19 as a yellow oil (10.7 g, 26%). ¹H NMR (400 MHz, CDCl₃): [symbol] 1.69-1.76 (m, 2H), 2.01-1.94 (m, 2H), 3.46 (t, J=6.6, 2H), 3.70 (t, J=6.4, 2H).

2-(4-Bromobutoxy)-tetrahydro-2H-pyran (20): 3,4-Dihydro-2H-pyran (8.5 mL, 90.96 mmol) was added dropwise to the dichloromethane (20 mL) solution of 19 (10.7 g, 69.93 mmol) and p-toluenesulfonic acid monohydrate (26.5 mg, 0.1372 mmol). The mixture was stirred at room temperature over night. After removing the solvent, the residue was purified by flash chromatography on silica gel with 5:1 hexanes/ethyl acetate as the eluent to yield product 20 as a colorless oil (15.3 g, 92%). ¹H NMR (400 MHz, CDCl₃): [symbol] 1.48-1.62 (m, 4H), 1.68-1.85 (m, 4H), 1.94-2.02 (m, 2H), 3.40-3.53 (m, 4H), 3.74-3.88 (m, 2H), 4.57-4.59 (m, 1H).

Tetraethyl 5-(2-Tetrahydro-2H-pyranyloxy)pentylene-1,1-bisphosphonate (21): To the suspension of sodium hydride (60%, 840.5 mg, 21.01 mmol) in 40 mL of THF was carefully added tetraethyl methylenebisphosphonate (6.16 g, 20.95 mmol) and the resultant pale yellow clear solution was stirred at room temperature for 45 min. Then the bromide 20 (4.97 g, 20.96 mmol) was introduced plus 5 mL of THF rinse. The reaction was brought to reflux overnight and allowed to cool to room temperature before being quenched with saturated ammonium chloride aqueous solution. Another small amount of water was required to dissolve the solid. The mixture was extracted with ethyl acetate (3×), dried over anhydrous sodium sulfate and concentrated in vacuo. Flash chromatography on silica gel with 20:1 (v/v) dichloromethane/methanol as the eluent afforded 7.3 g of impure product 21 as a slightly yellow oil. The material was used directly in the next step without further purification. Selected ¹H NMR signals (400 MHz, CDCl₃): δ 2.28 (tt, J=6.1, 24.3, 1H), 3.37-3.51 (m, 2H), 3.71-3.89 (m, 2H), 4.56-4.58 (m, 1H).

Tetraethyl 5-hydroxypentylene-1,1-bisphosphonate (22): The crude compound 21 was dissolved in 20 mL of methanol and 74.6 mg (0.3863 mmol) of p-toluenesulfonic acid monohydrate was added. After overnight stirring at room temperature, the mixture was concentrated and subjected to flash chromatography with gradient elution from 15:1 ethyl acetate/methanol to 8:1 then 6:1 to afford a colorless oil (3.1 g, 41% over two steps). ¹H NMR (400 MHz, CDCl₃): δ 1.24-1.36 (m, 12H), 1.55-1.72 (m, 4H), 1.89-2.03 (m, 2H), 2.16 (bs, 1H), 2.29 (tt, J=6.1, 24.3, 1H), 3.66 (bs, 2H), 4.11-4.22 (m, 8H).

Tetraethyl 5-iodopentylene-1,1-bisphosphonate (23): The alcohol 22 (1.419 g, 3.938 mmol), triphenylphosphine (1.25 g, 4.718 mmol) and imidazole (325.6 mg, 4.735 mmol) were dissolved in 15 mL of dry acetonitrile, and 1.196 g (4.703 mmol) of I₂ was added in several portions. After overnight stirring at room temperature, the solvent was removed in vacuo and the residue was taken up in ethyl acetate and saturated Na₂S₂O₃ aqueous solution. The mixture was stirred until the organic layer turned pale yellow and the two phases were separated. The organic phase was dried over anhydrous sodium sulfate and concentrated. Flash chromatography on silica gel with 15:1 ethyl acetate/methanol as the eluent afforded the product 23 as a yellow oil (1.26 g, 68%). ¹H NMR (400 MHz, CDCl₃): δ1.36 (t, J=7.0, 12H), 1.66-1.72 (m, 2H), 1.81-1.99 (m, 4H), 2.35 (tt, J=5.9, 24.1, 1H), 3.20 (t, J=6.9, 2H), 4.17-4.23 (m, 8H).

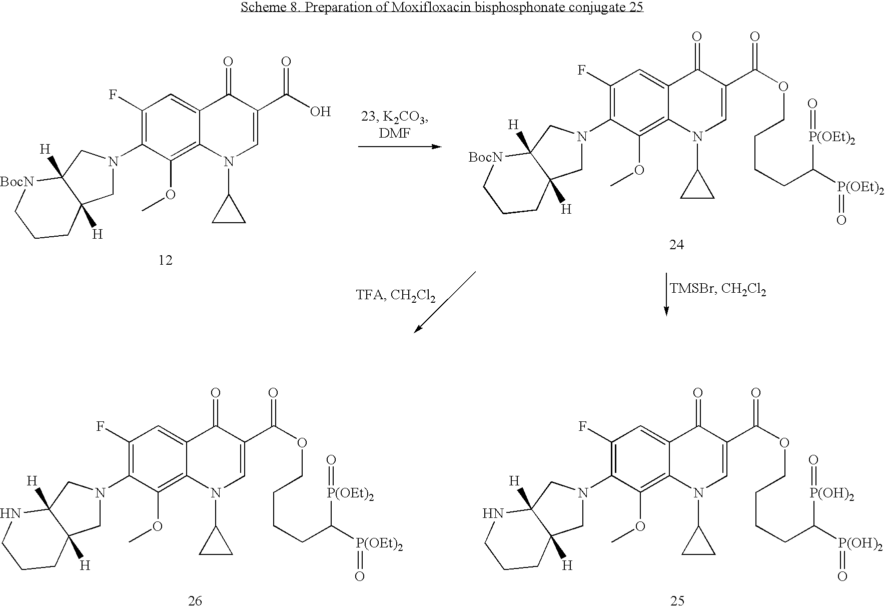

5,5-bis(diethylphosphono)pentyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (24): The mixture containing compound 12 (464.7 mg, 0.9265 mmol), iodo bisphosphonate 23 (435.5 mg, 0.9262 mmol) and potassium carbonate (129.3 mg, 0.9355 mmol) in 15 mL of anhydrous DMF was heated at 65° C. for 2 days. After cooling to room temperature, the reaction was diluted with water, extracted with ethyl acetate (3×), dried over anhydrous sodium sulfate and concentrated. Flash chromatography on silica gel with gradient elution from 15:1 ethyl acetate/methanol to 8:1 then 5:1 afforded 425.6 mg (54%) product 24 as a yellow foam. ¹H NMR (400 MHz, CDCl₃): [symbol] 0.72-0.80 (m, 1H), 1.00-1.08 (m, 2H), 1.24-1.36 (m, 13H), 1.48 (s, 9H), 1.63-1.81 (m, 8H), 1.92-2.04 (m, 2H), 2.20-2.28 (m, 1H), 2.36 (tt, J=5.8, 24.1, 1H), 2.82-2.92 (m, 1H), 3.16-3.24 (m, 1H), 3.30-3.38 (m, 1H), 3.56 (s, 3H), 3.80-3.85 (m, 1H), 3.89-3.94 (m, 1H), 4.01-4.08 (m, 2H), 4.12-4.21 (m, 8H), 4.29-4.36 (m, 2H), 4.77 (bs, 1H), 7.78 (d, J=14.4, 1H), 8.55 (s, 1H).

5,5-bisphosphonopentyl 7-((4aS,7aS)-octahydropyrrolo[3,4-b]pyridin-6-yl)-1-cyclopropyl-6-fluoro-1,4-dihydro-8-methoxy-4-oxoquinoline-3-carboxylate (25): To a solution of compound 24 (377.6 mg, 0.4475 mmol) in 5 mL of CH₂Cl₂ was added 0.61 mL (4.529 mmol) of bromotrimethylsilane. The mixture was stirred at room temperature overnight before being concentrated. The residue was kept on high vacuum for at least 30 min and then dissolved in water. The resulting solution was brought to pH 7.4 with 1 N sodium hydroxide aqueous solution and the solvent was removed. The solid was twice dissolved in water and the solvent removed. The solid obtained was subjected to a Waters® C18 Sep-Pak™ cartridge (20 cc) with gradient elution from neat water to 10:1 water/methanol to 5:1 to afford product 25 as an off-white solid (211 mg, 70%). ¹H NMR (400 MHz, D₂O): [symbol] 0.78-0.84 (m, 1H), 0.97-1.10 (m, 2H), 1.17-1.24 (m, 1H), 1.62-2.00 (m, 11H), 2.81 (bs, 1H), 3.04-3.10 (m, 1H), 3.39-3.43 (m, 1H), 3.54 (s, 3H), 3.63-3.68 (m, 2H), 3.80 (dt, J=3.3, 9.6, 1H), 3.92-3.94 (m, 1H), 4.05-4.08 (m, 2H), 4.25-4.28 (m, 2H), 7.47 (d, J=14.1, 1H), 8.76 (s, 1H); ³¹P NMR (162 MHz, D₂O): δ 21.45; ¹⁹F NMR (376 MHz, D₂O): [symbol] -121.64 (d, J=13.8); MS (m/e): 630 (M−H).

5,5-bis(diethylphosphono)pentyl 7-((4aS,7aS)-octahydropyrrolo[3,4-b]pyridin-6-yl)-1-cyclopropyl-6-fluoro-1,4-dihydro-8-methoxy-4-oxoquinoline-3-carboxylate, trifluoroacetate salt (26): Trifluoroacetic acid (0.5 mL) was added to a solution of compound 23 (90.7 mg, 0.1075 mmol) in 3 mL of CH₂Cl₂. The reaction mixture was stirred at room temperature for 1 h, quenched with saturated sodium bicarbonate aqueous solution and the aqueous layer was extracted with CH₂Cl₂ (2×). The combined organic phases were subsequently washed with 1 N sodium hydroxide solution (1×) and water (2×) and dried over anhydrous sodium sulfate. The pure product 26 was obtained from semi-preparative HPLC as a sticky oil. ¹H NMR (400 MHz, CDCl₃): [symbol] 0.79-0.83 (m, 1H), 1.00-1.07 (m, 2H), 1.16-1.20 (m, 1H), 1.33 (t, J=7.2, 12H), 1.71-1.84 (m, 8H), 1.93-2.01 (m, 2H), 2.22-2.36 (m, 2H), 2.69-2.74 (m, 1H), 3.05-3.08 (m, 1H), 3.31-3.34 (m, 1H), 3.38-3.44 (m, 2H), 3.55 (s, 3H), 3.86-3.97 (m, 3H), 4.13-4.22 (m, 8H), 4.30 (dt, J=2.7, 6.9, 2H), 7.77 (d, J=14.3, 1H), 8.50 (s, 1H); ¹⁹F NMR (376 MHz, CDCl₃): [symbol] -123.61, −75.80.

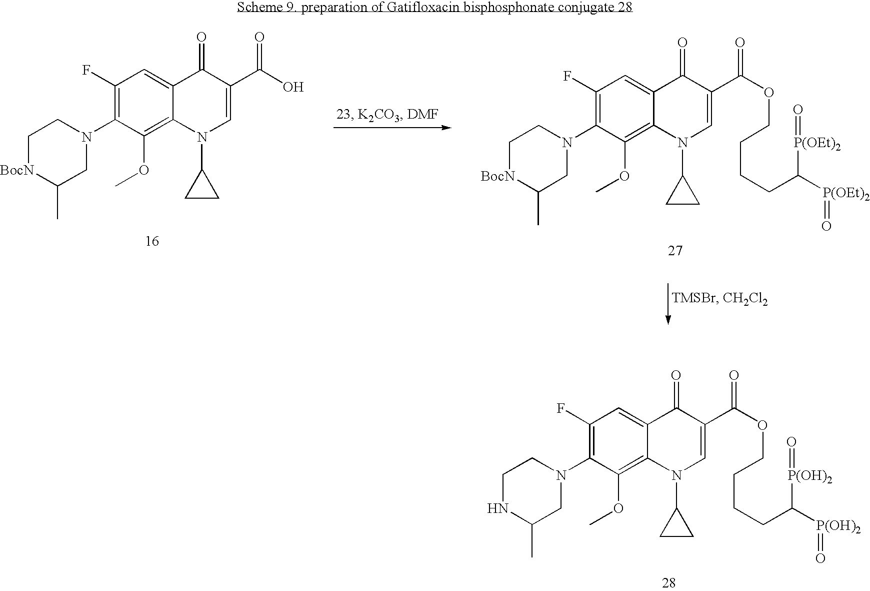

5,5-bis(diethylphosphono)pentyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (27): A mixture containing compound 16 (486 mg, 1.022 mmol), iodo bisphosphonate 23 (481.4 mg, 1.024 mmol) and potassium carbonate (144.3 mg, 1.044 mmol) in 15 mL of anhydrous DMF was heated at 65° C. for 2 days. After cooling to room temperature, the reaction was diluted with water, extracted with ethyl acetate (3×), dried over anhydrous sodium sulfate and concentrated. Flash chromatography on silica gel with gradient elution from 12:1 ethyl acetate/methanol to 10:1, 8:1 then 6:1 afforded 455.3 mg (54%) of product 27 as a brown oil. ¹H NMR (400 MHz, CDCl₃): [symbol] 0.88-0.96 (m, 2H), 1.10-1.17 (m, 2H), 1.32-1.40 (m, 15H), 1.50 (s, 9H), 1.67-1.82 (m, 4H), 1.93-2.05 (m, 2H), 2.30 (tt, J=6.1, 24.1, 1H), 3.18-3.45 (m, 5H), 3.72 (s, 3H), 3.86-3.96 (m, 2H), 4.14-4.22 (m, 8H), 4.29-4.38 (m, 3H), 7.88 (d, J=12.3, 1H), 8.56 (s, 1H).

5,5-bisphosphonopentyl 7-(3-methylpiperazin-1-yl)-1-cyclopropyl-6-fluoro-1,4-dihydro-8-methoxy-4-oxoquinoline-3-carboxylate (28): To a solution of compound 27 (479.4 mg, 0.5862 mmol) in 5 mL of CH₂Cl₂ was added 0.79 mL (5.866 mmol) of bromotrimethylsilane. The mixture was stirred at room temperature overnight before being concentrated. The residue was kept at high vacuum for at least 30 min and then dissolved in water. The resulting solution was brought to pH 7.1 with 1 N sodium hydroxide aqueous solution and the solvent was removed. The solid was twice dissolved in water and the solvent removed in vacuo. The solid obtained was subjected to a Waters® C18 Sep-Pak™ cartridge (20 cc) with gradient elution from neat water to 2:1 water/methanol to 1:2 to methanol to afford product 28 as an off-white solid (203 mg, 50%). ¹H NMR (400 MHz, D₂O): [symbol] 0.96-1.02 (m, 2H), 1.14-1.20 (m, 2H), 1.35 (d, J=6.5, 3H), 1.64-1.71 (m, 2H), 1.77-1.92 (m, 5H), 3.26-3.38 (m, 2H), 3.43-3.66 (m, 5H), 3.79 (s, 3H), 4.09-4.15 (m, 1H), 4.31 (t, J=6.9, 2H), 7.66 (d, J=12.3, 1H), 8.83 (s, 1H); ³¹P NMR (162 MHz, D₂O): [symbol] 21.24; ¹⁹F NMR (376 MHz, D₂O): [symbol] 121.86 (d, J=12.6); MS (m/e): 604 (M−H).

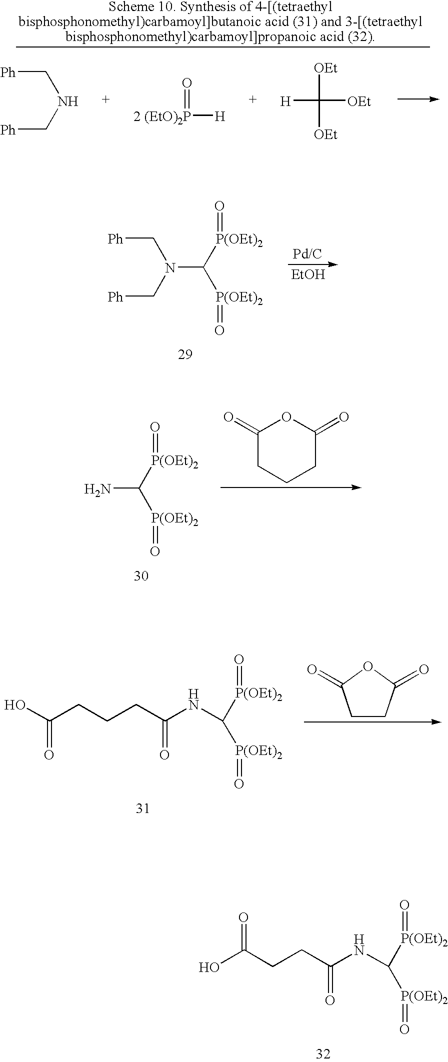

Tetraethyl N,N-dibenzyl-1-aminomethylenebisphosphonate (29): Compound 29 was prepared according to a modified protocol derived from Synth. Comm. 1996, 26, 2037-2043. Triethyl orthoformate (8.89 g, 60 mmol), diethyl phosphite (16.57 g, 120 mmol) and dibenzyl amine (11.80 g, 60 mmol) were combined in a 100 mL round bottom flask fitted with a distillation head. The reaction was heated to a temperature of 180-195° C. for 1 h under Ar. When EtOH evolution was complete, the reaction mixture was cooled to room temperature, diluted with CHCl₃ (300 mL), washed with aqueous NaOH (2M, 3×60 mL) and brine (2×75 mL), then dried over MgSO₄. After evaporation, a crude yield of 25.2 g (87%) was obtained. A 4.95 g portion of the crude oil was purified by chromatography (ethyl acetate:hexane:methanol 14:4:1) to yield pure 29 (2.36 g, 41%). ¹H NMR (400 MHz, CDCl₃) δ1.32 (dt, J=2.0, 7.0, 12H), 3.55 (t, J=25.0, 1H), 3.95-4.25 (m, 12H), 7.20-7.45 (m, 10H).

Tetraethyl 1-aminomethylenebisphosphonate (30): Compound 29 (2.00 g, 4.14 mmol) was dissolved in EtOH (40 mL). To this solution was added palladium on carbon (10%, 1.5 g) and cyclohexene (2.5 mL, 24.7 mmol). The reaction mixture was refluxed under argon for 15 hours, filtered through celite and evaporated to give 30 as a slightly impure pale yellow oil (1.50 g, 119%), which was used directly in the next step without further purification. ¹H NMR (400 MHz, CDCl₃) δ 1.35 (t, J=7.0, 12H), 3.58 (t, J=20.3, 1H), 3.65-3.90 (br s, 2H), 4.20-4.28 (m, 8H).

4-[(tetraethylbisphosphonomethyl)carbamoyl]butanoic acid (31): Compound 31 was prepared as described in J. Drug Targeting, 1997, 5, 129-138. It was obtained as an orange oil, in 85% crude yield from 30. The crude product could be purified by chromatography (10% AcOH/EtOAc) to give a white solid. ¹H NMR (400 MHz, CDCl₃): δ 1.30 (t, J=7.0, 6H), 1.34 (t, J=7.0, 6H), 1.92-2.02 (m, 2H), 2.38-2.44 (m, 2H), 2.54 (t, J=7.3, 1H), 4.04-4.28 (m, 8H), 5.16 (td, J=22.1, J=10.0, 1H), 8.45 (d, J=10.2, 1H).

3-[(tetraethylbisphosphonomethyl)carbamoyl]propanoic acid (32): Compound 32 was prepared as described in J. Drug Targeting, 1997, 5, 129-138. It was obtained as an oil which slowly solidified, in 57% crude yield from 30. The crude product could be purified by chromatography (10% AcOH/EtOAc) to give a white solid. ¹H NMR (400 MHz, CDCl₃): δ 1.31 (t, J=7.0, 6H), 1.33 (t, J=7.1, 6H), 2.61-2.73 (m, 4H), 4.05-4.28 (m, 8H), 5.07 (td, J=21.6, J=9.8, 1H), 7.90 (d, J=9.4, 1H).

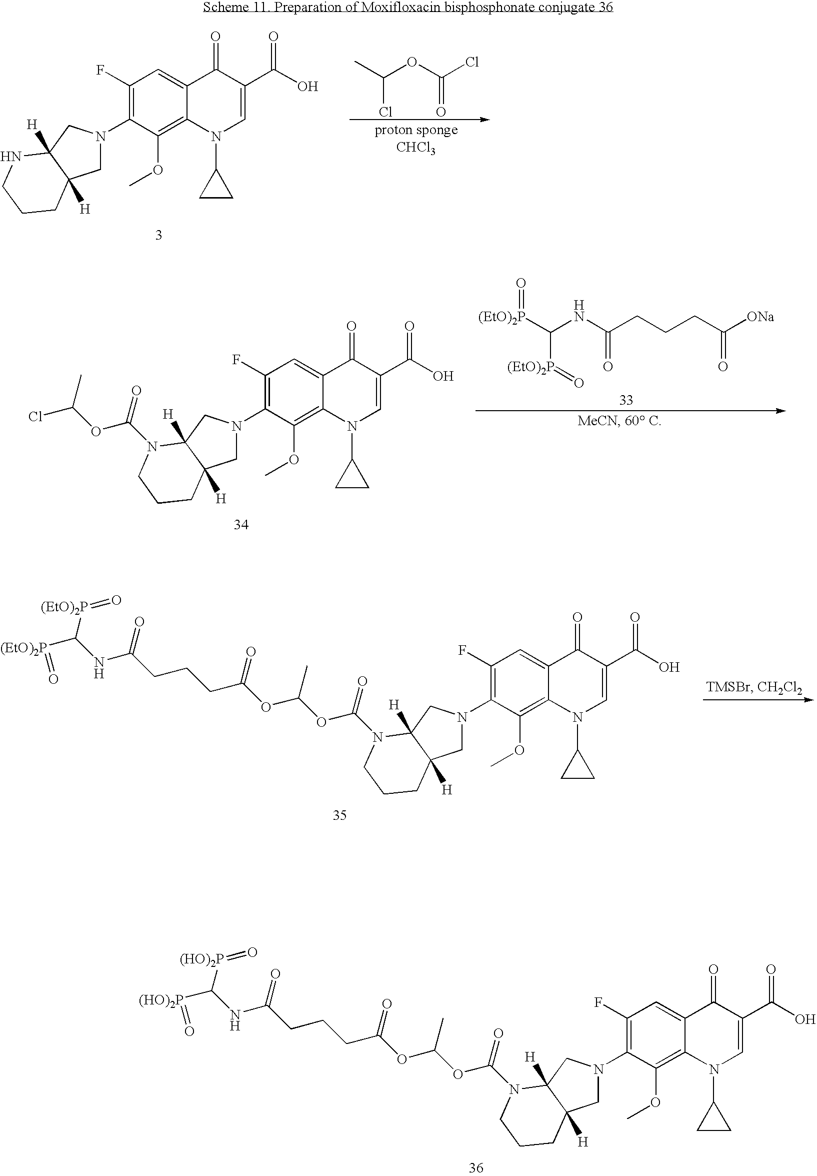

Sodium salt of 4-[((tetraethylbisphosphonomethyl)carbamoyl]butanoic acid (33): The carboxylic acid 31 (300.2 mg, 0.7193 mmol) was dissolved into 2 mL of THF and 0.72 mL (0.72 mmol) of 1 N sodium hydroxide aqueous solution was added. The mixture was stirred at room temperature for 4 h and the organic solvent was removed. The residual water was removed either by applying high vacuum overnight or by freeze drying. The resultant solid was used directly in the next step. 7-((4aS,7aS)-1-((1-chloroethoxy)carbonyl)-octahydropyrrolo[3,4-b]pyridin-6-yl)-1-cyclopropyl-6-fluoro-1,4-dihydro-8-methoxy-4-oxoquinoline-3-carboxylic acid (34): 1-Chloroethyl chloroformate (0.27 mL, 2.478 mmol) was added to a solution of moxifloxacin 3 (994.5 mg, 2.477 mmol) and 547.9 mg (2.557 mmol) of proton sponge in 25 mL chloroform. The clear yellow solution was stirred at room temperature for 5 h before being washed with water (3×) and dried over anhydrous sodium sulfate. Removal of the solvent yielded product 34 as a yellow foam (1.228 g, 98%). In the cases where proton sponge was still present after the water wash, the crude product was passed through a short silica gel column with the elution of 19:1 dichloromethane/methanol. ¹H NMR (400 MHz, CDCl₃): [symbol] 0.78-0.86 (m, 1H), 1.03-1.17 (m, 2H), 1.25-1.35 (m, 1H), 1.47-1.64 (m, 1H), 1.78-1.90 (m, 5H), 2.32 (bs, 1H), 2.95-3.05 (m, 1H), 3.24-3.64 (m, 2H), 3.58 (s, 3H), 3.86-4.03 (m, 2H), 4.04-4.22 (m, 2H), 4.70-4.98 (m, 1H), 6.63 (q, J=5.9, 1H), 7.82 (dd, J=1.6, 13.7, 1H), 8.79 (s, 1H).

Mixed acetal 35: A mixture of 34 (741.4 mg, 1.460 mmol) and 33 (1.454 mmol) in 7 mL of anhydrous acetonitrile was heated in a 60° C. oil bath for 2 days. After cooling to room temperature, the reaction mixture was filtered through a pad of celite. The filtrate was concentrated and subjected to a Waters® C18 Sep-Pak™ cartridge (35 cc) with gradient elution from neat water to 2:1 water/methanol to 1:2 to methanol. The pure product was obtained as a brown glassy solid (556.3 mg, 43%). ¹H NMR (400 MHz, CDCl₃): [symbol] 0.78-0.86 (m, 1H), 1.04-1.20 (m, 2H), 1.28-1.36 (m, 12H), 1.46-1.64 (m, 4H), 1.74-2.03 (m, 5H), 2.26-2.44 (m, 4H), 2.90-3.02 (m, 1H), 3.22-3.50 (m, 3H), 3.58 (s, 3H), 3.84-4.03 (m, 3H), 4.03-4.28 (m, 8H), 4.64-4.94 (bs, 1H), 5.02 (dt, J=10.0, 21.9, 1H), 6.15 (d, J=10.2, 1H), 6.84 (q, J=4.7, 1H), 7.82 (d, J=13.9, 1H), 8.79 (s, 1H).

Mixed acetal 36: To a solution of tetraester 35 (556 mg, 0.6256 mmol) in 5 mL of CH₂Cl₂ was added 0.83 mL (6.289 mmol) of bromotrimethylsilane. After stirring at room temperature for 6 h, the mixture was concentrated and the residue was kept at high vacuum for at least 30 min. The resulting material was dissolved in dilute sodium hydroxide solution (≦2 eq of NaOH, this process was fairly time-consuming and ultra-sound sonication was needed from time to time to maintain the solution) prior to the careful adjustment of pH to 7.20 with 1 N sodium hydroxide solution. The aqueous solution obtained was subjected to a Waters® C18 Sep-Pak™ cartridge (20 cc) with gradient elution from neat water to 10:1 water/methanol. All fractions containing the desired product were immediately combined and frozen in an acetone/dry ice cold bath. The solvents were removed by freeze drying and the material obtained was washed with CH₂Cl₂ to yield 90 mg (18%) of product 36 as an off-white powder. ¹H NMR (400 MHz, D₂O): [symbol] 0.75-0.83 (m, 1H), 0.96-1.04 (m, 1H), 1.04-1.13 (m, 1H), 1.18-1.27 (m, 1H), 1.46-1.60 (m, 2H), 1.52 (d, J=5.5, 3H), 1.73-1.86 (m, 2H), 1.93 (quint, J=7.6, 2H), 2.28-2.40 (m, 1H), 2.38 (t, J=7.1, 2H), 2.49 (t, J=7.2, 2H), 2.98-3.12 (m, 1H), 3.30 (d, J=9.4, 1H), 3.43-3.53 (m, 1H), 3.59 (s, 3H), 3.90-4.10 (m, 4H), 4.24 (t, J=19.4, 1H), 6.77 (q, J=5.5, 1H), 7.64 (d, J=14.5, 1H), 8.46 (s, 1H) ppm; ³¹P NMR (162 MHz, D₂O): [symbol] 14.23 ppm; ¹⁹F NMR (376 MHz, D₂O): [symbol] −123.52 (bs) ppm; MS (m/e): 777 (M+H).

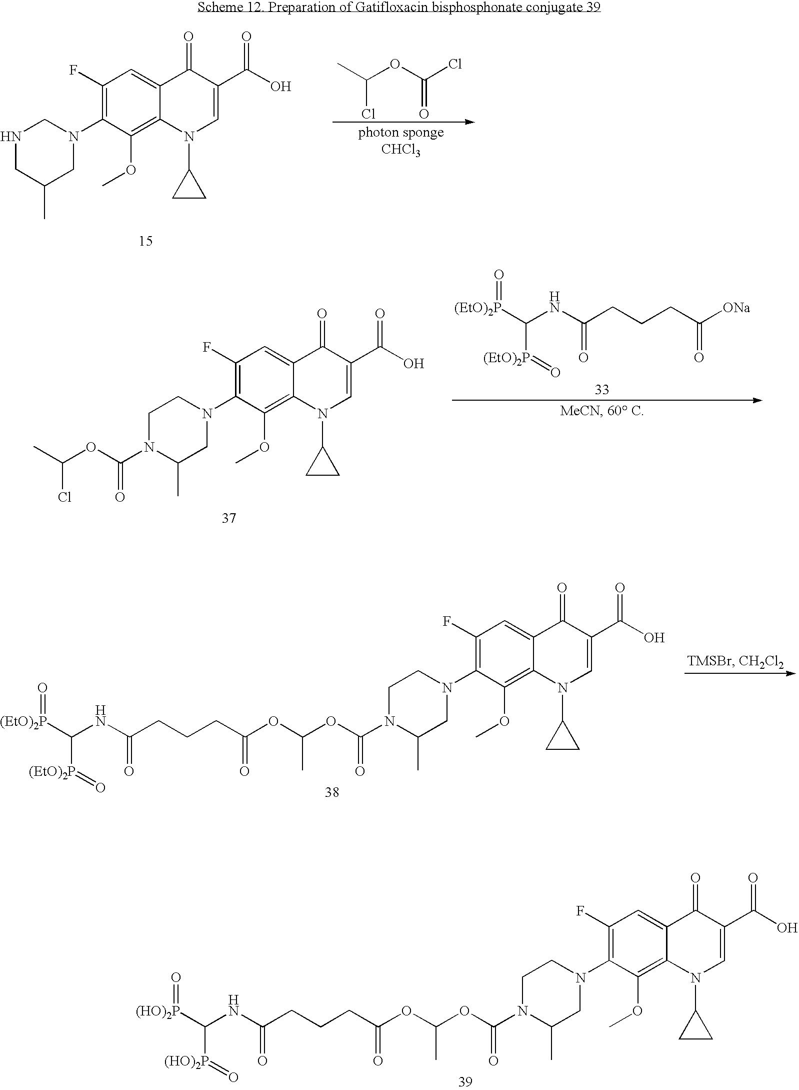

7-(4-((1-chloroethoxy)carbonyl)-3-methylpiperazin-1-yl)-1-cyclopropyl-6-fluoro-1,4-dihydro-8-methoxy-4-oxoquinoline-3-carboxylic acid (37): 1-Chloroethyl chloroformate (82 μL, 0.7525 mmol) was added to a solution of gatifloxacin 15 (282.8 mg, 0.7533 mmol) and proton sponge (166.6 mg, 0.7773 mmol) in 10 mL of CHCl₃. The white suspension quickly turned clear and was stirred at room temperature for another 2 h. The mixture was washed with water (3×) and dried over anhydrous sodium sulfate. Removal of the solvent yielded product 37 as a yellow solid (356.5 mg, 98%). In the cases where proton sponge was still present after water wash, the crude product was passed through a short silica gel column with the elution of 19:1 dichloromethane/methanol. ¹H NMR (400 MHz, CDCl₃): [symbol] 0.94-1.04 (m, 2H), 1.21-1.26 (m, 2H), 1.40 (d, J=7.1, 3H), 1.85 (d, J=5.9, 3H), 3.31-3.34 (m, 2H), 3.41-3.53 (m, 4H), 3.74 (s, 3H), 3.98-4.07 (m, 2H), 4.45 (bs, 1H), 6.65 (dq, J=1.8, 5.7, 1H), 7.92 (d, J=12.0, 1H), 8.84 (s, 1H).

Mixed acetal 38: A mixture of 37 (324.1 mg, 0.6725 mmol) and sodium carboxylate 33 (0.7193 mmol) in 5 mL of anhydrous acetonitrile was heated in a 60° C. oil bath for 2 days. After cooling to room temperature, the reaction mixture was filtered through a pad of celite. The filtrate was concentrated and subjected to a Waters® C18 Sep-Pak™ cartridge (20 cc) with gradient elution from neat water to 2:1 water/methanol to 1:2 to methanol. The pure product obtained was a brown sticky oil (247.6 mg, 43%). ¹H NMR (400 MHz, CDCl₃): [symbol] 0.96-1.04 (m, 2H), 1.16-1.29 (m, 2H), 1.34 (t, J=7.1, 12H), 1.52 (d, J=5.5, 3H), 1.69 (bs, 3H), 1.99 (quint, J=7.0, 2H), 2.35 (t, J=7.2, 2H), 2.42 (t, J=7.2, 2H), 3.22-3.53 (m, 5H), 3.74 (s, 3H), 3.98-4.04 (m, 2H), 4.14-4.24 (m, 8H), 4.36-4.44 (m, 1H), 5.03 (dt, J=10.2, 21.7, 1H), 6.18-6.21 (m, 1H), 6.86 (q, J=5.5, 1H), 7.91 (d, J=12.1, 1H), 8.83 (s, 1H).

Mixed acetal 39: To a solution of tetraester 38 (247.1 mg, 0.2864 mmol) in 6 mL of CH₂Cl₂ was added 0.40 mL (3.031 mmol) of bromotrimethylsilane. After stirring overnight at room temperature, the solvent was removed and the residue was kept on high vacuum for at least 30 min. The resulting material was dissolved in dilute sodium hydroxide solution (≦2 eq of NaOH, this process was fairly time-consuming and ultra-sound sonication was needed from time to time to help maintain the dissolution) prior to the careful adjustment of pH to 7.25 with 1 N sodium hydroxide solution. The aqueous solution obtained was subjected to a Waters® C18 Sep-Pak™ cartridge (20 cc) with gradient elution from neat water to 10:1 water/methanol. All fractions with the desired product were immediately combined and frozen in an acetone/dry ice cold bath. The solvents were removed by freeze drying and the material obtained was washed with dichloromethane to yield 65 mg (30%) of product 39 as an off-white powder. ¹H NMR (400 MHz, D₂O): [symbol] 0.89-1.00 (m, 2H), 1.08-1.17 (m, 2H), 1.37 (t, J=7.2, 3H), 1.53 (d, J=5.5, 3H), 1.93 (quint, J=7.2, 2H), 2.38 (t, J=7.6, 2H), 2.50 (t, J=7.0, 2H), 3.24-3.34 (m, 2H), 3.42-3.50 (m, 3H), 3.75 (s, 3H), 3.93 (bs, 1H), 4.06-4.12 (m, 1H), 4.22 (t, J=19.0, 1H), 4.34 (bs, 1H), 6.79 (q, J=5.5, 1H), 7.73 (d, J=12.7, 1H), 8.51 (s, 1H); ³¹P NMR (162 MHz, D₂O): [symbol] 14.27; ¹⁹F NMR (376 MHz, D₂O): [symbol] −122.32 (bs); MS (m/e): 751 (M+H).

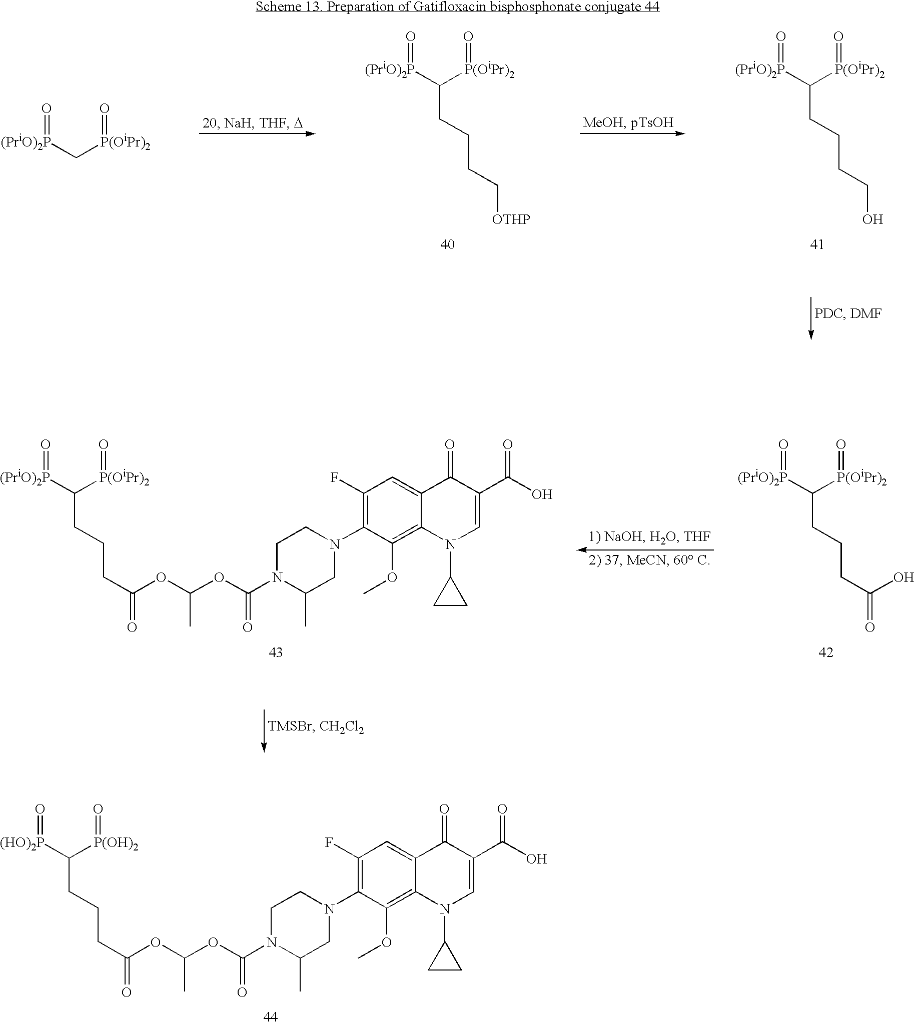

Tetraisopropyl 5-(2-tetrahydro-2H-pyranyloxy)-pentylene-1,1-bisphosphonate (40): To a suspension of sodium hydride (60%, 342.5 mg, 8.563 mmol) in 15 mL THF was carefully added tetraisopropyl methylenebisphosphonate (2.80 mL, 8.61 mmol), and the resultant pale yellow clear solution was stirred at room temperature for 30 min. Then neat compound 20 (2.0194 g, 8.516 mmol) was introduced by pipette plus 5 mL of THF rinse. The reaction was brought to reflux for 8 h and allowed to cool to room temperature before quenching with saturated NH₄Cl. The mixture was extracted with ethyl acetate (3×) and dried over sodium sulfate and concentrated in vacuo Flash chromatography with 10:1 EtOAc:MeOH as eluent recovered 760 mg of unreacted starting material 20. The desired product 40 was not isolable from the other unreacted starting material tetraisopropyl methylenebisphosphonate and the mixture was used directly in the next step. Selected ¹H NMR signals (400 MHz, CDCl₃) [symbol] 1.48-2.02 (m, 12H), 2.14 (tt, J=24.2, 5.9, 1H), 3.36-3.42 (m, 1H), 3.46-3.52 (m, 1H), 3.71-3.77 (m, 1H), 3.83-3.89 (m, 1H), 4.57-4.58 (m, 1H).

Tetraisopropyl 5-hydroxypentylene-1,1-bisphosphonate (41): The mixture from the flash chromatography in the previous step was dissolved in 4 mL of MeOH and 24.5 mg (0.127 mmol) p-toluenesulfonic acid monohydrate was added. After stirring overnight at room temperature, the mixture was concentrated and subjected to flash chromatography with 12:1 EtOAc:MeOH as eluent to afford 41 as a colorless oil (1.2 g, 50% over two steps). ¹H NMR (400 MHz, CDCl₃) δ 1.33-1.36 (m, 24H), 1.54-1.61 (m, 2H), 1.65-1.72 (m, 2H), 1.84-1.98 (m, 2H), 2.15 (tt, J=24.1, 6.1, 1H), 2.28 (t, J=5.7, 1H), 3.66 (q, J=6.1, 2H), 4.72-4.82 (m, 4H).

Tetraisopropyl 5-carboxypentylene-1,1-bisphosphonate (42): Compound 41 (365.5 mg, 0.9083 mmol) and pyridinium dichromate (1.22 g, 3.18 mmol) were dissolved in 3 mL N,N-dimethyl formamide and stirred at room temperature overnight. After the reaction was complete as monitored by TLC, the mixture was diluted with water and extracted with EtOAc (3×), dried over sodium sulfate and concentrated in vacuo. Flash chromatography on silica gel with 19:1 EtOAc:acetic acid afforded 42 as a colorless oil (246.8 mg, 65%). ¹H NMR (400 MHz, CDCl₃) δ 1.29-1.35 (m, 24H), 1.90-1.99 (m, 4H), 2.18 (tt, J=24.4, 5.5, 1H), 2.34 (t, J=6.8, 2H), 4.73-4.82 (m, 4H).

Mixed acetal 43: To 2 mL THF solution of tetraisopropyl bisphosphonate carboxylic acid 42 (277.4 mg, 0.6445 mmol) was added 0.65 mL (0.65 mmol) of 1N sodium hydroxide aqueous solution. After 4 h stirring at room temperature, the solvent was removed. A mixture of 244 mg (0.5394 mmoles) of the resultant solid and Gatifloxacin derivative 37 (239.6 mg, 0.4972 mmol) in 4 mL of anhydrous acetonitrile was heated on a 60° C. oil bath overnight. After cooling to room temperature, the mixture was filtered through a pad of celite. The filtrate was concentrated and subjected to a Waters® C18 Sep-Pak™ cartridge (20 cc) with gradient elution from neat water to 2:1 water/methanol to 1:2 to methanol. Removal of the solvent yielded product 43 as a sticky yellow oil (322 mg, 74%). ¹H NMR (400 MHz, CDCl₃): [symbol] 0.88-1.04 (m, 2H), 1.14-1.25 (m, 2H), 1.28-1.42 (m, 24H), 1.51 (d, J=5.5, 3H), 1.61 (bs, 3H), 1.86-2.00 (m, 4H), 2.13 (tt, J=4.7, 24.3, 1H), 2.35 (t, J=6.0, 2H), 3.22-3.53 (m, 5H), 3.73 (s, 3H), 3.96-4.04 (m, 2H), 4.34-4.44 (m, 1H), 4.78 (septet, J=6.1, 1H), 6.86 (q, J=5.3, 1H), 7.90 (d, J=12.2, 1H), 8.83 (s, 1H).

Mixed acetal 44: To a solution of tetraisopropyl ester 43 (319.7 mg, 0.3650 mmol) in 6 mL of CH₂Cl₂ was added 2.40 mL (18.18 mmol) of bromotrimethylsilane. After stirring overnight at room temperature, the solvent was removed and the residue was kept at high vacuum for at least 30 min. The resulting material was dissolved in dilute sodium hydroxide solution (≦2 eq of NaOH, this process was fairly time-consuming and ultra-sound sonication was needed from time to time to help maintain the dissolution) prior to the careful adjustment of pH to 7.47 with 1 N sodium hydroxide solution. The aqueous solution obtained was subjected to a Waters® C18 Sep-Pak™ cartridge (20 cc) with gradient elution from neat water to 10:1 water/methanol. All the fractions with the desired product were immediately combined and frozen in an acetone/dry ice cold bath. The solvents were removed by freeze drying and the material obtained was washed with dichloromethane to yield 93 mg (30%) of product 44 as an off-white powder. ¹H-NMR (400 MHz, D₂O): [symbol] 0.88-1.02 (m, 2H), 1.07-1.20 (m, 2H), 1.37 (t, J=7.3, 3H), 1.55 (d, J=5.5, 3H), 1.72-1.85 (m, 5H), 2.48 (t, J=6.4, 2H), 3.25-3.34 (m, 2H), 3.43-3.52 (m, 4H), 3.75 (s, 3H), 3.93 (bs, 1H), 4.10 (septet, J=3.5, 1H), 4.36 (bs, 1H), 6.80 (dq, J=0.8, 5.5, 1H), 7.73 (d, J=12.7, 1H), 8.52 (s, 1H); ³¹P NMR (162 MHz, D₂O): [symbol] 20.87; ¹⁹F NMR (376 MHz, D₂O): [symbol] -122.32 (bs); MS (m/e): 708 (M+H).

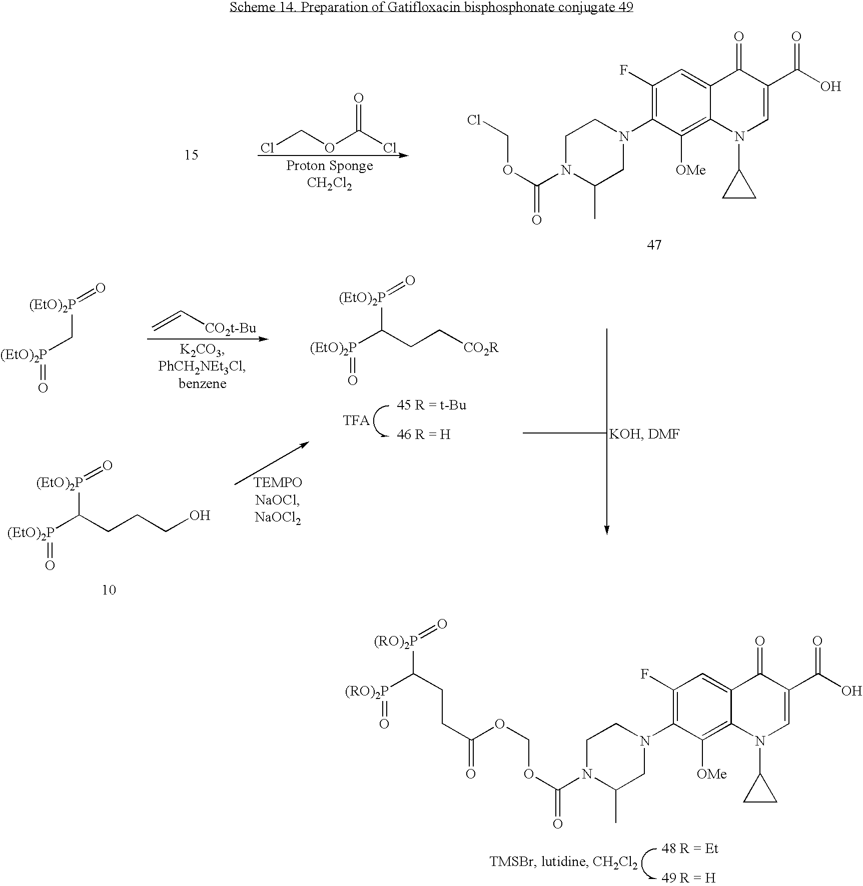

### Tetraethyl 3-(t-butoxycarbonyl)propylene-1,1-bisphosphonate (45)

To a solution of tetraethylmethylene bisphosphonate (10.0 g, 34.7 mmol) in benzene (56 mL) were added t-butyl acrylate (5.54 mL, 38.2 mmol), K₂CO₃ (4.79 g, 34.7 mmol) and benzyl triethylammonium chloride (0.79 g, 3.5 mmol). The mixture was stirred under reflux for 18 hours. After which it was filtered and the filtrate was concentrated. Purification by flash chromatography on silica gel using a gradient of 0-10% MeOH/EtOAc provided compound 45 as a colorless oil (3.8 g, 26%). ¹H-NMR (400 MHz, CDCl₃) δ 1.34 (t, J=7.0 Hz, 12H), 1.43 (s, 9H), 2.11-2.25 (m, 2H), 2.48 (tt, J=23.9, 6.5 Hz, 1H), 2.56 (t, J=7.4 Hz, 2H), 4.13-4.22 (m, 8H).

### Tetraethyl 3-carboxypropylene-1,1-bisphosphonate (46)

t-Butyl ester 45 (4.3 g, 10.3 mmol) was stirred in TFA (8.6 mL) for 15 min., then concentrated to dryness. Purification on reverse-phase Biotage 40M C18 column, using a gradient of 10-60% MeOH/H₂O provided compound 46 (3.7 g, 99%) as a colorless oil which solidified over time. ¹H-NMR (400 MHz, CDCl₃) δ 1.34 (t, J=7.0 Hz, 12H), 2.18-2.28 (m, 2H), 2.60 (tt, J=23.9, 6.5 Hz, 1H), 2.69 (t, J=7.3 Hz, 2H), 4.14-4.23 (m, 8H).

### Alternative Procedure:

To a solution of alcohol 10 (12.7 g, 36.7 mmol) in MeCN (200 mL) and phosphate buffer solution (200 mL, made from mixing equal volumes of 0.67M Na₂HPO₄ solution and 0.67M NaH₂PO₄ solution) at 35° C. was added a catalytic amount of TEMPO (430 mg, 2.75 mmol). The reaction flask, maintained at 35° C., was fitted with two addition funnels. One was filled with a solution of NaClO₂ (8.3 g, 91.7 mmol) in 75 mL H₂O. The other one was filled with a solution of household bleach (5.25%, 25 mL) in 250 mL H₂O. About ⅕ of the NaClO₂ solution was added, followed by about ⅕ of the bleach solution to initiate the reaction. The remainder of both solutions was added dropwise, simultaneously, with a rate adjusted so that both additions finished concurrently. The reaction mixture was stirred at 35° C. for 4 h, then at room temperature for 18 h. The reaction mixture was diluted with 300 mL H₂O and the pH of the solution was adjusted to 8.0 by adding 1M NaOH. The resulting solution was cooled to 0° C. and a cold solution of Na₂SO₃ (6.1% wt, 185 mL) was added slowly. The mixture was stirred at 0° C. during 30 min, after which a portion of Et₂O was added. After stirring vigourously, the mixture was poured into an extraction funnel and the Et₂O layer was separated and discarded. The aqueous layer was acidified to pH 3.4 with conc. HCl and extracted 3× with CHCl₃/i-PrOH mixture (4:1). The combined organic layers were dried over MgSO₄, filtered and concentrated to dryness, yielding 46 as a pale yellow oil (12.9 g, 98%), which could be used without further purification. The ¹H-NMR spectrum of this compound was consistent with that produced from hydrolysis of ester 45.

7-(4-(chloromethoxycarbonyl)-3-methylpiperazin-1-yl)-1-cyclopropyl-6-fluoro-1,4-dihydro-8-methoxy-4-oxoquinoline-3-carboxylic acid (47): A suspension of 15 (8.65 g, 22.9 mmol) and proton sponge (4.90 g, 22.9 mmol) in anhydrous CH₂Cl₂ (100 mL) was cooled in an ice-bath followed by the drop-wise addition of chloroformic acid chloromethyl ester (2.03 mL, 22.9 mmol).

The resulting mixture was stirred at the same temperature for four hours. The reaction mixture was diluted by the addition of CH₂Cl₂ (300 mL) and washed with cold aqueaous HCl (5%) and saturated NaCl then dried over anhydrous sodium sulfate. After filtering off the drying agent the organics were removed under reduced pressure to give 47 as a yellow coloured solid that was used without purification (10.2 g, 95%): ¹H NMR (400 MHz, CDCl₃): [symbol] 0.94-1.04 (m, 2H), 1.17-1.27 (m, 2H), 1.40 (d, J=6.7, 3H), 3.28-3.53 (m, 5H), 3.73 (s, 3H), 3.98-4.10 (m, 2H), 4.45 (bs, 1H), 5.85 (m, 2H), 7.92 (d, J=12.3, 1H), 8.83 (s, 1H).

Mixed acetal 48: Compound 46 (3.70 g, 10.3 mmol) was dissolved in CH₃CN (20 mL), followed by the addition of KOH (0.634 g, 11.3 mmol) in H₂O. The resulting solution was stirred for five minutes then concentrated under reduced pressure. The sodium salt of 46 was dissolved in DMF (25 mL) followed by the addition of 47 (2.09 g, 4.47 mmol). The resulting solution was stirred at room temperature for 3 hr then quenched by the addition of ice-cold H₂O (150 mL). The product was extracted with EtOAc (3×100 mL) and the combined organics were washed with water and brine then dried over Na₂SO₄. After filtering off the drying agent the organics were removed under reduced pressure resulting in 48 as yellow solid that was used without purification (3.3 g, 93%): ¹H NMR (400 MHz, CDCl₃): [symbol] 0.92-1.04 (m, 2H), 1.17-1.27 (m, 2H), 1.34 (t, J=6.9, 12H), 1.39 (d, J=10.5, 3H), 1.95 (bs, 2H), 2.19-2.33 (m, 2H), 2.47 (tt, J=7.5, 31.1, 1H), 2.77 (t, J=7.6, 2H), 3.28-3.52 (m, 5H), 3.73 (s, 3H), 3.98-4.23 (m, 8H), 4.42 (bs, 1H), 5.87 (m, 2H), 7.90 (d, J=11.9, 1H), 8.83 (s, 1H).

Mixed acetal 49: A solution of crude 48 (3.25 g, 4.11 mmol) and 2,6-lutidine (9.53 mL, 82.1 mmol) in CH₂Cl₂ (30 mL) was cooled in an ice-bath followed by the dropwise addition to TMSBr (8.13 mL, 61.6 mmol). The resulting yellow solution was stirred while warming to room temperature over 24 hr. The solvent and excess lutidine were then removed under reduced pressure. The residue was resuspended in H₂O and purified by reverse phase chromatography (0% to 60% CH₃CN in water) on a Biotage™ flash chromatography system. The CH₃CN was removed under reduced pressure and the water was removed by lyophisation to give the pale yellow coloured mono-2,6-lutidine salt of 49 (1.58 g, 49%): ¹H NMR (400 MHz, D₂O): δ 0.88-1.11 (m, 2H), 1.20-1.31 (m, 2H), 1.36 (d, J=6.9, 3H), 2.04-2.22 (m, 3H), 1.79 (t, J=7.7, 2H), 2.71 (s, 6H), 3.28-3.55 (m, 5H), 3.75 (s, 3H), 3.97 (bd, J=12.0, 1H), 4.20-4.26 (m, 1H), 4.38 (bs, 1H), 5.82 (s, 2H), 7.41 (d, J=12.8, 1H), 7.62 (d, J=8.2, 1H), 8.26 (t, J=7.7, 2H), 8.84 (s, 1H): ³¹P NMR (162 MHz, D₂O): [symbol] 20.94 (s, 1P): ¹⁹F NMR (376 MHz, D₂O): [symbol] −119.05 (d, J=10.5, 1F): LCMS −98.8% (254 nm), 98.8% (220 nm), 98.9% (320 nm): MS (MH⁺) 680.2.

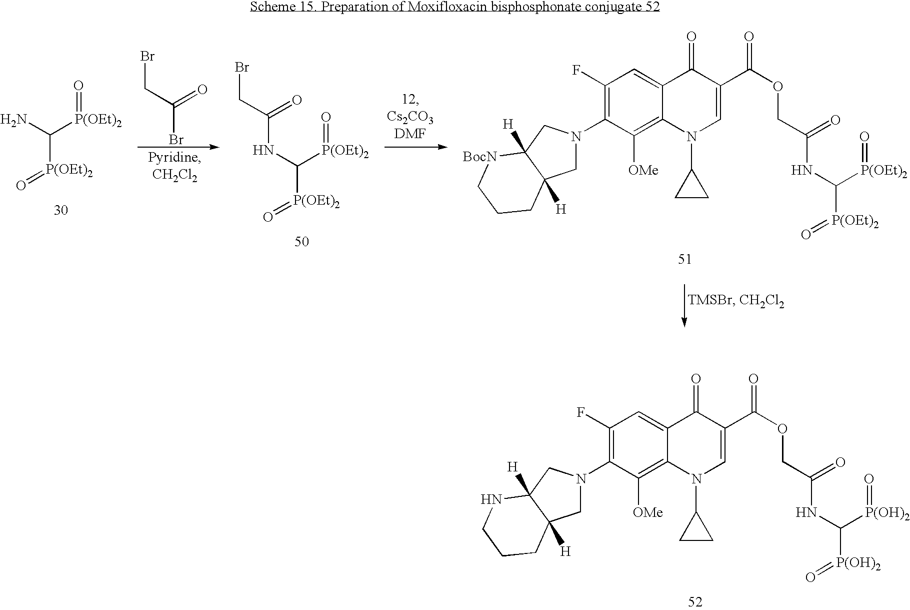

Tetraethyl 1-(N-2-bromoacetylamino)methylenebisphosphonate (50): A solution of bromoacetyl bromide (0.35 mL, 4.0 mmol) in CH₂Cl₂ (1 mL) was added dropwise to a stirred, cooled (ice-bath) solution of 30 (1.1 g, 3.6 mmol) and pyridine (0.59 mL, 7.3 mmol) in CH₂Cl₂ (10 mL). After stirring at the same temperature for 4 hours the reaction was quenched by the addition of water. The product was extracted with CH₂Cl₂ and the combined organics were washed with 10% aqueous HCl, brine, dried over sodium sulfate and concentrated at reduced pressure. The crude yellow oil was purified by silica gel column chromatography (0% to 3% MeOH in CH₂Cl₂) resulting in 50 as a colourless solid (0.58 g, 37%). ¹H NMR (400 MHz, CDCl₃) δ 1.35 (t, J=7.2, 12H), 3.92 (s, 2H), 4.12-4.28 (m, 8H), 4.92 (dt, J=10.2, 21.7, 1H), 6.91 (bd, J=10.0, 1H).

(1,1-Bis(diethylphosphono)methylcarbamoyl)methyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (51): A solution of 12 (1.1 g, 2.1 mmol), 50 (0.90 g, 2.1 mmol) and Cs₂CO₃ (0.76 g, 2.3 mmol) was stirred at room temperature for 12 hr. The mixture was then diluted with H₂O and extracted with CH₂Cl₂. The organics were washed with brine, dried over sodium sulfate, filtered and concentrated at reduced pressure resulting in a brown oil that was purified by silica gel chromatography (0% to 8% MeOH in CH₂Cl₂) to give 51 as beige solid (1.29 g, 72%). ¹H NMR (400 MHz, CDCl₃) δ 0.75-0.80 (m, 1H), 0.99-1.09 (m, 2H), 1.22-1.29 (m, 1H), 1.31-1.37 (m, 12H), 1.46-1.51 (m, 1H), 1.48 (s, 9H), 1.75-1.83 (m, 3H), 2.22-2.27 (m, 1H), 2.86-2.91 (m, 1H), 3.19-3.23 (m, 1H), 3.34-3.38 (m, 1H), 3.56 (s, 3H), 3.83 (dt, J=1.9, 10.0, 1H), 3.87-3.92 (m, 1H), 4.02-4.09 (m, 2H), 4.20-4.38 (m, 8H), 4.73-4.82 (bs, 1H), 4.83 (d, J=15.8, 1H), 4.91 (d, J=15.8, 1H), 5.22 (dt, J=10.0, 23.0, 1H), 7.75 (d, J=14.1, 1H), 8.49 (s, 1H), 9.24 (d, J=9.2, 1H).

(1,1-Bisphosphonomethylcarbamoyl)methyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (52): TMSBr (3.0 mL, 23 mmol) was added in one portion to a stirring solution of 51 (1.27 g, 1.50 mmol) in CH₂Cl₂. After 18 h the solvent was removed at reduced pressure and the yellow solid was re-suspended in H₂O and the pH was adjusted to 7.4 by the addition of NaOH. The resulting solution was the subjected to a Waters C18 Sep-Pak™ and the product was eluted in H₂O (150 mL) then 5% MeOH/H₂O (50 mL) to give 52 as yellow solid (744 mg, 69%). ¹H NMR (400 MHz, D₂O) δ 0.70-0.78 (m, 1H), 0.86-1.00 (m, 2H), 1.03-1.10 (m, 1H), 1.67-1.78 (m, 4H), 2.61 (bs, 1H), 2.90-2.95 (m, 1H), 3.23-3.26 (m, 1H), 3.40 (s, 3H), 3.51-3.65 (m, 3H), 3.75 (bs, 1H), 3.83-3.87 (m, 1H), 3.92-3.97 (m, 1H), 4.19 (t, J=18.0, 1H), 4.74 (s, 2H), 7.24 (d, J=14.5, 1H), 8.77 (s, 1H): ¹⁹F (376 MHz, D₂O) δ −121.76 (d, J=15.4, 1F): ³¹P (162 MHz, D₂O) δ 13.90 (s, 2P): LCMS: 94.8% (254 nm), 95.6% (220 nm), 97.0% (320 nm). MS: (MH⁺) 633.1.

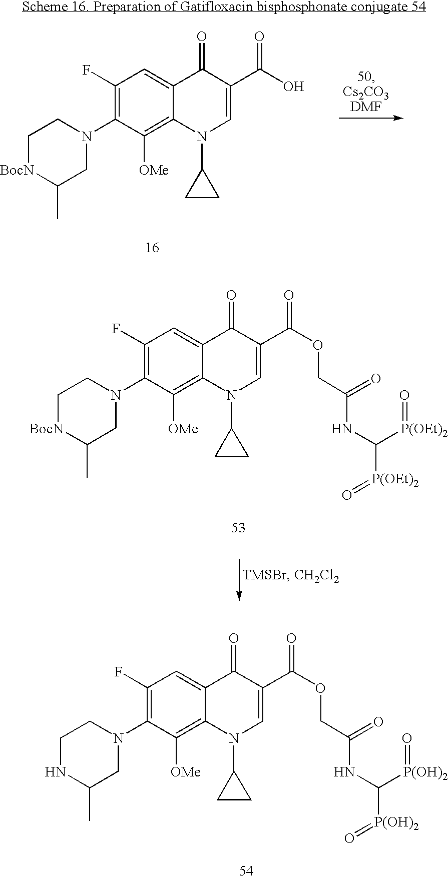

(1,1-Bis(diethylphosphono)methylcarbamoyl)methyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (53): The coupling reaction between 50 and 16 was carried out as described for the synthesis of 51 on a 1.3 mmol scale. The crude product was purified by silica gel chromatography (0% to 5% MeOH in CH₂Cl₂) to give 53 as a pale yellow coloured solid (0.672 g, 62%). ¹H NMR (400 MHz, CDCl₃) δ 0.90-0.95 (m, 2H), 1.13-1.19 (m, 2H), 1.32-1.36 (m, 15H), 1.50 (s, 9H), 3.19-3.46 (m, 5H), 3.72 (s, 3H), 3.89-3.97 (m, 2H), 4.21-4.37 (m, 9H), 4.87 (s, 2H), 5.21 (dt, J=9.9, 22.9, 1H), 7.81 (d, J=12.4, 1H), 8.53 (s, 1H), 9.10 (d, J=9.9, 1H): LCMS: 97.8% (254 nm), 96.1% (220 nm), 98.0% (320 nm). MS: (MH⁺) 819.3.

(1,1-Bisphosphonomethylcarbamoyl)methyl 7-(3-methylpiperazin-1-yl)-1-cyclopropyl-6-fluoro-1,4-dihydro-8-methoxy-4-oxoquinoline-3-carboxylate (54): The deprotection of 53 was completed as described for that of 51 on a 0.45 mmol scale. The crude product was purified by Waters C18 Sep-Pak™ column (150 mL H₂O then 50 mL of 5% MeOH/H₂O) to give 54 as a pale yellow coloured solid (235 mg, 75%). ¹H NMR (400 MHz, D₂O) δ 1.02-1.06 (m, 2H), 1.18-1.23 (m, 2H), 1.38 (d, J=6.7, 3H), 3.31-3.43 (m, 2H), 3.49-3.69 (m, 5H), 3.81 (s, 3H), 4.14-4.20 (m, 1H), 4.49 (t, J=20.1, 1H), 4.94 (s, 2H), 7.68 (d, J=12.3, 1H), 9.00 (s, 1H): ¹⁹F (376 MHz, D₂O) δ-119.20 (d, J=12.0, 1F): ³¹P (162 MHz, D₂O) δ 16.41 (s, 2P): LCMS: 97.5% (254 nm), 97.4% (220 nm), 98.4% (320 nm). MS: (MH⁺) 607.0.

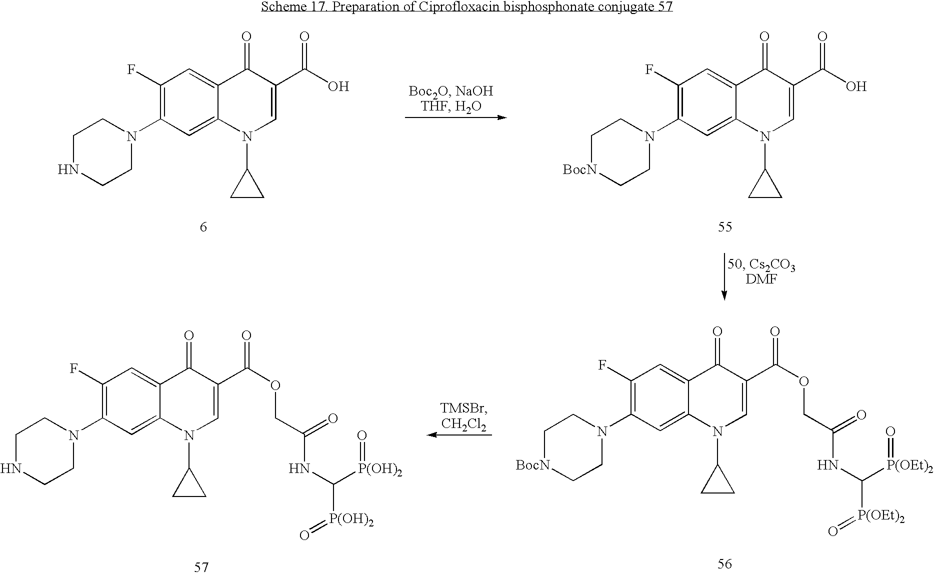

7-(4-(tert-butoxycarbonyl)piperazin-1-yl)-1-cyclopropyl-6-fluoro-1,4-dihydro-4-oxoquinoline-3-carboxylic acid (55): A suspension of ciprofloxacin hydrochloride 6 (3.95 g, 10.7 mmol), di-tert-butyl dicarbonate (2.46 g, 11.3 mmol) and NaOH (1.29 g, 32.2 mmol) in THF/H2O (105 mL; 2:1) was stirred at room temperature for 6 hr. The product was collected by filtration to give the colorless solid 55 (3.93 g, 78%) that was used without further purification. ¹H NMR (400 MHz, CDCl₃) δ 1.89-1.23 (m, 2H), 1.37-1.43 (m, 2H), 1.50 (s, 9H), 3.28 (bd, J=5.0, 4H), 3.51-3.56 (m, 1H), 3.67 (bt, J=5.0, 4H), 7.37 (d, J=7.3, 1H), 8.05 (d, J=13.0, 1H), 8.78 (s, 1H).

(1,1-Bis(diethylphosphono)methylcarbamoyl)methyl 7-(4-(tert-butoxycarbonyl)piperazin-1-yl)-1-cyclopropyl-6-fluoro-1,4-dihydro-4-oxoquinoline-3-carboxylate (56): A solution of 55 (0.472 g, 1.01 mmol), 50 (0.408 g, 0.962 mmol) and Cs₂CO₃ (0.345 g, 1.06 mmol) was stirred at room temperature for 3 hr. The mixture was then diluted with H₂O and extracted with EtOAc. The organics were washed with brine, dried over Na₂SO₄, filtered and concentrated at reduced pressure resulting in a brown oil that was purified by silica gel chromatography (0% to 7% MeOH in CH₂Cl₂) to give 56 as pale yellow coloured solid (0.737 g, 90%). ¹H NMR (400 MHz, CDCl₃) δ 1.12-1.17 (m, 2H), 1.31-1.36 (m, 14H), 1.50 (s, 9H), 3.26 (bt, J=4.9, 4H), 3.41-3.47 (m, 1H), 3.66 (bt, J=4.9, 4H), 4.20-4.38 (m, 8H), 4.88 (s, 2H), 5.22 (dt, J=0.3, 22.6, 1H), 7.31 (d, J=6.9, 1H), 7.99 (d, J=13.3, 1H), 8.49 (s, 1H), 9.21 (d, J=9.8, 1H). ¹⁹F (376 MHz, CDCl₃) δ-123.57 (dd, J=7.5, 13.2, 1F): ³¹P (162 MHz, CDCl₃) δ 17.35 (s, 2P).

(1,1-Bisphosphonomethylcarbamoyl)methyl 1-cyclopropyl-6-fluoro-1,4-dihydro-4-oxo-7-(piperazin-1-yl)quinoline-3-carboxylic acid (57): The deprotection of 56 was completed as described for that of 51 on a 0.938 mmol scale. The crude product was purified by Waters C18 Sep-Pak™ column after adjusting the pH to 8 (150 mL H₂O then 50 mL of 5% MeOH/H₂O) to give 57 as a colourless solid (350 mg, 66%). ¹H NMR (400 MHz, D₂O) δ 1.18-1.22 (m, 2H), 1.36-1.41 (m, 2H), 3.15 (m, 4H), 3.25 (m, 4H), 3.52 (bs, 1H), 4.22 (t, J=18.7, 1H), 4.79 (s, 2H), 7.36 (d, J=7.2, 1H), 7.60 (d, J=12.5, 1H), 8.80 (s, 1H): ¹⁹F (376 MHz, D₂O) δ-123.72 (dd, J=6.9, 12.0, IF): ³¹P (162 MHz, D₂O) δ 13.91 (s, 2P): LCMS: 97.9% (254 nm), 97.4% (220 nm), 98.2% (290 nm). MS: (MH⁺) 563.1.

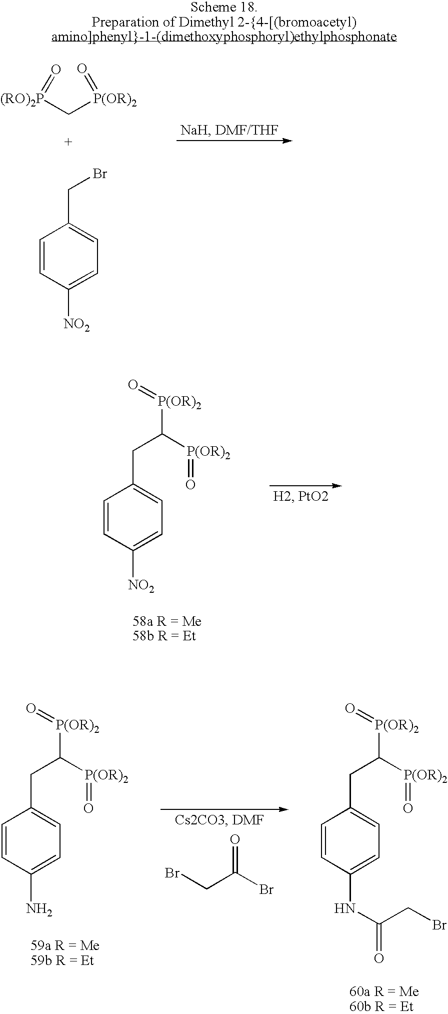

Dimethyl 1-(dimethoxyphosphoryl)-2-(4-nitrophenyl)ethylphosphonate (58a): Sodium hydride (1.02 g, 25.4 mmol) was added in portions to a stirring solution of tetramethyl methylenediphosphonate in DMF (40 mL). After 30 min a solution of 4-nitrobenzylbromide (5.00 g, 23.1 mmol) in THF (5 mL) was added and the resulting mixture was stirred at room temperature for 4.5 hr. The reaction was quenched by the addition of saturated aqueous NH₄Cl (20 mL). After the addition of water (100 mL) the product was extracted with EtOAc and the combined organics were washed with brine, dried over MgSO₄, filtered and concentrated at reduced pressure. The crude product was purified by silica gel chromatography (0% to 10% MeOH in EtOAc) resulting in 58a as a colorless solid (2.55 g, 30%). ¹H NMR (400 MHz, CDCl₃) δ 2.65 (tt, J=6.5, 23.8, 1H), 3.31 (dt, J=6.5, 16.5, 2H), 3.73 (d, J=7.0, 6H), 3.75 (d, J=7.0, 6H), 7.42 (d, J=8.9, 2H), 8.15 (d, J=8.9, 2H).

Diethyl 1-(diethoxyphosphoryl)-2-(4-nitrophenyl)ethylphosphonate (58b): Prepared as for 58a using tetraethyl methylenediphosphonate instead of the tetramethyl ester, resulting in 58b as a yellow oil (34% yield). ¹H NMR (400 MHz, CDCl₃) δ 1.27 (t, J=5.3, 12H), 2.62 (tt, J=6.5, 23.6, 1H), 3.33 (dt, J=6.2, 16.4, 2H), 4.11 (m, 8H), 7.44 (d, J=8.9, 2H), 8.14 (d, J=8.9, 2H). ³¹P NMR (162 MHz, CDCl₃) δ 23.256 (s, 2P)

Dimethyl 2-(4-aminophenyl)-1-(dimethoxyphosphoryl)ethylphosphonate (59a): A mixture of 59a (1.01 g, 2.75 mmol) and PtO₂ (0.035 g, 0.15 mmol) in EtOH (40 mL, 95%) was shaken in a PARR apparatus under 55 p.s.i of H₂ for 14 hr. The catalyst was removed by filtration through glass fiber filter paper and the solvent was removed under reduced pressure to give 59a as a pale yellow solid (0.959 g, 103%) that was used without purification. ¹H NMR (400 MHz, CDCl₃) δ 2.62 (tt, J=6.3, 23.9, 1H), 3.12 (dt, J=6.3, 16.2, 2H), 3.70 (d, J=1.9, 6H), 3.73 (d, J=1.9, 6H), 6.61 (d, J=8.5, 2H), 7.04 (d, J=8.5, 2H).

Diethyl 2-(4-aminophenyl)-1-(diethoxyphosphoryl)ethylphosphonate (59b): Prepared as for 59a but starting with 53b to afford 59b as a red oil (96%) that was used without purification. ¹H NMR (400 MHz, CDCl₃) δ 1.27 (t, J=5.3, 12H), 2.56 (tt, J=6.5, 23.6, 1H), 3.14 (dt, J=6.2, 16.4, 2H), 3.63 (s, 2H), 4.08 (m, 8H), 6.59 (d, J=8.9, 2H), 7.03 (d, J=8.9, 2H). ³¹P NMR (162 MHz, CDCl₃) δ 24.356 (s, 2P).

Dimethyl 2-{4-[(bromoacetyl)amino]phenyl}-1-(dimethoxyphosphoryl)ethylphosphonate (60a): A solution of 59a (0.959 g, 2.87 mmol) and pyridine (349 μL, 4.31 mmol) in CH₂Cl₂ was cooled in an ice-bath while stirring. A solution of bromoacetylbromide (250 μL, 2.87 mmol) in CH₂Cl₂ (5 mL) was added drop-wise and the resulting mixture was stirred for 4 h at that temperature. The reaction was quenched by the addition of water and the product was extracted with CH₂Cl₂. The combined organic layers were washed with brine, dried over sodium sulfate, filtered, and concentrated under reduced pressure. The crude yellow solid was purified by silica gel chromatography resulting in 60a as a colorless solid (0.897 g, 67%). ¹H NMR (400 MHz, CDCl₃) δ 2.65 (tt, J=6.2, 24.4, 1H), 3.22 (dt, J=6.2, 17.4, 2H), 3.72 (d, J=3.7, 6H), 3.75 (d, J=3.7, 6H), 4.01 (s, 2H), 7.26 (d, J=8.6, 2H), 7.47 (d, J=8.6, 2H), 8.15 (bs, 1H): ³¹P (162 MHz, CDCl₃) δ 26.33 (s, 2P).

Diethyl 2-{4-[(bromoacetyl)amino]phenyl}-1-(diethoxyphosphoryl)ethylphosphonate (60b): Prepared as for 60a but starting with 59b to furnish 60b as a red oil (82%). ¹H NMR (400 MHz, CDCl₃) δ 1.27 (t, J=5.3, 12H), 2.63 (tt, J=6.5, 23.6, 1H), 3.26 (dt, J=6.2, 16.4, 2H), 4.14 (m, 10H), 7.29 (d, J=8.9, 2H), 7.49 (d, J=8.9, 2H), 8.28 (s, 1H). ³¹P NMR (162 MHz, CDCl₃) δ 23.964 (s, 2P).

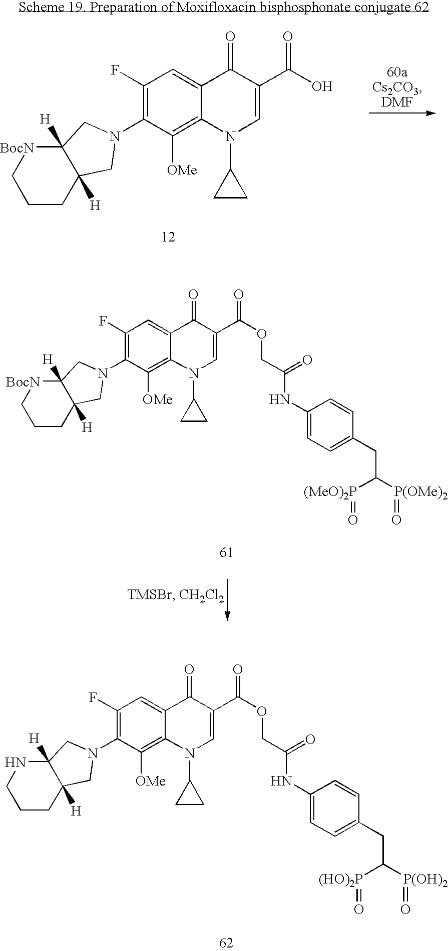

(4-(2,2-Bis(dimethylphosphono)ethyl)phenylcarbamoyl)methyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (61): The coupling reaction between 60a and 12 was carried out as described for the synthesis of 51 on a 0.97 mmol scale. The crude product was purified by silica gel chromatography (0% to 8% MeOH in CH₂Cl₂) to give 61 as a yellow coloured solid (0.562 g, 65%). ¹H NMR (400 MHz, CDCl₃) δ 0.73-0.84 (m, 1H), 0.97-1.11 (m, 2H), 1.20-1.28 (m, 2H), 1.41-1.52 (m, 1H), 1.46 (s, 9H), 1.70-1.85 (m, 2H), 2.19-2.29 (m, 1H), 2.67 (tt, J=6.3, 23.7, 1H), 2.82-2.93 (m, 1H), 3.20 (dt, J=6.2, 16.0, 3H), 3.36 (bs, 1H), 3.55 (s, 3H), 3.69 (d, J=3.1, 6H), 3.72 (d, J=3.1, 6H), 3.74-3.95 (m, 2H), 4.01-4.13 (m, 2H), 4.77 (bs, 1H), 4.89 (AB q, J=14.9, 2H), 7.24 (d, J=8.4, 2H), 7.88 (d, J=8.4, 2H), 7.89 (d, J=14.1, 1H), 8.49 (s, 1H), 11.0 (s, 1H).

(4-(2,2-Bisphosphonoethyl)phenylcarbamoyl)methyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (62): The deprotection of 61 was carried out as described for that of 52 on a 0.63 mmol scale. The crude product was purified by Waters C18 Sep-Pak™ column (0% to 10% MeOH in H₂O) to give 62 as a pale yellow coloured solid (40 mg, 9%). ¹H NMR (400 MHz, D₂O) δ 0.60-0.69 (m, 1H), 0.93-1.07 (m, 2H), 1.12-1.21 (m, 1H), 1.72-1.96 (m, 4H), 2.16 (tt, J=6.9, 20.6, 1H), 2.62-2.72 (m, 1H), 2.90-3.17 (m, 3H), 3.31-3.40 (m, 1H), 3.44-3.63 (m, 5H), 3.65-3.72 (m, 1H), 3.75-3.99 (m, 3H), 4.80 (s, 2H), 7.25-7.39 (m, 5H), 8.57 (s, 1H): ¹⁹F (376 MHz, D₂O) δ-121.42 (d, J=14.0, IF): ³¹P (162 MHz, D₂O) δ 20.25 (d, J=22.4, 2P): LCMS: 95.7% (254 nm), 95.4% (220 nm), 96.0% (290 nm). MS: (MH⁺) 633.1.

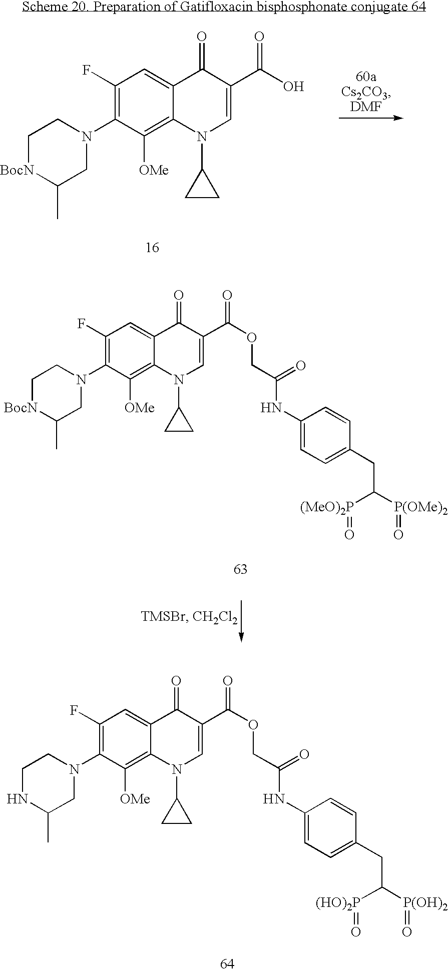

(4-(2,2-Bis(dimethylphosphono)ethyl)phenylcarbamoyl)methyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (63): The coupling reaction between 60a and 16 was carried out as described for the synthesis of 51 on a 1.84 mmol scale. The crude product was purified by silica gel chromatography (0% to 8% MeOH in CH₂Cl₂) to give 63 as a pale yellow coloured solid (1.10 g, 70%). ¹H NMR (400 MHz, CDCl₃) δ 0.92-1.00 (m, 2H), 1.16-1.25 (m, 2H), 1.35 (d, J=6.8, 3H), 1.50 (s, 9H), 2.69 (tt, J=6.4, 24.3, 1H), 3.17-3.50 (m, 7H), 3.71 (d, J=2.5, 6H), 3.74 (s, 3H), 3.75 (d, J=2.5, 6H), 3.93-3.99 (m, 2H), 4.36 (bs, 1H), 4.92 (s, 2H), 7.26 (d, J=8.8, 2H), 7.89 (d, J=8.8, 2H), 7.98 (d, J=12.6, 1H), 8.57 (s, 1H), 10.90 (s, 1H): ¹⁹F (376 MHz, CDCl₃) δ-120.65 (d, J=11.5, IF): ³¹P (162 MHz, CDCl₃) δ 26.5 (s, 2P).

(4-(2,2-Bisphosphonoethyl)phenylcarbamoyl)methyl 7-(3-methylpiperazin-1-yl)-1-cyclopropyl-6-fluoro-1,4-dihydro-8-methoxy-4-oxoquinoline-3-carboxylate (64): The deprotection of 63 was carried out as described for that of 52 on a 0.413 mmol scale. The crude product was purified by Waters C18 Sep-Pak™ column (0% to 10% MeOH in H₂O) to give 64 as a pale yellow coloured solid (141 mg, 43%). ¹H NMR (400 MHz, D₂O) δ 0.98-1.01 (m, 2H), 1.15-1.18 (m, 2H), 1.22 (d, J=6.3, 3H), 2.20 (tt, J=6.7, 21.0, 1H), 3.07-3.49 (m, 9H), 3.80 (s, 3H), 4.05-4.11 (m, 1H), 4.92 (s, 2H), 7.46 (AB q, J=8.4, 4H), 7.55 (d, J=11.9, 1H), 8.80 (s, 1H): ¹⁹F (376 MHz, D₂O) δ-121.16 (d, J=12.6, 1F): ³¹P (162 MHz, D₂O) δ 20.23 (s, 2P): LCMS: 89.3% (254 nm), 91.4% (220 nm), 94.0% (290 nm). MS: (MH⁺) 697.2.

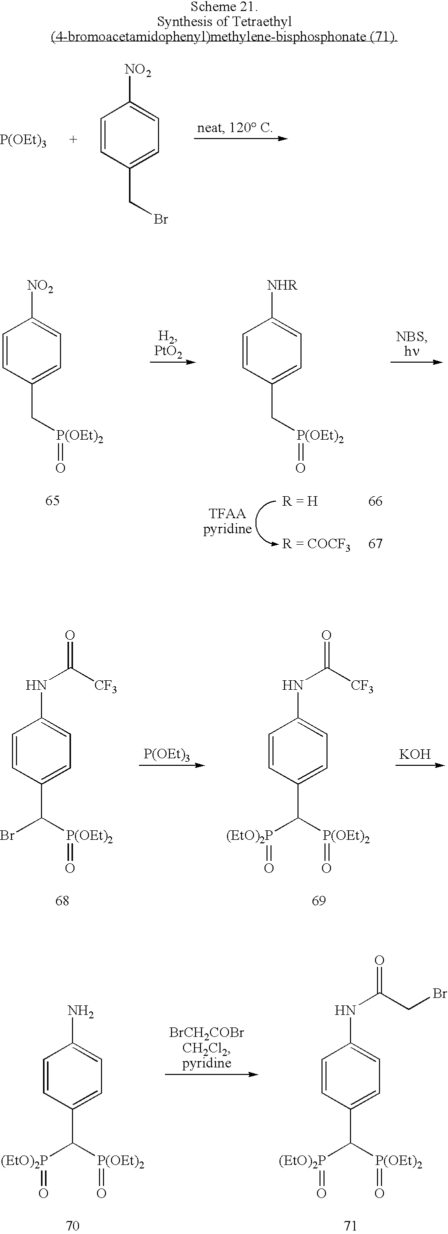

Diethyl (4-nitrophenyl)methylphosphonate (65): A neat solution of 4-nitrobenzylbromide (8.4 g, 39 mmol) and triethylphosphite (7.5 mL, 43 mmol) was stirred while heating to 120° C. in a sealed tube for 2 h. The mixture was then cooled and excess triethylphosphite was removed under high vacuum. The crude product was used without purification. ¹H NMR (400 MHz, CDCl₃) δ 1.26 (t, J=7.1, 6H), 3.24 (d, J=23.1, 2H), 4.01-4.10 (m, 4H), 7.47 (dd, J=8.7, 2.4, 2H), 8.18 (d, J=8.3, 2H).

Diethyl (4-(2,2,2-trifluoroacetamido)phenyl)methylphosphonate (67): Crude 65 was dissolved in abs. EtOH and hydrogenated over PtO₂ (200 mg) under H₂ (60 psi) for 4 h. The catalyst was filtered off and the solvent removed resulting in the pale-brown solid 66. ¹H NMR (400 MHz, CDCl₃) δ1.22 (t, J=7.2, 6H), 3.03 (d, J=23.1, 2H), 3.70 (bs, 2H), 3.95-4.03 (m, 4H), 6.61 (d, J=8.4, 2H), 7.15 (dd, J=8.5, 2.4, 2H).

The crude aniline 66 and pyridine (4.7 mL, 59 mmol) were dissolved in CH₂Cl₂ and the resulting solution was cooled to approximately 4° C. in an ice-bath. Trifluoroacetic anhydride (5.42 mL, 39 mmol) was then added dropwise while stirring and the resulting solution was stirred for a further 20 h while slowly warming to room temperature. The reaction was quenched by the addition of water (100 ml) and the product was extracted with CH₂Cl₂. The organic extracts were combined and washed with 10% HCl and brine followed by drying over Na₂SO₄. After filtration and concentration, the crude product was purified by flash column chromatography (gradient of 90-100% EtOAc in hexanes) resulting in the colorless solid 67 (9.41 g, 71% yield from 4-nitrobenzylbromide). ¹H NMR (400 MHz, CDCl₃) δ 1.23 (t, J=7.2, 6H), 3.15 (d, J=23.1, 2H), 3.98-4.07 (m, 4H), 7.18 (dd, J=8.5, 2.4, 2H), 7.54 (d, J=8.4, 2H), 9.98 (s, 1H).

Diethyl (4-(2,2,2-trifluoroacetamido)phenyl)bromomethylphosphonate (68): A solution of 67 (9.41 g, 27.7 mmol), NBS (7.5 g, 41.6 mmol) and azobis(cyclohexane carbonitrile) (70 mg, 0.29 mmol) in benzene was heated to reflux under the presence of a strong visible light for 5 h. After the addition of water the product was extracted with EtOAc. The organics were washed with saturated NaCl then dried over Na₂SO₄. The crude solid was purified by silica gel chromatography (1:1 EtOAc:hexanes) to give 68 as a pale yellow solid (4.0 g, 34% yield). ¹H NMR (CDCl₃, 400 MHz) δ 1.37 (t, J=8.3, 6H), 4.22-4.30 (m, 4H), 4.85, (d, J=13.6, 1H), 7.54 (dd, J=8.7, 1.7, 2H), 7.60 (d, J=8.6, 2H), 8.85 (s, 1H).

Tetraethyl (4-(2,2,2-trifluoroacetamido)phenyl)methylenebisphosphonate (69): A solution of 68 (4.0 g, 9.6 mmol) and triethylphosphite (1.6 ml, 9.6 mmol) in THF was heated to reflux for 20 h. The solution was cooled to room temperature and concentrated to approximately 5 mL then diethyl ether was added. The product 69 was collected as a colorless precipitate (0.6 g, 14% yield). ¹H NMR (CDCl₃, 400 MHz) δ 1.15 (t, J=7.5, 6H), 1.32 (t, J=7.5, 6H), 3.7 (t, J=24.8, 1H), 3.82-3.92 (m, 2H), 3.96-4.06 (m, 2H), 4.13-4.20 (m, 4H), 7.40 (m, 2H), 7.59 (d, J=8.9, 2H), 9.92 (s, 1H).

Tetraethyl (4-aminophenyl)methylenephosphonate (70): A suspension of 69 (0.45 g, 0.95 mmol) and KOH (64 mg, 1.05 mmol) in H₂O was stirred while warming to 50° C. for 5 h. The solution was diluted with H₂O and neutralized with 20 ml saturated NH₄Cl. The aqueous phase was extracted with CH₂Cl₂ and the combined organic extracts were dried over Na₂SO₄, filtered and concentrated to the pale yellow solid of 70 (330 mg, 92% crude yield). ¹H NMR (DMSO-d₆, 400 MHz) δ 1.13 (t, J=7.2, 6H), 1.25 (t, J=7.2, 6H), 3.59 (t, J=25.0, 1H), 3.70 (s, 2H), 3.84-3.94 (m, 4H), 3.97-4.13 (m, 4H), 6.61 (d, J=8.5, 2H), 7.20-7.23 (m, 2H).

Tetraethyl (4-bromoacetamidophenyl)methylenebisphosphonate (71): A solution of bromoacetyl bromide (0.36 mL, 4.2 mmol) in CH₂Cl₂ (1 mL) was added dropwise to a stirred, cooled (ice-bath) solution of 70 (1.05 g, 2.77 mmol) and pyridine (0.34 mL, 4.2 mmol) in CH₂Cl₂ (14 mL). After stirring at the same temperature for 4 h, the reaction was quenched by the addition of water. The product was extracted with CH₂Cl₂ and the combined organics were washed with 10% aqueous HCl, brine then dried over MgSO₄. After filtering the drying agent the organics were concentrated at reduced pressure and the crude brown solid was purified by silica gel flash column chromatography (0% to 6% MeOH in CH₂Cl₂) resulting in 71 as a pale yellow solid (1.16 g, 77%). ¹H NMR (400 MHz, CDCl₃) δ 1.15 (t, J=6.8, 6H), 1.28 (t, J=6.8, 6H), 3.70 (t, J=25.3, 1H), 3.99-4.17 (m, 6H), 7.42 (dt, J=1.6, 8.8, 2H), 7.50 (bd, J=8.2, 2H), 8.54 (bs, 1H). ³¹P (162 MHz, D₂O) δ 19.42 (s, 2P).

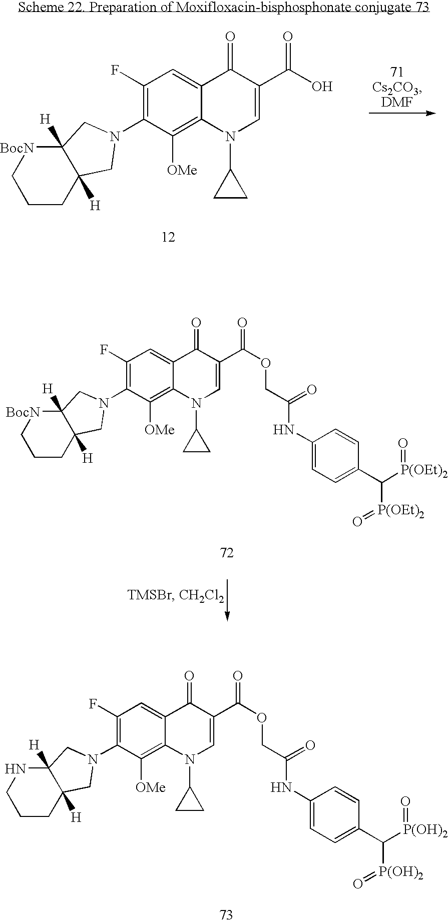

(4-Bis(diethylphosphono)methylphenylcarbamoyl)methyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (72): The coupling reaction between 70 and 12 was carried out as described for the synthesis of 51 on a 1.34 mmol scale. The crude product was purified by silica gel flash column chromatography (0% to 10% MeOH in CH₂Cl₂) to give 72 as a yellow coloured solid (0.539 g, 45%). ¹H NMR (400 MHz, CDCl₃) δ0.75-0.83 (m, 1H), 0.98-1.11 (m, 2H), 1.15 (t, J=7.2, 6H), 1.26 (t, J=7.2, 6H), 1.24-1.28 (m, 1H), 1.44-1.60 (m, 11H), 1.72-1.85 (m, 2H), 2.20-2.29 (m, 1H), 2.82-2.94 (m, 1H), 3.17-3.29 (m, 1H), 3.32-3.43 (m, 1H), 3.56 (s, 3H), 3.71 (t, J=25.8, 1H), 3.81-3.98 (m, 4H), 3.99-4.16 (m, 8H), 4.77 (bs, 1H), 4.91 (AB q, J=16.0, 2H), 7.45 (d, J=8.6, 2H), 7.91 (d, J=14.1, 2H), 7.97 (d, J=8.6, 2H), 8.49 (s, 1H), 11.10 (s, 1H): ³¹P (162 MHz, D₂O) δ 19.65 (s, 2P).

(4-Bisphosphonomethylphenylcarbamoyl)methyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (73): The deprotection of 72 was carried out as described for that of 51 on a 0.59 mmol scale. The crude product was purified by Waters C18 Sep-Pak™ column (0% to 20% MeOH in H₂O) to give 73 as a pale yellow coloured solid (110 mg, 27%). ¹H NMR (400 MHz, D₂O) δ 0.83-0.93 (m, 1H), 0.99-1.14 (m, 2H), 1.17-1.27 (m, 1H), 1.74-1.93 (m, 4H), 2.61-2.70 (m, 1H), 2.96-3.05 (m, 1H), 3.29-3.36 (m, 1H), 3.48 (t, J=23.2, 1H), 3.62 (s, 3H), 3.56-3.65 (m, 3H), 3.69-3.77 (m, 1H), 3.87-3.95 (m, 1H), 3.99-4.06 (m, 1H), 4.90 (AB q, J=15.4, 2H), 7.37-7.56 (m, 5H), 8.78 (s, 1H): ³¹P (162 MHz, D₂O) δ 16.68 (s, 2P): LCMS: 100% (254 nm), 100% (220 nm), 100% (290 nm). MS: (MH⁺) 633.1.

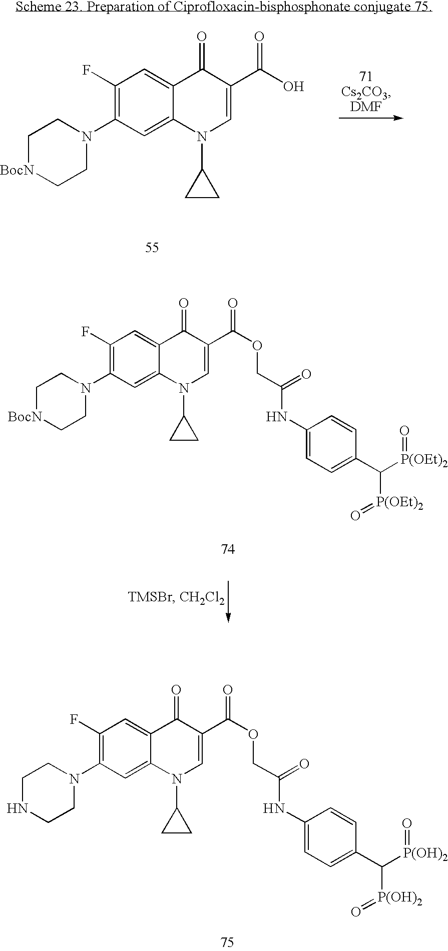

(4-Bis(diethylphosphono)methylphenylcarbamoyl)methyl 7-(4-(tert-butoxycarbonyl)piperazin-1-yl)-1-cyclopropyl-6-fluoro-1,4-dihydro-4-oxoquinoline-3-carboxylate (74): The coupling reaction between 55 and 71 was carried out as described for the synthesis of 51 on a 1.0 mmol scale. The crude product was purified by silica gel flash column chromatography (0% to 6% MeOH in EtOAc) to give 74 as a pale yellow coloured solid (0.53 g, 62%). ¹H NMR (400 MHz, CDCl₃) δ 1.15 (t, J=7.0, 6H), 1.21-1.30 (m, 8H), 1.32-1.39 (m, 2H), 1.48 (s, 9H), 3.21-3.28 (m, 4H), 3.42-3.49 (m, 1H), 3.62-3.69 (m, 4H), 3.72 (t, J=25.1, 1H), 3.87-4.16 (m, 8H), 4.91 (s, 2H), 7.31 (d, J=8.1, 1H), 7.55 (m, 2H), 7.98 (d, J=9.3, 2H), 8.15 (d, J=112.8, 1H), 8.49 (s, 1H), 11.07 (s, 1H): ¹⁹F (376 MHz, D₂O) δ-122.84 (dd, J=7.3, 12.9, 1F).

(4-Bisphosphonomethylphenylcarbamoyl)methyl 1-cyclopropyl-6-fluoro-1,4-dihydro-4-oxo-7-(piperazin-1-yl)quinoline-3-carboxylic acid (75): The de protection of 74 was carried out as described for that of 51 on a 0.62 mmol scale. The crude product was purified by Waters C18 Sep-Pak™ column (0% to 8% MeOH in H₂O) to give 75 as a pale yellow coloured solid (230 mg, 58%). ¹H NMR (400 MHz, D₂O) δ1.14-1.20 (m, 2H), 1.45-1.38 (m, 2H), 3.09-3.19 (m, 4H), 3.22-3.29 (m, 4H), 3.36 (t, J=22.7, 1H), 3.70-3.78 (m, 1H), 4.72 (s, 2H), 7.42 (d, J=8.5, 2H), 7.49 (d, J=7.0, 1H), 7.60 (d, J=8.5, 1H), 7.84 (d, J=14.0, 1H) 8.62 (bs, 1H): ¹⁹F (376 MHz, D₂O) δ-123.25 (dd, J=7.1, 13.3, 1F): ³¹P (162 MHz, D₂O) δ 16.74 (s, 2P): LCMS: 100% (254 nm), 100% (220 nm), 100% (290 nm). MS: (MH⁺) 639.1.

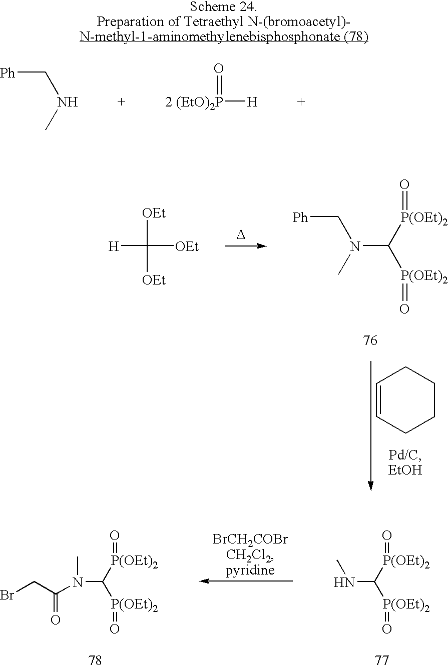

Tetraethyl N-benzyl-N-methyl-1-aminomethylenebisphosphonate (76): Compound 76 was prepared utilizing a modified procedure of that described in Synth. Comm. 1996, 26, 2037-2043. Triethyl orthoformate (13.8 g, 93.3 mmol), diethyl phosphite (32.2 g, 233 mmol) and N-benzylmethyl amine (9.42 g, 77.7 mmol) were heated in a 100 mL round bottom flask fitted with a distillation apparatus. The reaction was heated to a temperature of 180-190° C. for 3 h under Ar at which time the evolution of EtOH was complete. The reaction mixture was cooled to room temperature, diluted with CHCl₃ (400 mL), washed with aqueous NaOH (1 M) and brine then dried over Na₂SO₄. The solvent was removed at aspirator pressure resulting in the colourless oil 76 (31.7 g, 100%). ¹H NMR (400 MHz, CDCl₃) δ 1.34 (dt, J=1.6, 7.1, 12H), 2.66 (s, 3H), 3.48 (t, J=24.9, 1H), 3.99 (s, 2H), 4.07-4.24 (m, 8H), 7.24-7.39 (m, 5H).

Tetraethyl N-methyl-1-aminomethylenebisphosphonate (77): Compound 76 (12.4 g, 30.4 mmol) was dissolved in EtOH (150 mL) followed by the addition of palladium on carbon (10%, 5 g) and cyclohexene (9.0 mL, 88.7 mmol). The resulting mixture was heated to reflux under argon for 16 h. The cooled solution was filtered through glass fiber filter paper and concentrated at reduced pressure to give 77 as a pale yellow oil (8.7 g, 90%), which was used directly in the next step without further purification. ¹H NMR (400 MHz, CDCl₃) δ 1.36 (t, J=7.4, 12H), 2.69 (s, 3H), 4.20-4.31 (m, 8H): ³¹P (162 MHz, CDCl₃) δ 19.44 (s, 2P).

Tetraethyl N-(bromoacetyl)-N-methyl-1-aminomethylenebisphosphonate (78): A solution of bromoacetyl bromide (1.48 mL, 17.0 mmol) in CH₂Cl₂ (1 mL) was added dropwise to a stirred, cooled (ice-bath) solution of 77 (4.5 g, 14 mmol) and pyridine (1.78 mL, 21.3 mmol) in CH₂Cl₂ (25 mL). The reaction was stirred for 18 h while slowly warming to room temperature. After quenching the reaction by the addition of water the product was extracted with CH₂Cl₂ and the combined organics were washed with 10% aqueous HCl, brine, dried over sodium sulfate and concentrated at reduced pressure. The crude yellow oil was purified by silica gel HPFC (0% to 10% MeOH in EtOAc) resulting in 78 as a pale yellow liquid (2.93 g, 47%). ¹H NMR (400 MHz, CDCl₃) δ 1.32 (t, J=7.1, 12H), 3.38 (s, 3H), 3.92 (s, 2H), 4.15-4.25 (m, 8H), 5.69 (t, J=24.5, 1H): ³¹P (162 MHz, CDCl₃) δ 16.93 (s, 2P).

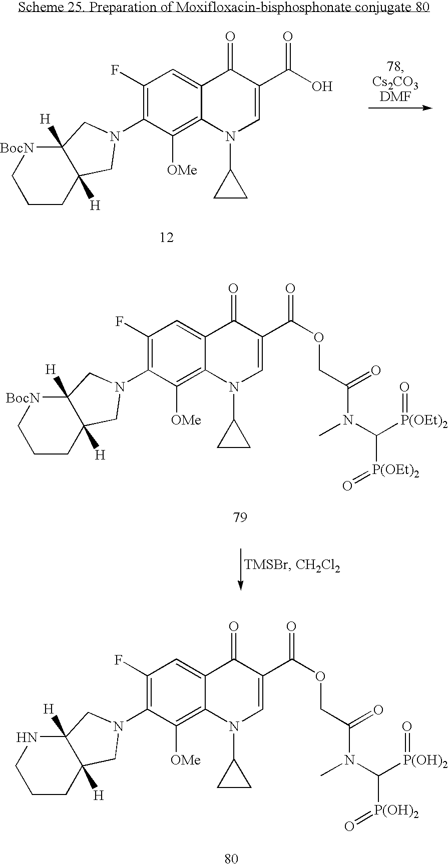

(N-Methyl-1,1-Bis(diethylphosphono)methylcarbamoyl)methyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (79): The coupling reaction between 12 and 78 was completed as described for the synthesis of 51 on a 3.27 mmol scale. The crude product was purified by silica gel flash column chromatography (0% to 10% MeOH in CH₂Cl₂) to give 79 as a yellow coloured solid (0.710 g, 25%). ¹H NMR (400 MHz, CDCl₃) δ 0.74-0.83 (m, 1H), 0.96-1.15 (m, 2H), 1.18-1.25 (m, 1H), 1.31-1.39 (m, 14H), 1.48 (s, 9H), 1.75-1.81 (m, 2H), 2.20-2.27 (m, 1H), 2.88 (bt, J=8.7, 1H), 3.21 (bs, 1H), 3.31 (s, 3H), 3.36 (bs, 1H), 3.54 (s, 3H), 3.80-3.91 (m, 2H), 4.01-4.08 (m, 2H), 4.13-4.26 (m, 8H), 4.77 (bs, 1H), 4.99 (AB q, J=15.8, 2H), 5.70 (t, J=24.8, 1H), 7.81 (d, J=7.8, 1H), 8.61 (s, 1H): ³¹P (162 MHz, CDCl₃) δ 17.10 (s, 2P).

(N-Methyl-1,1-Bisphosphonomethylcarbamoyl)methyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (80): The deprotection of 79 was carried out as described for that of 51 on a 0.815 mmol scale. The crude product was purified by Waters C18 Sep-Pak™ column (0% to 10% MeOH in H₂O) to give 80 as a pale yellow coloured solid (110 mg, 27%) that was a mixture of cis/trans rotamers. ¹H NMR (400 MHz, D₂O) δ 0.89-0.96 (m, 1H), 1.06-1.17 (m, 2H), 1.20-1.26 (m, 1H), 1.83-1.93 (m, 4H), 2.78 (bs, 1H), 3.10 (bs, 1H), 3.16 (s, ⅓-3H), 3.27 (s, ⅔-3H), 3.39 (bs, 1H), 3.55 (s, ⅓-3H), 3.57 (s, ⅔-3H), 3.67-3.83 (m, 3H), 3.88-4.14 (m, 3H), 4.92 (t, J=21.9, 1H), 5.12 (AB q, J=15.7, 2/3-2H), 5.16 (AB q, J=15.7, ⅓-2H), 7.37 (d, J=14.0, 2/3-1H), 7.44 (d, J=14.0, ⅓-1H), 8.96 (s, 1H): ¹⁹F (376 MHz, D₂O) δ-121.92 (d, J=14.0, 2/3-1F), −121.84 (d, J=14.0, ⅓-1F): ³¹P (162 MHz, D₂O) δ 12.31 (s, ⅓-2P), 13.08 (s, ⅔-2P): LCMS: 98.4% (254 nm), 99.2% (220 nm), 98.9% (320 nm). MS: (MH⁺) 647.1.

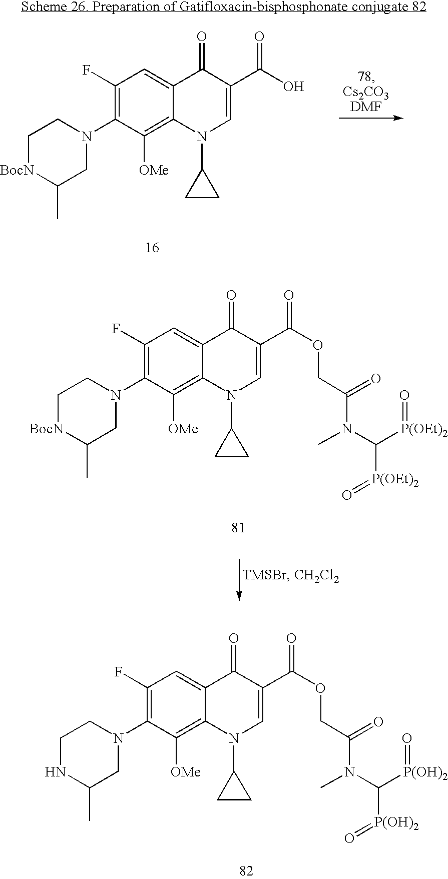

(N-Methyl-1,1-Bis(diethylphosphono)methylcarbamoyl)methyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (81): The coupling reaction between 78 and 16 was carried out as described for the synthesis of 51 on a 1.92 mmol scale. The crude product was purified by silica gel chromatography (0% to 10% MeOH in CH₂Cl₂) to give 81 as a pale yellow coloured solid (0.415 g, 30%). ¹H NMR (400 MHz, CDCl₃) δ 0.87 (bt, J=3.8, 2H), 1.13 (d, J=7.5, 2H), 1.30-1.39 (m, 15H), 1.50 (s, 9H), 3.19-3.27 (m, 3H), 3.31 (s, 3H), 3.42 (bt, J=13.4, 2H), 3.70 (s, 3H), 3.84-3.90 (m, 1H), 3.94 (d, J=12.2, 1H), 4.11-4.26 (m, 8H), 4.33 (bs, 1H), 4.97-5.01 (m, 2H), 5.70 (t, J=24.8, 1H), 7.88 (d, J=12.6, 1H), 8.63 (s, 1H): ¹⁹F (376 MHz, CDCl₃) δ-121.61 (d, J=13.0, 1F): ³¹P (162 MHz, CDCl₃) δ 17.11 (s, 2P).

(N-Methyl-1,1-Bisphosphonomethylcarbamoyl)methyl 7-(3-methylpiperazin-1-yl)-1-cyclopropyl-6-fluoro-1,4-dihydro-8-methoxy-4-oxoquinoline-3-carboxylate (82): The deprotection of 81 was completed as described for that of 51 on a 0.354 mmol scale. The crude product was purified by Waters C18 Sep-Pak™ column (0%-5% MeOH in H₂O) to give a mixture of cis/trans rotamers of 82 as a pale yellow coloured solid (135 mg, 54%). ¹H NMR (400 MHz, D₂O) δ1.06-1.11 (m, 2H), 1.19-1.23 (m, 2H), 1.33-1.36 (m, 3H), 3.16 (s, ⅓-3H), 3.27 (s, ⅔-3H), 3.21-3.30 (m, 1H), 3.32-3.36 (m, 1H), 3.40-3.48 (m, 1H), 3.53-3.64 (m, 3H), 3.74 (s, ⅔-3H), 3.79 (s, ⅓-3H), 3.89 (t, J=21.0, ⅓-1H), 4.13-4.17 (m, 1H), 4.92 (t, J=21.0, 2/3-1H), 5.13 (s, ⅔-2H), 5.19 (s, ⅓-2H), 7.45 (bd, J=12.0, 2/3-1H), 7.59 (bd, J=12.0, ⅓-1H), 9.02 (s, ⅔-1H), 9.03 (s, ⅓-1H): ¹⁹F (376 MHz, D₂O) δ-121.83 (d, J=12.0, ⅓-1F), −122.02 (d, J=12.0, ⅔-1F): ³¹P (162 MHz, D₂O) δ 12.32 (s, ⅓-2P), 13.11 (s, ⅔-2P): LCMS: 98.0% (254 nm), 97.3% (220 nm), 97.4% (290 nm). MS: (MH⁺) 621.1.

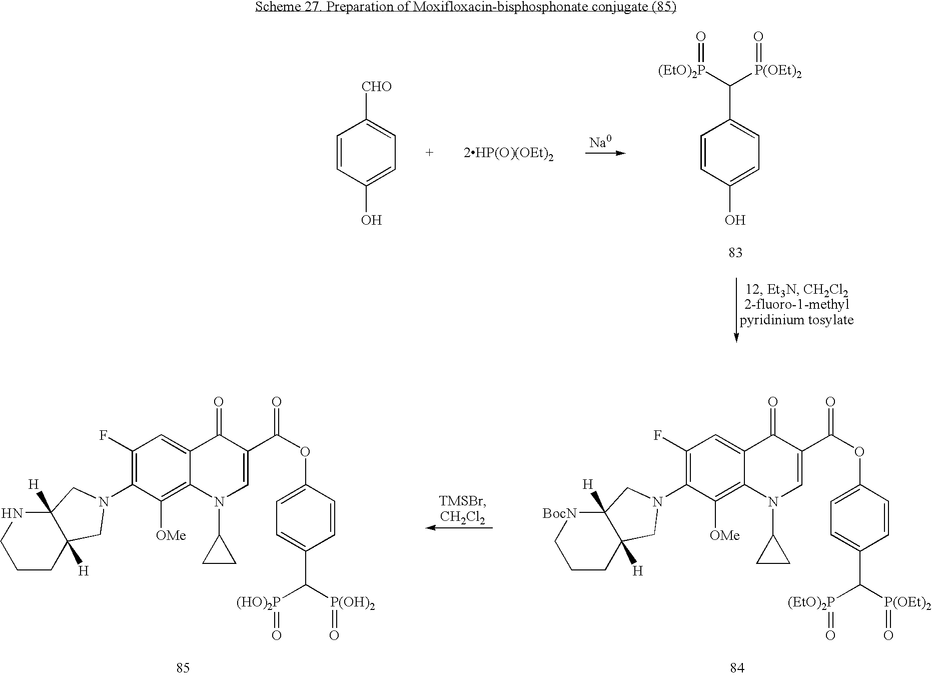

Tetraethyl (4-hydroxyphenyl)methylene bisphosphonate (83): This was prepared as described in Org. Biomol. Chem. (2004), 21:3162-3166. To diethyl phosphite (20 mL, 155 mmol) was cautiously added sodium metal (0.55 g, 23.9 mmol) in small portions at room temperature, ensuring that the reaction mixture never exceeds 50° C. 4-Hydroxybenzaldehyde (1.0 g, 8.2 mmol) was added to the resulting solution. The reaction mixture was stirred at room temperature for 48 h and then quenched with water (100 mL) and extracted with chloroform (3×100 ml). The chloroform layer was washed with brine, dried over Na₂SO₄ and concentrated under vacuum. The excess diethylphosphite was removed by bulb-to-bulb distillation. The resulting solid residue was washed with diethyl ether and filtered, to furnish 83 (2.42 g, 78%). ¹H NMR (CDCl₃, 400 MHz) δ 1.12 (t, J=7.0, 6H), 1.30 (t, J=7.0, 6H), 3.63 (t, J=25.4, 1H), 3.84-3.95 (m, 2H), 3.98-4.22 (m, 6H), 6.54 (d, J=8.2, 2H), 7.21 (bd, J=8.2, 2H), 8.42 (s, 1H): ³¹P (162 MHz, CDCl₃) δ 20.11 (s, 2P).

4-(Bis(diethylphosphono)methyl)phenyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (84): 2-Fluoro-1-methylpyridinium tosylate (0.340 g, 1.20 mmol) was added to a stirring solution of 12 (0.502 g, 1.00 mmol) in CH₂Cl₂ that was cooled in an ice-bath. Triethylamine (0.558 g, 4.00 mmol) was then added drop-wise and the resulting mixture was stirred at that temperature for 70 min. A solution of 83 (0.380 g, 1.00 mmol) in CH₂Cl₂ (1 mL) was then added and the resulting solution was stirred while warming to room temperature over 18 hr. After diluting with EtOAc, the organic layer was washed with 10% aqueous HCl, brine, 5% aqueous bicarbonate, brine then dried over Na₂SO₄. The crude product was purified by silica gel HPFC (0%-25% MeOH in EtOAc) to furnish 84 as a pale yellow solid (0.508 g, 59%).

4-(Bisphosphonomethyl)phenyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (85): The deprotection of 84 was completed as described for that of 51 on a 0.289 mmol scale. The crude product was purified by Waters C18 Sep-Pak™ column (40 mL H₂O then 40 mL 5% MeOH/H₂O) to give 85 as a pale yellow solid (214 mg, 54%): ¹H NMR (400 MHz, D₂O) δ 1.05-1.29 (m, 4H), 1.77-1.87 (m, 4H), 2.71 (bs, 1H), 2.96 (bt, J=10.7, 1H), 3.26-3.30 (m, 1H), 3.40 (t, J=21.9, 1H), 3.56-3.69 (m, 6H), 3.82-3.86 (m, 1H), 4.05-4.09 (m, 1H), 4.15-4.20 (m, 1H), 7.10 (d, J=7.6, 2H), 7.59-7.64 (m, 3H), 8.90 (d, J=2.3, 1H): ¹⁹F (376 MHz, D₂O) δ −120.98 (d, J=10.5, 1F): ³¹P (162 MHz, D₂O) δ 16.79 (s, 2P): LCMS: 100% (254 nm), 100% (220 nm), 100% (320 nm). MS: (MH⁺) 652.1.

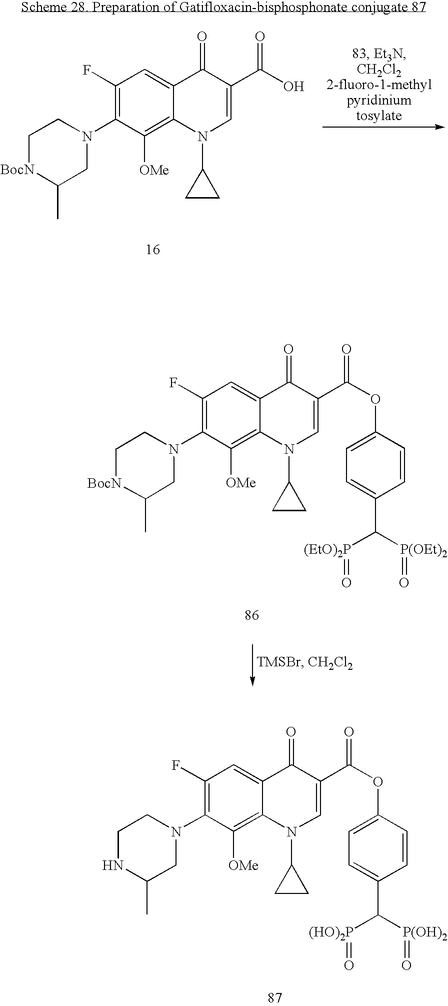

4-(Bis(diethylphosphono)methyl)phenyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (86): The coupling reaction between 16 and 83 was carried out as described for the synthesis of 84 on a 1.05 mmol scale. The crude product was purified by silica gel HPFC (0% to 30% MeOH in EtOAc) to resulting in 86 as a colourless solid (0.318 g, 36%). ¹H NMR (400 MHz, CDCl₃) δ 0.96 (t, J=3.9, 2H), 1.14-1.20 (m, 2H), 1.17 (t, J=6.8, 6H), 1.29 (t, J=6.8, 6H), 1.34 (d, J=6.7, 3H), 1.50 (s, 9H), 3.20-3.25 (m, 3H), 3.40-3.47 (m, 2H), 3.74 (s, 3H), 3.91-4.00 (m, 3H), 4.02-4.16 (m, 8H), 4.35 (bs, 1H), 7.20 (d, J=8.5, 2H), 7.40 (d, J=8.5, 2H), 7.93 (d, J=12.3, 1H), 8.71 (s, 1H): ¹⁹F (376 MHz, D₂O) δ-121.16 (d, J=12.5, 1F): ³¹P (162 MHz, CDCl₃) δ 19.35 (s, 2P).

4-(Bisphosphonomethyl)phenyl 7-(3-methylpiperazin-1-yl)-1-cyclopropyl-6-fluoro-1,4-dihydro-8-methoxy-4-oxoquinoline-3-carboxylate (87): The deprotection of 86 was completed as described for that of 51 on a 0.185 mmol scale. The crude product was purified by Waters C18 Sep-Pak™ column (40 mL H₂O) to give 87 as a colourless solid (80 mg, 61%). ¹H NMR (400 MHz, D₂O) δ 1.24 (d, J=6.2, 3H), 1.21-1.30 (m, 2H), 1.36-1.45 (m, 2H), 2.51-2.59 (m, 1H), 2.86 (bs, 1H), 3.04-3.14 (m, 2H), 3.20-3.26 (m, 1H), 3.42 (t, J=23.2, 1H), 3.46-3.56 (m, 2H), 3.65 (s, 3H), 4.02 (bs, 1H), 7.27 (d, J=7.7, 2H), 7.37 (d, J=12.0, 1H), 7.67 (d, J=7.0, 2H), 8.96 (s, 1H): ¹⁹F (376 MHz, D₂O) δ-121.76 (d, J=12.2, 1F): ³¹P (162 MHz, D₂O) δ 16.74 (s, 1P): LCMS: 100% (254 nm), 100% (220 nm), 100% (290 nm). MS: (MH⁺) 626.1.

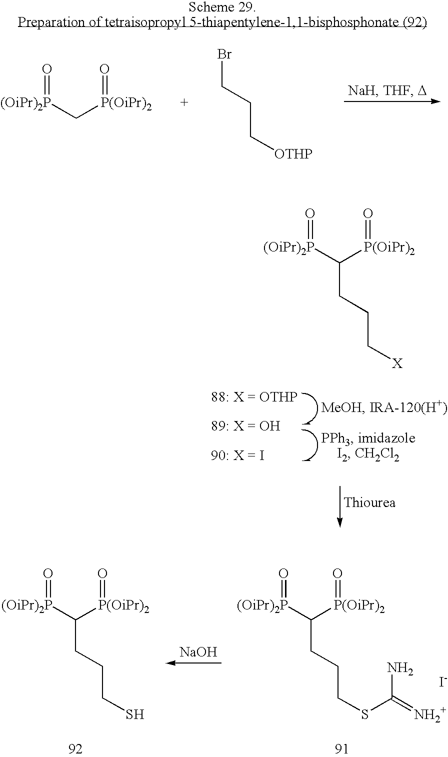

Tetraisopropyl 4-(2-Tetrahydro-2H-pyranyloxy)butylene-1,1-bisphosphonate (88): To a suspension of NaH (60% suspension in mineral oil, 1.43 g, 35.8 mmol) in dry THF (35 mL) was added dropwise tetraisopropyl methylenebisphosphonate (12.35 g, 35.9 mmol). The resulting clear solution was stirred 15 min at room temperature, after which 2-(3-bromopropoxy)tetrahydro-2H-pyran (8.0 g, 36 mmol) was added dropwise, rinsing the flask with 2×5 mL THF. The reaction mixture was heated to reflux for 6 h. The solvent was evaporated, and the residue taken up in ethyl acetate and washed with semi-saturated brine. The aqueous was extracted with ethyl acetate, the combined organics washed with brine, dried (MgSO₄) and evaporated. It was used as such in the following step.

Tetraisopropyl 4-hydroxybutylene-1,1-bisphosphonate (89): To a stirred solution of the crude product 88 (max. 36 mmol) in MeOH (70 mL) was added Amberlyst 15 (1.05 g). The reaction mixture was refluxed for 40 min, filtered and evaporated. The crude product was purified by flash chromatography on silica gel with gradient elution from 0-10% methanol/ethyl acetate to give pure 89 (7.0 g, 48% from tetraisopropyl methylenebisphosphonate). ¹H NMR (400 MHz, CDCl₃) δ 1.33-1.36 (m, 24H), 1.77-1.83 (m, 1H), 1.96-2.10 (m, 2H), 2.21 (tt, J=24.8, 5.4, 1H), 2.31-2.42 (m, 2H), 3.66 (t, J=5.9 2H), 4.70-4.83 (m, 4H).

Tetraisopropyl 4-iodobutylene-1,1-bisphosphonate (90): To a solution of 89 (7.0 g, 17 mmol) in CH₂Cl₂ (150 mL) were added triphenylphosphine (5.25 g, 20.0 mmol) and imidazole (1.78 g, 26.1 mmol). The reaction mixture was cooled to 0° C., before the addition of iodine (4.86 g, 19.1 mmol). The mixture was then removed from the cooling bath, stirred for 2 h, added to hexanes (300 mL) and filtered washing the precipitate with further hexanes (2×50 mL). The filtrate was evaporated and purified by flash chromatography on silica gel eluting with ethyl acetate to give pure 90 (7.6 g, 85%). ¹H NMR (400 MHz, CDCl₃) δ1.33-1.37 (m, 24H), 1.92-2.23 (m, 5H), 3.18 (t, J=6.7, 2H), 4.74-4.83 (m, 4H).

Tetraisopropyl 4-aminoisothioureidobutylene-1,1-bisphosphonate, hydroiodide salt (91): To a solution of 90 (3.8 g, 7.4 mmol) in ethanol (20 mL) was added thiourea (0.59 g, 7.75 mmol). The reaction mixture was refluxed for 18 h, evaporated and used as such in the following step. ¹H NMR (400 MHz, D₂O) δ 1.35-1.38 (m, 24H), 1.94-2.09 (m, 4H), 2.50-2.67 (m, 1H), 3.17 (t, J=6.1, 2H), 4.70-4.85 (m, 4H).

Tetraisopropyl 5-thiapentylene-1,1-bisphosphonate (92): To a solution of crude 91 (7.4 mmol) in water (30 mL) was added sodium hydroxide (0.396 g, 9.90 mmol). The reaction mixture was refluxed for 1.5 h, cooled to 0° C. and acidified with 1M HCl (10 mL). The product was extracted with CHCl₃ (3×50 mL), the organics washed with brine (70 mL), dried (MgSO₄) and evaporated to give a quantitative yield of crude 92 used as such in the following steps. ¹H NMR (400 MHz, CDCl₃) δ 1.33-1.36 (m, 24H), 1.88-2.19 (m, 5H), 2.50-2.56 (m, 2H), 4.74-4.83 (m, 4H).

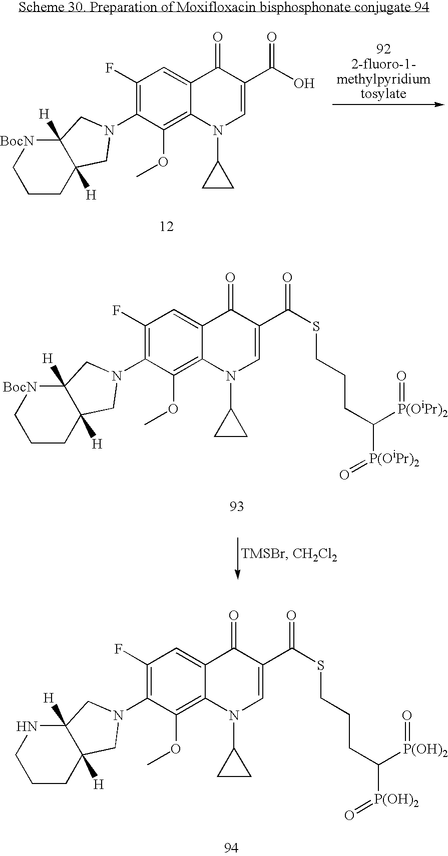

S-4,4-bis(diisopropylphosphono)butyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carbothioate (93): To a solution of 12 (200 mg, 0.400 mmol) in CH₂Cl₂ (3 mL) was added 2-fluoro-1-methylpyridinium tosylate (0.136 g, 0.480 mmol). The reaction mixture was cooled to 0° C., and triethylamine (0.20 mL, 1.43 mmol) was added via syringe. After stirring 1 h at 0° C. a solution of thiol 92 (0.208 g, 0.497 mmol) in CH₂Cl₂ (3 mL) was added. After a further 1 h at 0° C. the reaction was allowed to warm to room temperature overnight. The reaction mixture was diluted with ethyl acetate and washed with ice cold saturated NH₄Cl solution, 5% NaHCO₃, and water. After drying (MgSO₄) and evaporation the residue was purified by flash chromatography on silica gel with gradient elution from 2.5-5% methanol/CH₂Cl₂ to give pure 93 (0.2410 g, 67.0%). ¹H NMR (400 MHz, CDCl₃) δ 0.73-0.82 (m, 1H), 0.97-1.11 (m, 2H), 1.24-1.27 (m, 1H), 1.32-1.36 (m, 24H), 1.48 (s, 9H), 1.59-2.28 (m, 10H), 2.82-2.93 (m, 1H), 2.97 (t, J=7.2, 2H), 3.17-3.26 (m, 1H), 3.30-3.43 (m, 1H), 3.55 (s, 3H), 3.78-3.95 (m, 2H), 4.05-4.12 (m, 2H), 4.72-4.85 (m, 5H), 7.84 (d, J=13.9, 1H), 8.54 (s, 1H).

S-4,4-bisphosphonobutyl 7-((4aS,7aS)-octahydropyrrolo[3,4-b]pyridin-6-yl)-1-cyclopropyl-6-fluoro-1,4-dihydro-8-methoxy-4-oxoquinoline-3-carbothioate (94): To a solution of 93 (633 mg, 0.702 mmol) in CH₂Cl₂ (50 mL) was added TMSBr (0.93 mL, 7.05 mmol). The reaction mixture was stirred for 65 h, the solvent removed under reduced pressure and the solid dried under high vacuum for 1 h. The solid was suspended in H₂O (200 mL) and the pH was immediately adjusted to pH 8 by the addition of 1M NaOH, with concomitant dissolution of the product. The product solution was filtered washing the insoluble material with water and CHCl₃. The aqueous phase was evaporated, and purified by reverse-phase chromatography (gradient elution, 100% water −33% methanol/water). The pure product 94 was obtained as a yellowish white solid (236 mg, 47% recovery based on tetrasodium salt of product). ¹H NMR (400 MHz, D₂O) δ 1.02-1.11 (m, 1H), 1.13-1.22 (m, 2H), 1.27-1.36 (m, 1H), 1.64-1.98 (m, 9H), 2.42-2.52 (m, 1H), 2.59-2.69 (m, 1H), 2.74-2.84 (m, 1H), 2.95-3.25 (m, 1H), 3.34-3.44 (m, 1H), 3.56-3.70 (m, 2H), 3.61 (s, 3H), 3.83-3.96 (m, 2H), 4.08-4.18 (m, 2H), 7.53 (d, J=14.1, 1H), 8.59 (s, 1H). ¹⁹F (376 MHz, D₂O) δ-121.38 (d, J=13.2, 1F). ³¹P (162 MHz, D₂O) δ20.74 (s, 2P). MS: (MH⁺) 634.0.

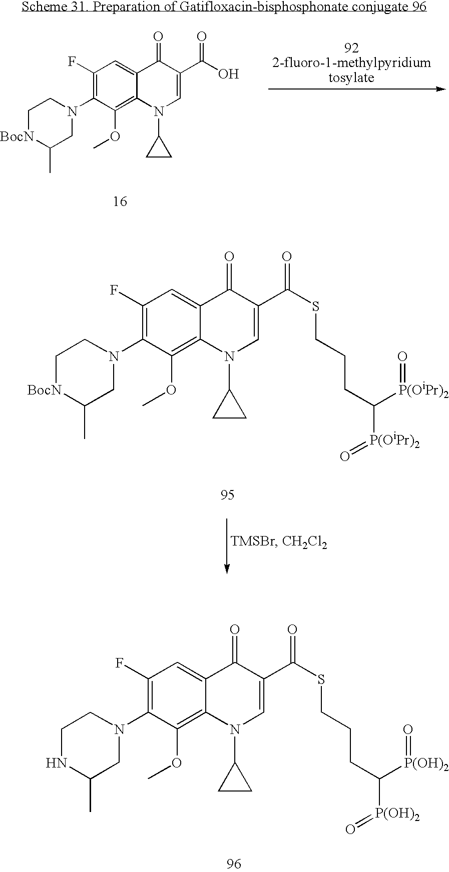

S-4,4-bis(diisopropylphosphono)butyl 7-(4-(tert-butoxycarbonyl)-3-methylpiperazin-1-yl)-1-cyclopropyl-6-fluoro-1,4-dihydro-8-methoxy-4-oxoquinoline-3-carbothioate (95): To a solution of 16 (601 mg, 1.26 mmol) in CH₂Cl₂ (5 mL) was added 2-fluoro-1-methylpyridinium tosylate (0.371 g, 1.31 mmol). The reaction mixture was cooled to 0° C., and triethylamine (0.63 mL, 4.52 mmol) was added via syringe. After stirring 80 min at 0° C. a solution of thiol 92 (0.575 g, 1.37 mmol) in CH₂Cl₂ (5 mL) was added. After a further 10 min at 0° C. the reaction was allowed to warm to room temperature overnight. The reaction mixture was diluted with ethyl acetate (50 mL) and washed with ice cold saturated NH₄Cl solution (2×25 mL), ice cold 5% NaHCO₃ (2×25 mL), water (25 mL) and brine (25 mL). After drying (MgSO₄) and evaporation the residue was purified by flash chromatography on silica gel with gradient elution from 2.5-5% methanol/CH₂Cl₂ to give 95 (0.663 g, 60.0%) contaminated with a small amount of 16. ¹H NMR (400 MHz, CDCl₃) δ 0.88-1.01 (m, 2H), 1.10-1.27 (m, 2H), 1.31-1.39 (m, 27H), 1.49 (s, 9H), 1.85-2.28 (m, 5H), 2.97 (t, J=7.4, 2H), 3.18-3.52 (m, 5H), 3.71 (s, 3H), 3.80-4.05 (m, 2H), 4.34 (bs, 1H), 4.72-4.87 (m, 4H), 7.91 (d, J=12.5, 1H), 8.57 (s, 1H).

S-4,4-bisphosphonobutyl 7-(3-methylpiperazin-1-yl)-1-cyclopropyl-6-fluoro-1,4-dihydro-8-methoxy-4-oxoquinoline-3-carbothioate (96): To a solution of 95 (663 mg, 0.757 mmol) in CH₂Cl₂ (50 mL) was added TMSBr (1.0 mL, 7.6 mmol). The reaction mixture was stirred for 92 h, the solvent removed under reduced pressure and the solid dried under high vacuum for 1 h. The solid was suspended in H₂O (200 mL) and the pH was immediately adjusted to pH 7.5 by the addition of 1M NaOH, with concomitant dissolution of the product. The product solution was washed with CHCl₃ (2×50 mL), evaporated, and purified by reverse-phase chromatography (gradient elution, 100% water−30% methanol/water). The pure product 96 was obtained as a white solid (103 mg, 20% recovery based on tetrasodium salt of product). ¹H NMR (400 MHz, D₂O) δ 0.96-1.04 (m, 2H), 1.16-1.25 (m, 2H), 1.33 (d, J=6.3, 3H), 1.76-2.02 (m, 5H), 2.99-3.08 (m, 2H), 3.16-3.59 (m, 7H), 3.76 (s, 3H), 4.06-4.14 (m, 1H), 7.34 (d, J=12.1, 1H), 8.66 (s, 1H). ¹⁹F (376 MHz, D₂O) δ-121.26 (d, J=12.0, 1F). ³¹P (162 MHz, D₂O) δ 20.80 (s, 2P). MS: (MH⁺) 608.1.

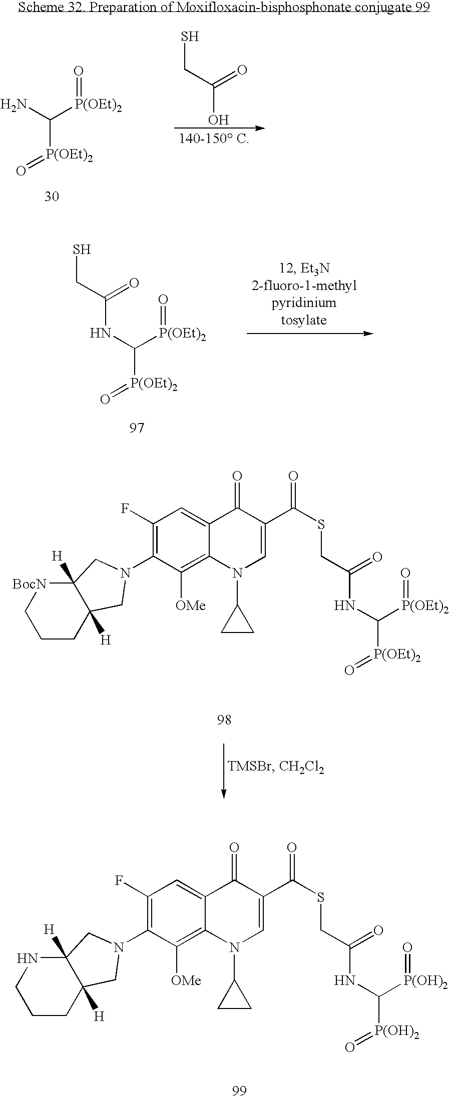

Tetraethyl 1-(N-3-thiapropionylamino)methylenebisphosphonate (97): A mixture of amine 30 (691 mg, 2.28 mmol) and mercaptoacetic acid (200 μL, 2.89 mmol) was heated to 140-150° C. under continuous purging with Ar. When steam evolution appeared complete the residue was purified by flash chromatography on silica gel eluting with 5% methanol/CH₂Cl₂ to give 97 (0.321 g, 37%). ¹H NMR (400 MHz, CDCl₃) δ 1.339 (t, J=7.0, 6H), 1.344 (t, J=7.0, 6H), 1.99 (t, J=8.8, 1H), 3.25-3.35 (m, 2H), 4.11-4.30 (m, 8H), 4.97 (td, J=21.4, J=110.1, 1H).

S-(1,1-Bis(diethylphosphono)methylcarbamoyl)methyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carbothioate (98): To a solution of 12 (427 mg, 0.851 mmol) in CH₂Cl₂ (6.5 mL) was added 2-fluoro-1-methylpyridinium tosylate (0.292 g, 1.03 mmol). The reaction mixture was cooled to 0° C., and triethylamine (0.43 mL, 3.09 mmol) was added via syringe. After stirring 1 h at 0° C. a solution of thiol 97 (0.32 g, 0.85 mmol) in CH₂Cl₂ (10 mL) was added. After a further 10 min at 0° C. the reaction was allowed to warm to room temperature overnight. The reaction mixture was diluted with CH₂Cl₂ and washed with ice cold saturated NH₄Cl solution, ice cold 5% NaHCO₃, water and brine. After drying (MgSO₄) and evaporation the residue was purified by flash chromatography on silica gel eluting with 4% methanol/CH₂Cl₂ to give slightly impure 98 (0.418 g, 57%) as a yellow foam. ¹H NMR (400 MHz, CDCl₃) δ 0.75-0.85 (m, 1H), 0.99-1.17 (m, 2H), 1.21-1.39 (m, 13H), 1.48 (s, 9H), 1.61 (s, 2H), 1.75-1.83 (m, 2H), 2.21-2.30 (m, 1H), 2.82-2.94 (m, 1H), 3.17-3.28 (m, 1H), 3.30-3.44 (m, 1H), 3.57 (s, 3H), 3.72 (s, 2H), 3.81-3.88 (m, 1H), 3.91-3.98 (m, 1H), 4.01-4.28 (m, 10H), 4.70-4.86 (bs, 1H), 4.98 (td, J=21.6, 9.9, 1H), 7.10 (d, J=10.3, 1H), 8.59 (s, 1H).

S-(1,1-Bisphosphonomethylcarbamoyl)methyl 1-cyclopropyl-6-fluoro-1,4-dihydro-7-((4aS,7aS)-octahydropyrrolo[3,4-b]pyridin-6-yl)-8-methoxy-4-oxoquinoline-3-carbothioate (99): To a solution of 98 (418 mg, 0.486 mmol) in CH₂Cl₂ (30 mL) was added TMSBr (0.64 mL, 4.8 mmol). The reaction mixture was stirred for 41 h, the solvent removed under reduced pressure and the solid dried under high vacuum for 1 h. The solid was suspended in H₂O (100 mL) and the pH was immediately adjusted to pH 7 by the addition of 1M NaOH, with concomitant dissolution of the product. The product solution was washed with CHCl₃ (2×50 mL), filtered, evaporated, and purified by reverse-phase chromatography (gradient elution, 100% water-15% methanol/water). The pure product 99 was obtained as a yellowish white solid (90 mg, 25% recovery based on tetrasodium salt of product. ¹H NMR (400 MHz, D₂O) δ 0.93-1.03 (m, 1H), 1.03-1.19 (m, 2H), 1.19-1.29 (m, 1H), 1.78-2.01 (m, 4H), 2.79 (bs, 1H), 3.02-3.12 (m, 1H), 3.36-3.44 (m, 1H), 3.61 (s, 3H), 3.57-3.85 (m, 3H), 3.78 (bs, 2H), 3.89-3.95 (m, 1H), 4.02-4.16 (m, 2H), 4.27 (t, J=18.7, 1H), 7.40 (d, J=13.9, 1H), 8.59 (s, 1H). ¹⁹F (376 MHz, D₂O) δ-94.67 (d, J=13.4, 1F). ³¹P (162 MHz, D₂O) δ 14.08 (d, J=22.0, 1P), 13.95 (d, J=22.0, 1P). MS: (MH⁺) 649.0.

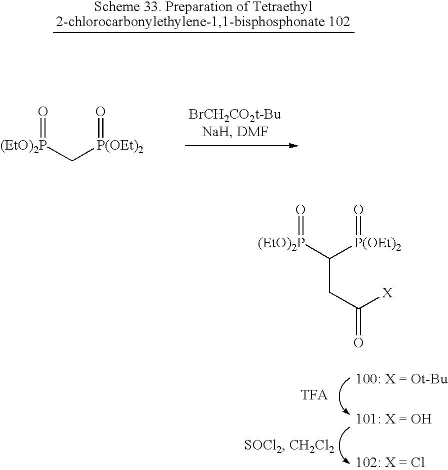

The compounds above were synthesized in a similar fashion to the compounds in Bioorg. Med. Chem. (1999), 7: 901-19.

Tetraethyl 2-t-Butoxycarbonylethylene-1,1-bisphosphonate (100): To a solution of tetraethyl methylenebisphosphonate (3.00 g, 10.4 mmol) in dry DMF (9 mL) was added NaH (60% suspension in mineral oil, 0.46 g, 11.5 mmol) portionwise. The resulting slurry was stirred for 30 min at room temperature, after which t-butyl bromoacetate (1.7 mL, 11.5 mmol) was quickly added neat. The reaction mixture was stirred for 1 h and quenched by adding 2 mL of a saturated solution of NH₄Cl. The reaction mixture was evaporated and purified by flash chromatography on silica gel eluting with 5% methanol/ethyl acetate to give pure 100 (2.1 g, 50%) as a clear colourless oil. ¹H NMR (400 MHz, CDCl₃) δ 1.33 (bt, J=7.0, 12H), 1.46 (s, 9H), 2H), 2.76 (td, J=16.0, 6.1, 2H), 3.07 (tt, J=24.0, 6.1, 1H), 4.10-4.25 (m, 8H).

Tetraethyl 2-carboxyethylene-1,1-bisphosphonate (101): Ester 100 (2.1 g, 5.2 mmol) was stirred in TFA (12 mL) for 2.5 min and concentrated under reduced pressure. Crude acid 101 was purified by flash chromatography (gradient elution 100% ethyl acetate −10% methanol/ethyl acetate). Acid 101 was obtained as a white solid (1.35 g, 75%). ¹H NMR (400 MHz, CDCl₃) δ1.28-1.39 (m, 12H), 2.86 (td, J=16.1, 6.3, 2H), 3.12 (tt, J=24.0, 6.3, 1H), 4.13-4.26 (m, 8H).

Tetraethyl 2-chlorocarbonylethylene-1,1-bisphosphonate (102): To acid 101 (1.02 g, 2.95 mmol) in CH₂Cl₂ (15 mL) was added freshly distilled SOCl₂ (0.84 mL, 11.6 mmol). The mixture was stirred at reflux for 3 h and concentrated to dryness to give crude 102 as a colourless oil (quantitative) which was immediately used for the next step without further purification. ¹H NMR (400 MHz, CDCl₃) δ 1.30-1.40 (m, 12H), 3.05 (tt, J=23.5, 6.2, 1H), 3.40 (td, J=14.8, 6.2, 2H), 3.12 (tt, J=24.0, 6.3, 1H), 4.13-4.27 (m, 8H).

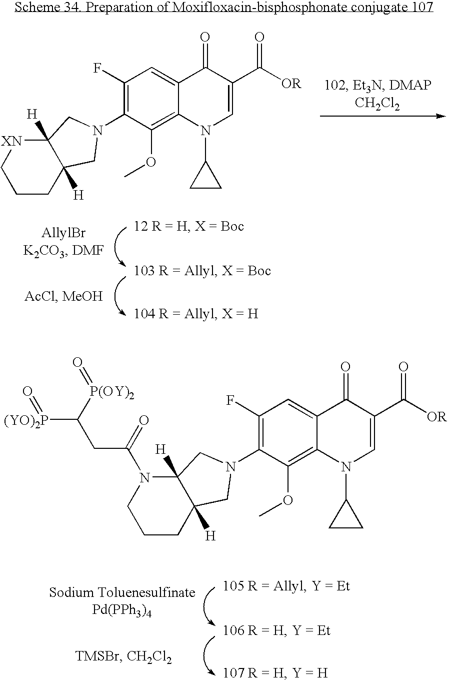

Allyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (103): To a solution of acid 12 (1.00 g, 2.0 mmol) in dry DMF (20 mL) was added K₂CO₃ (332 mg, 2.4 mmol) and allyl bromide (210 μL, 2.4 mmol). The reaction mixture was heated at 75-80° C. for 24 h, evaporated, and the residue taken up in water and ethyl acetate. The aqueous layer was extracted with ethyl acetate, and the combined organics washed with brine, dried (MgSO₄) and evaporated. Crude 103 (0.80 g, 74%) was used in the following step. ¹H NMR (400 MHz, CDCl₃) δ 0.73-0.83 (m, 1H), 0.88-1.12 (m, 2H), 1.18-1.29 (m, 1H), 1.48 (s, 9H), 1.58-1.85 (m, 4H), 2.19-2.28 (m, 1H), 2.82-2.93 (m, 1H), 3.15-3.26 (m, 1H), 3.30-3.40 (m, 1H), 3.56 (s, 3H), 3.78-3.92 (m, 2H), 3.99-4.11 (m, 2H), 4.70-4.90 (m, 3H), 5.27 (dd, J=10.4, 1.3, 1H), 5.48 (dd, J=17.2, 1.5, 1H), 6.00-6.11 (m, 1H), 7.84 (d, J=14.3, 1H), 8.56 (s, 1H).

Allyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (104): To a solution of protected amine 103 (0.80 g, 1.5 mmol) in dry methanol (25 mL) cooled to 0° C. was added acetyl chloride (5.33 mL, 74.6 mmol). The resulting solution was allowed to warm to room temperature over 1.5 h, concentrated, and the residue taken up in ice cold saturated NaHCO₃ and CH₂Cl₂. After drying and concentration crude 104 was obtained (0.62 g, 95%) sufficiently pure to use directly in the next step. ¹H NMR (400 MHz, CDCl₃) δ 0.75-0.85 (m, 1H), 0.95-1.10 (m, 2H), 1.14-1.24 (m, 1H), 1.53-1.68 (m, 1H), 1.72-1.90 (m, 3H), 2.38 (bs, 1H), 2.73-2.87 (m, 1H), 3.12-3.24 (m, 1H), 3.35-3.64 (m, 3H), 3.55 (s, 3H), 3.82-4.02 (m, 3H), 4.74-4.88 (m, 2H), 5.26 (dd, J=10.6, 1.1, 1H), 5.46 (dd, J=17.2, 1.5, 1H), 5.98-6.11 (m, 1H), 7.61 (d, J=13.9, 1H), 8.53 (s, 1H).

Allyl 7-((4aS,7aS)-1-(3,3-bis(diethylphosphono)propionyl)-octahydropyrrolo[3,4-b]pyridin-6-yl)-1-cyclopropyl-6-fluoro-1,4-dihydro-8-methoxy-4-oxoquinoline-3-carboxylate (105): To a solution of crude amine 104 (0.624 g, 1.41 mmol), triethylamine (0.24 mL, 1.69 mmol) and DMAP (17 mg, 0.14 mmol) in CH₂Cl₂ (20 mL) cooled to 0° C. was added dropwise a CH₂Cl₂ solution of crude acyl chloride 102 (1.76 mmol in 12.5 mL). The resulting mixture was allowed to warm to room temperature overnight, diluted with CH₂Cl₂, washed with saturated NaHCO₃, the aqueous back-extracted with CH₂Cl₂, the combined organics washed with brine, dried (MgSO₄) and concentrated. Pure amide 105 (0.80 g, 74%) was obtained by flash chromatography (5% methanol/CH₂Cl₂). ¹H NMR (400 MHz, CDCl₃, mixture of rotamers) δ 0.72-0.81 (m, 1H), 0.93-1.10 (m, 2H), 1.15-1.26 (m, 1H), 1.28-1.38 (m, 12H), 1.43-1.64 (m, 2H), 1.78-1.90 (m, 2H), 2.18-2.36 (m, 1H), 2.75-3.10 (m, 3H), 3.13-3.29 (m, 2H), 3.34-3.66 (m, 2H), 3.55 (s, 3H, major rotamer), 3.59 (s, 3H, minor rotamer), 3.74-4.26 (m, 12H), 4.53-4.66 (m, 1H), 4.76-4.88 (m, 2H), 5.17-5.35 (overlapping doublets of doublets, 1H), 5.42-5.54 (overlapping doublets of doublets, 1H), 5.98-6.10 (m, 1H), 7.82 (d, J=13.9, 1H, major rotamer), 7.84 (d, J=14.3, 1H, minor rotamer), 8.54 (s, 1H, major rotamer), 8.55 (s, 1H, minor rotamer).

7-((4aS,7aS)-1-(3,3-bis(diethylphosphono)propionyl)-octahydropyrrolo[3,4-b]pyridin-6-yl)-1-cyclopropyl-6-fluoro-1,4-dihydro-8-methoxy-4-oxoquinoline-3-carboxylic acid (106): To a solution of allyl ester 105 (0.80 g, 1.04 mmol) in THF (20 mL) was added Pd(PPh₃)₄ (24 mg, 0.02 mmol) and a water (2 mL) solution of sodium toluenesulfinate (204 mg, 1.14 mmol). The mixture was stirred at room temperature for 1.25 h, evaporated and purified by flash chromatography (gradient elution 5% methanol/CH₂Cl₂−10% methanol/CH₂Cl₂) to give 106 (0.60 g, 79%). ¹H NMR (400 MHz, CDCl₃, mixture of rotamers) δ 0.76-0.85 (m, 1H), 1.02-1.19 (m, 2H), 1.25-1.40 (m, 13H), 1.46-1.67 (m, 2H), 1.80-1.93 (m, 2H), 2.33-2.40 (m, 1H), 2.72-3.10 (m, 3H), 3.12-3.36 (m, 2H), 3.40-3.65 (m, 1H), 3.56 (s, 3H, major rotamer), 3.60 (s, 3H, minor rotamer), 3.78-4.28 (m, 11H), 4.57-4.70 (m, 1H), 5.20-5.30 (m, 1H), 7.81 (d, J=13.9, 1H, major rotamer), 7.84 (d, J=13.6, 1H, minor rotamer), 8.78 (s, 1H, major rotamer), 8.79 (s, 1H, minor rotamer).

7-((4aS,7aS)-1-(3,3-bisphosphonopropionyl)-octahydropyrrolo[3,4-b]pyridin-6-yl)-1-cyclopropyl-6-fluoro-1,4-dihydro-8-methoxy-4-oxoquinoline-3-carboxylic acid (107): To a solution of 107 (0.60 g, 0.82 mmol) in CH₂Cl₂ (40 mL) was added TMSBr (1.1 mL, 8.2 mmol). The reaction mixture was stirred for 38 h, the solvent removed under reduced pressure and the solid dried under high vacuum for 1 h. The solid was suspended in H₂O (80 mL) and the pH was immediately adjusted to pH 7 by the addition of 1M NaOH, with concomitant dissolution of the product. The product solution was concentrated, and purified by reverse-phase chromatography (gradient elution, 100% water-25% methanol/water). The pure product 107 was obtained as a yellow solid (189 mg, 32% recovery based on tetrasodium salt of product). ¹H NMR (400 MHz, D₂O, mixture of rotamers) δ 0.73-0.83 (m, 1H), 0.95-1.16 (m, 2H), 1.17-1.28 (m, 1H), 1.46-1.73 (m, 2H), 1.76-1.89 (m, 2H), 2.30-2.62 (m, 2H), 2.67-4.48 (m, 8H), 3.60 (s, 3H), 4.87-4.98 (m, 0.43H), 5.08-5.18 (m, 0.57H), 7.64 (d, J=14.3, 1H), 8.47 (s, 1H). ¹⁹F (376 MHz, D₂O) δ-96.88-96.73 (m, 1F). ³¹P (162 MHz, D₂O) δ 19.94-20.26 (m, 2P). MS: (MH⁺) 618.1.

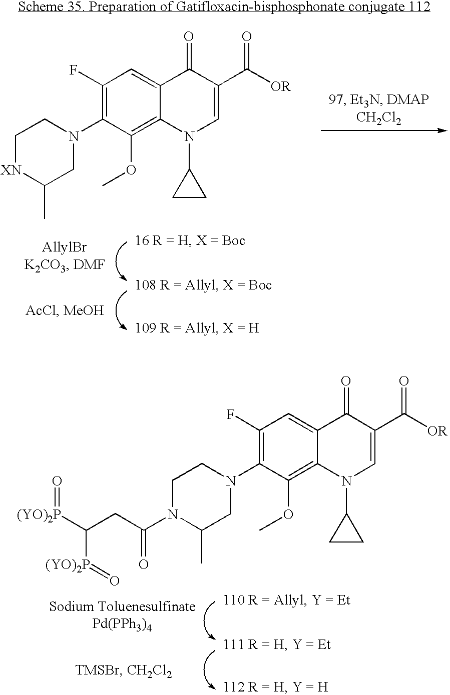

Allyl 7-(4-(tert-butoxycarbonyl)-3-methylpiperazin-1-yl)-1-cyclopropyl-6-fluoro-1,4-dihydro-8-methoxy-4-oxoquinoline-3-carboxylate (108): To a solution of acid 16 (0.60 g, 1.3 mmol) in dry DMF (14 mL) was added K₂CO₃ (221 mg, 1.6 mmol) and allyl bromide (140 μL, 1.6 mmol). The reaction mixture was heated at 75-80° C. for 24 h, evaporated, and the residue taken up in water and ethyl acetate. The aqueous layer was extracted with ethyl acetate, and the combined organics washed with brine, dried (MgSO₄) and evaporated. Crude 108 (0.51 g, 78%) was used in the following step. ¹H NMR (400 MHz, CDCl₃) δ 0.83-0.98 (m, 2H), 1.07-1.20 (m, 2H), 1.33 (d, J=6.6, 1H), 1.49 (s, 9H), 3.15-3.49 (m, 5H), 3.72 (s, 3H), 3.84-3.99 (m, 2H), 4.34 (bs, 1H), 4.83 (d, J=5.9, 2H), 5.28 (dd, J=10.4, 1.3, 1H), 5.48 (dd, J=17.2, 1.5, 1H), 6.00-6.11 (m, 1H), 7.90 (d, J=12.5, 1H), 8.59 (s, 1H).

Allyl 7-(3-methylpiperazin-1-yl)-1-cyclopropyl-6-fluoro-1,4-dihydro-8-methoxy-4-oxoquinoline-3-carboxylate (109): To a suspension of protected amine 108 (0.51 g, 0.99 mmol) in dry methanol (20 mL) cooled to 0° C. was added acetyl chloride (4.3 mL, 60.5 mmol). The resulting solution was allowed to warm to room temperature over 40 min, evaporated, and the residue taken up in ice cold saturated NaHCO₃ and CH₂Cl₂. After drying and evaporation crude 109 was obtained (0.38 g, 92%) sufficiently pure to use directly in the next step. ¹H NMR (400 MHz, CDCl₃) δ 0.86-1.00 (m, 2H), 1.08-1.18 (m, 5H), 2.87-2.97 (m, 1H), 3.00-3.14 (m, 3H), 3.21-3.39 (m, 3H), 3.77 (s, 3H), 3.86-3.95 (m, 1H), 4.80-4.86 (m, 2H), 5.25-5.30 (m, 1H), 5.45-5.51 (m, 1H), 6.00-6.10 (m, 1H), 7.88 (d, J=12.5, 1H), 8.58 (s, 1H).

Allyl 7-(4-(3,3-bis(diethylphosphono)propionyl)-3-methylpiperazin-1-yl)-1-cyclopropyl-6-fluoro-1,4-dihydro-8-methoxy-4-oxoquinoline-3-carboxylate (110): To a solution of crude amine 109 (0.378 g, 0.910 mmol), triethylamine (0.15 mL, 1.09 mmol) and DMAP (11 mg, 0.09 mmol) in CH₂Cl₂ (15 mL) cooled to 0° C. was added dropwise a CH₂Cl₂ solution of crude acyl chloride 102 (1.13 mmol in 8.5 mL). The resulting mixture was allowed to warm to room temperature overnight, diluted with CH₂Cl₂, washed with saturated NaHCO₃, the aqueous back-extracted with CH₂Cl₂, the combined organics washed with brine, dried (MgSO₄) and evaporated. Pure amide 110 (0.55 g, 81%) was obtained by flash chromatography (5% methanol/CH₂Cl₂). ¹H NMR (400 MHz, CDCl₃, mixture of rotamers) δ 0.87-0.99 (m, 2H), 1.10-1.21 (m, 2H), 1.29-1.43 (m, 15H), 2.78-3.05 (m, 2H), 3.16-3.82 (m, 6H), 3.72 (s, 3H), 3.85-3.95 (m, 1H), 4.12-4.30 (m, 9H), 4.47-4.59 (m, 0.5H), 4.80-4.92 (m, 2.5H), 5.28 (dd, J=10.4, 1.3, 1H), 5.45-5.55 (m, 1H), 6.00-6.12 (m, 1H), 7.91 (d, J=12.5, 1H), 8.59 (s, 1H).

7-(4-(3,3-bis(diethylphosphono)propionyl)-3-methylpiperazin-1-yl)-1-cyclopropyl-6-fluoro-1,4-dihydro-8-methoxy-4-oxoquinoline-3-carboxylic acid (111): To a solution of allyl ester 110 (0.55 g, 0.74 mmol) in THF (20 mL) was added Pd(PPh₃)₄ (20 mg, 0.02 mmol) and a water (1.6 mL) solution of sodium toluenesulfinate (158 mg, 0.89 mmol). The mixture was stirred at room temperature for 45 min, made slightly acidic by addition of 1M HCl (0.95 mL, 0.95 mmol) and evaporated. The residue was redissolved in CHCl₃, dried (MgSO₄) and evaporated, followed by flash chromatography (5% methanol/CHCl₃) to give 111 (0.35 g, 67%). ¹H NMR (400 MHz, CDCl₃, mixture of rotamers) δ 0.93-1.06 (m, 2H), 1.16-1.28 (m, 2H), 1.30-1.40 (m, 15H), 2.81-3.05 (m, 2H), 3.18-3.84 (m, 6H), 3.73 (s, 3H), 3.97-4.05 (m, 1H), 4.12-4.28 (m, 9H), 4.49-4.61 (m, 0.5H), 4.84-4.94 (m, 0.5H), 7.91 (d, J=12.5, 1H), 8.83 (s, 1H).

7-(4-(3,3-bisphosphonopropionyl)-3-methylpiperazin-1-yl)-1-cyclopropyl-6-fluoro-1,4-dihydro-8-methoxy-4-oxoquinoline-3-carboxylic acid (112): To a solution of 111 (0.35 g, 0.50 mmol) in CH₂Cl₂ (30 mL) was added TMSBr (0.66 mL, 5.0 mmol). The reaction mixture was stirred for 22 h, the solvent removed under reduced pressure and the solid dried under high vacuum for 1 h. The solid was suspended in H₂O (120 mL) and the pH was immediately adjusted to pH 7.5 by the addition of 1M NaOH, with concomitant dissolution of the product. The product solution was evaporated, and purified by repeated reverse-phase chromatography eluting with water. The pure product 112 was obtained as a pale yellow solid (108 mg, 34% recovery based on tetrasodium salt of product). ¹H NMR (400 MHz, D₂O, mixture of rotamers) δ 0.88-1.04 (m, 2H), 1.06-1.22 (m, 2H), 1.38 (d, J=7.0, 1.7H), 1.51 (d, J=6.6, 1.3H), 2.39-2.62 (m, 1H), 2.78-3.08 (m, 2H), 3.27-3.48 (m, 4H), 3.77 (s, 3H), 3.98-4.78 (m, 3H), 7.75 (d, J=12.8, 1H), 8.53 (s, 1H). ¹⁹F (376 MHz, D₂O) δ-95.77-95.63 (m, 1F). ³¹P (162 MHz, D₂O) δ 19.76-20.16 (m, 2P). MS: (M−H) 590.0.

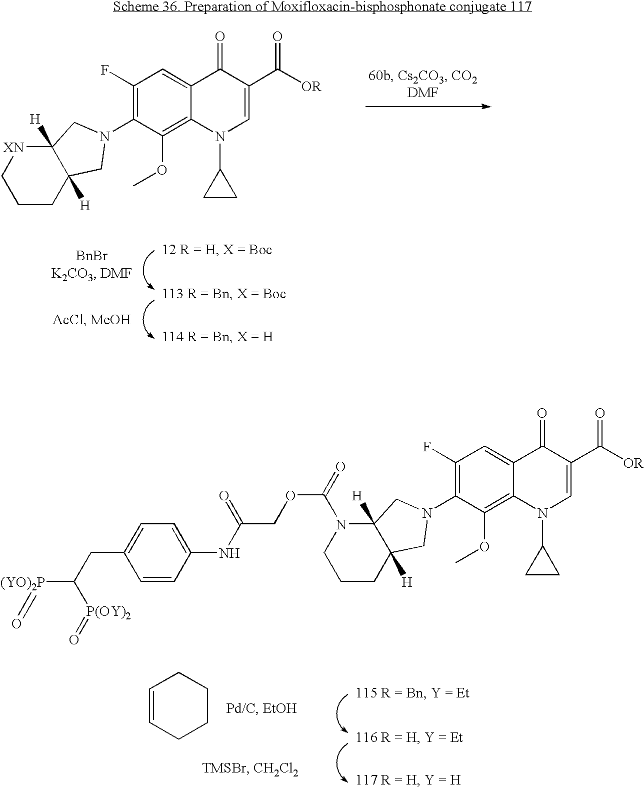

Benzyl 7-((4aS,7aS)-1-(tert-butoxycarbonyl)-octahydropyrrolo[3,4-b]pyridin-6-yl)-1-cyclopropyl-6-fluoro-1,4-dihydro-8-methoxy-4-oxoquinoline-3-carboxylate (113): Potassium carbonate (749 mg, 5.42 mmol) was added to a stirring solution of 12 (2.267 g, 4.52 mmol) in DMF (40 mL). After 10 min, benzylbromide (4.32 g, 20.0 mmol) was added and the resulting mixture was stirred at room temperature for 20 h. The mixture was concentrated under reduced pressure, then extracted with EtOAc (4×150 mL) and brine (200 mL). The combined organic layers were dried over MgSO₄, filtered and concentrated at reduced pressure to give 113 as a white solid (2.366 g, 89%) that was used without purification. ¹H NMR (400 MHz, CDCl₃) δ 0.74 (m, 1H), 1.02 (m, 2H), 1.23 (m, 1H), 1.48 (s, 11H), 1.77 (m, 2H), 2.22 (m, 1H), 2.88 (s, 1H), 3.21 (m, 1H), 3.35 (m, 1H), 3.54 (s, 3H), 3.86 (m, 2H), 4.03 (m, 2H), 4.76 (s, 1H), 5.39 (dd, J=112.6, 20.6, 2H), 7.33 (m, 3H), 7.51 (d, J=8.4, 2H), 7.83 (d, J=14.1, 1H), 8.54 (s, 1H).

Benzyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (114): Acetyl chloride (5.33 ml, 74.95 mmol) was added dropwise to 25 mL of dry methanol in a ice cold bath. After 15 min, 113 (2.668 g, 4.52 mmol) was added to that solution of 3M HCl in methanol and the resulting mixture turned yellow. After 30 min, the reaction was complete, then the mixture was concentrated under reduced pressure, extracted with EtOAc (4×150 mL) and a saturated solution of sodium bicarbonate (200 mL). The combined organic layers were dried over MgSO₄, filtered and concentrated at reduced pressure to give 114 as a white solid (1.791 g, 80%) that was used without purification. ¹H NMR (400 MHz, CDCl₃) δ 0.77 (m, 1H), 1.01 (m, 2H), 1.14 (m, 1H), 1.52 (m, 1H), 1.77 (m, 3H), 2.29 (m, 1H), 2.68 (t, J=10.3, 1H), 3.05 (d, J=12.1, 1H), 3.35 (m, 3H), 3.54 (s, 3H), 3.86 (m, 3H), 5.36 (dd, J=12.6, 20.6, 2H), 7.36 (m, 3H), 7.51 (d, J=8.4, 2H), 7.78 (d, J=14.1, 1H), 8.52 (s, 1H).

Benzyl 7-((4aS,7aS)-1-(((4-(2,2-bis(diethylphosphono)ethyl)phenylcarbamoyl)methoxy) carbonyl)-octahydropyrrolo[3,4-b]pyridin-6-yl)-1-cyclopropyl-6-fluoro-1,4-dihydro-8-methoxy-4-oxoquinoline-3-carboxylate (115): Carbon dioxide was bubbled for 1 h through a solution of 114 (100 mg, 0.203 mmol) and cesium carbonate (200 mg, 0.610 mmol) in 25 mL of dry DMF at room temperature. Then 60b (104 mg, 0.203 mmol) was added to that solution and the addition of carbon dioxide was continued for another 30 min. After 20 h, the reaction was complete, then the mixture was concentrated under reduced pressure, extracted with CH₂Cl₂ (3×100 mL) and brine (100 mL). The combined organic layers were dried over MgSO₄, filtered and concentrated at reduced pressure. The crude oil was purified by silica gel chromatography (5% methanol in CH₂Cl₂), resulting in 115 as a pale yellow oil (101 mg, 51%). ¹H NMR (400 MHz, CDCl₃) δ 0.74 (m, 1H), 0.99 (m, 2H), 1.23 (m, 13H), 1.52 (m, 2H), 1.78 (m, 2H), 2.28 (m, 1H), 2.57 (tt, J=6.3 23.8, 1H), 3.19 (m, 4H), 3.42 (t, J=9.2, 1H), 3.54 (s, 3H), 3.84 (m, 2H), 4.12 (m, 9H), 4.71 (m, 2H), 4.84 (q, J=8.8, 1H), 5.34 (dd, J=12.6, 20.6, 2H), 7.30 (m, 5H), 7.46 (d, J=7.3, 4H), 7.78 (d, J=14.1, 1H), 8.52 (s, 2H). ³¹P NMR (162 MHz, CDCl₃) δ 23.964 (s, 2P).

7-((4aS,7aS)-1-(((4-(2,2-bis(diethylphosphono)ethyl)phenylcarbamoyl)methoxy)carbonyl)-octahydropyrrolo[3,4-b]pyridin-6-yl)-1-cyclopropyl-6-fluoro-1,4-dihydro-8-methoxy-4-oxoquinoline-3-carboxylic acid (116): To a mixture of 115 (101 mg, 0.104 mmol) and Pd/C 10% (50 mg) in EtOH (10 mL) was added cyclohexene (2 mL, 20 mmol). The mixture is refluxed for 20 h. Then the catalyst was removed by filtration through glass fiber filter paper and the solvent was removed under reduced pressure to give 116 as a colorless oil (88 mg, 96%) that was used without purification. ¹H NMR (400 MHz, CDCl₃) δ 0.81 (m, 1H), 1.11 (m, 2H), 1.25 (m, 15H), 1.53 (m, 2H), 1.83 (m, 2H), 2.33 (m, 1H), 2.59 (tt, J=23.8, 6.3, 1H), 3.22 (m, 4H), 3.48 (t, J=9.2, 1H), 3.57 (s, 3H), 3.95 (m, 2H), 4.09 (m, 8H), 4.74 (s, 2H), 4.88 (q, J=8.8, 1H), 7.25 (m, 3H), 7.46 (d, J=7.3, 1H), 7.78 (d, J=14.1, 1H), 8.10 (s, 1H), 8.76 (s, 2H). ³¹P NMR (162 MHz, CDCl₃) δ 23.949 (s, 2P).

7-((4aS,7aS)-1-(((4-(2,2-bisphosphonoethyl)phenylcarbamoyl)methoxy)carbonyl)-octahydropyrrolo[3,4-b]pyridin-6-yl)-1-cyclopropyl-6-fluoro-1,4-dihydro-8-methoxy-4-oxoquinoline-3-carboxylic acid (117) To a solution of compound 116 (200 mg, 0.227 mmol) in 25 mL of CH₂Cl₂ was added 0.35 mL (2.724 mmol) of bromotrimethylsilane. The mixture was stirred at room temperature overnight before being concentrated. The residue was kept at high vacuum for at least 30 min and then dissolved in water. The resulting solution was brought to pH 7.1 with 1 N sodium hydroxide aqueous solution and the solvent was removed under reduced pressure. The solid obtained was subjected to a Waters C18 Sep-Pak™ column (20 cc) with gradient elution from neat water to 2:1 water/methanol to afford product 117 (75 mg, 43%) as an off-white solid after lyophilisation. ¹H NMR (400 MHz, CDCl₃) δ 0.61 (m, 1H), 0.84 (m, 1H), 0.95 (m, 1H), 1.03 (m, 1H), 1.43 (m, 2H), 1.70 (m, 2H), 2.03 (tt, J=23.8, 6.3, 1H), 2.21 (m, 1H), 2.95 (t, J=15.3, 3H), 3.17 (d, J=9.8, 1H), 3.38 (s, 1H), 3.45 (s, 3H), 3.85 (t, J=9.4, 1H), 3.93 (m, 3H), 4.65 (q, J=10.3, 2H), 4.77 (s, 1H), 7.18 (d, J=8.6, 2H), 7.26 (d, J=8.6, 2H), 7.49 (d, J=14.5, 1H), 8.31 (s, 1H), ³¹P NMR (162 MHz, CDCl₃) δ 20.279 (s, 2P).

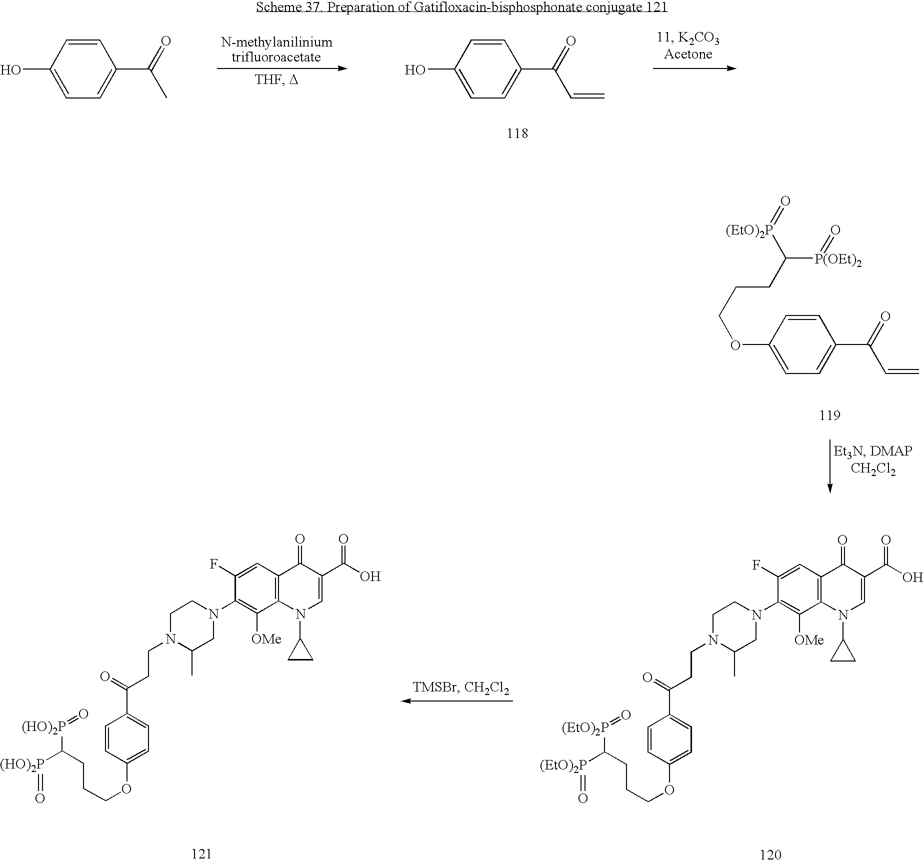

1-(4-hydroxyphenyl)prop-2-en-1-one (118): A mixture of 4′-hydroxyacetophenone (2.70 g, 19.9 mmol), paraformaldehyde (2.68 g, 89.3 mmol) and N-methylanilinium trifluoroacetate (6.51 g, 29.4 mmol) in THF (20 mL) was refluxed for 3 h. The mixture was cooled and added to diethyl ether (200 mL), rinsing the flask with further diethyl ether (100 mL). The product solution was decanted from the red gum and filtered. Evaporation gave crude 118 (2.0 g, 68%) which was used directly in the next step. ¹H NMR (400 MHz, CDCl₃) δ 5.69 (bs, 1H), 5.92 (dd, J=10.4, 1.7, 1H), 6.44 (dd, J=17.0, 1.7, 1H), 6.93 (d, J=8.8, 2H), 7.18 (dd, J=17.2, 10.6, 1H), 7.93 (d, J=8.8, 2H).

1-(4-(4,4-bis(diethylphosphono)butoxy)phenyl)prop-2-en-1-one (119): A mixture of iodide 11 (3.1 g, 6.8 mmol), phenol 118 (1.21 g, 8.17 mmol) and K₂CO₃ (1.033 g, 7.47 mmol) in acetone (75 mL) was refluxed for 6.5 h. The mixture was cooled, filtered and evaporated. The residue was redissolved in CH₂Cl₂ (170 mL), filtered through Celite and evaporated to give crude 119 (3.2 g, 99%) which was used directly in the next step. ¹H NMR (400 MHz, CDCl₃) δ 1.28-1.39 (m, 12H), 1.89-2.25 (m, 4H), 2.26-2.48 (m, 1H), 4.05 (t, J=5.7, 2H), 4.12-4.26 (m, 8H), 5.87 (dd, J=10.6, 1.8, 1H), 6.42 (dd, J=16.9, 1.8, 1H), 6.93 (d, J=8.8, 2H), 7.17 (dd, J=17.0, 10.4, 1H), 7.95 (d, J=8.8, 2H),

1-cyclopropyl-6-fluoro-1,4-dihydro-7-(4-(3-(4-(4,4-bis(diethylphosphono)butoxy)phenyl)-3-oxopropyl)-3-methylpiperazin-1-yl)-8-methoxy-4-oxoquinoline-3-carboxylic acid (120): A mixture of crude enone 119 (3.2 g, 6.7 mmol), gatifloxacin 15 (3.07 g, 8.18 mmol) DMAP (200 mg, 1.64 mmol) and triethylamine (1.4 mL, 10.0 mmol) in CH₂Cl₂ (200 mL) was stirred at room temperature for 20 h. The mixture was evaporated, followed by flash chromatography (gradient elution 5% methanol/CH₂Cl₂−10% methanol/CH₂Cl₂) to give 120 (3.6 g, 63%). ¹H NMR (400 MHz, CDCl₃) δ 0.94-1.04 (m, 2H), 1.12-1.28 (m, 5H), 1.30-1.37 (m, 12H), 2.06-2.24 (m, 4H), 2.27-2.45 (m, 1H), 2.55-3.50 (m, 10H), 3.74 (s, 3H), 3.97-4.08 (m, 3H), 4.13-4.24 (m, 8H), 6.92 (d, J=8.8, 2H), 7.86 (d, J=12.1, 1H), 7.94 (d, J=8.8, 2H), 8.80 (s, 1H).

1-cyclopropyl-6-fluoro-1,4-dihydro-7-(4-(3-(4-(4,4-bisphosphonobutoxy)phenyl)-3-oxopropyl)-3-methylpiperazin-1-yl)-8-methoxy-4-oxoquinoline-3-carboxylic acid (121): To a solution of 120 (3.6 g, 4.3 mmol) in CH₂Cl₂ (150 mL) was added TMSBr (5.6 mL, 42 mmol). The reaction mixture was stirred for 26.5 h, the solvent removed under reduced pressure and the solid dried under high vacuum for 1 h. The solid was suspended in H₂O (800 mL) and the pH was immediately adjusted to pH 8 by the addition of 1M KOH, with concomitant slow dissolution of the product. The product solution was evaporated at 30° C., and purified by reverse-phase chromatography (gradient elution, 100% water-30% methanol/water). The pure product 121 was obtained as a white fluffy solid (1.26 g, 33% recovery based on tetrapotassium salt of product). NMR (400 MHz, D₂O) δ 0.88-1.02 (m, 2H), 1.08-1.22 (m, 2H), 1.17 (d, J=6.2, 3H), 1.77-2.14 (m, 5H), 2.72-3.30 (m, 10H), 3.79 (s, 3H), 4.06-4.14 (m, 1H), 4.21 (t, J=6.4, 2H), 7.14 (d, J=8.8, 2H), 7.73 (d, J=12.8, 1H), 8.05 (d, J=8.8, 2H), 8.52 (s, 1H). ¹⁹F (376 MHz, D₂O) δ-122.44 (d, J=12.0, 1F). ³¹P (162 MHz, D₂O) δ 20.88 (s, 2P). MS: (MH⁺) 740.2.

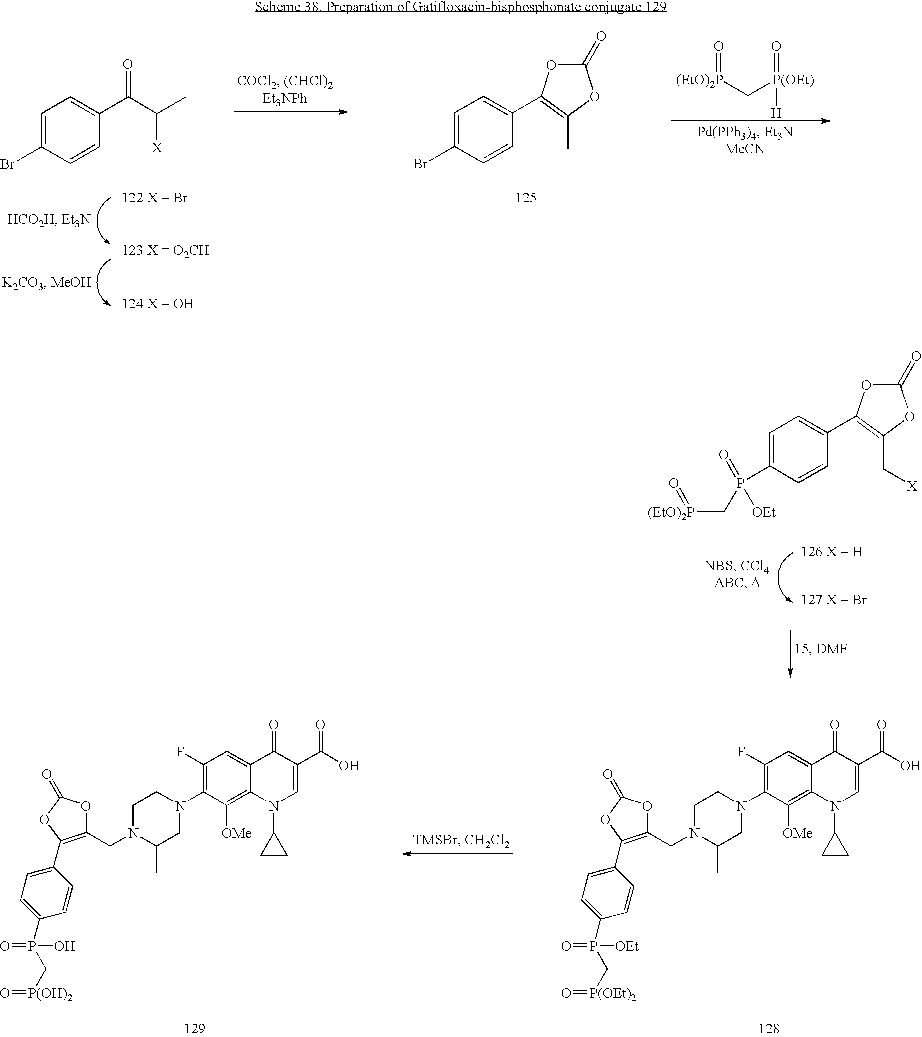

1-(4-Bromophenyl)-1-oxopropan-2-yl formate (123): A solution of formic acid (1.6 mL, 43 mmol) in acetonitrile (20 mL) was cooled in an ice-bath followed by the sequential drop-wise addition of TEA (6.0 mL, 43 mmol) then 2,4′-dibromopropriophenone (10.0 g, 34.2 mmol) in 10 mL of THF/acetonitrile (1:1). The resulting solution was stirred while warming to room temperature over 18 hr. The resulting colourless precipitate was filtered off and the organics were removed at reduced pressure. The residue was re-dissolved in EtOAc, re-filtered and concentrated to give 123 as yellow oil that was used without further purification: ¹H NMR (400 MHz, CDCl₃) δ 1.56 (d, J=7.0, 3H), 6.02 (q, J=7.0, 1H), 7.63 (d, J=8.7, 2H), 7.80 (d, J=8.7, 2H), 8.11 (s, 1H).

1-(4-Bromophenyl)-2-hydroxypropan-1-one (124): Crude 123 was dissolved in MeOH (100 mL) then 1 M NaOH (1.5 mL) was added and the resulting solution was stirred for 18 h. Approximately half on the methanol was removed at reduced pressure and the reaction was quenched by the addition of saturated aqueous NH₄Cl and the product was extracted with EtOAc. The organic layer was washed with brine, dried over Na₂SO₄ and concentrated to a yellow residue that was purified by flash silica gel chromatography (10% to 50% EtOAc in hexanes) resulting in 124 as a yellow oil (5.27 g, 68% over 2 steps): ¹H NMR (400 MHz, CDCl₃) δ 1.44 (d, J=7.0, 3H), 3.7 (bs, 1H), 5.11 (bq, J=6.9, 1H), 7.65 (d, J=8.5, 2H), 7.79 (d, J=8.5, 2H).

4-(4-Bromophenyl)-5-methyl-1,3-dioxol-2-one (125): A solution of 124 in 1,2-dichloroethane (DCE, 60 mL) was cooled in an ice bath followed by the addition of 20% phosgene (23.5 mL, 40.1 mmol) in toluene. After stirring for 15 min a solution of N,N-dimethylaniline (4.0 mL, 79 mmol) in DCE (10 mL) was added dropwise over a period of one hour at the same temperature. The ice-bath was removed and the reaction was heated to 70° C. for 20 hr. The solution was diluted with CH₂Cl₂ washed with water, 10% aqueous HCl, water, brine then dried over Na₂SO₄ and filtered. After removal of the solvent the product was recrystallized from EtOAc/Hexanes to furnish 125 (6.07 g, 63%) as a pale green solid: ¹H NMR (400 MHz, CDCl₃) δ 2.36 (s, 3H), 7.33 (d, J=8.7, 2H), 7.58 (d, J=8.7, 2H).

Ethyl (diethylphosphonomethyl)(4-(5-methyl-2-oxo-1,3-dioxol-4-yl)phenyl)phosphinate (126): A mixture of 125 (0.323 g, 1.27 mmol), diethyl(ethoxyphosphinyl)methylphosphonate (0.325 g, 1.33 mmol), TEA (0.530 mL, 3.80 mmol) and Pd(PPh₃)₄ (0.146 g, 0.127 mmol) in acetonitrile (3 mL) was heated 90° C. for 3 hr. The solvent was removed at reduced pressure and the product purified by silica gel flash column chromatography (0% to 6% MeOH in CH₂Cl₂) resulting in 126 (0.368 g, 70%) as a yellow solid: ¹H NMR (400 MHz, CDCl₃) δ 1.15-1.23 (m, 3H), 1.27-1.36 (m, 6H), 2.42 (s, 3H), 2.54-2.72 (m, 2H), 3.93-4.21 (m, 6H), 7.58 (dd, J=2.8, 8.5, 2H), 7.94 (dd, J=8.3, 12.2, 2H): ³¹P (162 MHz, CDCl₃) δ 17.41-17.54 (m, 1P), 30.86-31.08 (m, 1P).

Ethyl (diethylphosphonomethyl) (4-(5-(bromomethyl)-2-oxo-1,3-dioxol-4-yl)phenyl) phosphinate (127): A mixture of 126 (1.79 g, 4.28 mmol), NBS (0.761 g, 4.28 mmol) and 1,1′-azobis(cyclohexanecarbonitrile) (0.11 g, 0.43 mmol) in CCl₄ was heated to reflux under a strong visible light for 4 h at which time all 126 had been consumed as was evident by ¹H-NMR. The solvent was removed at aspirator pressure and the crude product was purified by silica gel flash column chromatography (0% to 5% MeOH in CH₂Cl₂) to furnish 127 (1.26 g, 60%) as a yellow oil: ¹H NMR (400 MHz, CDCl₃) δ 1.22 (t, J=7.1, 3H), 1.31 (t, J=7.1, 3H), 1.35 (t, J=7.1, 3H), 2.64 (ddd, J=2.8, 18.0, 20.4, 2H), 3.95-4.23 (m, 6H), 4.44 (s, 2H), 7.66 (dd, J=2.9, 8.4, 2H), 8.02 (dd, J=8.4, 12.2, 2H): ³¹P (162 MHz, CDCl₃) δ 19.63 (d, J=5.6, 1P), 32.93 (d, J=5.6, IP).

1-Cyclopropyl-6-fluoro-1,4-dihydro-8-methoxy-7-(3-methyl-4-((2-oxo-5-(4-(O-ethyl (diethylphosphonomethyl)phosphonoyl)phenyl)-1,3-dioxol-4-yl)methyl)piperazin-1-yl)-4-oxoquinoline-3-carboxylic acid (128): A solution of 15 and 127 in DMF was stirred at room temperature for 16 h. The reaction was quenched by the addition of saturated aqueous NH₄Cl and the product was extracted with ethyl acetate. The organic layer was washed with brine and dried over Na₂SO₄, filtered and concentrated at reduced pressure. The crude product was purified by silica gel HPFC (10% MeOH in EtOAc then 5% MeOH in CH₂Cl₂) to give 128 (44 mg, 28%) as pale yellow solid: ¹H NMR (400 MHz, CDCl₃) δ 0.97-1.03 (m, 2H) 1.17-1.38 (m, 14H), 2.61-2.70 (m, 3H), 2.79 (bs, 1H), 2.97 (bd, J=3.0, 1H), 3.16 (bt, J=9.2, 1H), 3.39-3.47 (m, 3H), 3.58 (d, J=14.7, 1H), 3.77 (s, 3H), 3.99-4.23 (m, 8H), 7.79 (dd, J=2.1, 8.3, 2H), 7.89 (d, J=7.9, 1H), 8.00 (dd, J=8.3, 11.4, 2H), 8.82 (s, 1H).

1-Cyclopropyl-6-fluoro-1,4-dihydro-8-methoxy-7-(3-methyl-4-((2-oxo-5-(4-(phosphonomethylphosphinoyl)phenyl)-1,3-dioxol-4-yl)methyl)piperazin-1-yl)-4-oxoquinoline-3-carboxylic acid (129): TMSBr (0.175 mL, 1.33 mmol) was added to a stirring solution of 128 (70 mg, 0.088 mmol) in CH₂Cl₂ (4 mL). The resulting solution was stirred at room temperature for 15 h then the solvent was removed at reduced pressure. The solid was suspended in 30 mM triethylammonium bicarbonate buffer (2 mL) then the pH was adjusted to approximately 6 by the addition of triethylamine. The solution was then subjected to C18 HPFC (5% to 50% CH₃CN in 30 mM triethylammonium bicarbonate). The isolated product was further purified by C18 HPFC (5% to 50% CH₃CN in water) to give 129 (20 mg, 32%) as the mono triethylammonium salt: ¹H NMR (400 MHz, D₂O) δ1.03-1.08 (m, 2H) 1.23 (d, J=7.6, 2H), 1.36 (d, J=5.8, 2H), 2.33 (d, J=18.2, 2H), 3.05-3.25 (m, 2H), 3.32-3.44 (m, 2H), 3.55-3.70 (m, 3H), 3.81 (s, 3H), 4.23-4.33 (m, 2H), 4.58 (d, J=14.8, 1H), 7.74-7.77 (m, 3H), 7.93 (dd, J=8.3, 11.1, 2H), 8.92 (s, 1H): ³¹P (162 MHz, D₂O) δ 12.82 (s, 1P), 29.37 (s, 1P): LCMS: 87.4% (254 nm), 87.1% (220 nm), 88.5% (290 nm). MS: (MH⁺) 708.2.

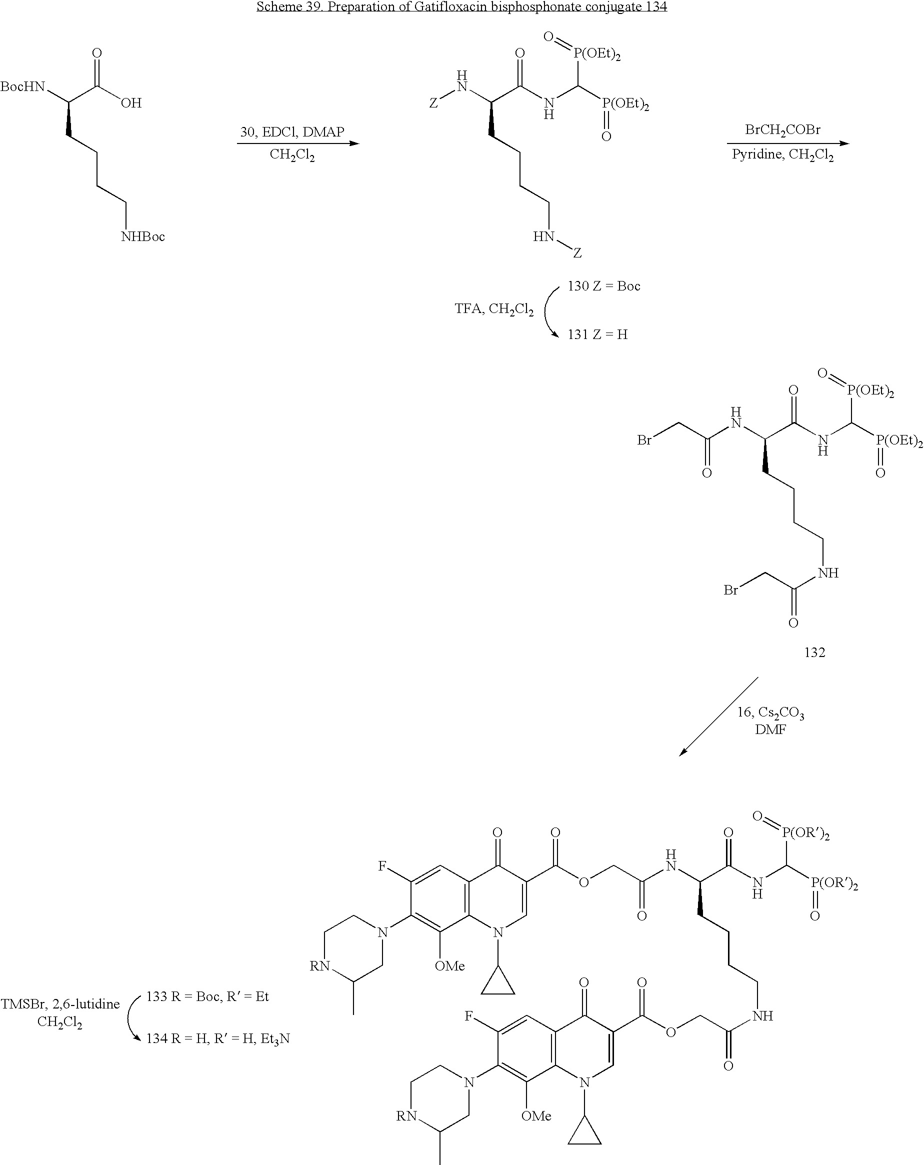

### Tetraethyl 1-(N—(N-α,ε-di-(t-butoxycarbonyl)lysinoyl)amino)methylenebisphosphonate (130)

To a solution of Boc-Lys(Boc)-OH dicyclohexylamine salt (1.57 g, 2.97 mmol) in CH₂Cl₂ (12 mL) was added amine 30 (900 mg, 2.97 mmol), EDCl (626 mg, 3.26 mmol) and DMAP (36 mg, 0.30 mmol). The mixture was stirred for 18 hours, after which the precipitate was removed by filtration and washed with a portion of CH₂Cl₂. The combined filtrates were washed with 1M HCl, saturated NaHCO₃ solution and brine. The organic layer was dried over Na₂SO₄, filtered and concentrated to dryness. The residue was purified by flash chromatography on silica gel using a gradient of 0-15% MeOH/EtOAc. Amide 130 was obtained as a white foam (1.35 g, 72%). ¹H NMR (400 MHz, CDCl₃) δ 1.30-1.34 (m, 12H), 1.43 (s, 18H), 1.38-1.53 (m, 4H), 1.59-1.69 (m, 1H), 1.79-1.87 (m, 1H), (3.07-3.12 (m, 2H), 4.11-4.24 (m, 8H), 4.71 (bs, 1H), 4.97 (dt, J=21.4, 10.1 Hz, 1H), 5.11 (bs, 1H), 6.76 (d, J=10.0 Hz, 1H).

### Tetraethyl 1-(N-lysinoylamino)methylenebisphosphonate (131)

To carbamate 130 (1.35 g, 2.14 mmol) was added a solution of TFA/CH₂Cl₂ (11 mL, 40% v/v). After stirring for 18 hours, the reaction mixture was concentrated to dryness and co-evaporated several times with Et₂O. The resulting deprotected material, a yellowish oil (2.1 g, >quant), was used without further purification. ¹H NMR (400 MHz, DMSO-d₆) δ 1.22-1.28 (m, 12H), 1.34-1.40 (m, 2H), 1.48-1.54 (m, 2H), 1.68-1.74 (m, 2H), 2.71-2.76 (m, 2H), 3.93-3.97 (m, 1H), 4.03-4.15 (m, 8H), 4.82 (dt, J=22.4, 9.8 Hz, 1H), 7.77 (bs, 3H), 8.25 (bd, J=4.2 Hz, 3H), 9.27 (d, J=9.8 Hz, 1H).

Tetraethyl 1-(N—(N-α,ε-di-(bromoacetyl)lysinoyl)amino)methylenebisphosphonate (132): To TFA salt 131 (max 2.14 mmol) in CH₂Cl₂ (27 mL) at 0° C. was added pyridine (1.73 mL, 21.4 mmol) and bromoacetyl bromide (390 μL, 4.49 mmol). The mixture was stirred for 1.5 h at 0° C. after which it was diluted with CH₂Cl₂ and washed with 5% HCl, saturated NaHCO₃ solution and brine. The organic layer was dried over MgSO₄, filtered and concentrated to dryness. Purification by flash chromatography on silica gel, using a gradient of 0-20% MeOH/EtOAc provided 132 as a white foam (574 mg, 40%). ¹H NMR (400 MHz, CDCl₃) δ 1.30-1.37 (m, 12H), 1.38-1.46 (m, 2H), 1.53-1.61 (m, 2H), 1.72-1.81 (m, 1H), 1.86-1.93 (m, 1H), 3.25-3.34 (m, 2H), 3.87 (s, 2H), 3.88 (s, 2H), 4.13-4.25 (m, 8H), 4.53 (q, J=6.2 Hz, 1H), 4.97 (dt, J=21.7, 9.9 Hz, 1H), 6.96 (bs, 1H), 7.01 (bd, J=9.8 Hz, 1H), 7.17 (d, J=7.8 Hz, 1H).

Bis(Gatifloxacin ester) conjugate 133:

To a solution of dibromide 133 (311 mg, 0.46 mmol) in DMF (5 mL) was added cesium carbonate (187 mg, 0.97 mmol) and BocGatifloxacin 16 (439 mg, 0.92 mmol). The mixture was stirred at room temperature for 18 h. It was then poured in H₂O and extracted with 3× EtOAc. The combined organic layers were washed with saturated NaHCO₃ solution, brine, dried over MgSO₄, filtered and concentrated to dryness. The crude product was purified by reverse phase flash chromatography on a C18 column, using a gradient of 20-100% MeCN/H₂O, followed by flash chromatography on silica gel using a gradient of 0-10% MeOH/CH₂Cl₂, yielding conjugate 133 as a light pink solid (316 mg, 47%). ¹H NMR (400 MHz, CDCl₃) δ 0.93-0.99 (m, 4H), 1.14-1.19 (m, 4H), 1.25-1.34 (m, 18H), 1.49 (s, 9H), 1.50 (s, 9H), 1.55-1.61 (m, 1H), 1.65-1.77 (m, 3H), 1.99-2.06 (m, 2H), 3.20-3.35 (m, 9H), 3.39-3.47 (m, 4H), 3.74 (s, 6H), 3.91-3.98 (m, 4H), 4.11-4.18 (m, 8H), 4.34 (bs, 2H), 4.49-4.68 (m, 5H), 5.03 (dt, J=21.9, 10.2 Hz, 1H), 7.12 (d, J=10.2 Hz, 1H), 7.86 (d, J=12.3 Hz, 2H), 8.47-8.50 (m, 2H), 8.72 (t, J=5.6 Hz, 1H), 9.51 (d, J=8.2 Hz, 1H). LCMS: 92.6% (254 nm), 95.2% (220 nm), 96.7% (320 nm), mass (ES⁻) calculated for C₆₇H₉₅F₂N₉O₂₁P₂ 1461, found 1460 (M−H)⁻.

Bis(Gatifloxacin ester) conjugate 134:

To a solution of protected conjugate 133 (391 mg, 0.27 mmol) in CH₂Cl₂ (5 mL) was added 2,6-lutidine (1.55 mL, 13.4 mmol). The mixture was cooled to −78° C. and trimethylsilylbromide (882 μL, 6.68 mmol) was added slowly. The mixture was brought to room temperature and stirred for 18 h, then was concentrated to dryness. Crude product was purified by 2 consecutive reverse phase flash chromatographies on a C18 column, using a gradient of 5-60% MeCN/50 mM Et₃NH₂CO₃ buffer, pH 7 for the first column, then a gradient of 5-50% MeCN/50 mM Et₃NH₂CO₃ buffer, pH 7 for the second column. Lyophilization of the combined pure fractions provided conjugate 134 as a white solid (16 mg, 5%). ¹H NMR (400 MHz, DMSO-d₆+TFA) δ 0.97 (bs, 4H), 1.07-1.10 (m, 4H), 1.17 (t, J=7.4 Hz, 9H), 1.26 (d, J=6.4 Hz, 6H), 1.34-1.49 (m, 4H), 1.61-1.64 (m, 1H), 1.72-1.76 (m, 1H), 3.10 (q, J=7.4 Hz, 6H), 3.17-3.26 (m, 4H), 3.38-3.52 (m, 10H), 3.81 (s, 6H), 4.01, 4.06 (m, 2H), 4.45-4.64 (m, 5H), 7.67 (d, J=12.1 Hz, 1H), 7.68 (d, J=12.1 Hz, 1H), 8.52 (s, 1H), 8.53 (s, 1H). LCMS: 98.1% (254 nm), 97.6% (220 nm), 99.1% (320 nm), mass (ES⁻) calculated for C₄₉H₆₃F₂N₉O₁₇P₂ 1149, found 1148.2 (M−H)⁻.

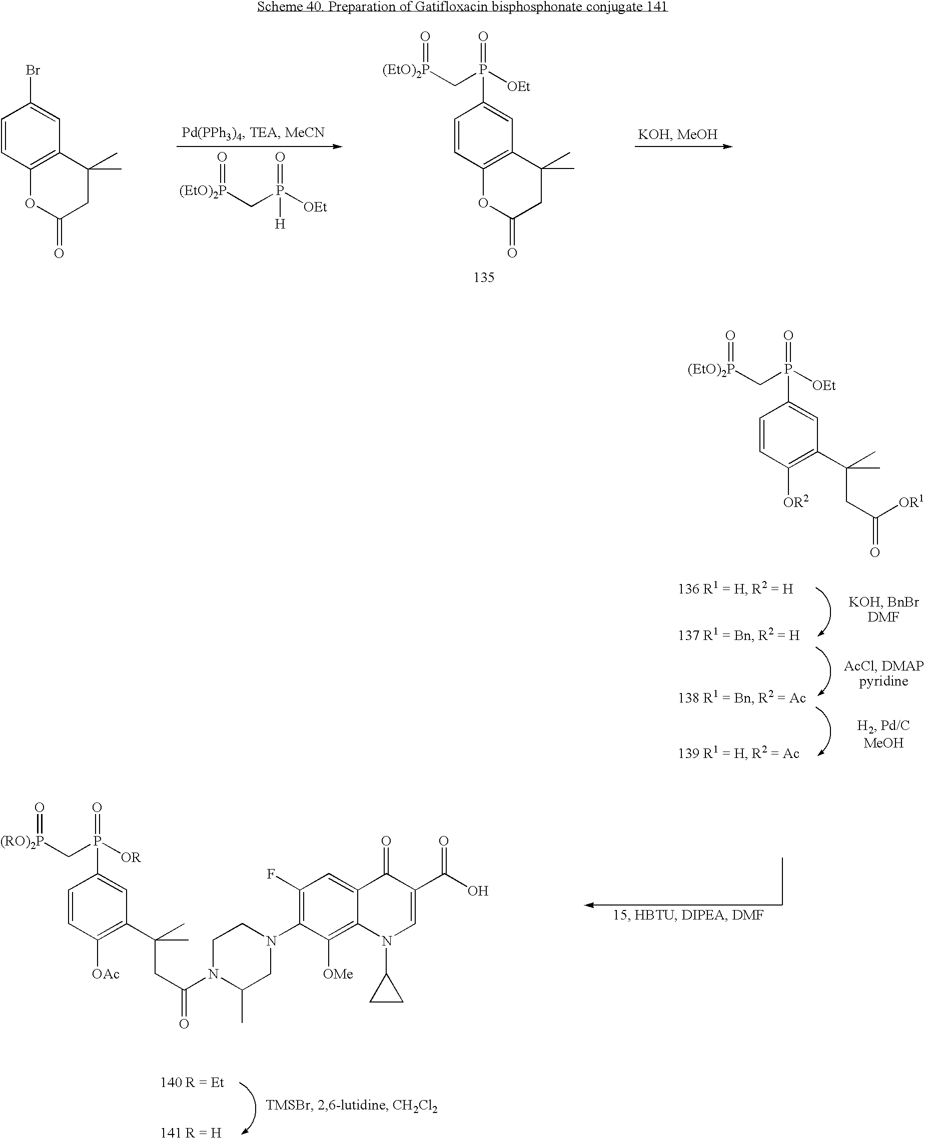

6-(Ethoxy(diethylphosphonomethyl)phosphinoyl)-3,4-dihydro-4,4-dimethylchromen-2-one (135): A mixture of 6-bromo-4,4-dimethylchroman-2-one (3.5 g, 9.7 mmol), diethyl(ethoxyphosphinyl)methylphosphonate (1.7 g, 9.7 mmol), triethylamine (4.1 mL, 29 mmol) and Pd(PPh₃)₄ (0.56 g, 0.48 mmol) in acetonitrile (20 mL) was heated to 100° C. for 18 hr. The reaction mixture was cooled and diluted with acetonitrile (50 mL) followed by washing with aqueous HCl (10%), water and saturated aqueous NaCl. The organic phase was dried over Na₂SO₄, filtered and concentrated. The crude product was purified by silica gel chromatography (0-10% MeOH in CH₂Cl₂) on a Biotage™ flash chromatography system, resulting in 135 as pale yellow oil (3.0 g, 73%): ¹H NMR (400 MHz, CDCl₃) δ 1.21 (t, J=7.2, 3H), 1.30-1.37 (m, 6H), 1.40 (s, 6H), 2.61 (dd, J=1.7, 17.2, 20.7, 2H), 2.66 (s, 2H), 3.95-4.08 (m, 2H), 4.11-4.21 (m, 4H), 7.16 (dd, J=3.1, 8.3, 2H), 7.73 (dd, J=3.1, 8.3, 2H): ³¹P (162 MHz, CDCl₃) δ 20.07 (d, J=7.7, 1P), 33.74 (d, J=7.7, 1P).

3-(2-Hydroxy-5-(Ethoxy(diethylphosphonomethyl)phosphinoyl)phenyl)-3-methylbutanoic acid (136): A solution of 135 (0.99 g, 2.4 mmol) and KOH (0.095 g, 2.4 mmol) in MeOH was stirred at room temperature for 2 hr. The solvent was removed removed under reduced pressure and the product was resuspended in water, the pH was adjusted to 4 by the addition of HCl, and the product was extracted with CH₂Cl₂. The organics were dried over Na₂SO₄, filtered and concentrated, resulting in 136 as a pale yellow oil (1.1 g, 105%) which was used without purification. ¹H NMR (400 MHz, CDCl₃) δ 1.26 (t, J=7.2, 3H), 1.29-1.37 (m, 6H), 1.45 (s, 3H), 1.48 (s, 3H), 2.63 (dd, J=17.7, 20.9, 2H), 2.93 (AB q, J=14.2, 2H), 4.00-4.20 (m, 6H), 6.74 (bs, 1H), 7.56 (ddd, J=1.6, 8.5, 12.2, 2H), 7.63 (d, J=13.3, 1H).

Benzyl 3-(2-hydroxy-5-(Ethoxy(diethylphosphonomethyl)phosphinoyl)phenyl)-3-methylbutanoate (137): An aqueous KOH solution (0.14 g, 2.5 mmol) was added to a stirring solution of 136 (1.1 g, 2.5 mmol) in acetonitrile (5 mL). After 10 min the solvent was evaporated under reduced pressure and the residue was dried under vacuum for 1 hr. The pale yellow solid was resuspended in DMF (10 mL) followed by the addition of benzylbromide (330 μL, 2.8 eq). The resulting solution was stirred at room temperature for 2 hr. The mixture was diluted with EtOAC (80 mL) and washed with H₂O and saturated aqueous NaCl, followed by drying over Na₂SO₄. The crude product was purified by silica gel chromatography (0-10% MeOH in CH₂Cl₂) on a Biotage™ flash chromatography system, resulting in 137 as a pale yellow liquid (0.64 g, 48%): ¹H NMR (400 MHz, CDCl₃) δ 1.23 (t, J=7.1, 3H), 1.26 (t, J=7.1, 3H), 1.30 (t, J=7.1, 3H), 1.45 (s, 3H), 1.49 (s, 3H), 2.60 (dd, J=17.4, 20.9, 2H), 3.00 (AB q, J=14.0, 2H), 3.78-3.88 (m, 2H), 3.99-4.15 (m, 4H), 4.93 (s, 2H), 6.75-6.78 (m, 1H), 7.14 (dd, J=2.0, 7.5, 2H), 7.25-7.31 (m, 3H), 7.58 (ddd, J=1.4, 8.0, 11.9, 1H), 7.64 (d, J=13.4, 1H): ³¹P (162 MHz, CDCl₃) δ 21.04 (d, J=4.6, 1P), 36.00 (d, J=4.6, 1P).

Benzyl 3-(2-acetoxy-5-(Ethoxy(diethylphosphonomethyl)phosphinoyl)phenyl)-3-methylbutanoate (138): A solution of 137 (0.64 g, 1.2 mmol) and DMAP (cat) in pyridine (10 mL) was cooled in an ice-bath followed by the drop-wise addition of acetyl chloride (94 μL, 1.3 mmol). The resulting solution was stirred for 2 hr at that temperature followed by dilution with EtOAc (80 mL). The organics were washed with aqueous HCl (10%), water, and saturated aqueous NaCl, followed by drying over Na₂SO₄. The crude product was purified by silica gel chromatography (0-10% MeOH in CH₂Cl₂) on a Biotage™ flash chromatography system, resulting in 138 as a pale yellow liquid (0.52 g, 75%): ¹H NMR (400 MHz, CDCl₃) δ1.20 (t, J=7.1, 3H), 1.30 (t, J=7.1, 6H), 1.49 (s, 3H), 2.33 (s, 3H), 2.56 (ddd, J=6.8, 17.3, 23.4, 2H), 2.84 (AB q, J=14.4, 2H), 3.95-4.04 (m, 2H), 4.06-4.18 (m, 4H), 4.95 (s, 2H), 7.14-7.20 (m, 3H), 7.28-7.34 (m, 3H), 7.73 (ddd, J=1.9, 8.2, 11.8, 1H), 7.89 (dd, J=1.9, 13.3, 1H): ³¹P (162 MHz, CDCl₃) δ20.20 (d, J=9.9, 1P), 33.88 (d, J=9.9, 1P).

3-(2-acetoxy-5-(Ethoxy(diethylphosphonomethyl)phosphinoyl)phenyl)-3-methylbutanoic acid (139): Compound 138 (0.50 g, 0.87 mmol) was dissolved in MeOH and hydrogenated over Pd/C (10%, 250 mg) under H₂ (1 atm) for 2 h. The catalyst was filtered off and the solvent removed resulting in the pale-yellow solid 139 (0.41 g, 98%): ¹H NMR (400 MHz, CDCl₃) δ 1.21 (dt, J=0.4, 7.1, 3H), 1.28 (dt, J=0.4, 7.1, 3H), 1.31 (t, J=7.1, 3H), 1.50 (s, 3H), 1.53 (s, 3H), 2.37 (s, 3H), 2.62 (ddd, J=5.4, 18.4, 22.4, 2H), 2.74 (AB q, J=13.9, 2H), 3.89-4.16 (m, 6H), 7.17 (dd, J=3.6, 8.2, 1H), 7.69 (ddd, J=1.9, 8.2, 11.9, 1H), 7.86 (dd, J=1.9, 13.7, 1H): ³¹P (162 MHz, CDCl₃) δ 20.17 (d, J=5.0, 1P), 34.51 (d, J=5.0, 1P).

7-(4-(3-(2-acetoxy-5-(Ethoxy(diethylphosphonomethyl)phosphinoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (140): A solution of 139 (400 mg, 0.836 mmol), 15 (310 mg, 0.84 mmol) and diisopropylethylamine (291 μL, 1.67 mmol) in DMF (5 mL) was cooled in an ice-bath followed by the addition of HBTU (317 mg, 0.836 mmol) in one portion. The resulting mixture was stirred while slowly warming to room temperature overnight. The reaction mixture was diluted with EtOAc (100 mL) and washed with aqueous HCl (10%), water, and saturated aqueous NaCl, followed by drying over Na₂SO₄. The crude beige solid was purified by silica gel chromatography (0-10% MeOH in CH₂Cl₂) on a Biotage™ flash chromatography system, resulting in 140 as a pale yellow solid (260 mg, 34%): ¹H NMR (400 MHz, CDCl₃) δ 0.92-0.94 (m, 2H), 1.12-1.27 (m, 14H), 1.43 (s, 3H), 1.46 (s, 3H), 2.31 (s, 3H), 2.53-5.63 (m, 1H), 2.74-2.85 (m, 3H), 3.00-3.40 (m, 5H), 3.65 (s, 3H), 3.88-4.08 (m, 6H), 4.27 (bs, 1H), 4.66 (bs, 1H), 7.07 (dd, J=3.2, 8.1, 1H), 7.60-7.65 (m, 1H), 7.75 (d, J=12.0, 1H), 7.82 (dd, J=5.2, 8.1, 1H), 8.72 (s, 1H): ³¹P (162 MHz, CDCl₃) δ 20.07 (d, J=8.7, 1P), 33.90 (d, J=8.7, 1P).

7-(4-(3-(2-acetoxy-5-((phosphonomethyl)phosphonoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (141): TMSBr (355 μL, 2.69 mmol) was added to a stirring solution of 140 (150 mg, 0.179 mmol) and 2,6-lutidine (416 μL, 3.59 mmol) in CH₂Cl₂ (5 mL). The resulting yellow couloured solution was stirred at room temperature for 23 h then the solvent was removed at reduced pressure. The yellow coloured solid was resuspended in water and the solution was adjusted to approximately pH 6.5 by the addition of 1M NaOH. The solution was then subjected to reverse-phase chromatography (0% to 60% CH₃CN in water) on a Biotage™ flash chromatography system, to give 141 as the mono 2,6-lutidine salt (80 mg, 60%): ¹H NMR (400 MHz, D₂O) δ 0.99-1.09 (m, 2H), 1.16-1.29 (m, 5H), 1.51 (s, 6H), 2.46 (s, 3H), 2.70 (s, 6H), 2.98-3.10 (m, 2H), 3.16-3.54 (m, 4H), 3.71 (s, 3H), 3.83 (d, J=12.7, 1H), 4.17 (d, J=12.9, 1H), 4.21-4.27 (m, 1H), 4.56 (bs, 1H), 7.20 (dd, J=2.9, 8.0, 1H), 7.56 (dd, J=3.7, 12.2, 1H), 7.62 (d, J=8.0, 2H), 7.71 (bd, J=9.2, 1H), 7.93 (dd, J=3.8, 12.2, 1H), 8.26 (t, J=8.0, 1H), 8.89 (s, 1H): MS (MH⁻) 750.1.

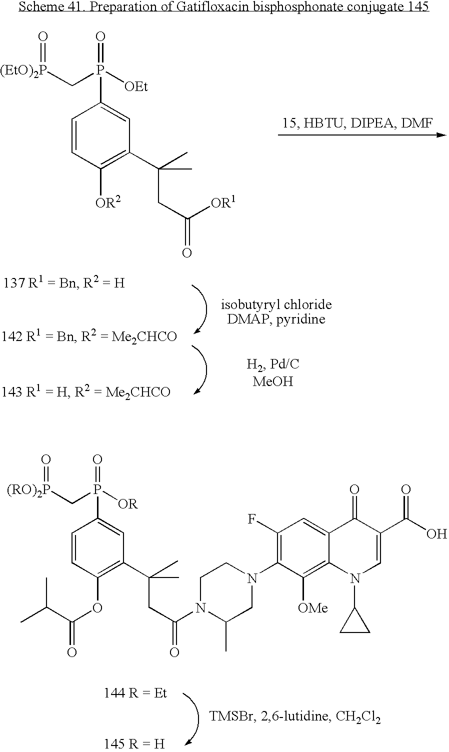

Benzyl 3-(2-(2,2-dimethylacetoxy)-5-(Ethoxy(diethylphosphonomethyl)phosphinoyl)phenyl)-3-methylbutanoate (142): Isobutyryl chloride (165 μL, 1.56 mmol) was added drop-wise to a stirred solution of 137 (825 mg, 1.56 mmol) and DMAP (cat) in pyridine (10 mL) at room temperature. The resulting solution was stirred for 2 hr followed by dilution with EtOAc (80 mL). The organics were washed with aqueous HCl (5%), water, and saturated aqueous NaCl, then dried over Na₂SO₄. The crude product was purified by silica gel chromatography (0-10% MeOH in CH₂Cl₂) on a Biotage™ flash chromatography system, resulting in 142 as a pale yellow liquid (728 mg, 78%): ¹H NMR (400 MHz, CDCl₃) δ 1.18-1.22 (m, 6H), 1.29-1.33 (m, 9H), 1.46 (s, 3H), 1.49 (s, 3H), 2.58 (ddd, J=6.9, 17.5, 23.5, 2H), 2.79-2.90 (m, 3H), 3.96-4.19 (m, 6H), 4.96 (s, 2H), 7.09 (dd, J=3.4, 8.3, 1H), 7.18-7.21 (m, 2H), 7.28-7.32 (m, 3H), 7.72 (ddd, J=1.9, 8.3, 11.8, 1H), 7.88 (dd, J=1.9, 13.3, 1H): ³¹P (162 MHz, CDCl₃) δ 20.28 (d, J=9.8, 1P), 34.04 (d, J=9.8, IP).

3-(2-(2,2-dimethylacetoxy)-5-(Ethoxy(diethylphosphonomethyl)phosphinoyl)phenyl)-3-methylbutanoic acid (143): Compound 142 (725 mg, 1.21 mmol) was dissolved in MeOH (20 mL) and hydrogenated over Pd/C (10%, 500 mg) under H₂ (1 atm) for 5 h. The catalyst was filtered off and the solvent removed resulting in the pale-yellow solid 143 (543 mg, 88%): ¹H NMR (400 MHz, CDCl₃) δ 1.21 (t, J=7.1, 3H), 1.29 (t, J=7.3, 3H), 1.31 (t, J=7.0, 3H), 1.36 (d, J=7.0, 6H), 1.50 (s, 3H), 1.52 (s, 3H), 2.63 (ddd, J=3.9, 17.2, 22.2, 2H), 2.76 (AB q, J=14.0, 2H), 2.86 (septet, J=7.1, 1H), 3.91-4.16 (m, 6H), 7.10 (dd, J=3.4, 8.2, 1H), 7.69 (ddd, J=1.8, 8.2, 11.7, 1H), 7.86 (dd, J=1.8, 13.6, 1H).

7-(4-(3-(2-(2,2-dimethylacetoxy)-5-(Ethoxy(diethylphosphonomethyl)phosphinoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (144): A solution containing 143 (398 mg, 0.790 mmol), 15 (296 mg, 0.790 mmol) and diisopropylethylamine (275 μL, 1.58 mmol) in DMF (5 mL) was cooled in an ice-bath followed by the addition of HBTU (300 mg, 0.790 mmol) in one portion. The resulting mixture was stirred while warming to room temperature overnight. The reaction mixture was diluted with EtOAc (100 mL) and washed with aqueous HCl (10%), water, and saturated aqueous NaCl, followed by drying over Na₂SO₄. The crude material was purified by silica gel chromatography (0-5% MeOH in EtOAc) on a Biotage™ flash chromatography system resulting in 144 as a pale yellow liquid (229 g, 34%): ¹H NMR (400 MHz, CDCl₃) δ 0.97-1.00 (m, 2H), 1.18-1.36 (m, 14H), 1.38 (d, J=7.1, 6H), 1.54 (s, 3H), 1.56 (s, 3H), 2.63 (ddd, J=5.6, 17.2, 22.0, 2H), 2.85-2.92 (m, 3H), 3.13 (bs, 1H), 3.27-3.51 (m, 5H), 3.71 (s, 3H), 3.98-4.19 (m, 6H), 4.43 (bs, 1H), 4.83 (bs, 1H), 7.08 (dd, J=3.5, 8.1, 1H), 7.67-7.74 (m, 1H), 7.89 (d, J=12.1, 1H), 7.95 (dd, J=1.7, 13.7, 1H), 8.83 (s, 1H): ³¹P (162 MHz, CDCl₃) δ 20.1. (d, J=9.0, 1P), 34.32 (d, J=9.0, 1P).

7-(4-(3-(2-(2,2-dimethylacetoxy)-5-((phosphonomethyl)phosphonoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (145): TMSBr (1.29 mL, 9.89 mmol) was added to a stirring solution of 144 (565 mg, 0.654 mmol) and 2,6-lutidine (1.52 mL, 13.1 mmol) in CH₂Cl₂ (15 mL). The resulting pale green coloured solution was stirred at room temperature for 18 h and then the solvent was removed at reduced pressure. The reddish coloured solid was resuspended in water (5 mL) and the solution was adjusted to approximately pH 7 by the addition of 1M NaOH. The solution was then subjected to two reverse-phase chromatographies (0% to 60% CH₃CN in water) on a Biotage™ flash chromatography system, to yield 145 as the disodium salt (130 mg, 16%): ¹H NMR (400 MHz, D₂O) δ 0.99-1.09 (m, 2H), 1.21 (d, J=7.3, 3H), 1.33-1.44 (m, 2H), 1.38 (d, J=7.0, 6H), 1.51 (s, 6H), 2.29 (AB q, J=16.4, 2H), 2.93-3.10 (m, 3H), 3.14-3.41 (m, 2H), 3.48-3.56 (m, 2H), 3.71 (s, 3H), 3.85 (d, J=113.1, 1H), 4.18 (d, J=13.1, 1H), 4.25 (septet, J=3.6, 1H), 4.58 (bs, 1H), 7.10-7.15 (m, 1H), 7.65 (d, J=12.1, 1H), 7.70 (t, J=9.4, 1H), 7.93 (bd, J=12.5, 1H), 8.89 (s, 1H): MS (MH⁻) 778.1.

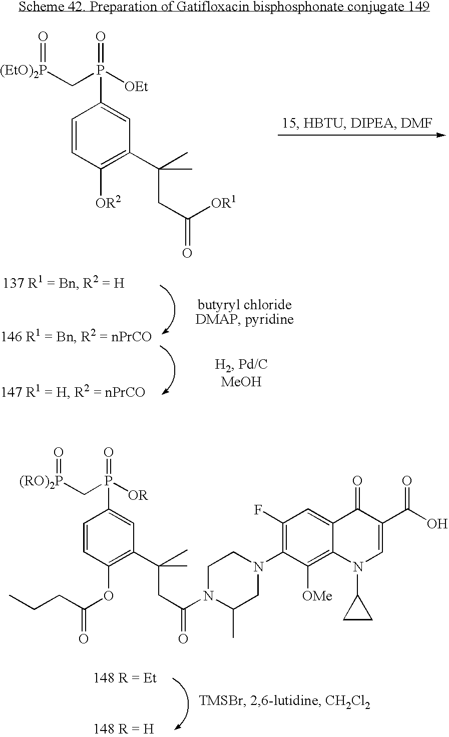

Benzyl 3-(2-butyroxy-5-(Ethoxy(diethylphosphonomethyl)phosphinoyl)phenyl)-3-methylbutanoate (146): Butyryl chloride (127 μL, 1.21 mmol) was added drop-wise to a stirred solution of 137 (640 mg, 1.21 mmol) and DMAP (cat) in pyridine (5 mL) at room temperature. The resulting solution was stirred for 2 hr followed by dilution with EtOAc (80 mL). The organics were washed with aqueous HCl (5%), water, and saturated aqueous NaCl, then dried over Na₂SO₄. The crude product was purified by silica gel chromatography (0-20% MeOH in EtOAc) on a Biotage™ flash chromatography system, resulting in 146 as a colourless liquid (360 mg, 49%): ¹H NMR (400 MHz, CDCl₃) δ1.02 (t, J=7.5, 3H), 1.20 (t, J=7.1, 3H), 1.30 (t, J=7.1, 3H), 1.31 (t, J=7.1, 3H), 1.46 (s, 3H), 1.49 (s, 3H), 1.78 (sextet, J=7.3, 2H), 2.50-2.62 (m, 4H), 2.84 (AB q, J=14.3, 2H), 3.88-4.19 (m, 6H), 4.96 (s, 2H), 7.14-7.32 (m, 6H), 7.73 (ddd, J=1.4, 8.7, 11.7, 1H), 7.89 (dd, J=1.4, 13.1, 1H): ³¹P (162 MHz, CDCl₃) δ 20.22 (d, J=9.8, 1P), 33.90 (d, J=9.8, 1P).

3-(2-butyroxy-5-(Ethoxy(diethylphosphonomethyl)phosphinoyl)phenyl)-3-methylbutanoic acid (147): Compound 146 (200 mg, 0.335 mmol) was dissolved in MeOH (20 mL) and hydrogenated over Pd/C (10%, 75 mg) under H₂ (1 atm) for 3 h. The catalyst was filtered off and the solvent removed resulting in the pale-yellow solid 143 (165 mg, 98%): ¹H NMR (400 MHz, CDCl₃) δ 1.06 (t, J=7.3, 3H), 1.23 (t, J=7.2, 3H), 1.29 (t, J=7.0, 3H), 1.32 (t, J=7.0, 3H), 1.51 (s, 3H), 1.54 (s, 3H), 1.82 (sextet, J=7.3, 2H), 2.57-2.68 (m, 4H), 2.73 (AB q, J=13.8, 2H), 3.90-4.1, 7 (m, 6H), 7.16 (d, J=3.6, 1H), 7.69 (ddd, J=1.6, 8.4, 11.7, 1H), 7.86 (dd, J=1.6, 13.6, 1H): ³¹P (162 MHz, CDCl₃) δ 20.05 (d, J=3.7, IP), 34.35 (d, J=3.7, IP).

7-(4-(3-(2-butyroxy-5-(Ethoxy(diethylphosphonomethyl)phosphinoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (148): A solution containing 147 (163 mg, 0.322 mmol), 15 (121 mg, 0.322 mmol) and diisopropylethylamine (112 μL, 0.644 mmol) in DMF (4 mL) was cooled in an ice-bath followed by the addition of HBTU (122 mg, 0.322 mmol). The resulting mixture was stirred while warming to room temperature overnight. The reaction mixture was diluted with EtOAc (100 mL) and washed with aqueous HCl (10%), water, and saturated aqueous NaCl, followed by drying over Na₂SO₄. The light brown coloured liquid of 148 (194 mg, 70%) was used with out purification.

7-(4-(3-(2-butyroxy-5-((phosphonomethyl)phosphonoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (149): TMSBr (1.29 mL, 9.89 mmol) was added to a stirred solution of crude 148 (194 mg, 0.225 mmol) and 2,6-lutidine (521 μL, 4.49 mmol) in CH₂Cl₂ (2 mL). The resulting pale yellow coloured solution was stirred at room temperature for 24 h and then the solvent was removed at reduced pressure. The reddish coloured solid was resuspended in water (2 mL) and the solution was adjusted to approximately pH 7 by the addition of 1M NaOH. The solution was then subjected to reverse-phase chromatography (0% to 60% CH₃CN in water) on a Biotage™ flash chromatography system, to yield 149 as the mono-2,6-lutidine salt (60 mg, 30%): MS (MH⁺) 780.2.

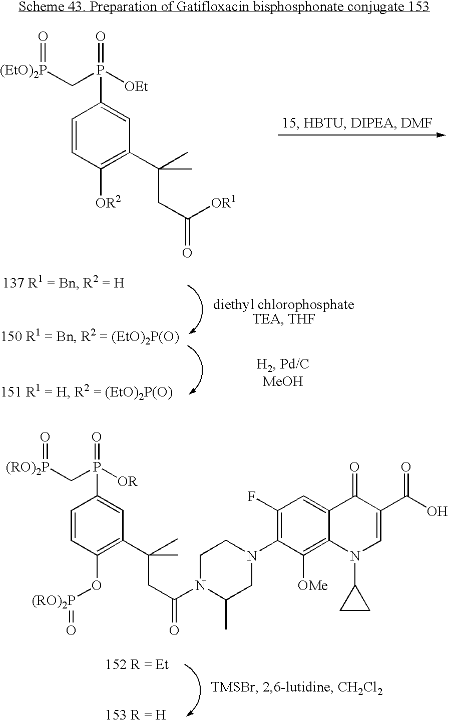

Benzyl 3-(2-(diethylphosphoryloxy)-5-(Ethoxy(diethylphosphonomethyl)phosphinoyl)phenyl)-3-methylbutanoate (150): Diethyl chlorophosphate (283 μL, 1.98 mmol) was added drop-wise to a stirring solution of 137 (695 mg, 1.32 mmol) and triethylamine (368 μL, 2.64 mmol) in THF. The resulting mixture was stirred for 24 hr at room temperature followed by dilution with EtOAc (80 mL). The organics were washed with aqueous HCl (5%), water, and saturated aqueous NaCl, followed by drying over Na₂SO₄. The crude product was purified by silica gel chromatography (0-20% MeOH in EtOAc) on a Biotage™ flash chromatography system, resulting in 150 as a pale yellow liquid (390 mg, 45%): ¹H NMR (400 MHz, CDCl₃) δ 1.21 (t, J=7.0, 3H), 1.24-1.36 (m, 12H), 1.51 (s, 3H), 1.53 (s, 3H), 2.50-2.61 (m, 2H), 2.94 (AB q, J=14.1, 2H), 3.86-4.24 (m, 10H), 4.93 (s, 2H), 7.13-7.17 (m, 2H), 7.26-7.29 (m, 3H), 7.59 (dd, J=2.9, 8.3, 1H), 7.71 (t, J=9.8, 1H), 7.83 (d, J=13.1, 1H).

3-(2-(diethylphosphoryloxy)-5-(Ethoxy(diethylphosphonomethyl)phosphinoyl)phenyl)-3-methylbutanoic acid (151): Compound 150 (430 mg, 0.335 mmol) was dissolved in MeOH (20 mL) and hydrogenated over Pd/C (10%, 200 mg) under H₂ (1 atm) for 2 h. The catalyst was filtered off and the solvent removed resulting in the colourless oil 151 (165 mg, 98%): ¹H NMR (400 MHz, CDCl₃) δ 1.19-1.39 (m, 15H), 1.57 (s, 3H), 1.59 (s, 3H), 2.62 (bt, J=19.4, 2H), 2.80 (AB q, J=113.9, 2H), 3.90-4.17 (m, 6H), 4.22-4.32 (m, 4H), 7.57 (dd, J=3.4, 8.4, 1H), 7.69 (ddd, J=1.7, 8.4, 11.8, 1H), 7.81 (bd, J=13.5, 1H).

7-(4-(3-(2-(diethylphosphoryloxy)-5-(Ethoxy(diethylphosphonomethyl)phosphinoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (152): HBTU (229 mg, 0.603 mmol) was added to a solution containing 151 (345 mg, 0.603 mmol), 15 (226 mg, 0.603 mmol) and diisopropylethylamine (210 μL, 1.21 mmol) in DMF (5 mL) that was cooled in an ice-bath. The resulting mixture was stirred while warming to room temperature overnight. The reaction mixture was diluted with EtOAc (100 mL) and washed with aqueous HCl (10%), water, and saturated aqueous NaCl, followed by drying over Na₂SO₄. The light brown coloured liquid of 152 (326 mg, 58%) was used with out purification.

7-(4-(3-(2-phosphoryloxy-5-((phosphonomethyl)phosphonoyl)phenyl)-3-methylbutanoyl)-3-methylpiperazin-1-yl)-1-cyclopropyl-6-fluoro-1,4-dihydro-8-methoxy-4-oxoquinoline-3-carboxylic acid (153): TMSBr (1.29 mL, 9.89 mmol) was added drop-wise to a stirred solution of crude 152 (326 mg, 0.351 mmol) and 2,6-lutidine (814 μL, 7.01 mmol) in CH₂Cl₂ (3 mL). The resulting pale green coloured solution was stirred at room temperature for 24 h and then the solvent was removed at reduced pressure. The brownish coloured solid was resuspended in triethylamime/carbonate buffer (30 mM, 2 mL) and the solution was adjusted to approximately pH 6.5 by the addition of 1M NaOH. The solution was then subjected to reverse-phase chromatography (0% to 60% CH₃CN in water) on a Biotage™ flash chromatography system, to yield 152 as the di-2,6-lutidine salt (110 mg, 31%): ¹H NMR (400 MHz, D₂O) δ 0.97-1.09 (m, 2H), 1.26-1.36 (m, 5H), 1.56 (s, 6H), 2.34 (t, J=16.7, 2H), 2.69 (s, 12H), 2.91-3.10 (m, 2H), 3.16-3.55 (m, 3H), 3.68 (s, 3H), 3.90 (d, J=13.5, 1H), 4.14 (d, J=13.5, 1H), 4.22-4.27 (m, 1H), 4.32 (bs, 1H), 4.57 (bs, 1H), 7.49 (bd, J=8.5, 1H), 7.61 (d, J=8.1, 4H), 7.64-7.66 (m, 1H), 7.73-7.81 (m, 2H), 8.25 (t, J=8.1, 2H), 8.94 (s, 1H): ¹⁹F NMR (376 MHz, D₂O): δ119.15 (d, J=12.8): MS (MH⁺) 790.2.

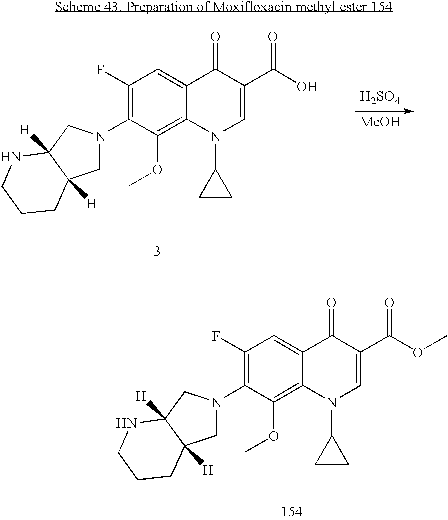

Methyl 1-cyclopropyl-6-fluoro-1,4-dihydro-7-((4aS,7aS)-octahydropyrrolo[3,4-b]pyridin-6-yl)-8-methoxy-4-oxoquinoline-3-carboxylate (154): Moxifloxacin (3, 113 mg, 0.2815 mmol) in 3 mL of methanol in the presence of 2 drops of concentrated sulfuric acid was refluxed for 5 h. After concentration, the residue was taken up in saturated sodium bicarbonate aqueous solution and was extracted with ethyl acetate (3×) before being dried over anhydrous sodium sulfate. Upon the removal of the solvent, the resultant mixture was subjected to a Waters® C18 Sep-Pak™ cartridge (6 cc) with gradient elution from neat water to 2:1 water/methanol to 1:2 to methanol to afford 12 mg of the ester 154 (10% yield) as an off-white powder. ¹H NMR (400 MHz, CDCl₃): [symbol] 0.79-0.84 (m, 1H), 0.99-1.05 (m, 2H), 1.16-1.20 (m, 1H), 1.69-1.81 (m, 3H), 2.26-2.32 (m, 1H), 2.67-2.72 (m, 1H), 3.04 (dt, J=4.1, 12.7, 1H), 3.29-3.32 (m, 1H), 3.36-3.42 (m, 2H), 3.56 (s, 3H), 3.86-3.98 (m, 3H), 3.91 (s, 3H), 7.82 (d, J=14.3, 1H), 8.55 (s, 1H).

### Example 2

### Determination of In Vitro Antibacterial Activity and Cytotoxicity

### In Vitro Antibacterial Activity

Susceptibility of S. aureus strains ATCC13709 and RN4220 to the commercial antibiotics and synthesized compounds was determined by following the guidelines set by the Clinical and Laboratory Standards Institute (formerly the National Committee for Clinical Laboratory Standards) (M26-A). Compounds were diluted two-fold serially in DMSO and transferred to cation-adjusted Mueller Hinton broth (CAMHB; Becton Dickinson). 50 μL of compounds diluted in CAMHB was mixed with 100 μL of bacteria diluted in CAMHB in 96-well microtiter plates. The final number of micro-organisms in the assay was 5×10⁵ c.f.u. per mL and the final concentration of DMSO in the assay was 1.25%. Assays were set up in duplicate and incubated at 37° C. for 18 h. The concentration of compound that inhibited visible growth was reported as the minimum inhibitory concentration (MIC).

Susceptibility testing experiments were also carried out in the presence of serum. These experiments were carried out similar to the susceptibility testing with the following modifications. 75 μL of compounds diluted in CAMHB was mixed with 75 μL of bacteria diluted in 100% serum from any given source (commercial pooled mouse serum (MS) and human serum (HS), Equitech-Bio Inc.) or diluted in 8% purified human serum albumin (HSA) (Calbiochem). The final concentration of animal serum in the assay was 50% and the final concentration of purified human serum albumin in the assay was 4%; the concentrations of all other components were identical to those described for susceptibility testing.

Although not shown, the results show the compounds can be categorized into two groups. The free fluoroquinolones display high potency in terms of antibacterial activities, with MICs generally less than 0.5-1 μg/mL, as shown with Moxifloxacin 3, Gatifloxacin 15 and Ciprofloxacin 6. The prodrugs comprising the phosphonated fluoroquinolones exhibit much weaker activities with MICs generally 10-100 fold higher, in the 8 to >128 μg/mL range, such as for compounds 44 (1-8 μg/mL), 49 (0.5-1 μg/mL), 54 (4-16 μg/mL) and 141 (4 μg/mL).

The presence of serum had little impact on the MIC values associated with the bisphosphonate conjugated drugs in their unprotected state and in the absence of bone mineral, which suggests participation from serum hydrolytic enzymes to be at least not required for cleavage if the antibacterial activity is due to the fluoroquinolone moiety released during the course of the assay. In essence, the release of the drug does not appear to be greater in aqueous buffer than in serum. In contrast, the protected bisphosphonate 26 (MIC 32 μg/mL shifts to 0.5 μg/mL in the presence of 50% mouse serum in the medium) sharply increases in activity. In fact, the comparison of 26 with its deprotected parent 25 (MIC 32 μg/mL unchanged in serum) clearly suggests the free bisphosphonates not to be substrates of serum hydrolytic enzymes in solution if the antibacterial activity is due to the fluoroquinolone moiety released during the course of the assay.

### Cytotoxicity Assays

Selected compounds were also tested for their ability to inhibit growth of mammalian cells so as to ascertain levels of cytotoxicity to the mammalian host, via an assay measuring the biological reduction of the inner salt of (3-(4,5-dimethylthiazol-2-yl)-5-(3-carboxymethoxyphenyl)-2-(4-sulfophenyl)-2H-tetrazolium (MTS reagent). Assays were performed in 96-well microtiter plates. Briefly, compounds at 100, 50, 25, and 12.5 μM concentrations were incubated with 2×10⁴ Hela cells per well in Dulbecco's Modified Eagle Medium (Invitrogen Corporation) containing 1% Bovine Growth Serum (HyClone) for 18 h at 37° C. under 5% CO₂. At the end of the incubation, the amount of reducing equivalents was determined by the reduction of MTS reagent (3-(4,5-dimethylthiazol-2-yl)-5-(3-carboxymethoxyphenyl)-2-(4-sulfophenyl)-2H-tetrazolium inner salt) to its parent formazan product ((4,5-dimethylthiazol-2-yl)-3-(3-carboxymethoxyphenyl)-5-(4-sulfophenyl)-formazan) as revealed by absorbance at 490 nm.

Compounds 4, 5, 7, 8, 14, 18, 26, 28, 39, 53, 54, 64 and 154 were assayed under the above mentioned conditions and displayed no signs of any cytotoxicity at concentrations up to 100 μM (data not shown).

### Example 3

### Stability of Fluoroquinolone-Bisphosphonate Conjugates

The stabilities of selected fluoroquinolone-bisphosphonate conjugates in solution and in different media were assessed using a methodology based on either LC/MS (liquid chromatography coupled with mass spectrometry) or detection by biological assay. For LC/MS detection, a 5 μL aliquot of 200 μM solution of the compound was added to 95 μL of the medium (100 mM PBS (pH 7.5), 100 mM Tris (pH 7.5) or rat plasma serum). The mixture was incubated for different time points, and was then diluted with 500 μL of methanol. The mixture was vortexed for 15 min and centrifuged at 10 000 g for 15 min. The supernatant was evaporated under a stream of argon, and the resulting residue was reconstituted in 100 μL of water. The resulting mixture was vortexed for 15 min and centrifuged at 10 000 g for 10 min. A 20 μL aliquot was then used to determine the concentration of parent drug by comparison with LC/MS standards. The LC/MS analytical method was based on an Agilent 1100™ series LC/MSD trap with a Zorbax™ SB-Aq column (2×30 mm, 3.5 μl) using 0.1% formic acid in water: 0.1% formic acid in acetonitrile (85:15) as the mobile phase at a flow rate of 0.3 ml/min. For bioassay detection, a solution of the individual compound at 1 mg/mL in PBS was diluted in an equal volume of the medium and incubated at ambient temperature. At the indicated time points, a 100 μL aliquot of the solution was added to 100 μL of a slurry of 20 mg/ml bone meal powder (Now Foods, Bloomingdale, Ill., USA) in PBS. The suspension of drug/prodrug in bone meal powder was incubated at ambient temperature for 10 min to allow for binding, and centrifuged at 16 000 g for 2 min. The supernatant was assessed for fluoroquinolone content by microbiological assay as follows: Isolated colonies of the indicator strain (E.coli LBB925 toIC) were resuspended in 0.85% saline to OD₆₀₀=0.2 and spread on Cation-adjusted Miller Hinton agar (CAMHA) plates. Known volumes of the supernatants were applied to discs and dried. The discs were then placed on the seeded CAMHA plates. The plates were incubated at 37° C. for 18 h after which the diameters of the zone of inhibition generated by the discs were measured. The amount of prodrug was deduced from standard curves of known amounts of free gatifloxacin 15 that were used as reference for each experiment. The results are displayed in Table 1.

TABLE 1
Regeneration of parent drug from prodrugs in solution

| | | | Parent Drug | | | |
|---|---|---|---|---|---|---|
| Compound | | | Regenerated (%) | | | |
| No. | Method | Medium | 20 Min | 40 Min | 1 h | 24 h |

Parent drug Moxifloxacin (3)

| | | | | | | |
|---|---|---|---|---|---|---|
| 14 | LC/MS | PBS | — | — | 1.1 | 2.2 |
| | | RS | — | — | 1.0 | 1.6 |
| 25 | LC/MS | PBS | — | — | 0.4 | 1.9 |
| | | RS | — | — | 1.8 | 2.4 |
| 52 | LC/MS | PBS | — | — | 2.1 | 15.7 |
| | | RS | — | — | 2.3 | 11.0 |

Parent drug Gatifloxacin (15)

| | | | | | | |
|---|---|---|---|---|---|---|
| 18 | LC/MS | Tris | — | — | 1.3 | 3.2 |
| | | RS | — | — | 4.2 | 10.0 |
| 28 | LC/MS | PBS | — | — | 2 | 1.6 |
| | | RS | — | — | 2.5 | 7.9 |
| 49 | Bioassay | PBS | 29.3 | 35.2 | 45.3 | 101.3 |
| | | RS | 74.7 | 88.0 | 106.7 | 106.7 |
| | | Inactivated | 90.4 | 98.8 | 104.2 | 120.8 |
| | | RS | | | | |
| 54 | LC/MS | Tris | — | — | 7.7 | 31.7 |
| | | RS | — | — | 14.3 | 115 |
| | Bioassay | PBS | 1.8 | 1.8 | 1.8 | 7.6 |
| | | RS | 3.9 | 3.9 | 3.8 | 16.9 |
| | | Inactivated | 5.1 | 5.6 | 5.6 | 15.0 |
| | | RS | | | | |
| 121 | Bioassay | PBS | 3.7 | 8.0 | 41.4 | 97.7 |
| | | RS | 14.4 | 25.9 | 71.8 | 97.7 |
| | | Inactivated | 12.1 | 20.1 | 83.3 | 92.0 |
| | | RS | | | | |

PBS: 100 mM PBS (pH 7.5);
RS: Rat plasma serum;
Tris: 100 mM Tris (pH 7.5).

The data collected and presented in Table 1 provides support for two trends. Firstly, in solution, there is regeneration of the parent drug from the pro-drug, and this regeneration is appears to be somewhat independent of serum hydrolases when comparing activated and inactivated rat sera (compounds 49, 54 and 121). Secondly, the rates observed for prodrugs 18, 28 and 54 of gatifloxacin 15 are unexpectedly markedly faster than those measured for the parent prodrugs, respectively 14, 25, and 52 of moxifloxacin 3 using same bisphosphonate linkers.

### Example 4

### Binding of Compounds to Bone Powder In Vitro and Subsequent Regeneration of the Parent Drug

### Bone Powder Binding

The ability of the example molecules to bind to bone powder was established in two parallel fashions, using either LC/MS or fluorometric detection. For LC/MS detection, a stock solution (5 mM) of the compound to be tested was added to 0.1M Tris-HCl buffer pH 7, 0.15M NaCl to reach a final concentration of 100 μM. Triplicate samples (1 mL) of the compound solutions were intensively shaken for 10 min with or without 25 mg of fresh or 20 mg of vacuum dried ground rat tibia bone. The samples were then centrifuged for 15 min at 10 000 g. The presence of unbound compound was measured by injection of 30 μL of each of the supernatants into an Agilent 1100™ LC/UV system. For fluorometric detection, an individual compound was dissolved in PBS or water (compound 52) and resuspended at a concentration of 1 mg/ml in a slurry of bone meal powder (Now Foods, Bloomingdale, Ill., USA) in PBS at 10 mg/ml. The suspension of drug/prodrug in bone meal powder was incubated at 37° C. for 1 h to allow for binding, and centrifuged at 13 000 rpm for 2 min, before recovering the supernatant. The bone meal powder pellet was then washed three times with 1 ml of PBS. All supernatants were saved and assessed for fluoroquinolone content by fluorescence measurements at excitation/emission wavelengths of 280/465 nm. The amount of fluoroquinolone was determined from standard curves generated for each experiment. Amount of drug/prodrug bound to bone powder was deduced from the difference between the input amount (typically 1 mg) and the amount recovered in the supernatants after binding. In all binding experiments, >99% of input drug was recovered in the supernatant for the parent drugs. The results are displayed in Table 2.

TABLE 2
Levels of Bone binding in vitro

| | | Binding to | |
|---|---|---|---|
| Compound | | bone (%) | |
| No. | Structure | Fluorescence | LC/MS |

Parent drug = Moxifloxacin (3)

| | | | |
|---|---|---|---|
| 3 | 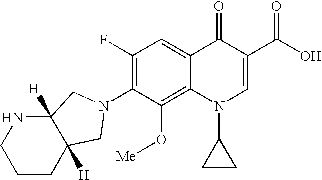 | 0 | — |
| 5 | 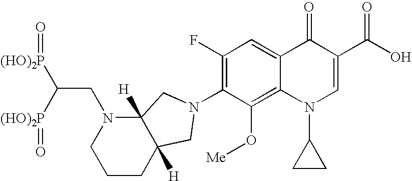 | 92.2 | — |
| 14 | 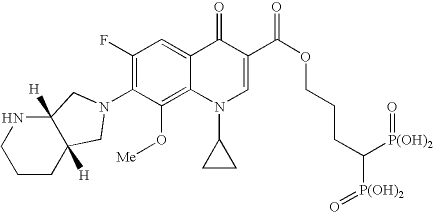 | 93.8 | — |
| 25 | 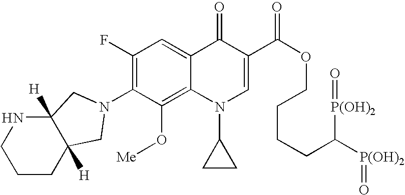 | 92.7 | 87.6 ± 11.5 |
| 36 | 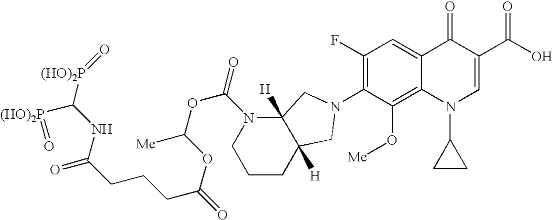 | 93.9 | — |
| 52 | 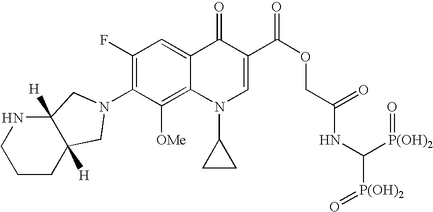 | 98.7 | 94.2 ± 3.2 |
| 62 | 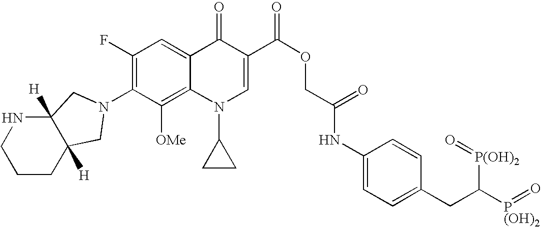 | 97.2 | — |
| 73 | 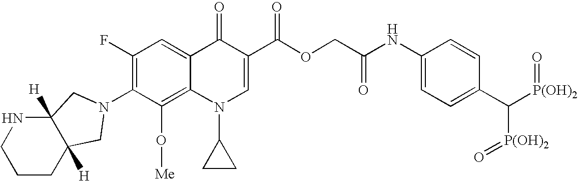 | 99.0 | — |
| 80 | 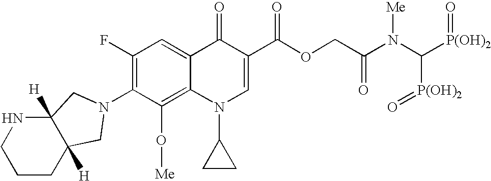 | 97.3 | — |
| 85 | 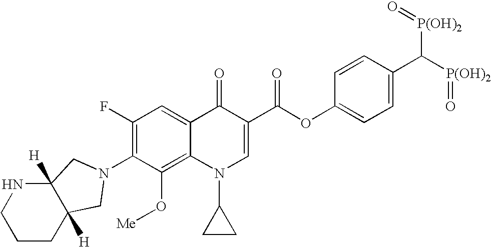 | 98.9 | — |
| 94 | 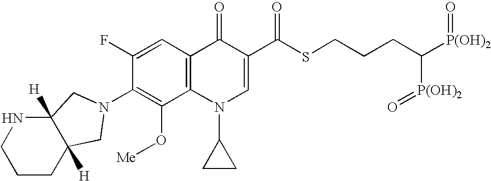 | 99.9 | — |
| 99 | 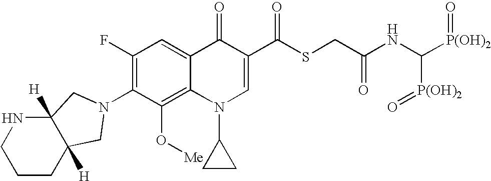 | 99.0 | — |
| 107 | 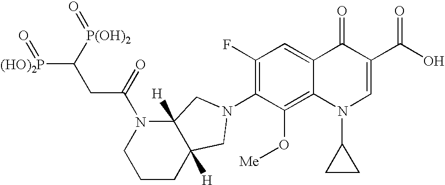 | 98.2 | — |
| 117 | 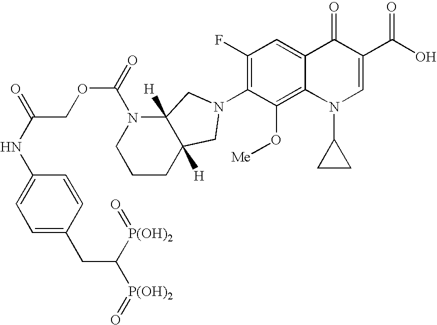 | 99.4 | — |

Parent drug = Gatifloxacin (15)

| | | | |
|---|---|---|---|
| 15 | 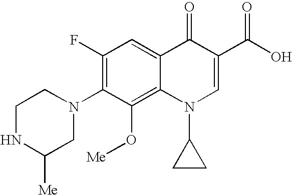 | 0 | — |
| 18 | 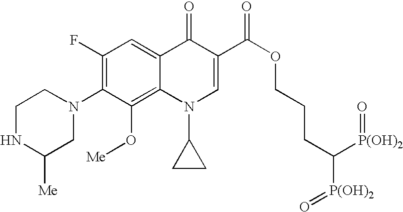 | — | 96.0 ± 2.4 |
| 28 | 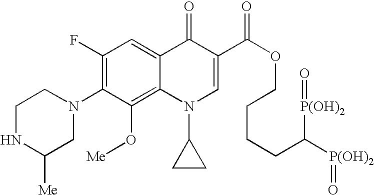 | — | 91.9 ± 5.5 |
| 39 | 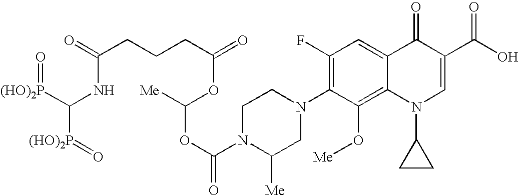 | 94.3 | — |
| 44 | 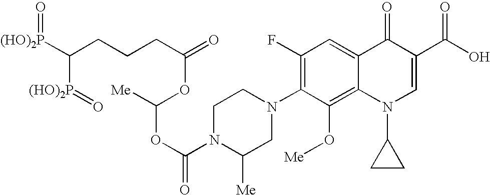 | 88.4 | — |
| 49 | 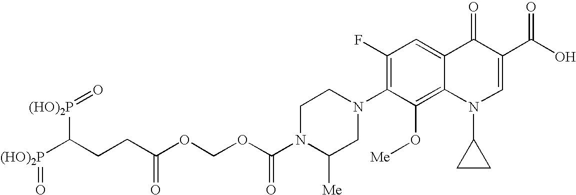 | 86.4 | |
| 54 | 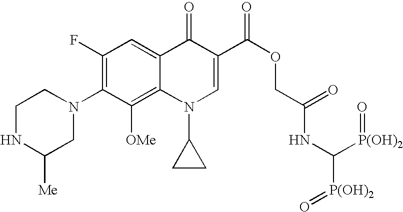 | 91 | — |
| 64 | 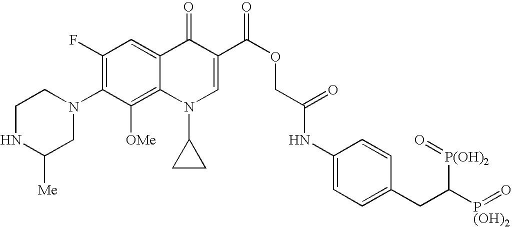 | 88.9 | — |
| 82 | 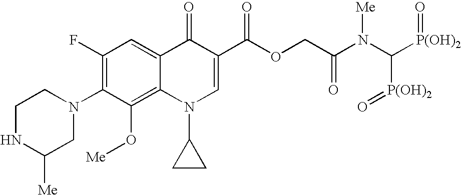 | 90.4 | — |
| 87 | 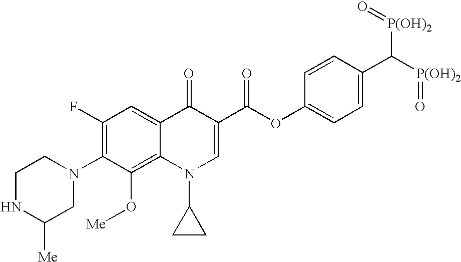 | 79.2 | — |
| 96 | 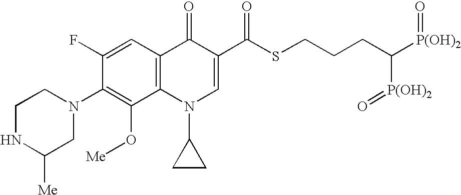 | 98.7 | — |
| 112 | 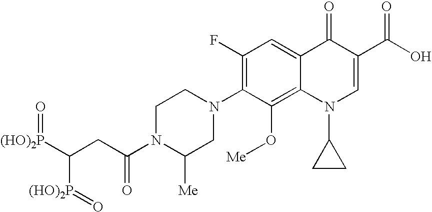 | 82.5 | — |
| 121 | 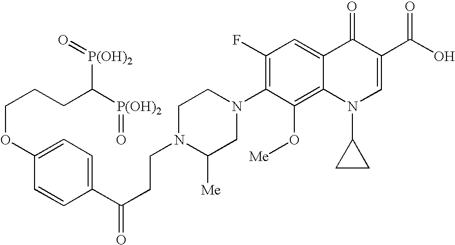 | 94.7 | — |
| 129 | 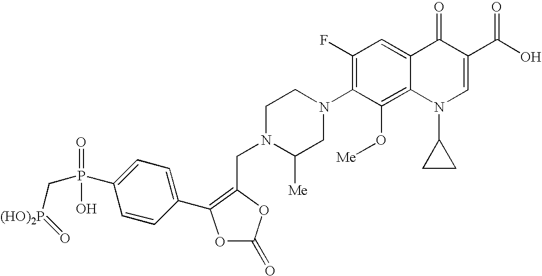 | 63.7 | — |
| 134 | 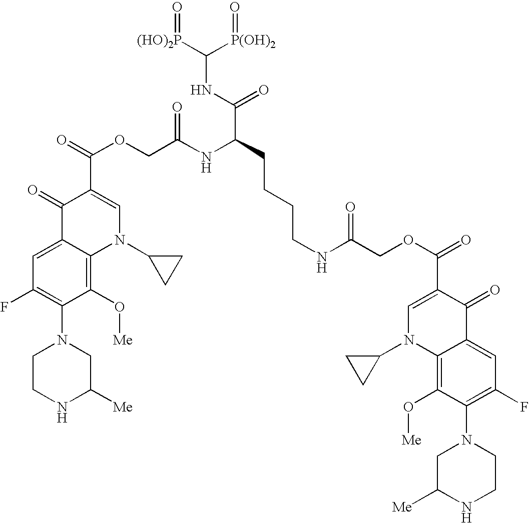 | 92.8 | — |
| 141 | 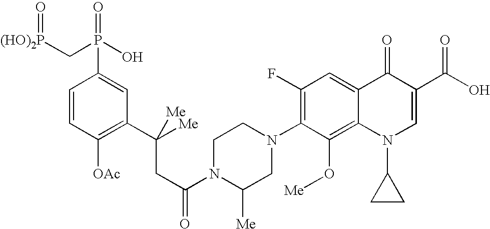 | 36.6 | — |
| 145 | 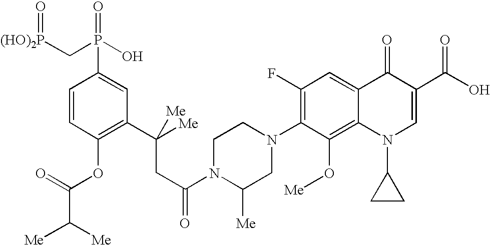 | 76.0 | — |
| 149 | 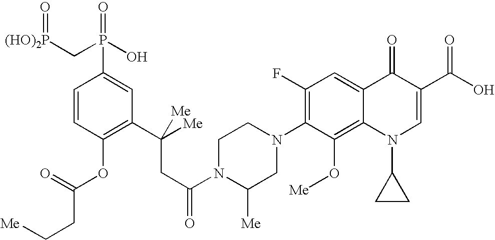 | 89.6 | — |
| 153 | 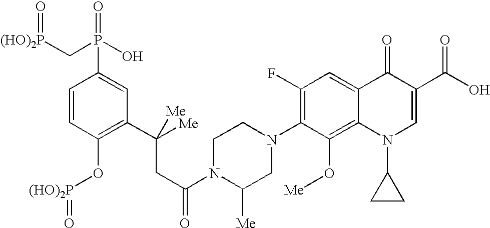 | 0 | — |

Parent drug = Ciprofloxacin (6)

| | | | |
|---|---|---|---|
| 6 | 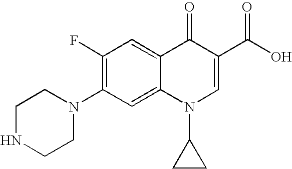 | 0 | — |
| 8 | 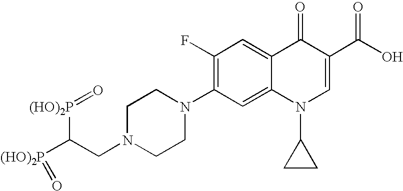 | 99.6 | — |
| 57 | 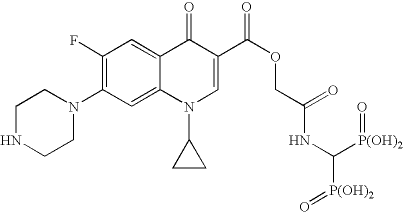 | 97.2 | — |
| 75 | 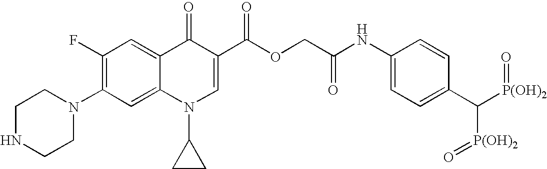 | — | — |

—: Not tested

The results presented in Table 2 confirm that the bisphosphonated prodrugs are very efficiently removed from solution by osseous matter. The results also undeniably lend credence to the use of bisphosphonates as mediators for bone delivery, by comparing prodrug (in general>85% bound) to parent drug (<2% bound in each case). It is reasonable to believe that a portion of the unbound material detected by fluorescence not to be bisphosphonated prodrug but contaminating or regenerated parent drug. Nevertheless, it is also probable that the extent of binding to the osseous matter is reflective of the kinetics of bone absorption/adsorption. There is a clear trend that the bisphosphonates bearing three hydroxy groups (compounds 129, 141, 145, 149 and 153) are less effectively bound. In fact compound 153 is not bound at all, a matter which is completely unexpected.

Regeneration of Drug from Bone Powder-Bound Prodrug

The ability of the prodrug to release the active entity at the site of infection is paramount for use in vivo. This can be partially predetermined by measuring the release of the drug from prodrug bound to osseous matter in vitro.

Amounts of drug “regenerated” from the phosphonated parent prodrug were measured as follows. Washed bone powder-bound prodrugs from the above section were resuspended in 400 μL PBS or in 400 μL 50% (v/v in PBS) human or rat serum. The suspension was incubated for either overnight, three days or six days at 37° C., centrifuged at 13 000 rpm for 2 min and the supernatant was recovered. Methanol (5× volume relative to supernatant) was added to each supernatant and the mixture was vortexed on a floor model vortex for 15 min to extract freed fluoroquinolone. The mixture was then centrifuged at 10 000 rpm for 15 min to pellet the insoluble material. The supernatant containing the extracted fluoroquinolone was recovered and evaporated to dryness in a speed vac. The dried pellets were resuspended in PBS and the amount of fluoroquinolone was determined by fluorescence measurements as described above. The percentage of drug regenerated was deduced from the difference between amount of bound prodrug and the amount of regenerated drug. The identity of regenerated drug was deduced by MIC determination; MICs of regenerated material matched those of parent drugs but not those of prodrugs.

TABLE 3
Regeneration of parent drug from bone-bound prodrug in vitro

| | | | % regenerated | | |
|---|---|---|---|---|---|
| Compound | | | drug after | | |
| No. | Structure | Medium | 24 h | 3 d | 7 d |

Parent drug = Moxifloxacin (3)

| | | | | | |
|---|---|---|---|---|---|
| 5 | 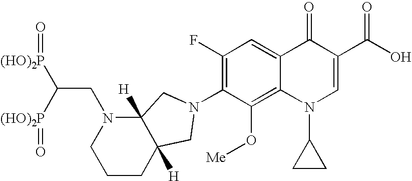 | PBS50% RS50% HS | 0 0 0 | 0.0100 | 0.1900.02 |
| 14 | 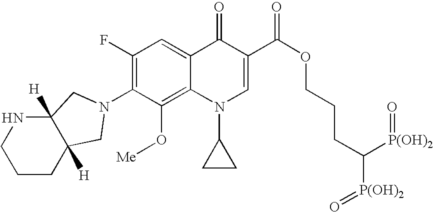 | PBS50% RS50% HS | 0 0 0 | 0.020.030.02 | 0.170.170.2 |
| 25 | 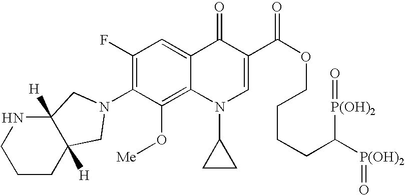 | PBS50% RS50% HS | 0.01 0 0 | ——— | ——— |
| 36 | 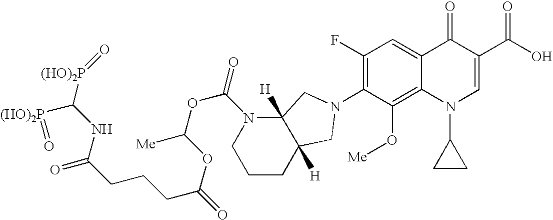 | PBS50% RS50% HS | 1.2 1.4 1.8 | ——— | ——— |
| 52 | 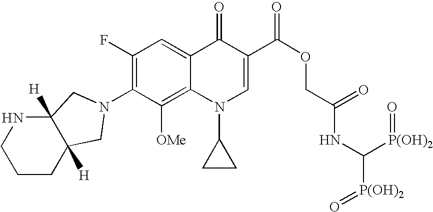 | PBS50% RS50% HS | 1.2 1.4 1.4 | 2.42.52.7 | 6.16.07.4 |
| 62 | 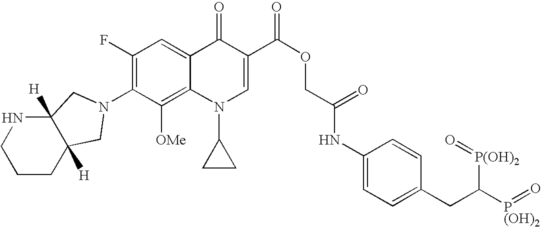 | PBS50% RS50% HS | 0.6 1.3 1.2 | ——— | ——— |
| 73 | 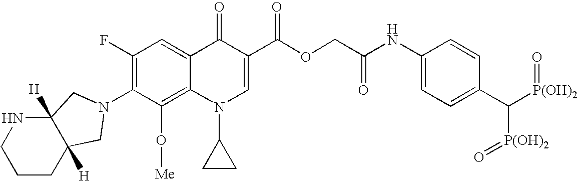 | PBS50% RS50% HS | 0.42 0.43 0.42 | ——— | ——— |
| 80 | 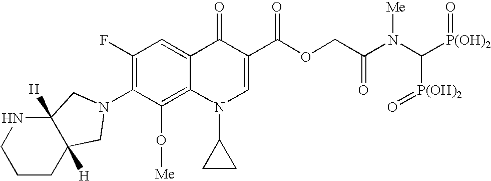 | PBS50% RS50% HS | 0.28 0.72 0.82 | ——— | ——— |
| 85 | 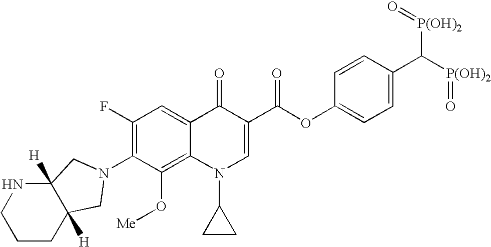 | PBS50% RS50% HS | 0.16 0.21 0.21 | ——— | ——— |
| 94 | 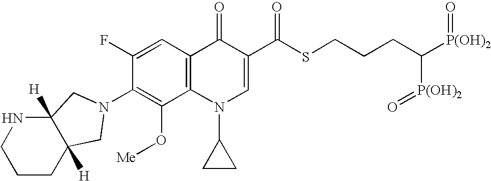 | PBS50% RS50% HS | 0.04 0.08 0.07 | ——— | ——— |
| 99 | 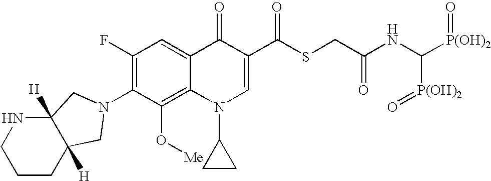 | PBS50% RS50% HS | 0.27 0.40 0.48 | ——— | ——— |
| 107 | 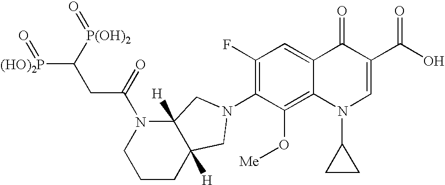 | PBS50% RS50% HS | 0 0 0 | ——— | ——— |
| 117 | 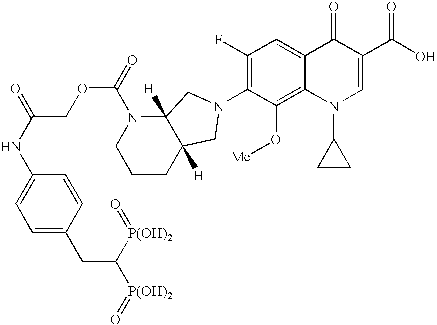 | PBS50% RS50% HS | 0 0 0 | ——— | ——— |

Parent drug = Gatifloxacin (15)

| | | | | | |
|---|---|---|---|---|---|
| 18 | 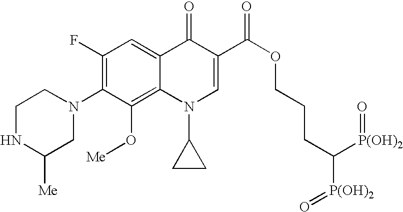 | PBS50% RS50% HS | ——— | ——— | ——— |
| 28 | 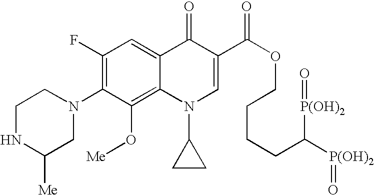 | PBS50% RS50% HS | ——— | ——— | ——— |
| 39 | 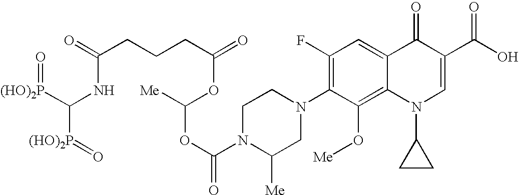 | PBS50% RS50% HS | 1.5 1.8 2.4 | ——— | ——— |
| 44 | 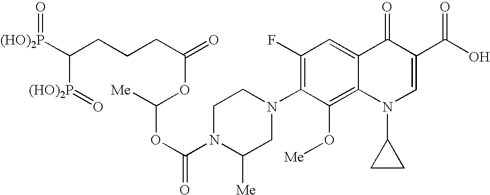 | PBS50% RS50% HS | 0.7 0.7 0.9 | ——— | ——— |
| 49 | 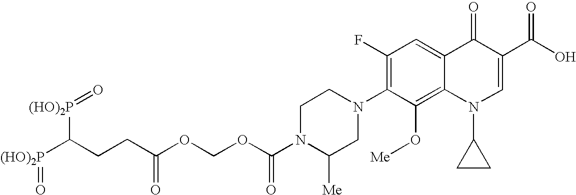 | PBS50% RS50% HS | 13.425.421.2 | ——— | ——— |
| 54 | 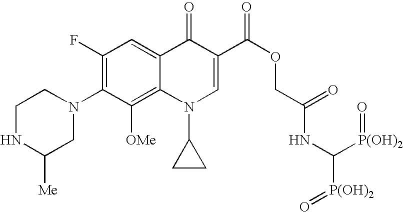 | PBS50% RS50% HS | 2.7 1.3 1.6 | 6.54.46.4 | 12.913.414 |
| 64 | 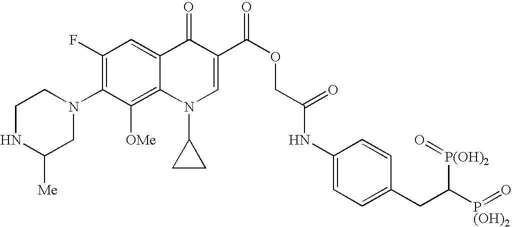 | PBS50% RS50% HS | 0.5 0.6 0.7 | ——— | ——— |
| 82 | 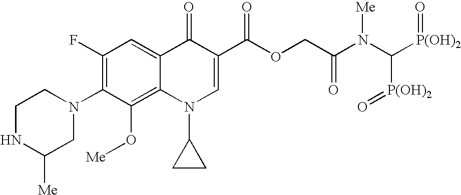 | PBS50% RS50% HS | 1.1 0.85 0.58 | ——— | ——— |
| 87 | 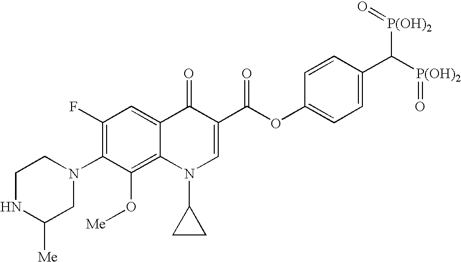 | PBS50% RS50% HS | 0.19 0.13 0.12 | ——— | ——— |
| 96 | 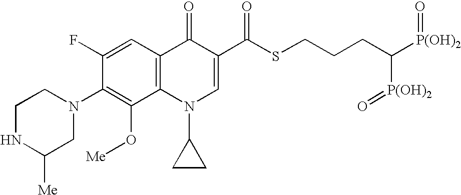 | PBS50% RS50% HS | 0.10 0.07 0.06 | ——— | ——— |
| 112 | 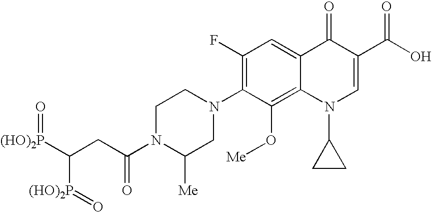 | PBS50% RS50% HS | 0 0 0 | ——— | ——— |
| 121 | 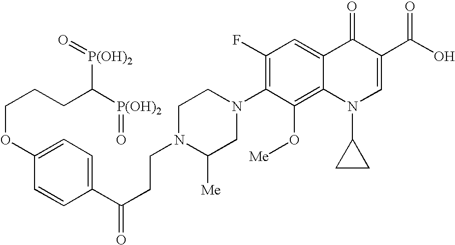 | PBS50% RS50% HS | 2.27 4.23 4.34 | ——— | ——— |
| 129 | 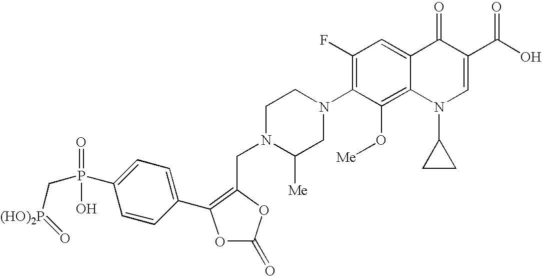 | PBS50% RS50% HS | 7.530.022.8 | ——— | ——— |
| 134 | 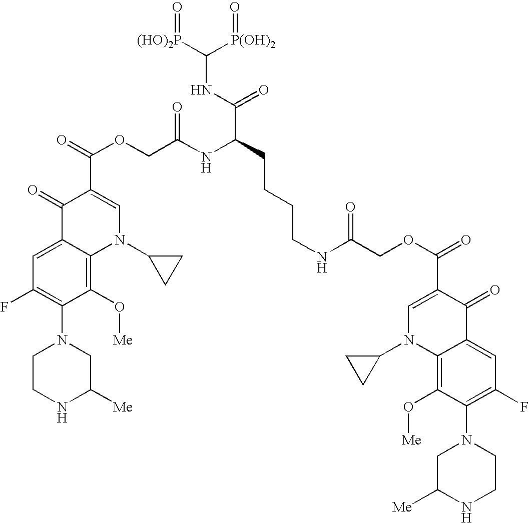 | PBS50% RS50% HS | 0.15 0.36 0.37 | ——— | ——— |
| 141 | 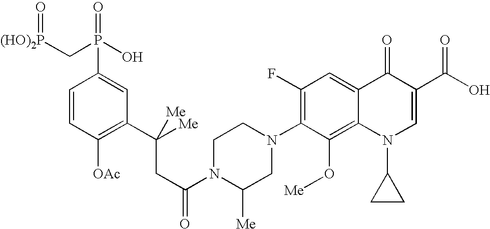 | PBS50% RS50% HS | 5.4 6.2 3.7 | ——— | ——— |
| 145 | 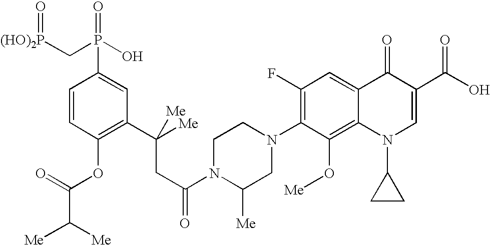 | PBS50% RS50% HS | 0.8 6.1 3.6 | ——— | ——— |
| 149 | 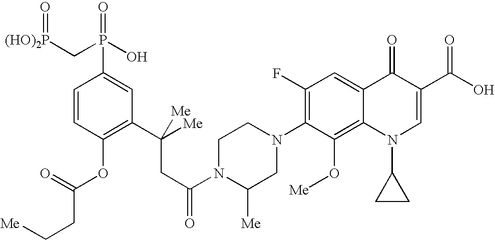 | PBS50% RS50% HS | 0.01 1.85 0.96 | ——— | ——— |
| 153 | 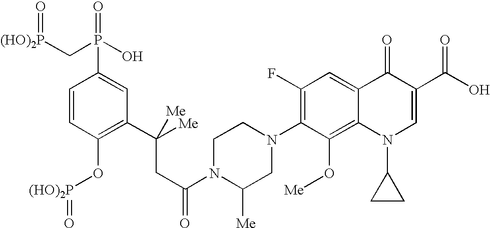 | PBS50% RS50% HS | ——— | ——— | ——— |

Parent drug = Ciprofloxacin (6)

| | | | | | |
|---|---|---|---|---|---|
| 8 | 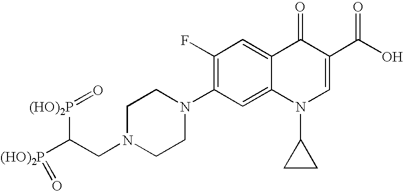 | PBS50% RS50% HS | 0 0 0 | ——— | ——— |
| 57 | 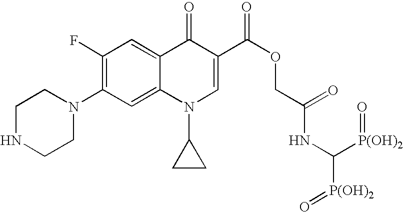 | PBS50% RS50% HS | 0.01 0.45 0.38 | ——— | ——— |
| 75 | 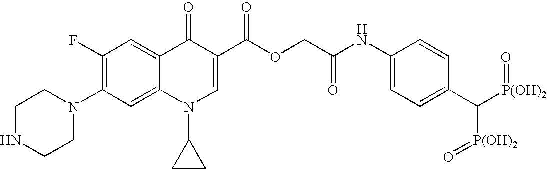 | PBS50% RS50% HS | ——— | ——— | ——— |

The data presented in Table 3 provides evidence as to the importance of the selection of an appropriate bisphosphonate linker on the ability of the prodrugs to release the parent active entity. Several trends are revealed by this data. First, with compounds 5 and 8, both containing a previously reported linker (J. Med. Chem. 2002; 45, 2338-2341), there is not measurable amount of drug released from the bone matter after 24 h, and only trace amounts are observed after 7 days of incubation for 5. Second, the data highlights the fact that the rates of hydrolysis are reduced considerably after immobilization on bone matter. Thus compounds 14 and 25 do not release moxifloxacin 3 to any measurable extent after 24 h once immobilized (Table 3), whereas they do in solution (Table 1), and signs of drug release only occur after 7 days of incubation. In much the same way, compound 52 immobilized on bone powder gives rise to 1.2% regenerated Moxifloxacin 3 (Table 3), whereas in solution it gave 18% of the same compound (Table 1). Compound 54 generated 2.7% gatifloxacin 15 (Table 3), whereas the same compound in solution provided 31.7% of its parent drug (Table 1). Third, the bisphosphonate linker alone is not predictive of the rate of release of parent drug from prodrug. This is evident considering both the solution-phase regeneration data and the bone powder-bound regeneration data for compounds 52 and 54: these two compounds share the same bisphosphonate linker and the same point of attachment (carboxyl) to their parent fluoroquinolone. However, gatifloxacin 15 is released from prodrug 54 at twice the rate of that for moxifloxacin 3 from prodrug 52, whether in solution (Table 1) or bound to bone powder (Table 3). Thus the suitable bisphosphonate linker must be empirically tailored for the selected parent drug so as to release it from prodrug at a rate appropriate for activity in vivo. Finally, the effect of the medium can be negligible, with fairly similar rates regardless of the presence or absence of serum. This is the case for compounds 36, 39, 44, 52, 64, 73, 82, 85, 87 and 141. Although this does not prohibit a role for serum hydrolases in the release of the active entity from bone-bound prodrug, it does demonstrate that the process at the very least needs not rely on their catalysis. On the other hand, for some compounds, such as 49, 57, 62, 80, 99, 121, 129, 134, 145, 149 and 153 there is a noticeable rate acceleration in the presence of serum, with 2-4 fold more regenerated fluoroquinolone regenerated after 24 h, whereas for 54 a rate deceleration is observed. This impact is insufficient evidence to conclude that there is biomolecular catalysis involved. Indeed, one cannot preclude certain medium effects, such as the impact of ionic strength, or the role of certain metal ions assisting cleavage through chelation. In fact, the behaviour of 54 is indicative of the predominance of a chemically, rather than biochemically, induced cleavage. In addition, the prodrugs involving glycolamide linkers display rather erratic behaviours, highly dependent on the substituents on the linker and the particular fluoroquinolone involved. Hence, within this group of compounds, 52, 64, 73 and 82 are not impacted by the medium change. Compounds 57, 62, 80, and 99 exhibit greater regeneration in the presence of serum and compound 54 displays lesser regeneration. The substitution patterns on the linker are not predictive of this behavior, and which can only be experimentally determined.

### Example 5

### Determination of Levels of Moxifloxacin-Bisphosphonate Conjugate No. 52 and Gatifloxacin-Bisphosphonate Conjugates No. 49 and 54 in Rat Tibia In Vivo

In order to investigate the bone binding and rate of decomposition of bisphosphonated fluoroquinolone prodrugs in vivo, rats were treated with bisphosphonated moxifloxacin prodrug 52 or bisphosphonated gatifloxacin prodrug 49 or 54 and the drug content of their tibiae was analyzed at different time points after injection. Female CD rats (age, 57 to 70 days; n=5/group; Charles River, St-Constant, Canada) were administered a single bolus intravenous (tail vein) dose of compound 49, 52 or 54 (dissolved in 0.85% NaCl) at respectively 18.8 mg/kg (equivalent to 9 mg/kg of 15), 15.8 mg/kg (equivalent to 10 mg/kg of 3) and 17.4 mg/kg (equivalent to 10 mg/kg of 15) of body weight. Animals were humanely sacrificed at specified time points after i.v. dosing to evaluate the levels of 49, 52 or 54 in the bone. Tibiae were recovered by dissection, cleaned from soft tissues, ground using a metal ball mill (Retsch MM301™) and kept at −80° C. before determination of the drug concentration of the bone.

### Determination of the Concentrations of Bisphosphonated Compounds 49, 52 and 54 in the Tibiae

The experimentally obtained ground bone powder was suspended in 5% formic acid in methanol (500 mg/1.6 mL). The mixture was vortexed for 10 min and centrifuged for 10 min at 10 000 g. The resulting pellet was dried, weighed and used for the determination of the amount of bisphosphonated prodrug.

For the dosage of the prodrug in the tibia, the standards, QCs (Quality Controls) and blanks were prepared (in duplicate) as follows: to 20 mg of dry blank tibia powder were added a spiking solution (10 μL) of prodrug and 990 μL of buffer (0.1M tris-HCl, pH 7, 0.15M NaCl); the mixture was vortexed for 10 min (RT), centrifuged for 15 min at 10 000 g (RT), the supernatant discarded and the pellet kept for the cleavage procedure. The range of the standards (6 levels) was from 0.05 to 10 μM and the QC levels were 0.075, 0.75 and 7.5 μM.

To each standard, QC, blank and experimental sample tibia bone pellet (20 mg of dry weight) was added 500 μL 6N NaOH for the cleavage of the prodrug into the drug (moxifloxacin 3 or gatifloxacin 15). After an incubation at 50° C. for 1 hour (for 49) or 2 hours (for 52 and 54), the mixture was acidified with 6N HCl (500 μL) and the internal standard (gatifloxacin 15 for 52 and moxifloxacin 3 for 49 and 54) was added. After a centrifugation of 15 min at 10 000 g (RT), the supernatant was extracted on a Strata™ cartridge (30 mg/l ml), using 100% methanol as the eluent. The eluent was evaporated to dryness under a stream of argon and the dried residue was reconstituted in 200 μL of the mobile phase used in the LC/MS analysis by vortexing for 15 min. After centrifugation for 15 min at 10 000 g, 20 μL of the supernatant was injected into the LC/MS analyser.

The quantity of drug resulting from the cleavage of the prodrug was analyzed on an Agilent 1100™ series LC/MSD trap. The supernatant was injected into a Zorbax™ SB-Aq column (2×30 mm, 3.5μ), using 0.1% formic acid in water: 0.1% formic acid in acetonitrile (85:15) as the mobile phase at a flow rate of 0.3 mL/min. The MS was set as follows: ESI probe, positive polarity, nebulizer 45 psi, dry gas temperature 350° C., dry gas flow 10 L/min, capillary exit 140V and skimmer 37V. For compounds 52 and 54, a run time of 8 min with the divert valve set to the waste for the first 1.2 min was selected. For compound 49, a run time of 14 min with the divert valve set to the waste for the first 2 min was selected. The detected ions were determined in single reaction mode (SRM).

For the determination of 52, moxifloxacin 3 was analyzed for m/z 402.2 and the internal standard (gatifloxacin 15) for m/z 376.1→332.1.

For the determination of 49 and 54, gatifloxacin 15 was analyzed for m/z 376.1→332.1 and the internal standard (moxifloxacin 3) was analyzed for m/z 402.2.

Concentrations of Bisphosphonated Compounds 52 and 54 in the Tibiae at 7-28 Days after Injection

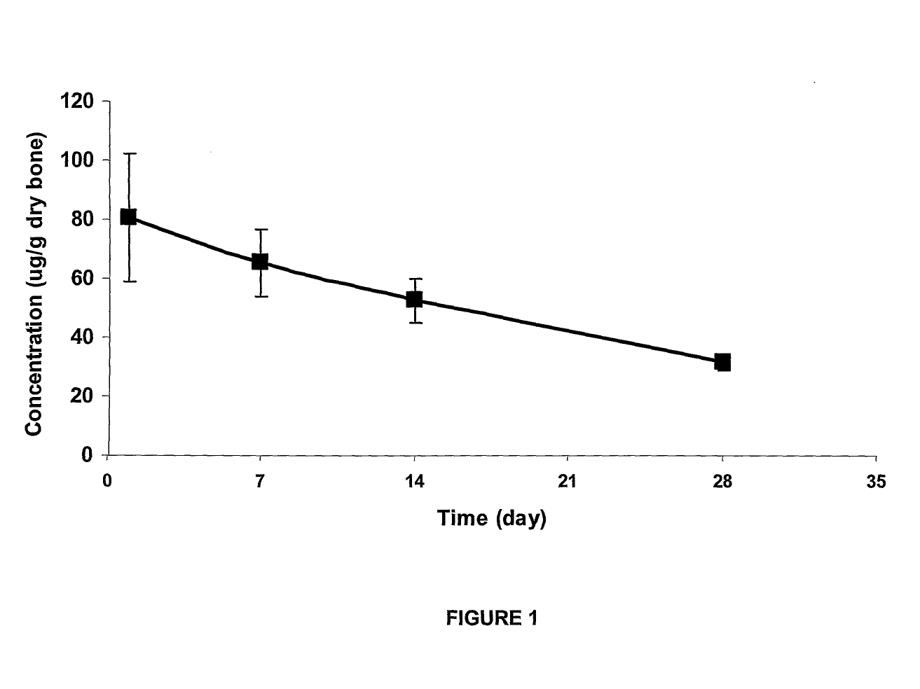

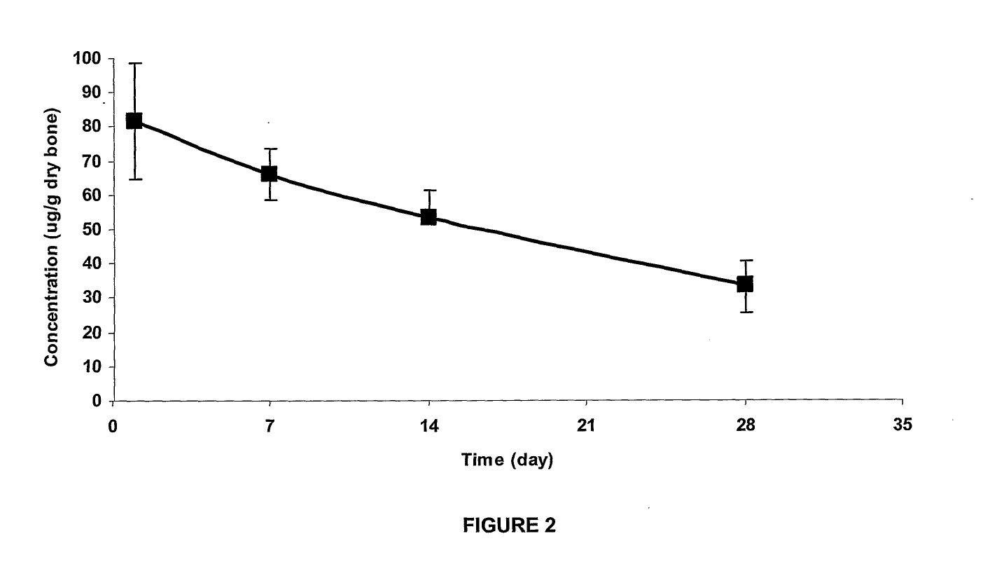

The results of the determination of 52 and 54 in rat tibiae are displayed in FIGS. 1 and 2 respectively.

The data indicates that high concentrations of prodrugs 49, 52 and 54 are present in bone, supporting the use of bisphosphonated moieties in prodrugs to transport fluoroquinolone antibacterials to osseous tissues. The compounds possess half-lives of 20 days for 52 and 21 days for 54. The results are in good agreement with an exponential decrease of the amounts of prodrugs from bone (data not shown).

Concentration of Bisphosphonated Compound 52 in the Tibiae at 5 Min to 24 h after Injection

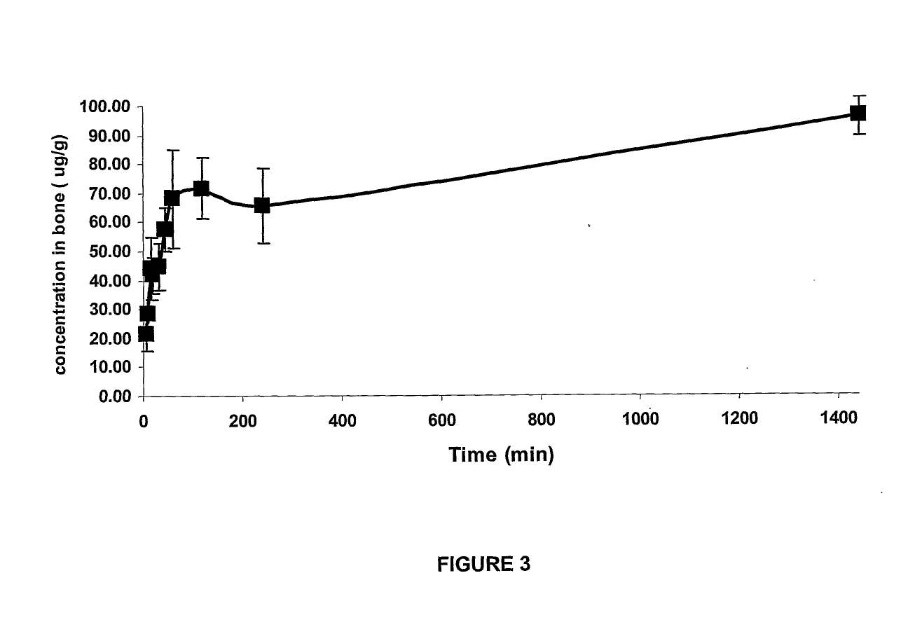

The results for the determination of 52 in rat tibiae over a short period of time are displayed in FIG. 3.

The data shows that the bisphosphonated prodrug accumulates extremely rapidly in bone, with a half-life for the accumulation process of less than 1 hour.

Concentrations of Bisphosphonated Compound 49 in the Tibiae at 0-120 Hours after Injection

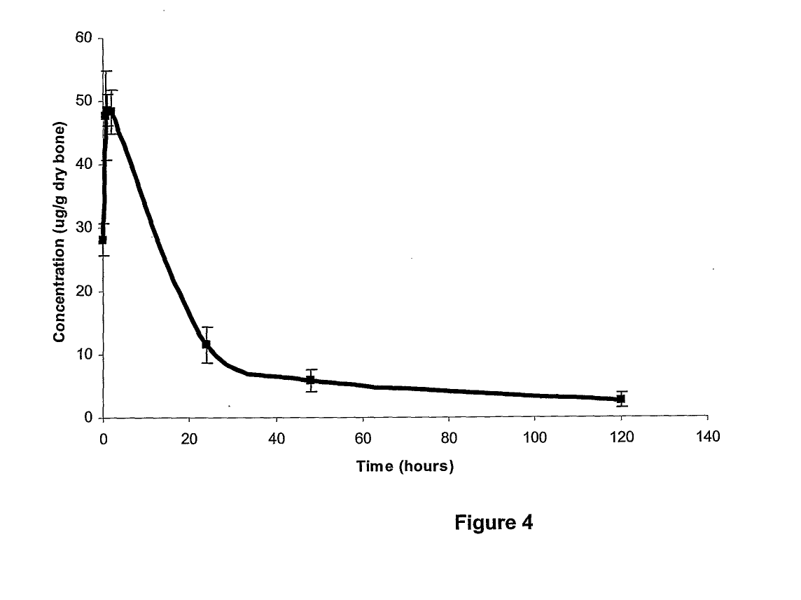

The results of the determination of 49 in rat tibiae are displayed in FIG. 4.

The data indicates that, although high, the concentration of the rapidly cleaving prodrug 49 present in bone is lower than those of the slower cleaving 52 and 54. This still supports the use of bisphosphonated moieties in prodrugs to transport fluoroquinolone antibacterials to osseous tissues. Compound 49 with a much higher rate of regeneration in vitro expectedly possesses a much shorter life span. The disappearance from tibiae occurs in two phases, an initial rapid phase (half-life of 11 h) and a slower second phase (half-life of 2 days).

### Example 6

### Determination of Levels of Moxifloxacin-Bisphosphonate Conjugate No. 52 in Rat Plasma In Vivo

In order to assess the kinetics of clearance of bisphosphonated prodrugs of fluoroquinolones from the blood circulation, the levels of bisphosphonated moxifloxacin prodrug 52 were determined in plasma, at short time intervals (5 min to 24 h) after injection. Female CD rats (age, 57 to 70 days; n=3/group; Charles River, St-Constant, Canada) were administered a single bolus intravenous (tail vein) dose of compound 52 (dissolved in 0.85% NaCl) at 15.8 mg/kg of body weight, corresponding to 10 mg/kg of moxifloxacin 3. Animals were sacrificed by CO₂ inhalation at the specified time points after i.v. dosing to evaluate the levels of 52 in plasma. Blood samples were collected by cardiac puncture and transferred in BD Vacutainer tubes (green cap) for plasma isolation. The plasma components were obtained by centrifugation of these samples and they were stored at −80° C. until analysis.

### Determination of the Concentrations of Bisphosphonated Compound 52 in Plasma by LC/MS

For the dosage of prodrug in the plasma, the standards and QCs were prepared (in duplicate) as follows: to 100 μl of blank plasma was added a spiking solution (5 μl) of the prodrug (52 in water). The range of the standards (8 levels) was from 0.06 to 25 μM and the QC levels were 0.18, 1.87 and 18.75 μM. Four samples of blank plasma without prodrug were also prepared.

To each standard, QC, blank and experimental plasma (100 μl) was added 10 μl of hydroxyapatite suspension (Sigma #H02520) for the binding of the prodrug. After vortexing (10 min) and centrifugation (10 min, 10000 g, RT), the supernatant was discarded and the pellet was washed twice with water (HPLC grade) followed by centrifugation (10 min, 10000 g, RT). Sodium hydroxide 6N (500 μl) was added to the washed pellet, and the mixture was vortexed and incubated at 37° C. for one hour in a water bath for the cleavage of the prodrug into the drug (moxifloxacin 3). After the incubation period, the mixture was acidified with 6N hydrochloric acid

(500 μl) and the internal standard (ciprofloxacin 6, 5 μl of a stock solution at 50 μM in water) was added. The internal standard was added to blank plasma samples but not to the double blank plasma samples. Samples were vortexed 10 minutes and extracted on a strata cartridge (30 mg/l ml), using formic acid:methanol (1:99) as the eluent. The eluent was evaporated to dryness, the dried residue was reconstituted in 200 μl mobile phase (initial conditions) and 20 μl were injected into the LC/MS.

Moxifloxacin 3 resulting from the cleavage of the prodrug was analyzed with the same method on an Agilent 1100™ series LC/MSD Trap, The extracted sample was injected into a Zorbax™ SB-Aq column (2×30 mm, 3.5μ), using 0.1% formic acid in water(aq) and 0.1% formic acid in acetonitrile(org) as the mobile phase, at a flow rate of 0.3 ml/min. The program used was: 12% org for the 2 first minutes, then switching to 20% org in 0.01 minute and maintaining those conditions for 5 minutes, then switching to 50% org in 0.01 minute and maintaining those conditions for 1 minute, before returning to the initial conditions and equilibrating. The MS was set as follows: ESI probe, positive polarity, nebulizer 45 psi, dry gas temperature 350° C., dry gas flow 10 L/min, capillary exit 125 V(1.8 to 4 minutes) or 140 V(4 to 13 minutes) and skimmer 37 V. The run time was 13 minutes with the divert valve set to the waste for the first 1.8 minutes. Moxifloxacin 3 was analyzed for m/z 402.2→358.1 at time 7.1 minutes and the internal standard (ciprofloxacin, 6) for m/z 332.1→288.0 at time 2.9 minutes, in single reaction mode (SRM).

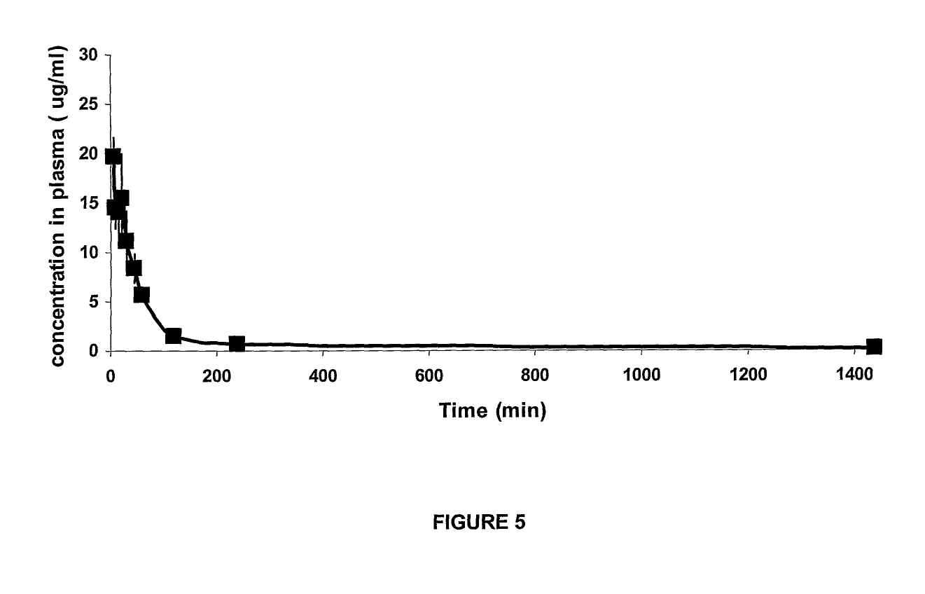

The results of the determination of the concentration of 52 in plasma are presented in FIG. 5. The prodrug 52 is rapidly cleared from the circulation with a half-life of less than one hour, in complete agreement with the complementary rate of accumulation in bone displayed in FIG. 3.

### Example 7

### Prophylactic Use of Prodrug Compounds 39, 44, 49, 52, 54, 107, 121, 129, 141, 145, 149 and 153 in Rats

To determine the activity in vivo of bisphosphonated prodrugs of fluoroquinolones, compounds 52 and 107, derivatives of parent compounds moxifloxacin 3 (for 52), and compounds 39, 44, 49, 54, 121, 129, 141, 145, 149, derivatives of parent compound gatifloxacin 15, were used in a prophylactic model for infection. Specifically, S. aureus ATCC 13709 cells were grown overnight at 37° C. in brain heart infusion broth (BHIB). Cells were subcultured into fresh BHIB and incubated for 4 to 5 h at 37° C. The cells were washed twice with phosphate-buffered saline (PBS) and resuspended in BHIB supplemented with 10% (vol./vol.) fetal bovine serum at a density of approximately 10¹⁰ colony forming units (CFU)/ml (based upon turbidimetry). The suspension was aliquoted and a portion was used to check the CFU count. The culture was stored frozen (−80° C.) and was used without subculture. For use as an inoculum the culture was thawed, diluted in PBS and kept in an ice bath until it was used.

Animals were infected as described by O'Reilly et al. (Antimicrobial Agents and Chemotherapy (1992), 36(12): 2693-97) to generate the bone infection. Female CD rats (age, 57 to 70 days; n=5/group; Charles River, St-Constant, Canada) were anaesthetized by isofluorane before and during the surgery. Following complete induction of anesthesia, the rat was placed ventral side up and hair was shaved from the surgical site. The skin over the leg was cleaned and disinfected (proviodine-ethanol 70%). A longitudinal incision below the knee joint was made in the sagital plane. The incision was made over the bone below the “knee joint” (tibia head or condyle) but not completely extending to the ankle. A high speed drill fitted with a 2 mm bulb bit was used to drill a hole into the medullar cavity of the tibia. Rats were injected intra-tibially with 0.05 ml 5% sodium morrhuate (sclerosing agent) and then with 0.05 ml of S. aureus suspension (ca. 5×10⁵ CFU/rat). The hole was sealed by applying a small amount of dry dental cement which immediately absorbs fluids and adheres to the site. The wound was closed using 3 metal skin clips. Moxifloxacin 3 (as a positive control) was injected once at 10 mg/kg intravenously 1 h postinfection in saline, while the fluoroquinolone prodrugs (prepared in 0.9% saline) were injected as a single intravenous bolus dose at different time points prior to the infection. For comparison, the parent drug (moxifloxacin 3 or gatifloxacin 15) was also injected intravenously once at a molar equivalent dose at the same time point prior the infection.

Infected rats were sacrificed by CO₂ asphyxiation 24 h postinfection to monitor the bacterial CFU count. Infected tibiae were removed, dissected free of soft tissue, and weighed. The bones were ground using a metal ball mill, resuspended in 5 ml 0.9% NaCl, serially diluted and processed for quantitative cultures. For compounds 39, 44, 49, 54, 141, 145, 149, 1 ml of the 0.9% NaCl solution was added to 50 mg of charcoal before serial dilutions. Treatment efficacies were measured in terms of Log viable bacteria (Log CFU per gram of bone). The results obtained for each group of rats were evaluated by calculating the mean Log CFU and standard deviation. The limit of detection is 2 Log CFU/g of bone. Statistical comparisons of viable bacterial counts for the different treated and untreated groups were performed with Dunnett's multiple-comparison test. Differences were considered significant when the P value was <0.05 when comparing treated infected animals to the untreated infected ones.

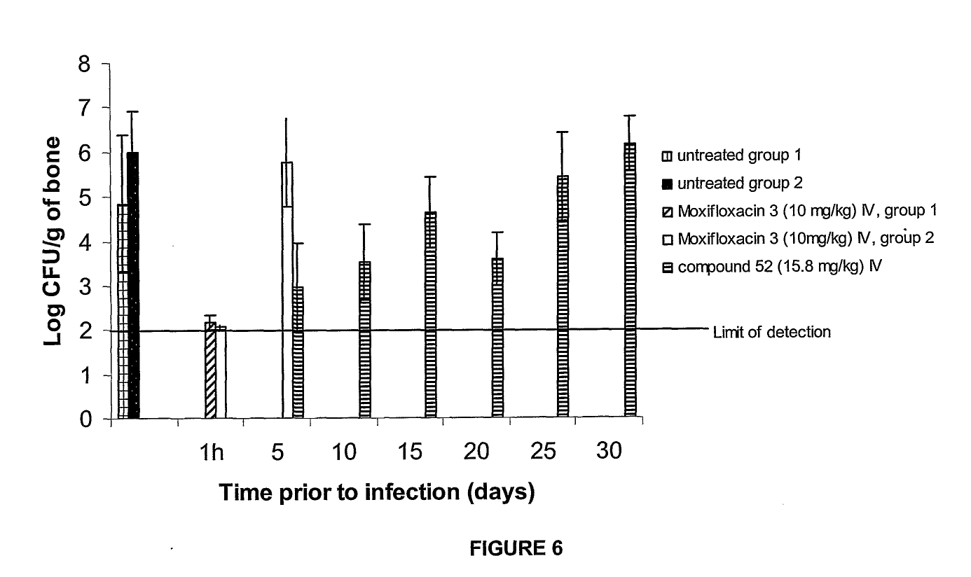

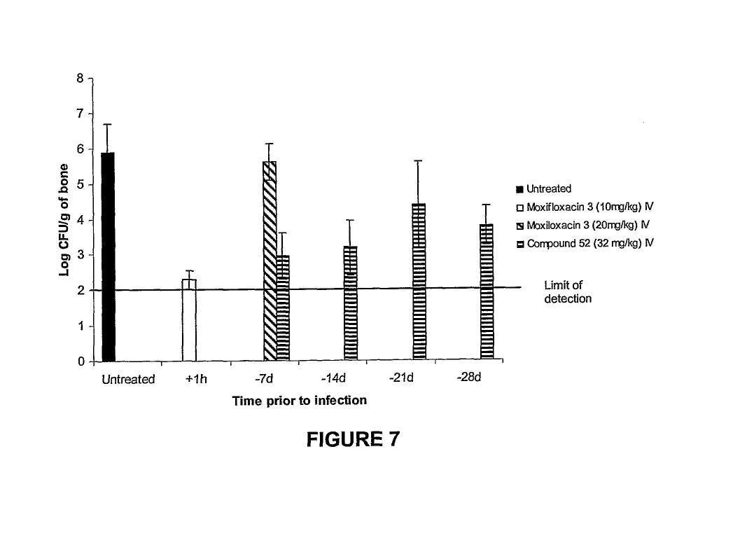

The experiment was performed using compound 52 (a bisphosphonated prodrug of moxifloxacin) at 15.8 mg/kg (equivalent to 10 mg/kg of moxifloxacin 3) injected intravenously at different time points (up to 30 days) prior to infection. The untreated group and the group treated with moxifloxacin 3 1 h post infection were repeated (both sets are shown). Comparison with the second untreated group demonstrated significant (p<0.05) decrease in bacterial titer for the prodrug 52 treated groups 5, 10, 15 and 20 days before infection as well as the moxifloxacin 3 treated groups at 1 h after. The results are displayed in FIG. 6. A parallel experiment was conducted using 52 at 32 mg/kg, corresponding to 20 mg/kg of moxifloxacin 3. In this case, the comparison (p-values) with the untreated group showed a significant decrease in bacterial titer in animals treated with 52 when administered 7, 14, 21 and even 28 days prior to infection. Those treated with 3 at 10 mg/kg 1 h after infection showed also a significant decrease in the bacterial titer, but not those treated with 3 at 20 mg/kg seven days before. The results are displayed in FIG. 7.

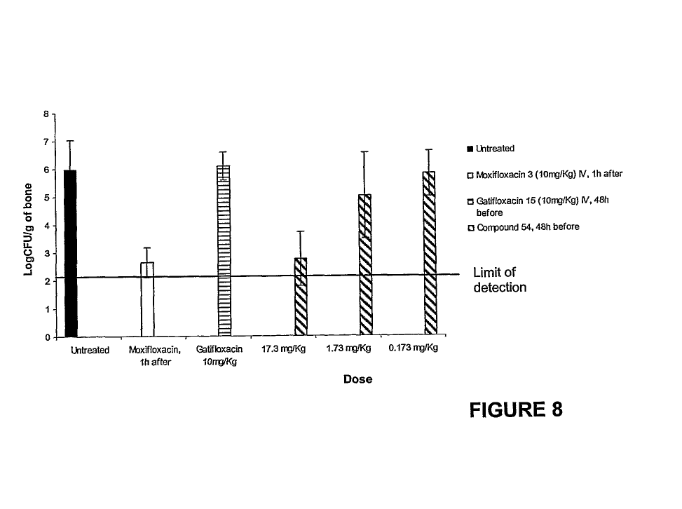

The experiment was also performed using compound 54 (a bisphosphonated prodrug of gatifloxacin prodrug) injected intravenously 48 h prior to infection, but at different doses. For comparison gatifloxacin 15 was also administered 48 h prior to infection, at 10 mg/Kg (equivalent to 17.3 mg/Kg of 54). Comparison with the untreated group demonstrated significant (p-value ≦0.0002) decrease in bacterial titer for the prodrug 54 treated group at 17.3 mg/Kg 48 h prior to infection as well as the moxifloxacin 3 treated group (positive control) administered 1 h after. The results are displayed in FIG. 8.

The prophylactic treatments of the rats with the other bisphosphonated prodrugs of moxifloxacin and gatifloxacin 15 are displayed in Table 4.

TABLE 4
Retrieved bacterial titers following prophylactic treatment in rat model of bone
infection

| | | Time of | Measured bacterial titer | | | |
|---|---|---|---|---|---|---|
| | | administration | (Log CFU/g of bone) | | | |
| Compound | Dose | (days prior to | Test | | Positive | Parent |
| No. | (mg/kg) | infection) | compound | Untreated | control^{a} | drug |

Moxifloxacin 3 prodrugs

| | | | | | | |
|---|---|---|---|---|---|---|
| 52 | 15.8 | 2 | 2.29 ± 0.30 | 6.29 ± 0.78 | 2.05 ± 0.16 | 5.39 ± 1.05 |
| 107 | 19.4 | 1 | 5.40 ± 0.93 | 6.32 ± 1.34 | 2.53 ± 0.39 | n.d.^{b} |

Gatifloxacin 15 prodrugs

| | | | | | | |
|---|---|---|---|---|---|---|
| 39 | 20 | 3 | 4.63 ± 0.39 | 5.18 ± 0.52 | 2.24 ± 0.40 | 5.89 ± 0.78 |
| 44 | 20 | 3 | 4.72 ± 0.83 | 5.18 ± 0.52 | 2.24 ± 0.40 | 5.89 ± 0.78 |
| 49 | 42 | 2 | 2.39 ± 0.27 | 5.19 ± 0.40 | 2.36 ± 0.42 | 5.48 ± 0.95 |
| 54 | 20 | 3 | 2.96 ± 0.69 | 5.83 ± 0.46 | 2.34 ± 0.38 | 5.58 ± 0.65 |
| 121 | 20.8 | 1 | 3.01 ± 0.47 | 6.32 ± 1.34 | 2.53 ± 3.39 | n.d. |
| 129 | 10.75 | 2 | 6.14 ± 1.05 | 5.53 ± 0.79 | 2.59 ± 0.56 | 5.58 ± 0.66 |
| 141 | 45.4 | 2 | 2.40 ± 0.44 | 5.19 ± 0.40 | 2.36 ± 0.42 | 5.48 ± 0.95 |
| 145 | 47.2 | 2 | 4.69 ± 0.78 | 5.76 ± 0.90 | 2.54 ± 0.44 | 5.98 ± 0.79 |
| 149 | 15 | 2 | 5.16 ± 0.72 | 5.41 ± 0.45 | 2.14 ± 0.08 | 4.97 ± 0.72 |
| 153 | 15 | 2 | 4.08 ± 0.54 | 5.41 ± 0.45 | 2.14 ± 0.08 | 4.97 ± 0.72 |

^{a}Moxifloxacin injection (10 mg/kg) after infection.
^{b}n.d.: not determined

The results clearly indicate an efficient prophylactic effect of bisphosphonated fluoroquinolone prodrugs 49, 52, 54, 121 and 141. The data in FIGS. 6 and 7 demonstrate that moxifloxacin prodrug 52 is able to reduce the bacterial titer of the infection when administered weeks before surgery. This correlates well with a half-life of 20 days measured previously for this prodrug (FIG. 1). It is notable that moxifloxacin 3 itself is unable to exhibit such efficient prophylaxis when used five or even two days prior to infection. These results and the bone binding data strongly support the ability of the bisphosphonated prodrug to target osseous matter in vivo, where it is able to release its active moiety at concentrations above those needed for antibacterial activity. This release can be sustained for weeks after a single injection.

A relationship between dose and antibacterial activity is clearly displayed by gatifloxacin prodrug 54. This compound is able to produce nearly sterile bone when used at 17.3 mg/Kg 48 h prior to infection, whereas gatifloxacin 15 is without effect at an equivalent dose (FIG. 8). Similarly, when compound 52 is used at 32 mg/kg, the prophylactic effect lasts longer, as shown in the comparison of FIGS. 6 and 7. The clear relationship between doses and prophylactic activity demonstrates the ability to modulate an in vivo effect of the phosphonated compounds of the invention by changing their dose. It also supplies further evidence as to a clear relationship between the phosphonated compounds of the invention as treatment agents and the treatment outcome in an in vivo model.

The prophylactic effect of prodrug 121 and the lack of effect of conjugate 107 at the same molar dose highlight the importance of the ability of the bisphosphonated entity to undergo a cleavage process to release the parent antibacterial. Simple delivery to the bone is not sufficient. This also demonstrates that the process of covering the bone surface with a bisphosphonated entity is largely inadequate in itself to produce prophylaxis.

More unexpected are the results of prodrugs 39, 44, 129, 145, 149 and 153. These compounds regenerate (Table 3) well in vitro but are unable to produce a positive result in vivo. Although in some instances, it may be argued that the regeneration process requires biocatalysis and that the adequate enzymes are not present in osseous tissues, this cannot be argued for compounds 39 and 44 which regenerate at rates which are similar to 54 and regenerate equally well in the absence and presence of serum (Table 3). On the other hand, it could be argued that the lack of activity is due to regeneration in plasma before being able to reach the bone. Yet compound 49 with extremely rapid regeneration in solution in plasma (Table 1) does provide a positive result.

The discrepancies between in vitro and in vivo results emphasize the need to experimentally determine the suitability of the particular prodrug.

These experiments also demonstrate that efficacious treatments require both delivery to the bone, which was earlier demonstrated to be extremely efficient as a result of the bisphosphonate functionality, and a subsequent decomposition of the bisphosphonated entity to its parent antimicrobial fluoroquinolone.

### Example 8

### Determination of the Amount of Moxifloxacin 3 Regenerated from Bisphosphonated Moxifloxacin Prodrug 52 in Infected and Uninfected Bone

In order to measure the impact of the infection on the access of the bisphosphonated prodrugs, in particular in light of the inflammation and the decreased circulation at the site of the surgical intervention, the levels of regenerated moxifloxacin in the tibiae of the infected and the uninfected hind limbs of infected rats were determined.

Rats were infected as in Example 7, and treated IV with either 15.8 or 31.6 mg/kg of body weight of prodrug 52 one day after surgery. One day and six days following the treatment, the tibiae were collected as described previously. The levels of regenerated moxifloxacin 3 were determined as follows.

### Determination of Regenerated Moxifloxacin in Tibia by LC/MS

The experimentally obtained whole tibia was ground to a powder which was suspended in 5% formic acid in methanol (500 mg/1.6 ml) to extract the released moxifloxacin 3. The mixture was vortexed for 10 minutes and centrifuged for 20 minutes at 1250 g. The supernatant (160 μl) was collected and spiked with the internal standard (ciprofloxacin 6), vortexed and evaporated to dryness under a stream of nitrogen.

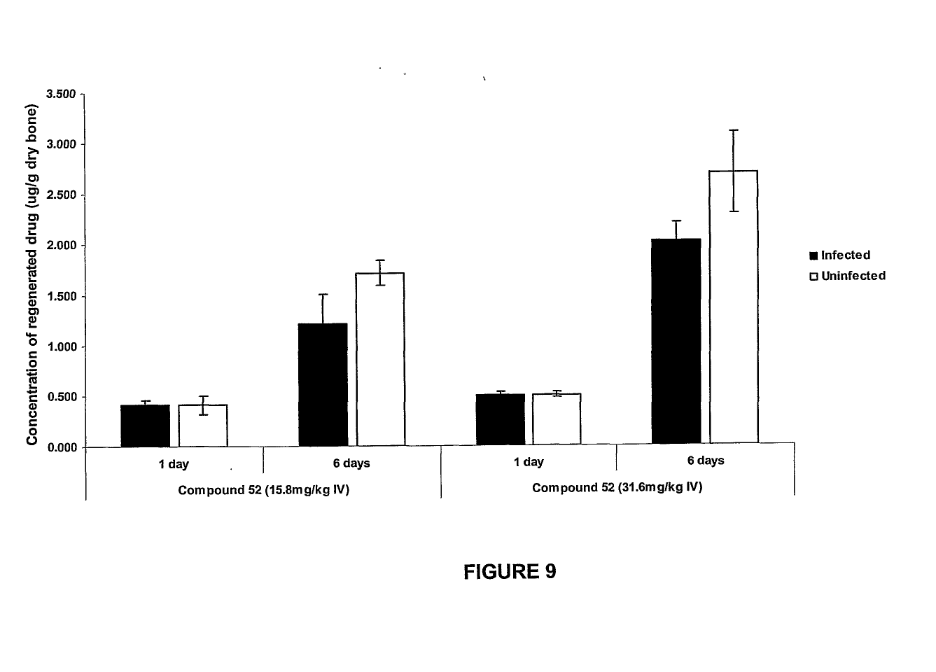

Standards (from 5 to 1000 ng), QCs (25 and 250 ng) and blanks were prepared (in duplicate) as follows: to blank tibia powder (50 mg, not dried) was added the 5% formic acid solution in methanol (160 μl) and the spiking solution of moxifloxacin 3 in water (10 μl). The mixtures were vortexed for 10 minutes and centrifuged for 10 minutes at 2500 g. The supernatant was transferred into another vial, spiked with the internal standard, vortexed and evaporated to dryness. In each case, the resulting dried residues (samples, standards, QCs and blanks) were reconstituted in 200 μl of water, vortexed for 15 min and centrifuged for 15 min at 10 000 g. The solution (20 μl) was injected into the LC/MS. The LC/MS method is as described in Example 6. The results are displayed in FIG. 9.

This experiment shows that the difference between infected and uninfected bones is negligible in terms of accessibility of the treating chemical entity. The reduced circulation and the increased inflammation in the infected limb do not significantly interfere with the distribution of the prodrug 52 since the level of regenerated moxifloxacin 3 is not significantly different between uninfected and infected tibiae, regardless of the dose of the bisphosphonated prodrug or the delay between its administration and collection of tibiae. This clearly highlights the efficiency of the bisphosphonates in delivering fluoroquinolone antibacterials to loci where their therapeutic activity is required.

### Example 9

### Tissue Distribution of Parent Drugs Regenerated from Bisphosphonated Moxifloxacin Prodrug 52 and Gatifloxacin Prodrug 54

The levels of moxifloxacin 3 regenerated from the bisphosphonated prodrug 52 and those of gatifloxacin 15 regenerated from the bisphosphonated prodrug 54 in different organs and bones were determined to assess the impact of the bisphosphonate functionalities on tissue distribution. These experiments were conducted using rats infected as described in Example 6, and treated with different dosing regimens of the investigated prodrugs.

### Determination of Moxifloxacin 3 and Gatifloxacin 15 by Microbiological Assay

Ground bone samples and tissue homogenates were suspended in PBS, vortexed and centrifuged at 13000 rpm for 2 min. The supernatant was collected and was applied in the subsequently described assay to determine the amount of regenerated drug 3 or 15.

The supernatants obtained from the bone samples and the tissue homogenates were assessed for the amount of prodrug by microbiological assay measurements as follows: Isolated colonies of the indicator strain (E. coli LBB925 toIC) were resuspended in 0.85% saline to OD₆₀₀=0.1 and streaked on Cation-adjusted Miller Hinton agar (CAMHA) plates. Known volumes of the supernatants were applied to discs and dried. The discs were then placed on the seeded CAMHA plates. The plates were incubated at 37° C. for 18 h after which the diameters of the zone of inhibition generated by the discs were measured. The amount of prodrug was deduced from standard curves of known amounts of free moxifloxacin 3 or free gatifloxacin 15 that were used as reference for each experiment.

This experiment was applied to rats treated with bisphosphonated moxifloxacin prodrug 52 at 32 mg/kg of body weight, on each of the 14^{th}, 15^{th}, 16^{th} and 17^{th} days after the surgery to induce infection. The rats were then sacrificed 43 days after the surgery, the desired tissues collected and the level of moxifloxacin 3 determined. The results are shown in Table 5.

TABLE 5
Concentration of moxifloxacin 3 (μg/g of tissue) in selected
bones and organs

| | Concentration of |
|---|---|
| Organ or bone | moxifloxacin 3 |
| Tibia | 0.98 ± 0 |
| Femur | 0.57 ± 0.09 |
| Mandible | 0.69 ± 0 |
| Kidney | 0.18 ± 0.02 |
| Liver | 0.07 ± 0.01 |
| Spleen | 0.82 ± 0.14 |
| Infected calf | Below limit of quantification |
| Uninfected calf | Below limit of quantification |

This experiment was also applied to rats treated with bisphosphonated gatifloxacin prodrug 54 at 34 mg/kg of body weight, using two different dosing regimens. Dosing regimen A involved treatment on each of the 14^{th}, 21^{st}, 28^{th} and 35^{th} days after the surgery to induce infection. Dosing regimen B involved treatment on each of the 14^{th}, 15^{th}, 16^{th} and 17^{th} days after the surgery to induce infection. In both cases, the rats were then sacrificed 43 days after the surgery, the desired tissues collected and the level of gatifloxacin 15 determined. The results are shown in Table 6.

TABLE 6
Concentration of gatifloxacin 15 (μg/g of tissue) in selected bones
and organs

Concentration of gatifloxacin 15

| Organ or bone | Dosing regimen A | Dosing regimen B |
|---|---|---|
| Tibia | 3.52 ± 0.70 | 2.67 ± 0.38 |
| Femur | 2.27 ± 0.04 | 1.13 ± 0.08 |
| Mandible | 2.86 ± 0.17 | 1.55 ± 0.01 |
| Kidney | 0.39 ± 0.04 | 0.26 ± 0.03 |
| Liver | 0.11 ± 0.03 | 0.09 ± 0.01 |
| Spleen | 1.21 ± 0.06 | 1.30 ± 0.17 |
| Infected calf | Below limit of | Below limit of |
| | quantification | quantification |
| Uninfected calf | Below limit of | Below limit of |
| | quantification | quantification |

Several trends are observed with the regenerated prodrugs 52 and 54. First, the presence of regenerated drug is detectable in all the selected bones, even weeks after treatment. Second, the distribution in bones is not homogeneous, with a clear preference for tibiae, followed by mandibles and femurs. This trend is observed for both prodrugs, which indicates that the anatomy and the physiology of each bone are key factors influencing the prodrug distribution. Third, lesser amounts of parent drugs are detected in liver, spleen and kidneys, but not in the tissues immediately surrounding the bones. This would be consistent with a phenomenon occurring at the time of injection, rather than as a result of regenerated material from bones diffusing into these organs. This can be explained by the formation of insoluble particles on injection, by complexation of the bisphosphonates by circulating metal ions (in particular calcium) which typically end up in kidneys, liver and spleen as described for other bisphosphonates (Adv. Drug Delivery Rev. (2000), 42:175-195).

An important observation from this in vivo experiment is that the tissue and bone concentrations of gatifloxacin 15 resulting from 54 which are consistently higher than those of moxifloxacin 3 resulting from 52, even though prodrugs 52 and 54 involve the same linker. While this observation could be a result of differential rates of elimination of the drug from bone, it also parallels the in vitro rates of regeneration observed previously (Table 3).

### Example 10

### Combination of Rifampicin and Bisphosphonated Gatifloxacin Prodrug 54 in the Treatment of Osteomyelitis Induced in Rats

The relatively slow release of gatifloxacin 15 from the bisphosphonated prodrug 54 prohibits the generation of a large concentration of 15 in bone. An advantage of this slow release mechanism is to allow prolonged exposure of the bacteria to the therapeutic agent, as was shown specifically in Examples 5 and 7. In this respect, the use of a combination of an antibacterial and a bisphosphonated prodrug of an antibacterial could prove attractive in supplying a high initial dose of the therapeutic agent followed by a prolonged exposure to the second one. This combination could prove particularly attractive in providing patients with a reduced frequency of treatment and the benefits of bone targeting, thus allowing fewer side effects associated with systemic exposure of the antibiotics.

In this respect, Rifampicin (U.S. Pat. No. 3,342,810) was chosen as a co-administered antibiotic, given its proven track record in the treatment of osteomyelitis, yet with reservations related to the high frequency of bacterial resistance associated with this antimicrobial (Antimicrob. Agents Chemother. (1992), 36:2693-7; J. Antimicrob. Chemother. (2004), 53:928-935).

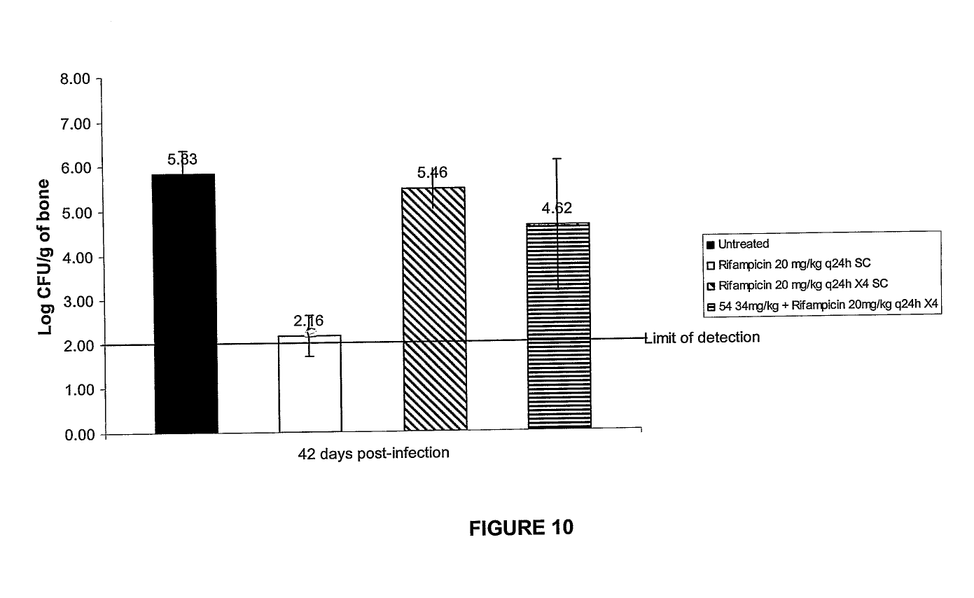

In this experiment, rats were infected as described in Example 7 and treated with either 20 mg/kg of body weight of Rifampicin subcutaneously, or with a combination of 34 mg/kg of prodrug 54 (corresponding to 20 mg/kg of gatifloxacin 15) intravenously and 20 mg/kg of Rifampicin subcutaneously on each of the 14^{th}, 15^{th}, 16^{th} and 17^{th} day after the surgery to induce infection. The standard controls involving no treatment and a treatment of 20 mg/kg of Rifampicin daily were also included. The rats were humanely sacrificed on the 43^{rd} day after the surgery and the bacterial titer in the infected tibiae determined. The results are described in FIG. 10.

The combination of Rifampicin and prodrug 54, but not Rifampicin alone under the same dosing regimen resulted in a statistically significant decrease (p=0.007) in the bacterial titer. This provides evidence of the potential of bisphosphonated fluoroquinolone prodrugs in prolonging the therapeutic effect of other antibacterial drugs in combinations which would prove valuable to the medical community in the treatment of osteomyelitis.

It is understood that the examples and embodiments described herein are for illustrative purposes only and that various modifications or changes in light thereof will be suggested to persons skilled in the art and are to be included within the spirit and purview of this application and scope of the appended claims.

All documents referred to herein, including patents, patent applications, publications, books, book chapters, journal article, manuals, guides and product literature, are expressly incorporated herein by reference.

What is claimed is:

1. A compound of Formula (I) or a pharmaceutically acceptable salt, metabolite, solvate or prodrug thereof: 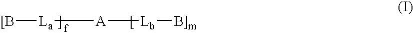
wherein:
f is 0 or 1;
m is 0 or 1;
A is a fluoroquinolone molecule or an antibacterial analog thereof;
B is a phosphonated group; and
Lₐ and L_{b} are cleavable linkers for coupling B to A.

2. The compound of claim 1, wherein the fluoroquinolone molecule or analog A is represented by Formulae A1a and A1b: 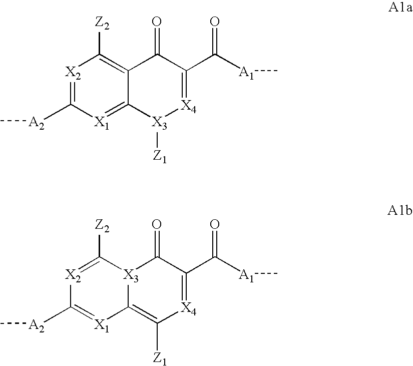
wherein:
said linker Lₐ is attached at A₂ when f=1, and linker L_{b} is attached at A₁ when m=1;
A₂ is an amino radical when f=1, and A₂ is hydrogen, halogen, alkyl, aryl, pyridinyl, —O-alkyl or an amino radical when f=0;
A₁ is O or S when m=1, and A₁ is OH when m=0;
Z₁ is alkyl, aryl or —O-alkyl;
Z₂ is hydrogen, halogen or an amino radical;
X₁ is N or —CY₁—, wherein Y₁ is hydrogen, halogen, alkyl, —O-alkyl, —S-alkyl, or X₁ forms a bridge with Z₁;
X₂ is N or —CY₂—, wherein Y₂ is hydrogen, halogen, alkyl, —O-alkyl, —S-alkyl, or
X₂ forms a bridge with A₂;
X₃ is N or CH; and
X₄ is N or CH.

3. The compound of claim 2, wherein Z, is cyclopropyl and X₂ is —CY₂—, wherein Y₂ is fluorine.

4. The compound of claim 1, wherein the fluoroquinolone molecule or analog A is represented by Formula A2: 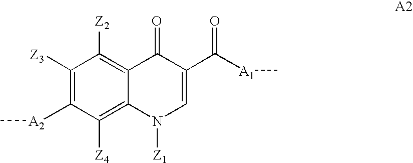
wherein:
said linker Lₐ is attached at A₂ when f=1, and linker L_{b} is attached at A₁ when m=1;
A₂ is an amino radical when f=1, and A₂ is hydrogen, halogen, alkyl, aryl, pyridinyl, —O-alkyl or an amino radical when f=0;
A₁ is O or S when m=1, and A₁ is OH when m=0;
Z₁ is alkyl, aryl or —O-alkyl;
Z₂ is hydrogen, halogen or an amino radical;
Z₃ is hydrogen or halogen; and
Z₄ is hydrogen, halogen, alkyl, —O-alkyl or —S-alkyl or forms a bridge with Z₁.

5. The compound of claim 4, wherein Z₁ is cyclopropyl and Z₃ is fluorine.

6. The compound of claim 1, wherein the fluoroquinolone molecule or analog A is represented by Formula A3: 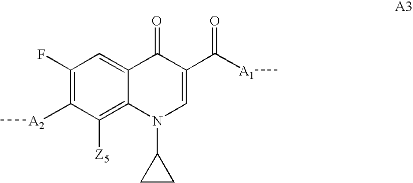
wherein:
said linker Lₐ is attached at A₂ when f=1, and linker L_{b} is attached at A₁ when m=1;
A₂ is an amino radical when f=1, and A₂ is hydrogen, halogen, alkyl, aryl, pyridinyl, —O-alkyl or an amino radical when f=0;
A₁ is O or S when m=1, and A₁ is OH when m=0;
Z₅ is hydrogen, halogen, alkyl or —O-alkyl.

7. The compound of claim 2, 4 or 6, wherein the amino radical is a N-linked substituted nitrogenous heterocyclic radical.

8. The compound of claim 7, wherein the N-linked substituted nitrogenous heterocyclic radical is a radical selected from the group consisting of pyrroles, pyrrolidines, piperidines, piperazines, morpholines, thiomorpholines, 1,4-diazepanes, dihydropyrrolidines, dihydropyridines and tetrahydropyridines.

9. The compound of claim 1, wherein B is a bisphosphonate.

10. The compound of claim 9, wherein each bisphosphonate is independently 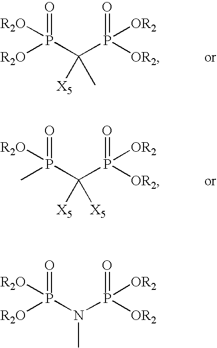
wherein:
each R₂ is independently H, lower alkyl, cycloalkyl, aryl or heteroaryl, with the proviso that at least two R₂ are H;
each X₅ is independently H, OH, NH₂, or a halo group.

11. The compound of claim 1, wherein L_{b} is a cleavable linker selected from the group consisting of: 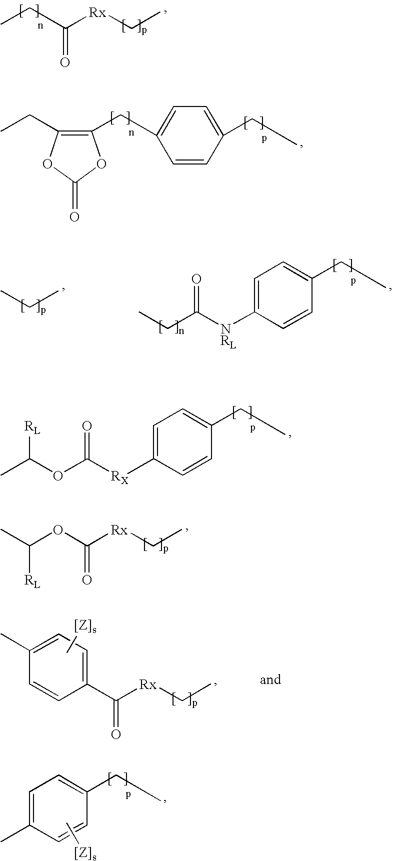
and Lₐ is a cleavable linker selected from the group consisting of: 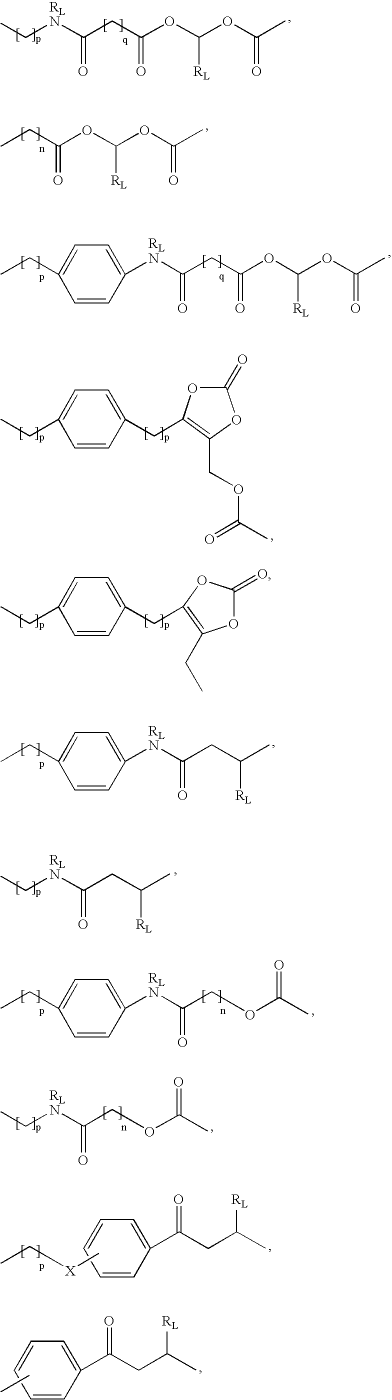 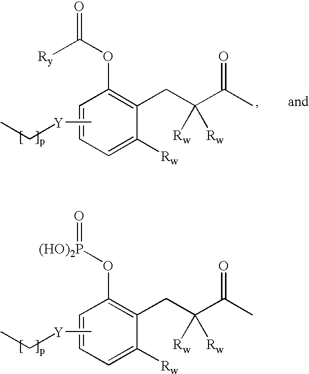
wherein:
n is an integer ≦10;
each p is independently 0 or an integer ≦10;
R_{L} is H, ethyl or methyl;
Rₓ is S, NR_{L} or O;
each Z is independently selected from the group consisting of hydrogen, halogen, alkyl, alkoxy, acyl, acyloxy, carboxy, carbamoyl, sulfuryl, sulfinyl, sulfenyl, sulfonyl, mercapto, amino, hydroxyl, cyano and nitro, and s is 1, 2, 3 or 4;
q is 2 or 3;
each R_{w} is independently H or methyl;
R_{y} is CₐH_{b} such that a is an integer from 0 to 20 and b is an integer between 1 and 2a+1;
X is CH₂, —CONR_{L}—, —CO—O—CH₂—, or —CO—O—; and
Y is O, S, S(O), SO₂, C(O), CO₂, CH₂ or absent.

12. The compound of claim 11, wherein each n is independently 1 or 2, each p is independently 0 or 1, R_{L} is H, and Rₓ is NH.

13. The compound of claim 1, wherein the fluoroquinolone molecule or analog A is ciprofloxacin or an antibacterial analog thereof.

14. The compound of claim 1, wherein the fluoroquinolone molecule or analog A is gatifloxacin or an antibacterial analog thereof.

15. The compound of claim 1, wherein the fluoroquinolone molecule or analog A is moxifloxacin or an antibacterial analog thereof.

16. A compound of Formula (II) or a pharmaceutically acceptable salt, metabolite, solvate or prodrug thereof: 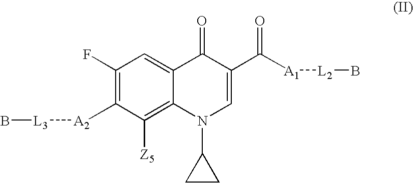
wherein:
the dashed lines represent bonds to optional groups B-L₃ and L₂-B, wherein at least one of B-L₃ and L₂-B is present;
Z₅ is hydrogen, halogen, alkyl or —O-alkyl;
A₁ is a O or S when L₂-B is attached at A₁, and A₁ is OH when L₂-B is not attached at A₁;
A₂ is an amino radical when B-L₃ is attached at A₂, and A₂ is hydrogen, halogen, alkyl, aryl, pyridinyl, —O-alkyl or an amino radical when B-L₃ is not attached at A₂;
each B is independently a phosphonated group of the formula: 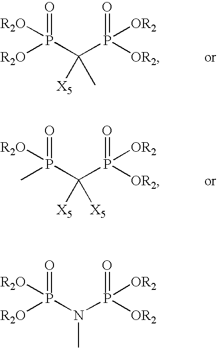
wherein:
each R₂ is independently H, lower alkyl, cycloalkyl, aryl or heteroaryl, with the proviso that at least two R₂ are H;
each X₅ is independently H, OH, NH₂, or a halo group; and
L₂ is a linker of the formula: 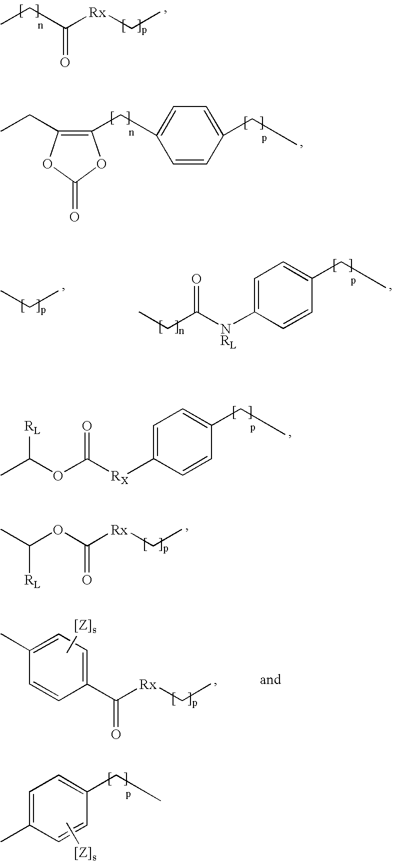
wherein:
n is an integer ≦10;
p is 0 or an integer ≦10;
R_{L} is H, ethyl or methyl;
Rₓ is S, NR_{L} or O; and
each Z is independently selected from the group consisting of hydrogen, halogen, alkyl, alkoxy, acyl, acyloxy, carboxy, carbamoyl, sulfuryl, sulfinyl, sulfenyl, sulfonyl, mercapto, amino, hydroxyl, cyano and nitro, and s is 1, 2, 3 or 4;
L₃ is a linker of the formula: 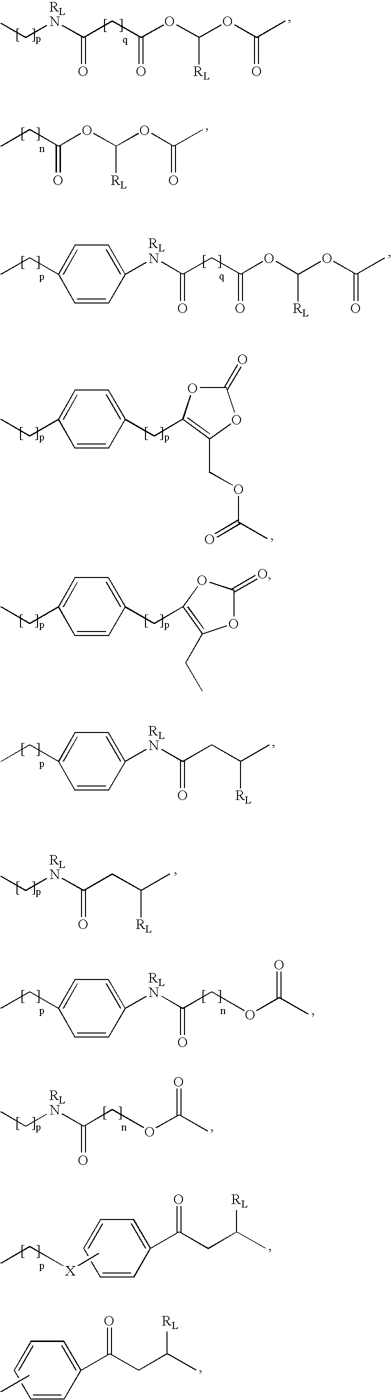 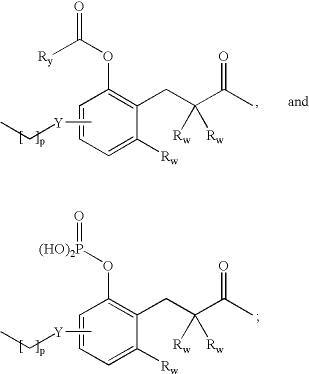
wherein:
n is an integer ≦10;
each p is independently 0 or an integer ≦10;
q is 2 or 3;
R_{L} is H, ethyl or methyl;
each R_{w} is independently H or methyl;
R_{y} is CₐH_{b} such that a is an integer from 0 to 20 and b is an integer between 1 and 2a+1;
X is CH₂, —CONR_{L}—, —CO—O—CH₂—, or —CO—O—; and
Y is O, S, S(O), SO₂, C(O), CO₂, CH₂ or absent.

17. The compound of claim 16, wherein for each linker n is 1 or 2, each p is independently 0 or 1, R_{L} is H, and Rₓ is NH.

18. The compound of claim 16, wherein the amino radical is a N-linked substituted nitrogenous heterocyclic radical.

19. The compound of claim 18, wherein the N-linked substituted nitrogenous heterocyclic radical is a radical selected from the group consisting of pyrroles, pyrrolidines, piperidines, piperazines, morpholines, thiomorpholines, 1,4-diazepanes, dihydropyrrolidines, dihydropyridines and tetrahydropyridines.

20. A compound represented by a formula selected from the group consisting of: 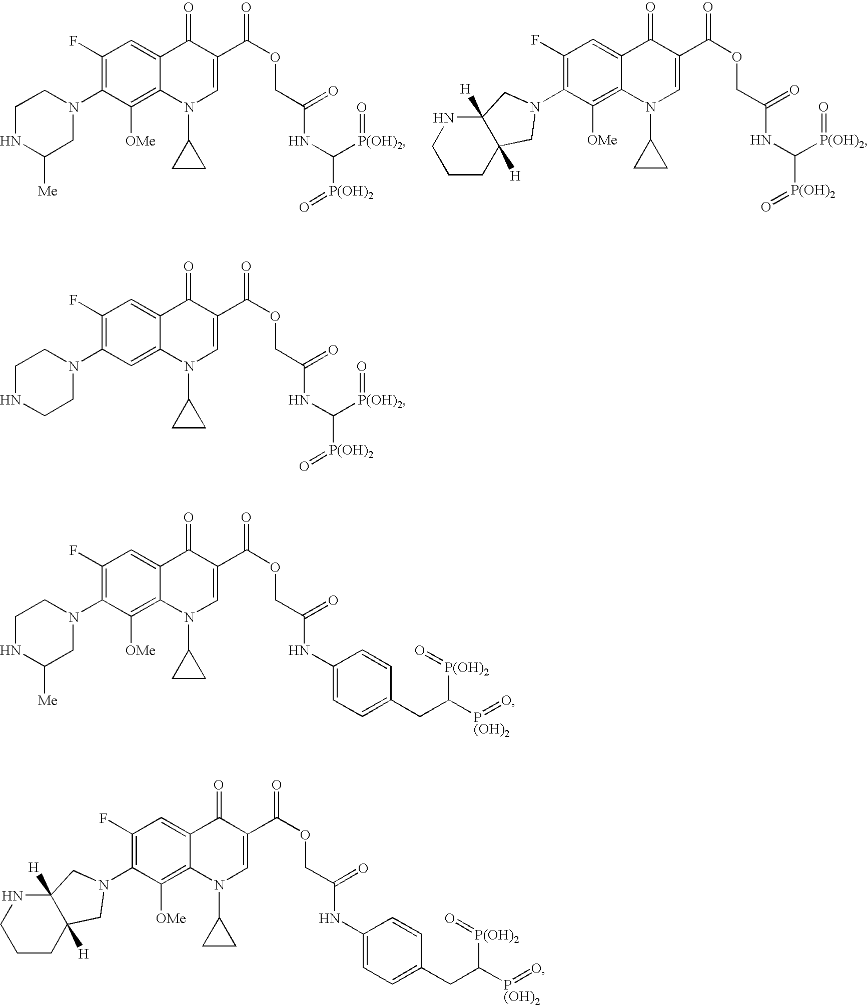 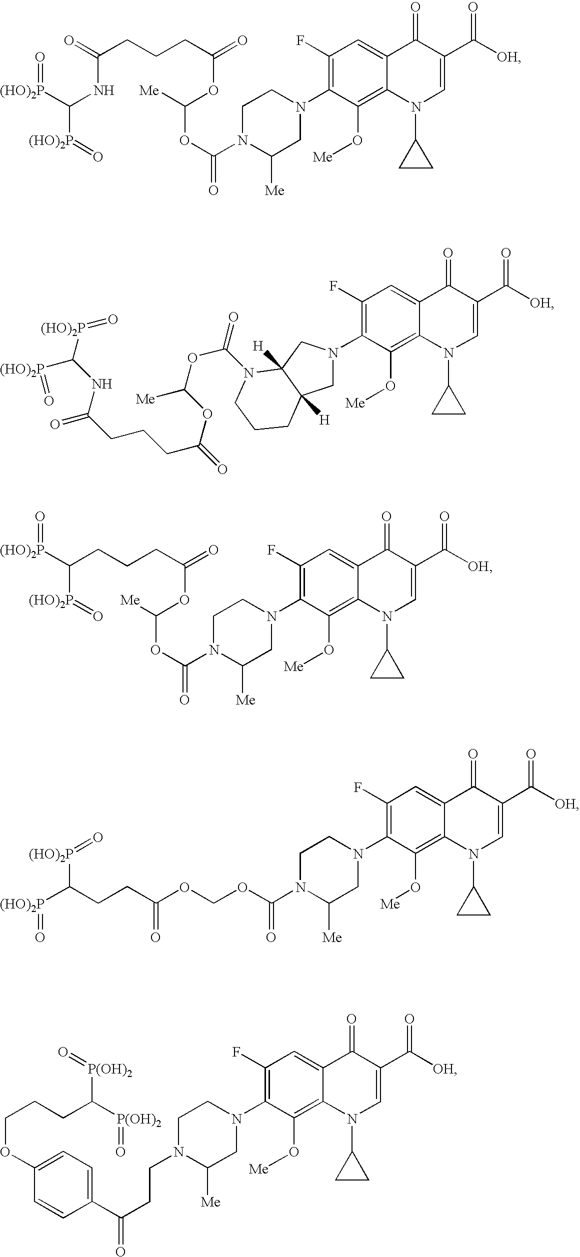 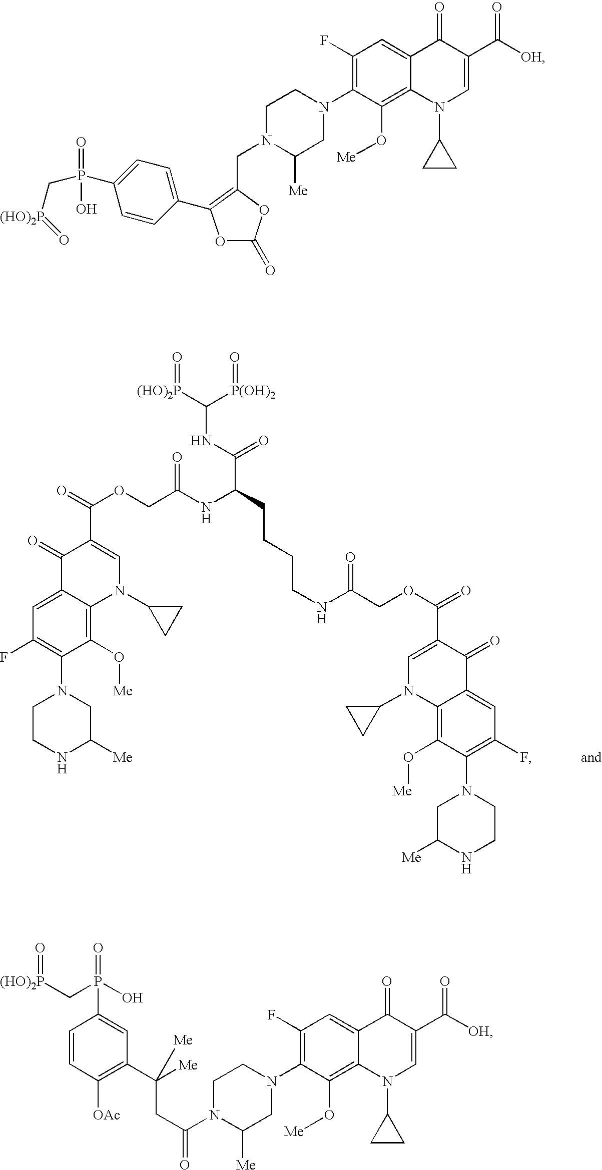
or pharmaceutically acceptable salt, metabolite, solvate or prodrug thereof.

21. A pharmaceutical composition comprising a compound selected from claims 1, 16 and 20, and a pharmaceutically acceptable carrier or excipient.

22. A method for treating a bacterial infection in a subject, said method comprising administering to a subject in need of such treating a pharmaceutical composition comprising a pharmaceutically effective amount of a first antibiotic compound selected from claims 1, 16 and 20.

23. The method of claim 22, wherein a second antibiotic compound is included in said pharmaceutical composition.

24. The method of claim 23, wherein said second antibiotic compound is a rifamycin analog.

25. The method of claim 23, wherein said second antibiotic compound is tetracycline, tygecycline, or a tetracycline, glycycycline or minocycline analog.

26. The method of claim 22, wherein said subject is a human.

27. A method for preventing a bacterial infection in a subject, said method comprising administering to a subject in need of prevention a pharmaceutical composition comprising a pharmaceutically effective amount of an antibiotic compound selected from claims 1, 16 and 20.

28. The method of claim 27, wherein said pharmaceutical composition is administered to said subject prior to, during, or after an invasive medical treatment.

29. A method for accumulating a fluoroquinolone molecule or analog thereof in a bone of a subject, comprising administering to a subject a compound of any one of claims 1, 16 and 20.
